SEQUENCE LISTING (9 sequences)

SEQ ID NO: 1 - DNA, 274 bp, Lactobacillus sp.
tacgtaggtggcaagcgttgtccggatttattgggcgtaaagcgagcgcaggcggattgataagtctgatgtgaaagccttcggctcaaccgaagaactgcatcagaaactgtcaatcttgagtgcagaagaggagagtggaactccatgtgtagcggtggaatgcgtagatatatggaagaacaccagtggcgaaggcggctctctggtctgtaactgacgctgaggctcgaaagcatgggtagcgaacaggattagataccctggtagtcca

SEQ ID NO: 2 - DNA, 254 bp, Micrococcus sp.
tacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacagga

SEQ ID NO: 3 - DNA, 1442 bp, Lactobacillus sp.
gcctaatacatgcaagtcgagcgagcttgcctatagaaattcttcggaatggacatagatacaagctagcggcggatgggtgagtaacgcgtgggtaacctgcccttaagtctgggataccatttggaaacagatgctaataccggataaaagctactttcgcatgaaagaagtttaaaaggcggcgtaagctgtcgctaaaggatggacctgcgatgcattagctagttggtaaggtaacggcttaccaaggcgatgatgcatagccgagttgagagactgatcggccacattgggactgagacacggcccaaactcctacgggaggcagcagtagggaatcttccacaatggacgaaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtgccagcagccgcggtaatacgtaggtggcaagcgttgtccggatttattgggcgtaaagcgagcgcaggcggattgataagtctgatgtgaaagccttcggctcaaccgaagaactgcatcagaaactgtcaatcttgagtgcagaagaggagagtggaactccatgtgtagcggtggaatgcgtagatatatggaagaacaccagtggcgaaggcggctctctggtctgtaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccacctaagagattaggttttcccttcggggacaaagagacaggtggtgcatggctgtcgtcagctcgtgtcgtgagatgttgggttaagtcccgcaacgagcgcaacccttgttaatagttgccagcattaagttgggcactctattgagactgccggtgacaaaccggaggaaggtggggatgacgtcaagtcatcatgccccttatgacctgggctacacacgtgctacaatgggcagtacaacgagaagcgaacctgtgaaggcaagcgaatctcttaaagctgttctcagttcggactgtaggctgcaactcgcctacacgaagctggaatcgctagtaatcgcggatcagcacgccgcggtgaatacgttcccgggccttgtacacaccgcccgtcacaccatgagagtttgtaacacccaaagtcggtgaggtaactttgagccagccgccaa

SEQ ID NO: 4 - DNA, 1376 bp, Micrococcus sp.
agtcgaacgatgaagcccagcttgctgggtggattagtggcgaacgggtgagtaacacgtgagtaacctgcccttaactctgggataagcctgggaaactgggtctaataccggataggagcgtccaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtagagatacggtttcccctttggggcgggttcacaggtggtgcatggttgtcgtcagctcgtgtcgtgagatgttgggttaagtcccgcaacgagcgcaaccctcgttccatgttgccagcacgtagtggtggggactcatgggagactgccggggtcaactcggaggaaggtgaggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagtcggtaacacccgaagccggggcctaacccttgtgg

SEQ ID NO: 5 - DNA, 1445 bp, Lactobacillus sp.
gcctaatacatgcaagtcgagcgagcttgcctatagaaattcttcggaatggacatagatacaagctagcggcggatgggtgagtaacgcgtgggtaacctgcccttaagtctgggataccatttggaaacagatgctaataccggataaaagctactttcgcatgaaagaagtttaaaaggcggcgtaagctgtcgctaaaggatggacctgcgatgcattagctagttggtaaggtaacggcttaccaaggcgatgatgcatagccgagttgagagactgatcggccacattgggactgagacacggcccaaactcctacgggaggcagcagtagggaatcttccacaatggacgaaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtgccagcagccgcggtaatacgtaggtggcaagcgttgtccggatttattgggcgtaaagcgagcgcaggcggattgataagtctgatgtgaaagccttcggctcaaccgaagaactgcatcagaaactgtcaatcttgagtgcagaagaggagagtggaactccatgtgtagcggtggaatgcgtagatatatggaagaacaccagtggcgaaggcggctctctggtctgtaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccacctaagagattaggttttcccttcggggacaaagagacaggtggtgcatggctgtcgtcagctcgtgtcgtgagatgttgggttaagtcccgcaacgagcgcaacccttgttaatagttgccagcattaagttgggcactctattgagactgccggtgacaaaccggaggaaggtggggatgacgtcaagtcatcatgccccttatgacctgggctacacacgtgctacaatgggcagtacaacgagaagcgaacctgtgaaggcaagcgaatctcttaaagctgttctcagttcggactgtaggctgcaactcgcctacacgaagctggaatcgctagtaatcgcggatcagcacgccgcggtgaatacgttcccgggccttgtacacaccgcccgtcacaccatgagagtttgtaacacccaaagtcggtgaggtaactttggagccagccgcctaag

SEQ ID NO: 6 - DNA, 253 bp, Lactobacillus sp.
tacgtaggtggcaagcgttgtccggatttattgggcgtaaagcgagtgcaggcggctcgataagtctgatgtgaaagccttcggctcaaccggagaattgcatcagaaactgtcgagcttgagtacagaagaggagagtggaactccatgtgtagcggtgaaatgcgtagatatatggaagaacaccggtggcgaaggcggctctctggtctgttactgacgctgaggctcgaaagcatgggtagcgaacagg

SEQ ID NO: 7 - DNA, 253 bp, Micrococcus sp.
tacgtagggtgcaagcgttatccggaattattgggcgtaaagagctcgtaggcggtttgtcgcgtctgtcgtgaaagtccggggctcaactccggatctgcggtgggtacgggcagactagagtgatgtaggggagactggaattcctggtgtagcggtggaatgcgcagatatcaggaggaacaccgatggcgaaggcaggtctctgggcattaactgacgctgaggagcgaaagcatggggagcgaacagg

SEQ ID NO: 8 - DNA, 18 bp, Artificial Sequence
  Synthetic construct.
gctgcctcccgtaggagt

SEQ ID NO: 9 - DNA, 18 bp, Artificial Sequence
  Synthetic construct.
actcctacgggaggcagc

(19) United States
(12) Patent Application Publication
(10) Pub. No.: US 2022/0047653 A1
(43) Pub. Date: Feb. 17, 2022

(54) METHODS AND COMPOSITIONS FOR TREATING AND PREVENTING INFLAMMATORY DISEASES

(71) Applicant: The Regents of the University of California, Oakland, CA (US)

(72) Inventors: Susan V. LYNCH, Piedmont, CA (US); Elze RACKAITYTE, San Francisco, CA (US)

(21) Appl. No.: 17/266,952

(22) PCT Filed: Aug. 6, 2019
(86) PCT No.: PCT/US19/45354

§ 371 Date: Feb. 8, 2021

Related U.S. Application Data
(60) Provisional application No. 62/717,464, filed on Aug. 10, 2018.

(51) Int. Cl. A61K 35/747; A61K 35/741; A61P 37/06; C12N 1/20

(52) U.S. Cl. CPC A61K 35/747; A61K 35/741; C12N 2502/30; C12N 1/20; C12N 2502/1157; A61P 37/06

(57) ABSTRACT

Provided herein are, inter alia, methods and compositions for treating, preventing, or reducing the risk of dysbiosis, inflammation, inflammatory diseases, childhood obesity, and premature birth. Included are methods and compositions for increasing or promoting healthy or normal immune system maturation. In aspects, provided herein are methods and compositions for detecting and isolating bacterial strains. Isolated bacterial strains and culture methods are also provided.

### CROSS-REFERENCE

This application claims the benefit of priority to U.S. Provisional Patent Application Ser. No. 62/717,464, filed Aug. 10, 2018, which is hereby incorporated by reference in its entirety for all purposes.

### ACKNOWLEDGEMENT OF GOVERNMENT SUPPORT

This invention was made with government support under grant no. F31 AI136336 awarded by the National Institutes of Health. The government has certain rights in the invention.

### SEQUENCE LISTING

The material in the accompanying Sequence Listing is hereby incorporated by reference in its entirety. The accompanying Sequence Listing file, named “048536-624001WO_SEQUENCE_LISTING_ST25.txt”, was created on Jul. 24, 2019 and is 8.22 Kb in size.

### BACKGROUND OF THE INVENTION

Mucosal immunity is evident in the human fetal intestine by the end of the first trimester [1,2]. The developing intestine is populated by migrating dendritic cells capable of responding to microbial stimuli and initiating robust T cell responses [3]. By week 13 of gestation, memory T cells are abundant in the human fetal intestine [2,4-8], possess pro-inflammatory potential [6], and influence epithelial maturation [7]. These cells also exhibit clonal expansion to foreign antigens [8].

Recent evidence for bacterial presence in utero comes from DNA-based, culture-independent studies of the placenta [9-11] and amniotic fluid [10], though other studies have refuted the presence of bacteria at these sites [12,13]. Neonatal meconium, the first stool of infants, is comprised of amniotic fluid ingested during gestation and contains a simple microbiota [14,15]. Heightened risk of chronic inflammatory disease in childhood, such as asthma, is associated with a distinct and perturbed neonatal meconium and early-life microbiota [15], the metabolic products of which induce inflammation ex vivo [16]. Whether initial intestinal encounters with viable microbes occur in utero has not been investigated.

### BRIEF SUMMARY OF THE INVENTION

Provided herein are, inter alia, methods and compositions for treating, preventing, or reducing the risk of dysbiosis, inflammation, inflammatory diseases, childhood obesity, and premature birth. Included are methods and compositions for increasing or promoting healthy or normal immune system maturation. In aspects, provided herein are methods and compositions for detecting and isolating bacterial strains. Isolated bacterial strains and culture methods are also provided.

In an aspect, provided herein is a method of treating, preventing, or reducing the risk of an inflammatory disease in a subject in need thereof. In embodiments, the method comprises administering to the subject an effective amount of a fetal Micrococcus sp. bacterium and/or a fetal Lactobacillus sp. bacterium.

In an aspect, provided herein is a method of treating, preventing, or reducing the risk of dysbiosis in a subject in need thereof. In embodiments, the method comprises administering to the subject an effective amount of a fetal Micrococcus sp. bacterium and/or a fetal Lactobacillus sp. bacterium.

In an aspect, provided herein is a method of treating, preventing, or reducing the risk of inflammation in an unborn subject. In embodiments, the method comprises administering to the pregnant mother of the subject an effective amount of a fetal Micrococcus sp. bacterium and/or a fetal Lactobacillus sp. bacterium.

In an aspect, provided herein is a method of promoting or increasing immune system maturation in an unborn subject. In embodiments, the method comprises administering to the pregnant mother of the subject an effective amount of a fetal Micrococcus sp. bacterium and/or a fetal Lactobacillus sp. bacterium.

In an aspect, provided herein is a method of treating, preventing, or reducing the risk of dysbiosis in an unborn subject. In embodiments, the method comprises administering to the pregnant mother of the subject an effective amount of a fetal Micrococcus sp. bacterium and/or a fetal Lactobacillus sp. bacterium.

In an aspect, provided herein is a method of reducing the risk that an unborn subject will develop an inflammatory disease after birth. In embodiments, the method comprises administering to the pregnant mother of the subject an effective amount of a fetal Micrococcus sp. bacterium and/or a fetal Lactobacillus sp. bacterium.

In an aspect, provided herein is a method of treating, preventing, or reducing the risk of childhood obesity in an unborn subject. In embodiments, the method comprises administering to the pregnant mother of the subject an effective amount of a fetal Micrococcus sp. bacterium and/or a fetal Lactobacillus sp. bacterium.

In an aspect, provided herein is a method of treating, preventing, or reducing the risk of dysbiosis in a neonatal subject. In embodiments, the method comprises administering to the subject an effective amount of a fetal Micrococcus sp. bacterium and/or a fetal Lactobacillus sp. bacterium.

In an aspect, provided herein is a method of reducing the risk that a neonatal subject will develop an inflammatory disease after birth. In embodiments, the method comprises administering to the subject an effective amount of a fetal Micrococcus sp. bacterium and/or a fetal Lactobacillus sp. bacterium.

In an aspect, provided herein is a method of treating, preventing, or reducing the risk of childhood obesity in a neonatal subject. In embodiments, the method comprises administering to the subject an effective amount of a fetal Micrococcus sp. bacterium and/or a fetal Lactobacillus sp. bacterium.

In an aspect, provided herein is a method of reducing the risk that a pregnant subject will give birth less than 37 completed weeks of gestation. In embodiments, the method comprises administering to the subject an effective amount of a fetal Micrococcus sp. bacterium and/or a fetal Lactobacillus sp. bacterium.

In an aspect, provided herein is a method of promoting tolerogenic immunity in a subject in need thereof. In embodiments, the method comprises administering to the subject an effective amount of a fetal Micrococcus sp. bacterium and/or a fetal Lactobacillus sp. bacterium.

In an aspect, provided herein is a method of detecting a polynucleotide in (i) a fetal intestine, meconium, amniotic fluid, or a placenta, (ii) infant stool, (iii) a meternal sample, or (iv) a combination thereof. In embodiments, the method comprises detecting whether a polynucleotide having a sequence that is at least 95, 96, 97, 98, 99, or 100% identical to SEQ ID NO: 1, SEQ ID NO: 2, SEQ ID NO: 3, SEQ ID NO: 4, SEQ ID NO: 5, SEQ ID NO: 6, or SEQ ID NO: 7 is present in a bacterium or biological sample obtained from the fetal intestine, meconium, amniotic fluid, or placenta.

In an aspect, provided herein is a method of detecting a polynucleotide in a bacterium. In embodiments, the method comprises detecting whether a polynucleotide having a sequence that is at least 95%, 96%, 97%, 98%, 99%, or 100% identical to SEQ ID NO: 1, SEQ ID NO: 2, SEQ ID NO: 3, SEQ ID NO: 4, SEQ ID NO: 5, SEQ ID NO: 6, or SEQ ID NO: 7 is present in a bacterium obtained from fetal intestine, meconium, amniotic fluid, or a placenta.

In an aspect, provided herein is a method of culturing an isolated bacterium. In embodiments, the method comprises obtaining a bacterium comprising a 16S rRNA gene V4 region comprising a sequence that is at least 95%, 96%, 97%, 98%, 99%, or 100% identical to SEQ ID NO: 1 or SEQ ID NO: 2, wherein the bacterium has been isolated from amniotic fluid, meconium, or a placenta, and culturing the bacterium.

In an aspect, provided herein is an isolated fetal Micrococcus sp. bacterium and/or an isolated fetal Lactobacillus sp. bacterium.

In an aspect, provided herein is a composition comprising an isolated fetal Micrococcus sp. bacterium and/or an isolated fetal Lactobacillus sp. bacterium disclosed and a carrier that is suitable for oral or vaginal administration.

In an aspect, provided herein is an artificial culture comprising an isolated fetal Micrococcus sp. bacterium and/or an isolated fetal Lactobacillus sp. bacterium disclosed herein and a medium.

In an aspect, provided herein is a method of culturing a fetal Micrococcus sp. bacterium and/or a fetal Lactobacillus sp. bacterium. In embodiments, the method comprises incubating the bacterium in or on a medium comprising a eukaryotic cell, and/or a placental hormone.

In an aspect, provided herein is a method of isolating a fetal Micrococcus sp. bacterium and/or a fetal Lactobacillus sp. bacterium. In embodiments, the method comprises (i) incubating a culture medium comprising (a) a biological sample suspected of containing the bacterium and (b) a eukaryotic cell, and/or a placental hormone, thereby producing a pre-isolate culture; (ii) selecting the fetal Micrococcus sp. bacterium and/or a fetal Lactobacillus sp. bacterium. In embodiments, selecting the fetal Micrococcus sp. bacterium and/or a fetal Lactobacillus sp. bacterium comprises streaking a portion of the pre-isolate culture onto a selection plate (e.g., an plate comprising medium that comprises a gel-like or solid state such as a medium comprising agarose), and selecting a single colony of the fetal Micrococcus sp. bacterium and/or a fetal Lactobacillus sp. bacterium from the plate.

In an aspect, provided herein is a method of culturing a fetal Micrococcus sp. bacterium and/or a fetal Lactobacillus sp. bacterium. In embodiments, the method comprises incubating the bacterium in or on a medium comprising an epithelial cell, and/or a placental hormone.

In an aspect, provided herein is a method of isolating a fetal Micrococcus sp. bacterium and/or a fetal Lactobacillus sp. bacterium. In embodiments, the method comprises (i) incubating a culture medium comprising (a) a biological sample suspected of containing the bacterium and (b) a epithelial cell, and/or a placental hormone, thereby producing a pre-isolate culture; (ii) selecting the fetal Micrococcus sp. bacterium and/or a fetal Lactobacillus sp. bacterium. In embodiments, selecting the fetal Micrococcus sp. bacterium and/or a fetal Lactobacillus sp. bacterium comprises streaking a portion of the pre-isolate culture onto a selection plate (e.g., an plate comprising medium that comprises a gel-like or solid state such as a medium comprising agarose), and selecting a single colony of the fetal Micrococcus sp. bacterium and/or a fetal Lactobacillus sp. bacterium from the plate.

In an aspect, provided herein is a method of culturing a fetal Micrococcus sp. bacterium and/or a fetal Lactobacillus sp. bacterium. In embodiments, the method comprises incubating the bacterium in or on a medium comprising a monocyte or a macrophage, and/or a placental hormone.

In an aspect, provided herein is a method of isolating a fetal Micrococcus sp. bacterium and/or a fetal Lactobacillus sp. bacterium. In embodiments, the method comprises (i) incubating a culture medium comprising (a) a biological sample suspected of containing the bacterium and (b) a monocyte or a macrophage, and/or a placental hormone, thereby producing a pre-isolate culture; (ii) selecting the fetal Micrococcus sp. bacterium and/or a fetal Lactobacillus sp. bacterium. In embodiments, selecting the fetal Micrococcus sp. bacterium and/or a fetal Lactobacillus sp. bacterium comprises streaking a portion of the pre-isolate culture onto a selection plate (e.g., an plate comprising medium that comprises a gel-like or solid state such as a medium comprising agarose), and selecting a single colony of the fetal Micrococcus sp. bacterium and/or a fetal Lactobacillus sp. bacterium from the plate.

### BRIEF DESCRIPTION OF THE DRAWINGS

FIGS. 1A-G present data showing that Lactobacillus or Micrococcaceae are relatively enriched in fetal meconium. FIG. 1A is a box plot depicting that total 16S copy number per ng 100 gDNA in meconium from mid-section of the fetal small intestine, fetal kidney, and procedural, air, or blank swab was quantified by qPCR of DNA extracts using a standard curve; linear mixed effects model to test for significance. FIG. 1B is a line graph that depicts bacterial relative abundance ranks in fetal meconium, post-natal meconium, and procedural swab. Geometric and log series model fitting of absolute abundance ranks was determined by Bayesian Information Criterion (BIC). FIG. 1C is a bar graph that depicts relative abundance of select genera among samples dominated by OTU12, OTU10, or other OTUs. Symbols indicate samples with paired immunological datasets. FIG. 1D is a scatter plot that depicts principal coordinates analysis (PCoA) of Bray Curtis distances on mid-section samples delineated by dominant taxon, Micrococcaceae meconium (MM), Lactobacillus meconium (LM), other meconium (OM), or procedural swab. PERMANOVA test for significant variation in bacterial composition. FIG. 1E is a box plot that depicts normalized read counts for Lactobacillus OTU12 and Micrococcocaceae OTU10 in LM, MM, OM, swab, and fetal kidney control samples. Linear mixed effects modeling correcting for paired samples indicated by grey line. FIG. 1F is a scatter plot that depicts significantly enriched taxa (DESEQ2, Log 2-fold change >2, false discovery rate <0.05,) in meconium as compared to procedural swabs and kidney controls. Dots represent differential taxa and are scaled by percent relative abundance in meconium; top two taxa by abundance are labeled. FIG. 1G depicts representative scanning electron micrographs of fetal intestinal lumen, arrowheads indicate pockets of bacterial-like morphology in meconium at 3 000 (left) and mucin embedded structures at 50 000 (right) times magnification, scale bars below indicate size (20 μm (left), and 1 μm (right)). Each dot represents one biological replicate, unless otherwise noted.

FIGS. 2A-H present data showing that divergent immune cell phenotypes are associated with Lactobacillus or Micrococcaceae relative enrichment in fetal meconium. FIG. 2A is a scatter plot that depicts principal components (PC) analysis of euclidean distances of top 10000 variable genes (by coefficient of variation) in LM associated epithelium (LM-E) and MM associated epithelium (MM-E) as determined by RNA sequencing. PERMANOVA test for significance. FIG. 2B is a Venn diagram depicting top differentially expressed genes between LM-E (log 2 fold change >1, FDR <0.05) and MM-E (log 2 fold change<1, FDR <0.05). FIG. 2C is a heatmap depicting top differentially expressed genes between LM-E (log 2 fold change >1, FDR <0.05) and MM-E (log 2 fold change<1, FDR <0.05) with immune pathway transcripts labeled. FIG. 2D is a volcano plot depicting top differentially expressed genes between LM-E (log 2 fold change >1, FDR <0.05) and MM-E (log 2 fold change<1, FDR <0.05). FIG. 2E is a bar graph depicting normalized enrichment scores of gene set enrichment analysis of transcripts associated with epithelial cell states FIG. 2F is a box plot depicting proportion of PLZF+CD161+ T cells among live, TCRβ+, Vα7.2−, CD4+ cells in intestinal lamina propria (LP), mesenteric lymph node (MLN), and spleen (SPL). FIG. 2G shows representative flow plots of mesenteric lymph node (top panel, gating control) or intestinal lamina propria (bottom panel) associated with MM and LM. FIG. 2H is a box plot that depicts the proportion of PLZF+CD161+ T cells among live, CD4+ TCRβ+Vα7.2− cells in lamina propria paired with LM or MM (LM-LP or MM-LP, respectively). Numbers indicate means and standard error of the mean (SEM). Kruskal-Wallis ANOVA, with Dunnet's correction for multiple comparisons was used for FIG. 2F; Wilcoxon rank sum test was used for FIG. 2H. Each dot represents one transcript in FIG. 2D, one cell in FIG. 2G, and one biological replicate in FIG. 2A, FIG. 2F, FIG. 2H.

FIGS. 3A-G present data showing that Lactobacillus and Micrococcus isolates from fetal meconium exhibit adaptation to the fetal environment. FIG. 3A is phylogenetic tree of 16S V4 rRNA gene sequences from Lactobacillus-enriched meconium (LM), Micrococcaceae-enriched meconium (MM), or procedural swab, enriched OTUs (circles), and primary isolates (squares) from fetal meconium (Micro36, Lacto166, Lacto167) and reference strains for Micrococcus luteus (MicroRef1, MicroRef2) and Lactobacillus iners (LactoRef). Branch lengths scaled to the mean number of nucleotide substitutions per site and bootstrap values are represented for each node. FIG. 3B is a line graph showing the effects of 10⁻⁵M progesterone (P4) and 10⁻⁶M β-Estradiol (E2) on the growth of Micro36 compared to ethanol vehicle control in indicated carbon-rich media (brain heat infusion (BHI)). FIG. 3C is a line graph showing the effects of 10⁻⁵M progesterone (P4) and 10⁻⁶M β-Estradiol (E2) on the growth of Lacto166 (left panel) or Lacto167 (right panel) compared to ethanol vehicle control in indicated carbon-rich media (chopped-meat carbohydrate (CMC)). FIG. 3D is a line graph showing the effects of 10⁻⁵M progesterone (P4) and 10⁻⁶M β-Estradiol (E2) on the growth of Lacto166 (left panel) or Lacto167 (right panel) compared to ethanol vehicle control in indicated carbon-rich media (De Man, Rogosa, Sharpe (MRS)). FIG. 3E is a line graph showing the effects of 10⁻⁵M progesterone (P4) and 10⁻⁶M β-Estradiol (E2) on the growth of Micro36 compared to ethanol vehicle control in carbon limiting media mineral salt media (MSM) at 37° C. For FIGS. 3B-E, representative growth curves of three independent experiments measured by optical density at 600 nm (OD600), error bars denote standard error of the mean (SEM) between three technical experiments. For carbon-rich media conditions, integral of logistic regression model fitting was used to calculate area under the curve (auc) and difference between test conditions and vehicle control is reported as Δauc. FIG. 3F shows a line graph depicting intracellular survival of Micro36, MicroRef1, MicroRef2 in primary human antigen presenting cells isolated from the fetal intestine. Representative data of three independent biological experiments, error bars indicate SEM of three technical replicates. Generalized linear model of log(CFU+1) against respective reference strain (MicroRef1) for each time point was used to calculate significance. FIG. 3G shows a line graph depicting intracellular survival of Lacto166, Lacto167, or LactoRef in primary human antigen presenting cells isolated from the fetal intestine. Representative data of three independent biological experiments, error bars indicate SEM of three technical replicates. Generalized linear model of log(CFU+1) against respective reference strain (LactoRef) for each time point was used to calculate significance.

FIGS. 4A-B present data showing the resolved taxonomy of fetal Lactobacillus and Micrococcus isolates. FIG. 4A is a diagram showing whole genome average nucleotide identity (ANI) of all available genomes in Micrococcus genus and Micro36 isolate. When available strain origin is represented, hierarchical clustering was performed on average nucleotide identity, asterisk (*) indicates a reference or a representative genome for the taxon. FIG. 4B is a diagram showing whole genome average nucleotide identity (ANI) of all available genomes in all available genomes of Lactobacillus jensenii (L.j.), select Lactobacillus reference genomes, and Lacto166 and Lacto167 isolates. When available strain origin is represented, hierarchical clustering was performed on average nucleotide identity, asterisk (*) indicates a reference or a representative genome for the taxon.

FIGS. 5A-J present data showing that fetal Lactobacillus and Micrococcus isolates drive divergent immune phenotypes in vitro. FIG. 5A is a box plot showing normalized read counts of NOS2 (left) and ADRA2A (right) in epithelial cells treated with Lacto166, Micro36, or media control for four hours obtained by RNAseq. Significance was calculated using DESEQ2, correcting for false-discovery rate, n=2 for each condition. FIG. 5B presents a box plot showing proportions of CD83+ CD86+ cells (left panel) and representative flow plots of CD83 and CD86 expression (right) among live, lin-, CD45+, HLA-DR+ following four hours of exposure to Lactobacillus (Lacto166, Lacto167, LactoRef) or Micrococcus (Micro36, MicroRef) strains. FIG. 5C is a box plot showing concentrations of IL-10 in supernatants of fetal splenic antigen presenting cells following four hours of exposure to Lactobacillus (Lacto166, Lacto167, LactoRef) or Micrococcus (Micro36, MicroRef) strains. FIG. 5D is a box plot showing concentrations of GM-CSF in supernatants of fetal splenic antigen presenting cells following four hours of exposure to Lactobacillus (Lacto166, Lacto167, LactoRef) or Micrococcus (Micro36, MicroRef) strains. FIG. 5E is a box plot showing concentrations of TNFα in supernatants of fetal splenic antigen presenting cells following four hours of exposure to Lactobacillus (Lacto166, Lacto167, LactoRef) or Micrococcus (Micro36, MicroRef) strains. FIG. 5F is a box plot showing the concentrations of IL-17F in supernatants of bacterial pre-exposed fetal splenic antigen presenting cells co-cultured with lamina propria T cells for five days. FIG. 5G depicts intracellular INFγ production among sorted intestinal effector memory T cells after three days of mixed lymphocyte reactions with sorted lin-, CD45+, HLA-DR+ antigen presenting cells that were pre-exposed to media or Micrococcus (Micro36, MicroRef1) strains. On the left panel, a box plot depicts the percent IFNγ+ T cells among live, TCRβ+, CD4+, Vα7.2−, PLZF+ after four hours of treatment with Brefeldin A. On the right panel are example flow plots of sorted effector memory T cells composed primarily of PLZF+ T cells (top) and intracellular cytokine, IFNγ and TNFα, expression (bottom); numbers indicate mean proportion and standard error of the mean (SEM). FIG. 5H is a box plot depicting mean fluorescence intensity (MFI) of LLT1 expression of live, lin-, CD45+, HLA-DR+ splenocytes after four hours of exposure to media, Micro36, MicroRef, Lacto166, Lacto167, or LactoRef or unstimulated lamina propria (LP) antigen presenting cells ex vivo. FIG. 5I is a line graph plot depicting example histograms of LLT1 expression of live, lin-, CD45+, HLA-DR+ splenocytes after four hours of exposure to media, Micro36, MicroRef, Lacto166, Lacto167, or LactoRef or unstimulated lamina propria (LP) antigen presenting cells ex vivo. FIG. 5J is a line graph showing the multiplicity of infection (MOI) of Micro36 relative to proportion of LLT1+ live, lin-, CD45+, HLA-DR+ splenocytes, each dot represents mean of n=3 samples and error bars indicate standard error of the mean. Linear mixed effects (LME) modeling was used to evaluate significance between strains, controlling for repeated measures of cell donor; LME residuals are plotted for c-f Each dot represents an independent fetal sample.

FIGS. 6A-D present data related to low-burden bacterial signal detected in fetal meconium. FIG. 6A is a box plot depicting total 16S copy number per gram frozen sample in meconium from proximal, mid, and distal sections of the fetal small intestine or extraction buffer was quantified by qPCR of DNA extracts using a standard curve; Wilcoxon rank sum test for significance compared to buffer control. FIG. 6B depicts fluorescent in situ hybridization probes targeting eubacteria (EUB) or non-targeting probe (NEUB) in 0.5 μm cryosections of human fetal (top panel) or murine (bottom panel) terminal ileum at 400× magnification. Representative images of three experiments. Arrowheads indicate EUB-positive findings in fetal sections. Scale bar corresponds to 50 μm. FIG. 6C is a box plot depicting quantification of independent fields of view (FOV) per μm of human fetal intestinal length. Wilcoxon rank sum test for significance. FIG. 6D is a box plot depicting quantification of independent fields of view (FOV) per μm of murine intestinal length. Wilcoxon rank sum test for significance.

FIGS. 7A-C present data showing that depletion of mtDNA by Cas9 does not alter bacterial composition after 30 cycles of amplification. 16S rRNA V4 profiling of a subset (n=10) of banked fetal meconium samples using different library preparation methods: gel extraction and 30 or 35 cycles of amplification, or 30 cycles combined with DASH performed on individual samples (Individual DASH) or on the library pool (Pooled DASH). FIG. 7A is a bar graph showing the expansion in Enterobacteriaceae family is detected in 35-cycle amplification method, while small expansion of Pseudomonadaceae is detected post-DASH. The legend at the right lists families as they appear in each column from top to bottom; however, there is no band for Tissierellaceae in the Gel extraction (35 cycles) column. FIG. 7B is a scatter plot showing principal coordinates analysis of Bray Curtis distances of libraries using 30 cycles of amplification, latter to provide an outgroup known to skew bacterial composition. Ellipses indicate 95% confidence intervals. All p-values were calculated using Linear Mixed Effects (LME) modeling to correct for n=10 paired samples that underwent multiple library preparation methods. FIG. 7C is a scatter plot showing principal coordinates analysis of Bray Curtis distances using 30 and 35 cycles of amplification, latter to provide an outgroup known to skew bacterial composition. Ellipses indicate 95% confidence intervals. All p-values were calculated using Linear Mixed Effects (LME) modeling to correct for n=10 paired samples that underwent multiple library preparation methods.

FIGS. 8A-I present data showing that sparse bacterial signal distinct from background is detected in fetal meconium. FIG. 8A is a bar graph showing the number of operational taxonomic units (OTUs) per sample detected in fetal meconium from proximal-, mid-, or distal-segments of the small intestine after technical control filtering. FIG. 8B is a scatter plot showing principal coordinates analysis (PCoA) of Bray Curtis distances of rareified bacterial profiles of proximal-, mid-distal-sections of the intestine. The color legend is the same as shown in FIG. 8A. FIG. 8C is a box plot showing inter- and intra-sample Bray Curtis distances between indicated comparisons of intestinal sections. FIG. 8D is a scatter plot showing PCoA of Bray Curtis distances of Lactobacillus-meconium (LM), Micrococcaceae-meconium (MM), or Other-meconium (OM) compared to fetal kidney control. FIG. 8E is a line graph showing bacterial abundance ranks in fetal meconium, post-natal meconium, and kidney control. FIG. 8F is a three dimensional scatter plot showing PCoA of Bray Curtis distances of unrareified and unfiltered bacterial profiles of mid-sections of meconium with technical negative controls (extraction buffer, room air swab, pre-moistened swabs). LM and MM samples identified in later analyses are highlighted; significance was measured by linear mixed effects modeling (LME) to correct for repeated measures in FIG. 8B and FIG. 8F, t-test was used for FIG. 8C, PERMANOVA was used in FIG. 8D. FIG. 8G is a scatter plot showing significantly enriched taxa (Log 2-fold change 2, false discovery rate <0.05) in meconium as compared to both kidney and procedural environment swab. Dots represent differential taxa and are scaled by percent relative abundance in meconium; top abundant taxa are labeled. DESEQ2 of unnormalized reads was used to find differentially abundant taxa. FIG. 8H is a scatter plot showing significantly enriched taxa (Log 2-fold change 2, false discovery rate <0.05) in meconium as compared to kidney swab controls. Dots represent differential taxa and are scaled by percent relative abundance in meconium; top abundant taxa are labeled. DESEQ2 of unnormalized reads was used to find differentially abundant taxa. FIG. 8I is a scatter plot showing significantly enriched taxa (Log 2-fold change 2, false discovery rate <0.05) in meconium as compared to procedural swab controls. Dots represent differential taxa and are scaled by percent relative abundance in meconium; top abundant taxa are labeled. DESEQ2 of unnormalized reads was used to find differentially abundant taxa.

FIGS. 9A-C present data showing correlation of bacterial signal in fetal meconium with gestational age. FIG. 9A is a graph showing correlation of gestational age with total number of OTUs in mid-section meconium samples with gestational age in all samples. Pearson correlation coefficient and p-values. FIG. 9B is a graph showing correlation of gestational age with Lactobacillus OTU12 count with gestational age in all samples or among Lactobacillus meconium (LM), Micrococaceae meconium (MM), or Other meconium (OM) samples. Pearson correlation coefficient and p-value. FIG. 9C is a graph showing correlation of gestational age with Micrococcaceae OTU10 count with gestational age in all samples or among Lactobacillus meconium (LM), Micrococaceae meconium (MM), or Other meconium (OM) samples. Pearson correlation coefficient and p-value.

FIGS. 10A-C present data related to scanning electron micrographs of fetal intestinal lumen. FIG. 10A is a diagram showing a sample preparation method of fetal intestines: terminal ileum was ligated with sterile suture to avoid exposing lumen, fixed, and critical point dried. Intestinal internal contents were exposed immediately prior to imaging, mounted, and coated with 15-30 nm of iridium. Specimens were imaged with Zeiss ULTRA55 FE-SEM and kept under vacuum between imaging sessions. FIGS. 10B-C are panels of scanning electron micrographs of four fetal intestinal specimens (i.) at low magnification, (ii.-iii.) two independent regions within intestinal lumen, and (iv.) sub-epithelial region, outside of the lumen. Scale bars indicate size, from left to right for each specimen as follows: Specimen 1 (200 μm, 1 μm, 1 μm, 1 μm); Specimen 2 (200 μm, 2 μm, 2 μm, 2 μm); Specimen 3 (200 μm, 10 μm, 2 μm, 1 μm); and Specimen 4 (100 μm, 1 μm, 1 μm, 1 μm).

FIGS. 11A-C present data showing divergent epithelial transcriptome and lamina propria T cells in samples associated with Lactobacillus meconium (LM), Micrococaceae meconium (MM), or Other meconium (OM). FIG. 11A is a scatter plot showing principal components (PC) analysis of euclidean distances of top 10000 variable genes (by coefficient of variation) in LM associated epithelium (LM-E), MM associated epithelium (MM-E), or OM associated epithelium (OM-E) as determined by RNA sequencing. PERMANOVA test for significance. FIG. 11B shows, on the left panel, a heat map depicting the expression of genes significantly enriched in MM-E and LM-E with respect to OM-E. On the right panel are boxplots of mean normalized read counts for each kmeans cluster among MM-E, LM-E, and OM-E as determined by RNAseq. Log 2-fold change |1| and false discovery rate <0.05 was used as a cut-off. FIG. 11C is a box plot showing proportion of PLZF+CD161-T cells T cells in intestinal lamina propria paired with LM, MM, or OM (LM-LP; MM-LP; OM-LP) among live, TCRβ+, Vα7.2−, CD4+ cells. Kruskal-Wallis ANOVA, with Dunnet's correction for multiple comparisons was used for FIGS. 11B-C. Each dot represents a biological replicate.

FIGS. 12A-B present alignments showing that Lactobacillus and Micrococcus fetal isolates exhibit high 16S rRNA V4 sequence identity to fetal meconium OTUs. FIG. 12A shows sequence alignment of 16S V4 rRNA gene sequences of Lacto166, Lacto167 to OTU12 Percentages indicate identity to respective reference OTU sequence. The sequences illustrated for OTU12, Lacto166, and Lacto167 correspond to nucleotides 1-253 of SEQ ID NO: 6, nucleotides 511-763 of SEQ ID NO: 3, and nucleotides 511-763 of SEQ ID NO: 5, respectively. FIG. 12B shows sequence alignment of 16S V4 rRNA gene sequences of Micro36 to OTU10. Percentages indicate identity to respective reference OTU sequence. The sequences illustrated for OTU10 and Micro36 correspond to nucleotides 1-253 of SEQ ID NO: 4, and nucleotides 451-703 of SEQ ID NO: 4, respectively.

FIGS. 13A-P present data showing that Lactobacillus and Micrococcus isolates from fetal meconium exhibit adaptation to the fetal environment. FIG. 13A is a line graph showing the effects of 10⁻⁵M progesterone (P4) and 10⁻⁶M β-Estradiol (E2) on the growth of MicroRef1 compared to ethanol vehicle control, in carbon-rich media at 37° C. FIG. 13B is a line graph showing the effects of 10⁻⁵M P4 and 10⁻⁶M E2 on the growth of MicroRef2 compared to ethanol vehicle control, in carbon-rich media at 37° C. FIG. 13C is a line graph showing the effects of P4 and E2 on the growth of Micro36 with indicated concentrations of P4 and E2 compared to ethanol vehicle control, in carbon-rich media at 37° C. From top to bottom at the right end of the graph, the curves are as follows: vehicle, 10⁻⁵M P4, 2.5×10⁻⁵M P4, 5×10⁻⁵M P4. FIG. 13D is a line graph showing the effects of 10⁻⁵M P4 and 10⁻⁶M E2, alone or in combination, on the growth of Micro36 compared to ethanol vehicle control, in carbon-rich media at 37° C. From top to bottom at the right end of the graph, the curves are as follows: vehicle, E2, P4, and P4 E2. FIG. 13E is a line graph showing the growth of Lacto166 at varying concentrations of P4 and E2 in chopped-meat carbohydrate (CMC). From top to bottom at the right end of the graph, the curves are as follows: 1×10⁻⁵M P4, 5×10⁻⁶M P4, and vehicle. FIG. 13F is a line graph showing the growth of Lacto167 at varying concentrations of P4 and E2 in CMC. From top to bottom at the right end of the graph, the curves are as follows: 1×10⁻⁵M P4, 5×10⁻⁶M P4, and vehicle. FIG. 13G is a line graph showing the growth of LactoRef with 10⁻⁵M P4 and 10⁻⁶M E2 in CMC. FIG. 13H is a line graph showing the effects of 10⁻⁵M progesterone (P4) and 10⁻⁶M β-Estradiol (E2) on the growth of MicroRef1, in carbon limiting media at 37° C.

FIG. 13I is a line graph showing the effects of 10⁻⁵M progesterone (P4) and 10⁻⁶M β-Estradiol (E2) on the growth of MicroRef2, in carbon limiting media at 37° C. FIG. 13J is a line graph showing the effects of 10⁻⁵M progesterone (P4) and 10⁻⁶M β-Estradiol (E2) on the growth of Lacto166, in carbon limiting media at 37° C. FIG. 13K is a line graph showing the effects of 10⁻⁵M progesterone (P4) and 10⁻⁶M β-Estradiol (E2) on the growth of Lacto167, in carbon limiting media at 37° C. FIG. 13L is a line graph showing the effects of 10⁻⁵M progesterone (P4) and 10⁻⁶M β-Estradiol (E2) on the growth of LactoRef, in carbon limiting media at 37° C. For FIGS. 13A-L, representative growth curves of three independent experiments measured by optical density at 600 nm (OD600), error bars denote standard error of the mean (SEM) between three technical experiments. For carbon-rich media conditions, integral of logistic regression model fitting was used to calculate area under the curve (auc) and change with respect to vehicle control is reported as Δauc. FIG. 13M is line graph showing the intracellular survival of Micro36, Lacto166, Lacto167 in RAW264.3 cells. Generalized linear model of log(CFU+1) against E. coli for each timepoint was used to calculate significance. FIG. 13N is a line graph showing the intracellular survival of MicroRef1, LactoRef in RAW264.3 cells. Generalized linear model of log(CFU+1) against E. coli for each timepoint was used to calculate significance. FIG. 13O is a bar graph showing the growth of indicated strains on media with (+) or without (−) gentamycin (10 μg mL-1) following 24-50 hours of intracellular growth in RAW264.7 cells. Example data from three independent experiments, error bars indicate SEM of three technical replicates. FIG. 13P is a bar graph showing the growth of indicated strains on media with (+) or without (−) gentamycin (10 μg mL-1) following 24-50 hours of intracellular growth in primary human fetal intestinal antigen presenting cells. Example data from three independent experiments, error bars indicate SEM of three technical replicates.

FIG. 14 is a diagram showing the genomic features of fetal Micrococcus isolate. Alignment of all publically available Micrococus genomes; single copy Micrococcus genes used for phylogeny (inset) and genes unique Micro36 isolate are highlighted. Each radial layer represents a genome; representative or reference genomes are colored in black indicated with asterisk; inner dendogram represents hierarchical clustering of amino acid sequences based on their sequence composition and distribution across genomes; genomes are organized based on gene clusters they share using Euclidian distance and Ward ordination; outer ring represents single copy genes predicted using hidden markov model in Anvi'o package. Inset is a phylogenetic tree of single-copy conserved genes across all publically available genomes within Micrococcus and fetal meconium isolate Micro36.

FIG. 15 is a diagram showing the genomic features of fetal Lactobacillus isolates. Alignment of select publically available Lactobacillus genomes; Lactobacillus genes used for subsequent phylogeny (inset) are highlighted. Each radial layer represents a genome; representative or reference genomes are colored in black and indicated by an asterisk; inner dendogram represents hierarchical clustering of amino acid sequences based on their sequence composition and distribution across genomes; genomes are organized based on gene clusters they share using Euclidian distance and Ward ordination; outer ring represents single copy genes predicted using hidden markov model in Anvi'o package. Inset is a phylogenetic tree of single-copy conserved genes across select publically available genomes within Lactobacillus and fetal meconium isolates Lacto166 and Lacto167.

FIGS. 16A-C present data showing prevalence of L. jensenii and M. luteus in infant and mothers. FIG. 16A is graph showing percent identity of samples to 16S rRNA gene of Lacto166 or Micro36 in three independent infant stool cohorts. Each symbol represents a sample with a positive hit (>97% sequence identity); symbol shape indicates cohort. FIG. 16B shows line graphs showing relative abundance of Micrococcus luteus (top plots) and Lactobacillus jensenii (bottom plots) in metagenomic sequencing cohorts across body sites at delivery mother and infant within four months after birth. Metagenomic sequences obtained from two independent studies were classified using a custom kraken2 database including fetal M. luteus Micro 36 and L. jensenii Lacto166 and Lacto167 genomes. FIG. 16C shows line graphs showing relative abundance of Micrococcus luteus (top plots) and Lactobacillus jensenii (bottom plots) in metagenomic sequencing cohorts across in maternal stool around delivery and infant stool within the first three months of life. Metagenomic sequences obtained from two independent studies were classified using a custom kraken2 database including fetal M. luteus Micro 36 and L. jensenii Lacto166 and Lacto167 genomes.

FIGS. 17A-E present data showing Lactobacillus and Micrococcus isolates induce differential epithelial transcriptomes in vitro. FIG. 17A is a volcano plot showing significantly (false discovery rate (FDR)<0.05) and differentially (Log 2FoldChange |1|) expressed genes in primary human fetal intestinal epithelial cells a. Lacto166 versus Micro36 treatment comparison. FIG. 17B is a volcano plot showing significantly (false discovery rate (FDR)<0.05) and differentially (Log 2FoldChange|1|) expressed genes in primary human fetal intestinal epithelial cells Micro36 treatment versus media control. FIG. 17C is a volcano plot showing significantly (false discovery rate (FDR)<0.05) and differentially (Log 2FoldChange |1|) expressed genes in primary human fetal intestinal epithelial cells Lacto166 treatment versus media control. FIG. 17D is a bar graph showing normalized enrichment scores of gene set enrichment analysis of transcripts associated with epithelial cell states in Lacto166 or Micro36 treatment. All results are filtered on a nominal p-value of 0.1 and FDR is indicated. FIG. 17E is a heatmap of significantly and differentally enriched genes (FDR<0.05, Log 2 Fold Change 1) in epithelial cells treated with Lacto166, Micro36, or media control. Genes that were also enriched in LM-E or MM-E are highlighted.

FIGS. 18A-C present data showing the effects of fetal Lactobacillus and Micrococcus isolates on antigen presenting cell phenotypes. FIG. 18A is a box plot showing proportions of live cells after four hours of treatment with live Micrococcus (Micro36, MicroRef1, MicroRef2; left) or Lactobacillus (Lacto166, Lacto167, LactoRef; right) strains. ANOVA test for significance. FIG. 18B shows flow plots and box plots. On the left panel are example flow plots of CD103 expression among CD11c+ HLA-DR+, CD45+, lin−, live splenocytes that were exposed to either media, Lactobacillus (Lacto166, Lacto167, LactoRef) or Micrococcus (Micro36, MicroRef1) strains. Numbers indicate mean proportion and standard error of the mean (SEM). On the right panel are the proportions of CD103+ among CD11c+ HLA-DR+, CD45+, lin−, live splenocytes. FIG. 18C is a box plot showing the concentrations of G-CSF in supernatants of fetal splenic antigen presenting cells following four hours of exposure to Lactobacillus (Lacto166, Lacto167, LactoRef) or Micrococcus (Micro36, MicroRef) strains. Linear mixed effects (LME) modeling was used to evaluate significance between strains, controlling for repeated measures of cell donor. LME residuals are plotted for FIG. 18C. Each dot represents an independent fetal sample.

FIGS. 19A-L present data showing that fetal Lactobacillus and Micrococcus isolates promote distinct T cell phenotypes. Boxplots illustrates results of concentration measurements in culture supernatants of lamina propria T cell five day co-culture with splenic antigen presenting cells pre-exposed to Lactobacillus (Lacto166, Lacto167, LactoRef) or Micrococcus (Micro36, MicroRef1, MicroRef2) strains for concentration of IL-17A (FIG. 19A), IL-2 (FIG. 19B), GM-CSF (FIG. 19C), IL-4 (FIG. 19D), IL-10 (FIG. 19E), IL-13 (FIG. 19F), and TNFα (FIG. 19G). FIG. 19H shows, on the left panel, a box plot showing proportion of CD25^{hi} FoxP3+ regulatory T cells (Tregs); on the right panel are representative flow plots of FoxP3 and CD25 expression after five days of exposure to splenic APCs pretreated with media, Lactobacillus (Lacto167) or Micrococcus (Micro36) strains. FIG. 19I is a box plot showing proportions of PLZF+ T cells among intestinal live, TCRβ+, CD4+, Vα7.2−, cells after five days of exposure to splenic APCs pretreated with media, Lactobacillus (Lacto166, Lacto167, LactoRe) or Micrococcus (Micro36, MicroRef1) strains. FIG. 19J presents flow plots depicting HLA-DR+CD45+ lin− cells pre- (left panel) and post- (right panel) fluorescence activated cell sorting (FACS). FIG. 19K presents flow plots depicting the proportion of naïve (CD45RA+ CCR7+), central memory (TCM, CD45RA− CCR7+), and effector memory T cells (TEM, CD45RA− CCR7−) among live, TCRβ+, CD4+ cells (left panel) and PLZF and CD161 expression among memory subsets, numbers indicate proportion in TEM (right panel). FIG. 19L presents flow plots depicting pre- (left panel) and post- (right panel) FACS of effector memory T cells. Numbers indicate mean proportion and standard error of the mean (SEM). Linear mixed effects (LME) modeling was used to evaluate significance between strains, controlling for repeated measures of cell donor. LME residuals are plotted for FIGS. 19A-G. Each dot represents an independent fetal sample, unless otherwise indicated.

FIG. 20 is a diagram showing an example collection method for a fetal intestinal sample bank. Uninterrupted small intestine sections were divided into equal thirds and internal contents (meconium) cryopreserved for either genomic DNA extraction (in RNAlater) or bacterial isolation (in 50% v/v glycerol). Remaining intestinal tissue from all three sections was pooled and washed with EDTA to recover epithelium (preserved in RNAlater for subsequent RNAseq analysis) and enzymatically digested to isolate lamina propria cells (for immediate analysis by flow cytometry). Internal kidney punch biopsies and surgical environmental swabs served as procedural or environmental controls. Extraction buffer, pre-moistened swabs, and pre-moistened swabs held in the surgical room air for 30 seconds served as technical negative controls.

FIG. 21 presents flow plots depicting gating strategy for T cell profile assessment. Gating strategy for identification of PLZF+ CD161+ CD4+ αβT cells. Cells were gated on 1—lymphocytes, 2—singlets, 3—live cells expressing TCRβ, 4—CD4 expressing cells that were excluded of the dominant invariant chain expressed on mucosa-associated invariant T cells, Vα7.2. 5—PLZF+, PLZF+ CD161+ or PLZF+ CD161− cells. All gating was set on mesenteric lymph node (MLN) internal controls and when available, splenic internal controls (SPL).

FIG. 22 presents flow plots depicting gating strategy for identification of fetal splenic antigen presenting cells. Cells were gated on panel 1 for lymphocytes, on panel 2 for singlets, on panel 3 for live cells, on panel 4 for lineage (CD3, CD56, CD20, CD19)− and CD45+, and on panel 5 for HLA-DR+ cells.

### DETAILED DESCRIPTION OF THE INVENTION

Included herein are, inter alia, methods and compositions for treating, preventing, or reducing the risk of dysbiosis, inflammation, inflammatory diseases, childhood obesity, and premature birth, as well as methods and compositions for increasing or promoting healthy or normal immune system maturation or Treg function. Also provided are methods detecting, isolating, and culturing bacterial strains, as well as isolated bacterial strains. In embodiments, provided herein are Lactobacillus and Micrococcus species that promote tolerogenic immunity.

Asthma is the most common chronic disease worldwide. It disproportionately affects children, families living below the poverty line, and minorities. Risk is greatest between birth and 4. Childhood allergic asthma specifically refers to the development of severe asthma before age 12. These patients are often have a history of allergic sensitization (atopy) and a family history of asthma. Premature birth, defined as childbirth occurring at less than 37 completed weeks of gestation, is the number one cause of morbidity and mortality in children under 5 globally. Complications associated with prematurity extend into later life, resulting in enormous physical, psychological, and economic costs. The fetal inflammatory response is a known causal factor resulting in premature birth and studies in animals suggest that this inflammation originates in the fetal intestine. There is no preventative treatment for premature labor and few treatment options for its associated co-morbidities. Strategies to control inflammatory response in the fetal intestine, such as through supplementation with beneficial bacteria have not been investigated.

Asthma prevention therapeutics do not currently exist in the clinic. While infants may be identified as high risk for asthma prior to birth on the basis of maternal/paternal asthma status, there is no intervention to prevent the development of asthma. Because bacterial colonization patterns in early life have been identified as an important risk factor, probiotic investigative therapies have emerged. However, current probiotic therapies in-development have not been evaluated for impact on the developing human intestine. Furthermore, we have identified fetal intestinal bacteria species in the human fetal intestine that may shape lifelong immunity through generation of T cell memory. These fetal intestinal bacteria, isolated from fetal meconium, are distinct from their phylogenetic relatives, several of which are used in current probiotic on the market. Thus these species are likely to exhibit an even greater protective as live biotherapeutics.

Effective therapeutics to prevent preterm labor and its co-morbidities do not exist. While women may be identified as high risk for pre-term labor, there is no treatment for fetal inflammation that will eventually result in pre-term birth and co-morbidities such as neonatal sepsis, necrotizing enterocolitis, cerebral palsy, and respiratory illnesses. Without being limited by any scientific theory, Micrococcus and Lactobacillus are associated with a decreased inflammatory state of the fetal intestine. In embodiments, supplementation with these bacteria or their products in pregnant women lowers fetal intestinal inflammation that contributes to preterm birth and its co-morbidities. In embodiments, Micrococcus and Lactobacillus disclosed herein promote tolerogenic immunity.

The neonatal period has been identified as a high-risk window for developing chronic inflammatory diseases such as asthma. In embodiments, neonates at heightened risk of childhood atopy and asthma are characterized by metabolic dysfunction and inter-kingdom perturbation of their fecal microbiota. During this period, bacteria and fungi begin to colonize the infant intestine and shape lifelong immunity. Microbial interventions during the early life period have been an area of active investigation. We investigated whether bacterial presence in the human fetal intestine in utero shapes developing immunity. We discovered that the presence of two fetal intestinal bacteria bacteria belonging to the Micrococcus and Lactobacillus genera, isolated from human fetal meconium are highly correlated with intestinal immune cell profiles. Without being bound by any scientific theory, we further found that Micrococcus promotes fetal antigen presenting cells to express immunosuppressive molecules that result in reduced activation of autologous fetal intestinal memory T cells (immune tolerance). In parallel, Lactobacillus promotes known tolerance promoting ligands on fetal antigen presenting cells. We also found that our fetal isolates of Lactobacillus and Micrococcus exert significantly different effects on fetal immunity than publically available, phylogenetically related strains. Thus, without being bound by any scientific theory, we have demonstrated that bacterial presence in the human intestine occurs earlier than previously appreciated and that these fetal intestinal bacterial strains promote immune tolerance development through immune tolerance in humans.

We have isolated several strains of Micrococcus and Lactobacillus from human fetal intestines and evaluated their effect at reducing inflammation on human fetal intestinal antigen presenting cells and T cells ex vivo. In embodiments, the presence of Micrococcus and Lactobacillus directly shapes T cell immunity in the fetal intestine. In embodiments, Micrococcus and Lactobacillus colonize the intestines of a fetus (e.g., after administration). In embodiments, the colonization is transient. In embodiments, the colonization persists at least until after birth.

In embodiments, a combined fetal intestinal bacterial therapy is more biologically relevant (e.g., effective at reducing a disease or disorder such as asthma or inflammation, or the risk thereof) than other therapies. Included herein is preventative care for asthma and interventional care for women undergoing or at high-risk for preterm labor, as well as potential for therapy in established inflammatory disease. In embodiments, supplementation with Micrococcus and Lactobacillus to fetuses (via maternal introduction) or neonates at high risk for chronic inflammatory diseases, such as asthma, will result in lifelong immune tolerance and reduced disease severity. In embodiments, therapeutic oral supplementation with Micrococcus and/or Lactobacillus strains as disclosed herein in high-risk for asthma newborns and infants increases immune system maturation and/or Treg function. In embodiments, therapeutic vaginal supplementation with Micrococcus and/or Lactobacillus strains as disclosed herein in pregnant women increases immune system maturation and/or Treg function in the fetus. In embodiments, therapeutic vaginal/oral supplementation with Micrococcus and/or Lactobacillus strains as disclosed herein in pregnant women decreases inflammation in the fetus to prevent premature birth. In embodiments, therapeutic vaginal/oral supplementation with Micrococcus and/or Lactobacillus strains as disclosed herein in pregnant women decreases inflammation in the fetus to prevent childhood obesity, which we have demonstrated is associated with gut microbiome perturbation in the earliest phases of post-natal life. In embodiments, therapeutic oral supplementation with Micrococcus and/or Lactobacillus strains as disclosed herein to subjects with chronic inflammatory disease down-regulates inflammation. In embodiments, the combination cocktail reduces airway inflammation. In embodiments, the combination reduces inflammation in a subject, or in a child of a subject to whom the combination is administered while pregnant. In embodiments, the combination cocktail reduces airway inflammation. In embodiments, the combination reduces inflammatory bowel disease in a subject, or in a child of a subject to whom the combination is administered while pregnant. In embodiments, oral supplementation with a combination of strains as disclosed herein reduces airway inflammation in a a subject who has allergic asthma. In embodiments, vaginal supplementation with a combination of strains in pregnant mice results in decreased airway inflammation in offspring. In embodiments, a combination of strains can be utilized during pregnancy to reduce inflammation. These embodiments are exemplary. Additional embodiments are disclosed herein.

### I. DEFINITIONS

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure.

Depending on context, the term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “isolated”, when applied to a bacterium, refers to a bacterium that has been (1) separated from at least some of the components with which it was associated when initially produced (whether in nature or in an experimental setting), and/or (2) produced, prepared, purified, and/or manufactured by the hand of man, e.g. using artificial culture conditions such as (but not limited to) culturing on a plate, in a flask, and/or in a fermenter. Isolated bacteria include those bacteria that are cultured, even if such cultures are not monocultures. In embodiments, isolated bacteria are in a monoculture. In embodiments, isolated bacteria are in a coculture or have been cocultured with one or more eukaryotic (e.g., mammalian such as human) cells (such as monocytes, macrophages, or epithelial cells). Isolated bacteria may be separated from at least about 10%, about 20%, about 30%, about 40%, about 50%, about 60%, about 70%, about 80%, about 90%, or more of the other components with which they were initially associated (e.g., by weight). In embodiments, isolated bacteria are more than about 80%, about 85%, about 90%, about 91%, about 92%, about 93%, about 94%, about 95%, about 96%, about 97%, about 98%, about 99%, or more than about 99% pure (e.g., by weight). In embodiments, a bacterial population provided herein includes isolated bacteria. In embodiments, a composition provided herein includes isolated bacteria. In embodiments, the bacteria that are administered are isolated bacteria.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In embodiments, a “patient” or “subject in need thereof” refers to a living member of the animal kingdom who has or that may have or develop (e.g., is at risk of or is suspected of suffering from) the indicated disorder or disease. In embodiments, a subject or patient is a member of a species that includes individuals who naturally suffer from the disorder or disease. In embodiments, the subject is a mammal. Non-limiting examples of mammals include rodents (e.g., mice and rats), primates (e.g., lemurs, bushbabies, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. In embodiments, the subject is a human. In embodiments, the subject is a non-mammalian animal such as a turkey, a duck, or a chicken. In embodiments, a subject is a living organism suffering from or prone to a disease or condition that can be treated by administration of a composition or pharmaceutical composition as provided herein. The terms “subject,” “patient,” “individual,” etc. can be generally interchanged. In embodiments, an individual described as a “patient” does not necessarily have a given disease or disorder, but may, e.g., be merely seeking medical advice.

As used herein, a “symptom” of a disease includes any clinical or laboratory manifestation associated with the disease, and is not limited to what a subject can feel or observe.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; and/or improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. In embodiments, the administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

A “effective amount” is an amount sufficient for an agent to accomplish a stated purpose relative to the absence of the compound (e.g. achieve the effect for which it is administered, treat a disease, reduce gene expression, increase gene expression, reduce immune activation, increase immune tolerance, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of an agent is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the agent being employed. In embodiments, the dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

In embodiments, administration may be oral administration, vaginal administration, rectal administration, administration as a suppository (e.g. rectally), or topical administration.

As used herein the term “dysbiosis” means a difference in the microbiota compared to a general or healthy population. In embodiments, the dysbiosis is gastrointestinal dysbiosis (e.g., dysbiosis in a small intestine or large intestine). In embodiments, gastrointestinal dysbiosis includes a difference in gastrointestinal microbiota commensal species diversity compared to a general or healthy population. In embodiments, gastrointestinal dysbiosis includes a decrease of beneficial microorganisms and/or increase of pathobionts (pathogenic or potentially pathogenic microorganisms) and/or decrease of overall microbiota species diversity. Many factors can harm the beneficial members of the gastrointestinal microbiota leading to dysbiosis, including (but not limited to) infection, antibiotic use, psychological and physical stress, radiation, and dietary changes. In embodiments, the dysbiosis includes a reduced amount (absolute number or proportion of the total microbial population) of bacterial or fungal cells of a species or genus (e.g., 5%, 10%, 15%, 20%, 25%, 30%, 35%, 40%, 45%, 50%, 55%, 60%, 65%, 70%, 75%, 80%, 85%, 90%, 95% or more lower) compared to a healthy subject (e.g., a corresponding subject who has not been administered an antibiotic within about 1, 2, 3, 4, 5, or 6 months, and/or compared to a general or healthy population). In embodiments, the dysbiosis includes an increased amount (absolute number or proportion of the total microbial population) of bacterial or fungal cells within a species or genus (e.g., 5%, 10%, 15%, 20%, 25%, 30%, 35%, 40%, 45%, 50%, 55%, 60%, 65%, 70%, 75%, 80%, 85%, 90%, 95% or more higher) compared to a healthy subject (e.g., a corresponding subject has not been administered an antibiotic within about 1, 2, 3, 4, 5, or 6 months, and/or compared to a general or healthy population). In embodiments, antibiotic administration (e.g., systemically, such as by intravenous injection or orally) is causing or has caused a major alteration in the normal microbiota. Thus, as used herein, the term “antibiotic-induced dysbiosis” refers to dysbiosis caused by or following the administration of an antibiotic.

A “control” or “standard control” refers to a sample, measurement, or value that serves as a reference, usually a known reference, for comparison to a test sample, measurement, or value. For example, a test sample can be taken from a patient suspected of having a given disease or disorder (e.g. dysbiosis or an inflammatory disease) and compared to a known normal (non-diseased) individual (e.g. a standard control subject). In embodiments, a standard control can represent an average measurement or value gathered from a population of similar individuals (e.g. standard control subjects) that do not have a given disease (e.g. standard control population), e.g., healthy individuals with a similar medical background, same age, weight, etc. In embodiments, a standard control is a proportion, level, or amount (e.g., an average proportion, level, or amount) in a general or healthy population of subjects. In embodiments, a standard control is a proportion, level, or amount (e.g., an average proportion, level, or amount) in a general population of subjects. In embodiments, a standard control is a proportion, level, or amount (e.g., an average proportion, level, or amount) in a healthy population of subjects. In embodiments, a general population of subjects is a general population of subjects in a geographical area (such as a country or continent, e.g., Asia, Australia, Africa, North America, South America, or Europe). In embodiments, a general population of subjects is a general population of subjects in (e.g., that self-identify as being within) an ethnic group such as caucasian (e.g., white), African, of African descent (e.g., African American), Native American, Asian, or of Asian descent. In embodiments, a general population of subjects is a general population of subjects without an inflammatory disease. In embodiments, a general population of subjects is a general population of subjects with an inflammatory disease. In embodiments, a standard control value can be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. microbiome, RNA levels, protein levels, specific cell types, specific bodily fluids, specific tissues, metabolites, etc.).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“Biological sample” or “sample” refers to materials obtained from or derived from a subject or patient. In embodiments, a biological sample is or includes a bodily fluid such as meconium, blood, amniotic fluid, or a fluid from a placenta. In embodiments, a biological sample is or includes blood, serum, or plasma. In embodiments, a biological samples is or includes blood, a blood fraction, or product (e.g., serum, plasma, platelets, red blood cells, and the like). In embodiments, a biological sample is or includes tissue, such as tissue from an intestine. In embodiments, a sample is obtained from a eukaryotic organism, such as a mammal such as a primate e.g., chimpanzee or human; cow; dog; cat; a rodent, e.g., guinea pig, rat, or mouse; rabbit; or a bird; reptile; or fish. In embodiments, a biological sample includes sections of tissues such as biopsy and autopsy samples, and frozen sections taken for histological purposes.

As used herein the abbreviation “sp.” for species means at least one species (e.g., 1, 2, 3, 4, 5, or more species) of the indicated genus. The abbreviation “spp.” for species means 2 or more species (e.g., 2, 3, 4, 5, 6, 7, 8, 9, 10 or more) of the indicated genus. In embodiments, methods and compositions provided herein include a single species within an indicated genus or indicated genera, or 2 or more (e.g., a plurality including more than 2) species within an indicated genus or indicated genera. In embodiments, 1, 2, 3, 4, 5, or more or all or the indicated species is or are isolated. In embodiments, the indicated species are administered together. In embodiments, each of the indicated species is present in a single composition that includes each of the species. In embodiments, each of the species is administered concurrently, e.g., within about 1, 2, 3, 4, 5, 6, 7, 8, 9, 10, 30, or 60, 1-5, 1-10, 1-30, 1-60, or 5-15 seconds or minutes of each other.

A “fetal” bacterium is a bacterium from a species that has been identified in amniotic fluid, a fetal intestine, fetal meconium, neonate meconium, or a placenta. In embodiments, not all strains of the species are naturally present in amniotic fluid, a fetal intestine, fetal meconium, neonate meconium, or a placenta. In embodiments, the fetal bacterium is a bacterium from a strain that has been identified in amniotic fluid, a fetal intestine, fetal meconium, neonate meconium, or a placenta. In embodiments, a fetal bacterium is from a species or strain that has been identified (e.g., found or detected) in amniotic fluid. In embodiments, a fetal bacterium is from a species or strain that has been identified (e.g., found or detected) a fetal intestine (e.g., a proximal, mid, and/or distal portion of the intestine). In embodiments, a fetal bacterium is from a species or strain that has been identified (e.g., found or detected) in fetal meconium. In embodiments, a fetal bacterium is from a species or strain that has been identified (e.g., found or detected) in neonate meconium. In embodiments, a fetal bacterium is from a species or strain that has been identified (e.g., found or detected) in a placenta (e.g., in placental tissue or a fluid obtained from a placenta). In embodiments, a fetal bacterium has been isolated from amniotic fluid. In embodiments, a fetal bacterium has been isolated from a fetal intestine (e.g., a proximal, mid, and/or distal portion of the intestine). In embodiments, a fetal bacterium has been isolated from fetal meconium. In embodiments, a fetal bacterium has been isolated from neonate meconium. In embodiments, a fetal bacterium has been isolated from a placenta (e.g., in placental tissue or a fluid obtained from a placenta). In embodiments, a fetal bacterium has been isolated from amniotic fluid. In embodiments, the neonate is less than 30, 25, 20, 15, 10, 5, 4, 3, or 2 days old. In embodiments, the neonate is less than 1 day old. In embodiments, fetal bacteria comprise, consist essentially of, or consist of fetal Micrococcus sp. bacteria and/or a fetal Lactobacillus sp. bacteria. In embodiments, a fetal bacterium is a fetal Micrococcus sp. bacterium. In embodiments, a fetal bacterium is a fetal Lactobacillus sp. bacterium. Non-limiting examples of fetal Micrococcus sp. bacteria and fetal Lactobacillus sp. bacteria are described herein. However, the present subject matter is not limited to the specific strains exemplified. Additional fetal Micrococcus sp. bacteria and fetal Lactobacillus sp. bacteria strains useful in methods and compositions disclosed herein are may be obtained using methods disclosed herein.

The phrase “stringent hybridization conditions” refers to conditions under which a primer or probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at T, 50% of the probes are occupied at equilibrium).

Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

In embodiments, nucleic acids hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous references, e.g., Current Protocols in Molecular Biology, ed. Ausubel, et al., supra.

In embodiments, detecting includes an assay. In embodiments, the assay is an analytic procedure to qualitatively assess or quantitatively measure the presence, amount, or functional activity of an entity, element, or feature (e.g., a bacterium, a genomic sequence, a compound such as a polynucleotide, a level of gene expression, a bacterial type or taxon, or a bacterial population such as in a microbiome). In embodiments, assaying the level of a compound includes using an analytic procedure (such as an in vitro procedure) to qualitatively assess or quantitatively measure the presence or amount of the compound.

In this disclosure, “comprises,” “comprising,” “containing” and “having” and the like can have the meaning ascribed to them in U.S. Patent law and can mean “includes,” “including,” and the like. “Consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

As used in the description herein and throughout the claims that follow, the meaning of “a,” “an,” and “the” includes plural reference unless the context clearly dictates otherwise.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “identical” or percent “identity,” in the context of two or more nucleic acids or polypeptide sequences, refer to two or more sequences or subsequences that are the same or have a specified percentage of amino acid residues or nucleotides that are the same (e.g., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire polypeptide sequence or an individual domain thereof), when compared and aligned for maximum correspondence over a comparison window, or designated region as measured using a sequence comparison algorithm or by manual alignment and visual inspection. In embodiments, two sequences are 100% identical. In embodiments, two sequences are 100% identical over the entire length of one of the sequences (e.g., the shorter of the two sequences where the sequences have different lengths). In embodiments, identity may refer to the complement of a test sequence. In embodiments, the identity exists over a region that is at least about 10 to about 100, about 20 to about 75, about 30 to about 50 amino acids or nucleotides in length. In embodiments, the identity exists over a region that is at least about 50 amino acids or nucleotides in length, or more preferably over a region that is 100 to 500, 100 to 200, 150 to 200, 175 to 200, 175 to 225, 175 to 250, 200 to 225, 200 to 250 or more amino acids or nucleotides in length.

For sequence comparison, typically one sequence acts as a reference sequence, to which test sequences are compared. In embodiments, when using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window” refers to a segment of any one of the number of contiguous positions (e.g., least about 10 to about 100, about 20 to about 75, about 30 to about 50, 100 to 500, 100 to 200, 150 to 200, 175 to 200, 175 to 225, 175 to 250, 200 to 225, 200 to 250) in which a sequence may be compared to a reference sequence of the same number of contiguous positions after the two sequences are optimally aligned. In embodiments, a comparison window is the entire length of one or both of two aligned sequences. In embodiments, two sequences being compared comprise different lengths, and the comparison window is the entire length of the longer or the shorter of the two sequences. In embodiments relating to two sequences of different lengths, the comparison window includes the entire length of the shorter of the two sequences. In embodiments relating to two sequences of different lengths, the comparison window includes the entire length of the longer of the two sequences.

Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Non-limiting examples of algorithms that are suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 may be used, with the parameters described herein, to determine percent sequence identity for nucleic acids and proteins. Software for performing BLAST analyses is publicly available through the National Center for Biotechnology Information (NCBI), as is known in the art. An exemplary BLAST algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. In embodiments, the NCBI BLASTN or BLASTP program is used to align sequences. In embodiments, the BLASTN or BLASTP program uses the defaults used by the NCBI. In embodiments, the BLASTN program (for nucleotide sequences) uses as defaults: a word size (W) of 28; an expectation threshold (E) of 10; max matches in a query range set to 0; match/mismatch scores of 1, -2; linear gap costs; the filter for low complexity regions used; and mask for lookup table only used. In certain embodiments, the BLASTP program (for amino acid sequences) uses as defaults: a word size (W) of 3; an expectation threshold (E) of 10; max matches in a query range set to 0; the BLOSUM62 matrix (see Henikoff & Henikoff, Proc. Natl. Acad. Sci. USA 89:10915 (1992)); gap costs of existence: 11 and extension: 1; and conditional compositional score matrix adjustment.

In embodiments, the SILVA database and its associated aligner SINA (the “SILVA Incremental Aligner”) is used for determining sequence similarity, e.g., to a highly curated 16S rRNA gene database. See, e.g., Pruesse, E., Peplies, J. and Glockner, F.O. (2012) SINA: accurate high-throughput multiple sequence alignment of ribosomal RNA genes. Bioinformatics, 28, 1823-1829, the entire content of which is incorporated herein by reference. In embodiments, the SINA is SINA 1.2.11. In embodiments, the SINA is available at www.arb-silva.de/aligner. In embodiments, the default settings at this website are: Gene=SSU; Bases remaining unalighed at the ends should be=“attached to the last aligned base”; Min identity with query sequence=0.95; Number of neighbours per query sequence=10; Program to use for tree computation=FastTree; Model for tree computation=GTR; Rate model for likelihoods=Gamma; Reject sequences below identity (%)=70.

Each embodiment disclosed herein is contemplated as being applicable to each of the other disclosed embodiments. Thus, all combinations of the various elements described herein are within the scope of the invention.

### II. METHODS OF TREATMENT, PREVENTION, AND RISK REDUCTION

In an aspect, provided herein is a method of treating, preventing, or reducing the risk of an inflammatory disease in a subject in need thereof. In embodiments, the method comprises administering to the subject an effective amount of a fetal Micrococcus sp. bacterium and/or a fetal Lactobacillus sp. bacterium.

In an aspect, provided herein is a method of treating, preventing, or reducing the risk of dysbiosis in a subject in need thereof. In embodiments, the method comprises administering to the subject an effective amount of a fetal Micrococcus sp. bacterium and/or a fetal Lactobacillus sp. bacterium.

In embodiments, the subject is pregnant. In embodiments, the subject has an increased risk for developing the inflammatory disease compared to a general population of healthy subjects. In embodiments, the subject has an inflammatory disease. In embodiments, the inflammatory disease is an allergy.

In embodiments, the allergy is an allergy to milk, eggs, fish, shellfish, a tree nut, peanuts, wheat, dander from a cat, dog, or rodent, an insect sting, pollen, latex, dust mites, or soybeans. In embodiments, the allergy is an allergy to milk. In embodiments, the allergy is an allergy to eggs. In embodiments, the allergy is an allergy to fish. In embodiments, the allergy is an allergy to shellfish. In embodiments, the allergy is an allergy to tree nut. In embodiments, the allergy is an allergy to peanuts. In embodiments, the allergy is an allergy to wheat. In embodiments, the allergy is an allergy to dander from a cat. In embodiments, the allergy is an allergy to dander from a dog. In embodiments, the allergy is an allergy to dander from a rodent. In embodiments, the allergy is an allergy to an insect sting. In embodiments, the allergy is an allergy to pollen. In embodiments, the allergy is an allergy to latex. In embodiments, the allergy is an allergy to dust mites. In embodiments, the allergy is an allergy to soybeans.

In embodiments, the allergy is pediatric allergic asthma, hay fever, or allergic airway sensitization. In embodiments, the allergy is a pediatric allergic asthma. In embodiments, the allergy is hay fever. In embodiments, the allergy is an allergic airway sensitization.

In embodiments, the inflammatory disease is a chronic inflammatory disease. In embodiments, the chronic inflammatory disease is asthma.

In embodiments, the inflammatory disease is an allergy, atopy, asthma, an autoimmune disease, an autoinflammatory disease, a hypersensitivity, pediatric allergic asthma, allergic asthma, inflammatory bowel disease, Celiac disease, Crohn's disease, colitis, ulcerative colitis, collagenous colitis, lymphocytic colitis, diverticulitis, irritable bowel syndrome, short bowel syndrome, stagnant loop syndrome, chronic persistent diarrhea, intractable diarrhea of infancy, Traveler's diarrhea, immunoproliferative small intestinal disease, chronic prostatitis, postenteritis syndrome, tropical sprue, Whipple's disease, Wolman disease, arthritis, rheumatoid arthritis, Behçet's disease, uveitis, pyoderma gangrenosum, erythema nodosum, traumatic brain injury, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, bullous pemphigoid, sarcoidosis, ichthyosis, Graves ophthalmopathy, Addison's disease, Vitiligo, acne vulgaris, pelvic inflammatory disease, reperfusion injury, sarcoidosis, transplant rejection, interstitial cystitis, atherosclerosis, and atopic dermatitis. In embodiments, the inflammatory disease is an allergy. In embodiments, the inflammatory disease is atopy. In embodiments, the inflammatory disease is asthma. In embodiments, the inflammatory disease is an autoimmune disease. In embodiments, the inflammatory disease is an autoinflammatory disease. In embodiments, the inflammatory disease is a hypersensitivity. In embodiments, the inflammatory disease is pediatric allergic asthma. In embodiments, the inflammatory disease is allergic asthma. In embodiments, the inflammatory disease is inflammatory bowel disease. In embodiments, the inflammatory disease is Celiac disease. In embodiments, the inflammatory disease is Crohn's disease. In embodiments, the inflammatory disease is colitis. In embodiments, the inflammatory disease is ulcerative colitis. In embodiments, the inflammatory disease is collagenous colitis. In embodiments, the inflammatory disease is lymphocytic colitis. In embodiments, the inflammatory disease is diverticulitis. In embodiments, the inflammatory disease is irritable bowel syndrome. In embodiments, the inflammatory disease is short bowel syndrome. In embodiments, the inflammatory disease is stagnant loop syndrome. In embodiments, the inflammatory disease is chronic persistent diarrhea. In embodiments, the inflammatory disease is intractable diarrhea of infancy. In embodiments, the inflammatory disease is Traveler's diarrhea. In embodiments, the inflammatory disease is immunoproliferative small intestinal disease. In embodiments, the inflammatory disease is chronic prostatitis. In embodiments, the inflammatory disease is postenteritis syndrome. In embodiments, the inflammatory disease is tropical sprue. In embodiments, the inflammatory disease is Whipple's disease. In embodiments, the inflammatory disease is Wolman disease. In embodiments, the inflammatory disease is arthritis. In embodiments, the inflammatory disease is rheumatoid arthritis. In embodiments, the inflammatory disease is Behçet's disease. In embodiments, the inflammatory disease is uveitis. In embodiments, the inflammatory disease is pyoderma gangrenosum. In embodiments, the inflammatory disease is erythema nodosum. In embodiments, the inflammatory disease is traumatic brain injury. In embodiments, the inflammatory disease is psoriatic arthritis. In embodiments, the inflammatory disease is juvenile idiopathic arthritis. In embodiments, the inflammatory disease is multiple sclerosis. In embodiments, the inflammatory disease is systemic lupus erythematosus (SLE). In embodiments, the inflammatory disease is myasthenia gravis. In embodiments, the inflammatory disease is juvenile onset diabetes. In embodiments, the inflammatory disease is diabetes mellitus type 1. In embodiments, the inflammatory disease is Guillain-Barre syndrome. In embodiments, the inflammatory disease is Hashimoto's encephalitis. In embodiments, the inflammatory disease is Hashimoto's thyroiditis. In embodiments, the inflammatory disease is ankylosing spondylitis. In embodiments, the inflammatory disease is psoriasis. In embodiments, the inflammatory disease is Sjogren's syndrome. In embodiments, the inflammatory disease is vasculitis. In embodiments, the inflammatory disease is glomerulonephritis. In embodiments, the inflammatory disease is auto-immune thyroiditis. In embodiments, the inflammatory disease is bullous pemphigoid. In embodiments, the inflammatory disease is sarcoidosis. In embodiments, the inflammatory disease is ichthyosis. In embodiments, the inflammatory disease is Graves ophthalmopathy. In embodiments, the inflammatory disease is Addison's disease. In embodiments, the inflammatory disease is Vitiligo. In embodiments, the inflammatory disease is acne vulgaris. In embodiments, the inflammatory disease is pelvic inflammatory disease. In embodiments, the inflammatory disease is reperfusion injury. In embodiments, the inflammatory disease is sarcoidosis. In embodiments, the inflammatory disease is transplant rejection. In embodiments, the inflammatory disease is interstitial cystitis. In embodiments, the inflammatory disease is atherosclerosis. In embodiments, the inflammatory disease is atopic dermatitis.

In embodiments, the subject has at least 1, 2, 3, or 4 cousins, grandparents, parents, aunts, uncles, and/or siblings who have been diagnosed with the inflammatory disease. In embodiments, the subject has at least 1 cousin, grandparent, parent, aunt, uncle, and/or sibling who have been diagnosed with the inflammatory disease. In embodiments, the subject has at least 2 cousins, grandparents, parents, aunts, uncles, and/or siblings who have been diagnosed with the inflammatory disease. In embodiments, the subject has at least 3 cousins, grandparents, parents, aunts, uncles, and/or siblings who have been diagnosed with the inflammatory disease. In embodiments, the subject has at least 4 cousins, grandparents, parents, aunts, uncles, and/or siblings who have been diagnosed with the inflammatory disease.

In embodiments, the mother of the subject has or has had asthma.

In embodiments, the subject has been in a room with a cat or a dog 0 times during the first month after the subject was born.

In embodiments, the subject has not lived in a residence with a cat or a dog for at least 7, 14, or 21 days of the first month after the subject was born. In embodiments, the subject has not lived in a residence with a cat or a dog for at least 7 days of the first month after the subject was born. In embodiments, the subject has not lived in a residence with a cat or a dog for at least 14 days of the first month after the subject was born. In embodiments, the subject has not lived in a residence with a cat or a dog for at least 21 days of the first month after the subject was born.

In embodiments, the subject's mother has not lived in a residence with a cat or a dog for at least 30, 60, 90, 120, 150, 180, 210, 240, or 270 days between when the subject was conceived and when the subject was born. In embodiments, the subject's mother has not lived in a residence with a cat or a dog for at least 30 days between when the subject was conceived and when the subject was born. In embodiments, the subject's mother has not lived in a residence with a cat or a dog for at least 60 days between when the subject was conceived and when the subject was born. In embodiments, the subject's mother has not lived in a residence with a cat or a dog for at least 90 days between when the subject was conceived and when the subject was born. In embodiments, the subject's mother has not lived in a residence with a cat or a dog for at least 120 days between when the subject was conceived and when the subject was born. In embodiments, the subject's mother has not lived in a residence with a cat or a dog for at least 150 days between when the subject was conceived and when the subject was born. In embodiments, the subject's mother has not lived in a residence with a cat or a dog for at least 180 days between when the subject was conceived and when the subject was born. In embodiments, the subject's mother has not lived in a residence with a cat or a dog for at least 210 days between when the subject was conceived and when the subject was born. In embodiments, the subject's mother has not lived in a residence with a cat or a dog for at least 240 days between when the subject was conceived and when the subject was born. In embodiments, the subject's mother has not lived in a residence with a cat or a dog for at least 270 days between when the subject was conceived and when the subject was born.

In embodiments, the subject's mother has smoked at least once on a total of at least about 30, 60, 90, 120, 150, 180, 210, 240, or 270 days between when the subject was conceived and when the subject was born. In embodiments, the subject's mother has smoked at least once on a total of at least about 30, 60, 90, 120, 150, 180, 210, 240, or 270 days between when the subject was conceived and when the subject was born. In embodiments, the subject's mother has smoked at least once on a total of at least about 30 days between when the subject was conceived and when the subject was born. In embodiments, the subject's mother has smoked at least once on a total of at least about 60 days between when the subject was conceived and when the subject was born. In embodiments, the subject's mother has smoked at least once on a total of at least about 90 days between when the subject was conceived and when the subject was born. In embodiments, the subject's mother has smoked at least once on a total of at least about 120 days between when the subject was conceived and when the subject was born. In embodiments, the subject's mother has smoked at least once on a total of at least about 150 days between when the subject was conceived and when the subject was born. In embodiments, the subject's mother has smoked at least once on a total of at least about 180 days between when the subject was conceived and when the subject was born. In embodiments, the subject's mother has smoked at least once on a total of at least about 210 days between when the subject was conceived and when the subject was born. In embodiments, the subject's mother has smoked at least once on a total of at least about 240 days between when the subject was conceived and when the subject was born. In embodiments, the subject's mother has smoked at least once on a total of at least about 270 days between when the subject was conceived and when the subject was born.

In embodiments, the days are consecutive days.

In embodiments, the subject has been fed formula in the first month of life.

In embodiments, the subject has not been fed breast milk in the first month of life.

In embodiments, the subject has a fecal level of 12,13 DiHOME of least about >398 ng/g.

In embodiments, the subject has a fecal level of 9,10 DiHOME of at least about >425 ng/g.

In embodiments, wherein the subject is a neonate.

In embodiments, the subject is less than about 1, 2, 3, 4, 5, 6, 7, 8, 9, 12, 18, or 24 months old. In embodiments, the subject is less than about 1 month old. In embodiments, the subject is less than about 2 months old. In embodiments, the subject is less than about 3 months old. In embodiments, the subject is less than about 4 months old. In embodiments, the subject is less than about 5 months old. In embodiments, the subject is less than about 6 months old. In embodiments, the subject is less than about 7 months old. In embodiments, the subject is less than about 8 months old. In embodiments, the subject is less than about 9 months old. In embodiments, the subject is less than about 12 months old. In embodiments, the subject is less than about 18 months old. In embodiments, the subject is less than about 24 months old.

In embodiments, the subject is between about 2 and about 18 years old, or is at least about 18 years old. In embodiments, the subject is between about 2 and about 18 years old. In embodiments, the subject is at least about 18 years old.

In embodiments, the subject is less than 1, 2, 3, 4, or 5 years old. In embodiments, the subject is less than 1 year old. In embodiments, the subject is less than 2 year old. In embodiments, the subject is less than 3 year old. In embodiments, the subject is less than 4 year old. In embodiments, the subject is less than 5 year old.

In embodiments, the subject is from 0 to 1 month old, from 0.5 to 2 months old, from 0 to 3 months old, 0.5 to 3 months old, from 3 to 6 months old, or from 0 to 6 months old. In embodiments, the subject is from 0 to 1 month old. In embodiments, the subject is from 0.5 to 2 months old. In embodiments, the subject from 0 to 3 months old. In embodiments, the subject is from 0.5 to 3 months old. In embodiments, the subject is from 3 to 6 months old. In embodiments, the subject is from 0 to 6 months old.

In an aspect, provided herein is a method of treating, preventing, or reducing the risk of dysbiosis in a neonatal subject. In embodiments, the method comprises administering to the subject an effective amount of a fetal Micrococcus sp. bacterium and/or a fetal Lactobacillus sp. bacterium.

In an aspect, provided herein is a method of reducing the risk that a neonatal subject will develop an inflammatory disease after birth. In embodiments, the method comprises administering to the subject an effective amount of a fetal Micrococcus sp. bacterium and/or a fetal Lactobacillus sp. bacterium.

In an aspect, provided herein is a method of treating, preventing, or reducing the risk of childhood obesity in a neonatal subject. In embodiments, the method comprises administering to the subject an effective amount of a fetal Micrococcus sp. bacterium and/or a fetal Lactobacillus sp. bacterium.

In embodiments, the neonatal subject was born by caesarean section.

In embodiments, the neonatal subject was born after less than 40, 39, 38, 37, 36, 35, 34, 33, 32, 31, or 30 weeks of gestation. In embodiments, wherein the neonatal subject was born after less than 40 weeks of gestation. In embodiments, wherein the neonatal subject was born after less than 39 weeks of gestation. In embodiments, wherein the neonatal subject was born after less than 38 weeks of gestation. In embodiments, wherein the neonatal subject was born after less than 37 weeks of gestation. In embodiments, wherein the neonatal subject was born after less than 36 weeks of gestation. In embodiments, wherein the neonatal subject was born after less than 35 weeks of gestation. In embodiments, wherein the neonatal subject was born after less than 34 weeks of gestation. In embodiments, wherein the neonatal subject was born after less than 33 weeks of gestation. In embodiments, wherein the neonatal subject was born after less than 32 weeks of gestation. In embodiments, wherein the neonatal subject was born after less than 31 weeks of gestation. In embodiments, wherein the neonatal subject was born after less than 30 weeks of gestation.

In embodiments, the neonatal subject is less than 1 month old.

In embodiments, the subject has an increased risk for developing the inflammatory disease compared to a general population of healthy subjects.

In embodiments, the subject has an inflammatory disease.

In embodiments, the inflammatory disease is an allergy.

In embodiments, the allergy is an allergy to milk, eggs, fish, shellfish, a tree nut, peanuts, wheat, dander from a cat, dog, or rodent, an insect sting, pollen, latex, dust mites, or soybeans. In embodiments, the allergy is an allergy to milk. In embodiments, the allergy is an allergy to eggs. In embodiments, the allergy is an allergy to fish. In embodiments, the allergy is an allergy to shellfish. In embodiments, the allergy is an allergy to tree nut. In embodiments, the allergy is an allergy to peanuts. In embodiments, the allergy is an allergy to wheat. In embodiments, the allergy is an allergy to dander from a cat. In embodiments, the allergy is an allergy to dander from a dog. In embodiments, the allergy is an allergy to dander from a rodent. In embodiments, the allergy is an allergy to an insect sting. In embodiments, the allergy is an allergy to pollen. In embodiments, the allergy is an allergy to latex. In embodiments, the allergy is an allergy to dust mites. In embodiments, the allergy is an allergy to soybeans.

In embodiments, the allergy is pediatric allergic asthma, hay fever, or allergic airway sensitization. In embodiments, the allergy is pediatric allergic asthma. In embodiments, the allergy is hay fever. In embodiments, the allergy is allergic airway sensitization.

In embodiments, the inflammatory disease is a chronic inflammatory disease. In embodiments, the chronic inflammatory disease is asthma.

In embodiments, the inflammatory disease is an allergy, atopy, asthma, an autoimmune disease, an autoinflammatory disease, a hypersensitivity, pediatric allergic asthma, allergic asthma, inflammatory bowel disease, Celiac disease, Crohn's disease, colitis, ulcerative colitis, collagenous colitis, lymphocytic colitis, diverticulitis, irritable bowel syndrome, short bowel syndrome, stagnant loop syndrome, chronic persistent diarrhea, intractable diarrhea of infancy, Traveler's diarrhea, immunoproliferative small intestinal disease, chronic prostatitis, postenteritis syndrome, tropical sprue, Whipple's disease, Wolman disease, arthritis, rheumatoid arthritis, Behçet's disease, uveitis, pyoderma gangrenosum, erythema nodosum, traumatic brain injury, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, bullous pemphigoid, sarcoidosis, ichthyosis, Graves ophthalmopathy, Addison's disease, Vitiligo, acne vulgaris, pelvic inflammatory disease, reperfusion injury, sarcoidosis, transplant rejection, interstitial cystitis, atherosclerosis, and atopic dermatitis. In embodiments. In embodiments, the inflammatory disease is the inflammatory disease is an allergy. In embodiments, the inflammatory disease is atopy. In embodiments, the inflammatory disease is asthma. In embodiments, the inflammatory disease is an autoimmune disease. In embodiments, the inflammatory disease is an autoinflammatory disease. In embodiments, the inflammatory disease is a hypersensitivity. In embodiments, the inflammatory disease is pediatric allergic asthma. In embodiments, the inflammatory disease is allergic asthma. In embodiments, the inflammatory disease is inflammatory bowel disease. In embodiments, the inflammatory disease is Celiac disease. In embodiments, the inflammatory disease is Crohn's disease. In embodiments, the inflammatory disease is colitis. In embodiments, the inflammatory disease is ulcerative colitis. In embodiments, the inflammatory disease is collagenous colitis. In embodiments, the inflammatory disease is lymphocytic colitis. In embodiments, the inflammatory disease is diverticulitis. In embodiments, the inflammatory disease is irritable bowel syndrome. In embodiments, the inflammatory disease is short bowel syndrome. In embodiments, the inflammatory disease is stagnant loop syndrome. In embodiments, the inflammatory disease is chronic persistent diarrhea. In embodiments, the inflammatory disease is intractable diarrhea of infancy. In embodiments, the inflammatory disease is Traveler's diarrhea. In embodiments, the inflammatory disease is immunoproliferative small intestinal disease. In embodiments, the inflammatory disease is chronic prostatitis. In embodiments, the inflammatory disease is postenteritis syndrome. In embodiments, the inflammatory disease is tropical sprue. In embodiments, the inflammatory disease is Whipple's disease. In embodiments, the inflammatory disease is Wolman disease. In embodiments, the inflammatory disease is arthritis. In embodiments, the inflammatory disease is rheumatoid arthritis. In embodiments, the inflammatory disease is Behçet's disease. In embodiments, the inflammatory disease is uveitis. In embodiments, the inflammatory disease is pyoderma gangrenosum. In embodiments, the inflammatory disease is erythema nodosum. In embodiments, the inflammatory disease is traumatic brain injury. In embodiments, the inflammatory disease is psoriatic arthritis. In embodiments, the inflammatory disease is juvenile idiopathic arthritis. In embodiments, the inflammatory disease is multiple sclerosis. In embodiments, the inflammatory disease is systemic lupus erythematosus (SLE). In embodiments, the inflammatory disease is myasthenia gravis. In embodiments, the inflammatory disease is juvenile onset diabetes. In embodiments, the inflammatory disease is diabetes mellitus type 1. In embodiments, the inflammatory disease is Guillain-Barre syndrome. In embodiments, the inflammatory disease is Hashimoto's encephalitis. In embodiments, the inflammatory disease is Hashimoto's thyroiditis. In embodiments, the inflammatory disease is ankylosing spondylitis. In embodiments, the inflammatory disease is psoriasis. In embodiments, the inflammatory disease is Sjogren's syndrome. In embodiments, the inflammatory disease is vasculitis. In embodiments, the inflammatory disease is glomerulonephritis. In embodiments, the inflammatory disease is autoimmune thyroiditis. In embodiments, the inflammatory disease is bullous pemphigoid. In embodiments, the inflammatory disease is sarcoidosis. In embodiments, the inflammatory disease is ichthyosis. In embodiments, the inflammatory disease is Graves ophthalmopathy. In embodiments, the inflammatory disease is Addison's disease. In embodiments, the inflammatory disease is Vitiligo. In embodiments, the inflammatory disease is acne vulgaris. In embodiments, the inflammatory disease is pelvic inflammatory disease. In embodiments, the inflammatory disease is reperfusion injury. In embodiments, the inflammatory disease is sarcoidosis. In embodiments, the inflammatory disease is transplant rejection. In embodiments, the inflammatory disease is interstitial cystitis. In embodiments, the inflammatory disease is atherosclerosis. In embodiments, the inflammatory disease is atopic dermatitis.

In embodiments, the subject has at least 1, 2, 3, or 4 cousins, grandparents, parents, aunts, uncles, and/or siblings who have been diagnosed with the inflammatory disease. In embodiments, the subject has at least 1 cousin, grandparent, parent, aunt, uncle, and/or sibling who have been diagnosed with the inflammatory disease. In embodiments, the subject has at least 2 cousins, grandparents, parents, aunts, uncles, and/or siblings who have been diagnosed with the inflammatory disease. In embodiments, the subject has at least 3 cousins, grandparents, parents, aunts, uncles, and/or siblings who have been diagnosed with the inflammatory disease. In embodiments, the subject has at least 4 cousins, grandparents, parents, aunts, uncles, and/or siblings who have been diagnosed with the inflammatory disease.

In embodiments, the mother of the subject has or has had asthma.

In embodiments, the subject has been in a room with a cat or a dog 0 times during the first month after the subject was born.

In embodiments, the subject has not lived in a residence with a cat or a dog for at least 7, 14, or 21 days of the first month after the subject was born. In embodiments, the subject has not lived in a residence with a cat or a dog for at least 7 days of the first month after the subject was born. In embodiments, the subject has not lived in a residence with a cat or a dog for at least 14 days of the first month after the subject was born. In embodiments, the subject has not lived in a residence with a cat or a dog for at least 21 days of the first month after the subject was born.

In embodiments, the subject's mother has not lived in a residence with a cat or a dog for at least 30, 60, 90, 120, 150, 180, 210, 240, or 270 days between when the subject was conceived and when the subject was born. In embodiments, the subject's mother has not lived in a residence with a cat or a dog for at least 30 days between when the subject was conceived and when the subject was born. In embodiments, the subject's mother has not lived in a residence with a cat or a dog for at least 60 days between when the subject was conceived and when the subject was born. In embodiments, the subject's mother has not lived in a residence with a cat or a dog for at least 90 days between when the subject was conceived and when the subject was born. In embodiments, the subject's mother has not lived in a residence with a cat or a dog for at least 120 days between when the subject was conceived and when the subject was born. In embodiments, the subject's mother has not lived in a residence with a cat or a dog for at least 150 days between when the subject was conceived and when the subject was born. In embodiments, the subject's mother has not lived in a residence with a cat or a dog for at least 180 days between when the subject was conceived and when the subject was born. In embodiments, the subject's mother has not lived in a residence with a cat or a dog for at least 210 days between when the subject was conceived and when the subject was born. In embodiments, the subject's mother has not lived in a residence with a cat or a dog for at least 240 days between when the subject was conceived and when the subject was born. In embodiments, the subject's mother has not lived in a residence with a cat or a dog for at least 270 days between when the subject was conceived and when the subject was born.

In embodiments, the subject's mother has smoked at least once on a total of at least about 30, 60, 90, 120, 150, 180, 210, 240, or 270 days between when the subject was conceived and when the subject was born. In embodiments, the subject's mother has smoked at least once on a total of at least about 30, 60, 90, 120, 150, 180, 210, 240, or 270 days between when the subject was conceived and when the subject was born. In embodiments, the subject's mother has smoked at least once on a total of at least about 30 days between when the subject was conceived and when the subject was born. In embodiments, the subject's mother has smoked at least once on a total of at least about 60 days between when the subject was conceived and when the subject was born. In embodiments, the subject's mother has smoked at least once on a total of at least about 90 days between when the subject was conceived and when the subject was born. In embodiments, the subject's mother has smoked at least once on a total of at least about 120 days between when the subject was conceived and when the subject was born. In embodiments, the subject's mother has smoked at least once on a total of at least about 150 days between when the subject was conceived and when the subject was born. In embodiments, the subject's mother has smoked at least once on a total of at least about 180 days between when the subject was conceived and when the subject was born. In embodiments, the subject's mother has smoked at least once on a total of at least about 210 days between when the subject was conceived and when the subject was born. In embodiments, the subject's mother has smoked at least once on a total of at least about 240 days between when the subject was conceived and when the subject was born. In embodiments, the subject's mother has smoked at least once on a total of at least about 270 days between when the subject was conceived and when the subject was born.

In embodiments, the days are consecutive days.

In embodiments, the subject has been fed formula in the first month of life.

In embodiments, the subject has not been fed breast milk in the first month of life.

In embodiments, the subject has a fecal level of 12,13 DiHOME of least about >398 ng/g.

In embodiments, the subject has a fecal level of 9,10 DiHOME of at least about >425 ng/g.

In embodiments, the subject, or the mother of the subject, has been identified as at risk of atopy or asthma according to, e.g., a method described in Levan et al. (2018) Neonatal gut-microbiome-derived 12,13 DiHOME impedes tolerance and promotes childhood atopy and asthma, bioRxiv (preprint) 311704; doi: doi.org/10.1101/311704, the entire content of which (including the supplementary material thereof) is incorporated herein by reference.

In an aspect, provided herein is a method of reducing the risk that a pregnant subject will give birth less than 37 completed weeks of gestation. In embodiments, the method comprises administering to the subject an effective amount of a fetal Micrococcus sp. bacterium and/or a fetal Lactobacillus sp. bacterium.

In embodiments, the subject has an increased risk of pre-term labor compared to a healthy population of pregnant subjects.

In embodiments, the subject has given birth less than 37 completed weeks of gestation during a previous pregnancy.

In embodiments, the subject is pregnant with multiple gestations.

In embodiments, the subject is less than 18 years old or more than 35 years old. In embodiments, the subject is less than 18 years old. In embodiments, the subject is more than 35 years old.

In embodiments, the subject has a urinary tract infection, has a sexually transmitted infection, has bacterial vaginosis, has trichomoniasis, has high blood pressure, has bleeding from the vagina, has a pregnancy resulting from in vitro fertilization, gave birth less than 6 months before the current pregnancy, has placenta previa, has diabetes, or has abnormal blood clotting. In embodiments, the subject has a urinary tract infection. In embodiments, the subject has a sexually transmitted infection. In embodiments, the subject has bacterial vaginosis. In embodiments, the subject has trichomoniasis. In embodiments, the subject has high blood pressure. In embodiments, the subject has bleeding from the vagina. In embodiments, the subject has a pregnancy resulting from in vitro fertilization. In embodiments, the subject gave birth less than 6 months before the current pregnancy. In embodiments, the subject has placenta previa. In embodiments, the subject has diabetes. In embodiments, the subject has abnormal blood clotting.

In an aspect, provided herein is a method of treating, preventing, or reducing the risk of inflammation in an unborn subject. In embodiments, the method comprises administering to the pregnant mother of the subject an effective amount of a fetal Micrococcus sp. bacterium and/or a fetal Lactobacillus sp. bacterium.

In an aspect, provided herein is a method of promoting or increasing immune system maturation or Treg function in an unborn subject. In embodiments, the method comprises administering to the pregnant mother of the subject an effective amount of a fetal Micrococcus sp. bacterium and/or a fetal Lactobacillus sp. bacterium.

In an aspect, provided herein is a method of treating, preventing, or reducing the risk of dysbiosis in an unborn subject. In embodiments, the method comprises administering to the pregnant mother of the subject an effective amount of a fetal Micrococcus sp. bacterium and/or a fetal Lactobacillus sp. bacterium.

In an aspect, provided herein is a method of reducing the risk that an unborn subject will develop an inflammatory disease after birth. In embodiments, the method comprises administering to the pregnant mother of the subject an effective amount of a fetal Micrococcus sp. bacterium and/or a fetal Lactobacillus sp. bacterium.

In an aspect, provided herein is a method of treating, preventing, or reducing the risk of childhood obesity in an unborn subject. In embodiments, the method comprises administering to the pregnant mother of the subject an effective amount of a fetal Micrococcus sp. bacterium and/or a fetal Lactobacillus sp. bacterium.

In embodiments, an unborn subject is a fetus.

In embodiments, the fetal Micrococcus sp. bacterium and/or the fetal Lactobacillus sp. bacterium is administered orally. In embodiments, the fetal Micrococcus sp. bacterium and the fetal Lactobacillus sp. bacterium is administered orally. In embodiments, the fetal Micrococcus sp. bacterium or the fetal Lactobacillus sp. bacterium is administered orally.

In embodiments, the subject is a female and the fetal Micrococcus sp. bacterium and/or the fetal Lactobacillus sp. bacterium is administered vaginally. In embodiments, the subject is a female and the fetal Micrococcus sp. bacterium and the fetal Lactobacillus sp. bacterium is administered vaginally. In embodiments, the subject is a female and the fetal Micrococcus sp. bacterium or the fetal Lactobacillus sp. bacterium is administered vaginally.

In embodiments, less than about 10, 9, 8, 7, 6, 5, 4, 3, or 2 different species of bacteria are administered. In embodiments, less than about 10 different species of bacteria are administered. In embodiments, less than about 9 different species of bacteria are administered. In embodiments, less than about 8 different species of bacteria are administered. In embodiments, less than about 7 different species of bacteria are administered. In embodiments, less than about 6 different species of bacteria are administered. In embodiments, less than about 5 different species of bacteria are administered. In embodiments, less than about 4 different species of bacteria are administered. In embodiments, less than about 3 different species of bacteria are administered. In embodiments, less than about 2 different species of bacteria are administered.

In embodiments, the nucleotide sequence of the 16S rRNA gene of the fetal Lactobacillus sp. bacterium is at least 95, 96, 97, 98, 99, or 99.5% identical to SEQ ID NO: 3. In embodiments, the nucleotide sequence of the 16S rRNA gene of the fetal Lactobacillus sp. bacterium is at least 95% identical to SEQ ID NO: 3. In embodiments, the nucleotide sequence of the 16S rRNA gene of the fetal Lactobacillus sp. bacterium is at least 96% identical to SEQ ID NO: 3. In embodiments, the nucleotide sequence of the 16S rRNA gene of the fetal Lactobacillus sp. bacterium is at least 97% identical to SEQ ID NO: 3. In embodiments, the nucleotide sequence of the 16S rRNA gene of the fetal Lactobacillus sp. bacterium is at least 97.1% identical to SEQ ID NO: 3. In embodiments, the nucleotide sequence of the 16S rRNA gene of the fetal Lactobacillus sp. bacterium is at least 97.2% identical to SEQ ID NO: 3. In embodiments, the nucleotide sequence of the 16S rRNA gene of the fetal Lactobacillus sp. bacterium is at least 97.3% identical to SEQ ID NO: 3. In embodiments, the nucleotide sequence of the 16S rRNA gene of the fetal Lactobacillus sp. bacterium is at least 97.4% identical to SEQ ID NO: 3. In embodiments, the nucleotide sequence of the 16S rRNA gene of the fetal Lactobacillus sp. bacterium is at least 97.5% identical to SEQ ID NO: 3. In embodiments, the nucleotide sequence of the 16S rRNA gene of the fetal Lactobacillus sp. bacterium is at least 97.6% identical to SEQ ID NO: 3. In embodiments, the nucleotide sequence of the 16S rRNA gene of the fetal Lactobacillus sp. bacterium is at least 97.7% identical to SEQ ID NO: 3. In embodiments, the nucleotide sequence of the 16S rRNA gene of the fetal Lactobacillus sp. bacterium is at least 97.8% identical to SEQ ID NO: 3. In embodiments, the nucleotide sequence of the 16S rRNA gene of the fetal Lactobacillus sp. bacterium is at least 97.9% identical to SEQ ID NO: 3. In embodiments, the nucleotide sequence of the 16S rRNA gene of the fetal Lactobacillus sp. bacterium is at least 98% identical to SEQ ID NO: 3. In embodiments, the nucleotide sequence of the 16S rRNA gene of the fetal Lactobacillus sp. bacterium is at least 98.1% identical to SEQ ID NO: 3. In embodiments, the nucleotide sequence of the 16S rRNA gene of the fetal Lactobacillus sp. bacterium is at least 98.2% identical to SEQ ID NO: 3. In embodiments, the nucleotide sequence of the 16S rRNA gene of the fetal Lactobacillus sp. bacterium is at least 98.3% identical to SEQ ID NO: 3. In embodiments, the nucleotide sequence of the 16S rRNA gene of the fetal Lactobacillus sp. bacterium is at least 98.4% identical to SEQ ID NO: 3. In embodiments, the nucleotide sequence of the 16S rRNA gene of the fetal Lactobacillus sp. bacterium is at least 98.5% identical to SEQ ID NO: 3. In embodiments, the nucleotide sequence of the 16S rRNA gene of the fetal Lactobacillus sp. bacterium is at least 98.6% identical to SEQ ID NO: 3. In embodiments, the nucleotide sequence of the 16S rRNA gene of the fetal Lactobacillus sp. bacterium is at least 98.7% identical to SEQ ID NO: 3. In embodiments, the nucleotide sequence of the 16S rRNA gene of the fetal Lactobacillus sp. bacterium is at least 98.8% identical to SEQ ID NO: 3. In embodiments, the nucleotide sequence of the 16S rRNA gene of the fetal Lactobacillus sp. bacterium is at least 98.9% identical to SEQ ID NO: 3. In embodiments, the nucleotide sequence of the 16S rRNA gene of the fetal Lactobacillus sp. bacterium is at least 99% identical to SEQ ID NO: 3. In embodiments, the nucleotide sequence of the 16S rRNA gene of the fetal Lactobacillus sp. bacterium is at least 99.1% identical to SEQ ID NO: 3. In embodiments, the nucleotide sequence of the 16S rRNA gene of the fetal Lactobacillus sp. bacterium is at least 99.2% identical to SEQ ID NO: 3. In embodiments, the nucleotide sequence of the 16S rRNA gene of the fetal Lactobacillus sp. bacterium is at least 99.3% identical to SEQ ID NO: 3. In embodiments, the nucleotide sequence of the 16S rRNA gene of the fetal Lactobacillus sp. bacterium is at least 99.4% identical to SEQ ID NO: 3. In embodiments, the nucleotide sequence of the 16S rRNA gene of the fetal Lactobacillus sp. bacterium is at least 99.5% identical to SEQ ID NO: 3. In embodiments, the nucleotide sequence of the 16S rRNA gene of the fetal Lactobacillus sp. bacterium is at least 99.6% identical to SEQ ID NO: 3. In embodiments, the nucleotide sequence of the 16S rRNA gene of the fetal Lactobacillus sp. bacterium is at least 99.7% identical to SEQ ID NO: 3. In embodiments, the nucleotide sequence of the 16S rRNA gene of the fetal Lactobacillus sp. bacterium is at least 99.8% identical to SEQ ID NO: 3. In embodiments, the nucleotide sequence of the 16S rRNA gene of the fetal Lactobacillus sp. bacterium is at least 99.9% identical to SEQ ID NO: 3. In embodiments, the nucleotide sequence of the 16S rRNA gene of the fetal Lactobacillus sp. bacterium is identical to SEQ ID NO: 3.

In embodiments, the nucleotide sequence of the 16S rRNA gene of the fetal Lactobacillus sp. bacterium is at least 95, 96, 97, 98, 99, or 99.5% identical to SEQ ID NO: 5. In embodiments, the nucleotide sequence of the 16S rRNA gene of the fetal Lactobacillus sp. bacterium is at least 95% identical to SEQ ID NO: 5. In embodiments, the nucleotide sequence of the 16S rRNA gene of the fetal Lactobacillus sp. bacterium is at least 96% identical to SEQ ID NO: 5. In embodiments, the nucleotide sequence of the 16S rRNA gene of the fetal Lactobacillus sp. bacterium is at least 97% identical to SEQ ID NO: 5. In embodiments, the nucleotide sequence of the 16S rRNA gene of the fetal Lactobacillus sp. bacterium is at least 97.1% identical to SEQ ID NO: 5. In embodiments, the nucleotide sequence of the 16S rRNA gene of the fetal Lactobacillus sp. bacterium is at least 97.2% identical to SEQ ID NO: 5. In embodiments, the nucleotide sequence of the 16S rRNA gene of the fetal Lactobacillus sp. bacterium is at least 97.3% identical to SEQ ID NO: 5. In embodiments, the nucleotide sequence of the 16S rRNA gene of the fetal Lactobacillus sp. bacterium is at least 97.4% identical to SEQ ID NO: 5. In embodiments, the nucleotide sequence of the 16S rRNA gene of the fetal Lactobacillus sp. bacterium is at least 97.5% identical to SEQ ID NO: 5. In embodiments, the nucleotide sequence of the 16S rRNA gene of the fetal Lactobacillus sp. bacterium is at least 97.6% identical to SEQ ID NO: 5. In embodiments, the nucleotide sequence of the 16S rRNA gene of the fetal Lactobacillus sp. bacterium is at least 97.7% identical to SEQ ID NO: 5. In embodiments, the nucleotide sequence of the 16S rRNA gene of the fetal Lactobacillus sp. bacterium is at least 97.8% identical to SEQ ID NO: 5. In embodiments, the nucleotide sequence of the 16S rRNA gene of the fetal Lactobacillus sp. bacterium is at least 97.9% identical to SEQ ID NO: 5. In embodiments, the nucleotide sequence of the 16S rRNA gene of the fetal Lactobacillus sp. bacterium is at least 98% identical to SEQ ID NO: 5. In embodiments, the nucleotide sequence of the 16S rRNA gene of the fetal Lactobacillus sp. bacterium is at least 98.1% identical to SEQ ID NO: 5. In embodiments, the nucleotide sequence of the 16S rRNA gene of the fetal Lactobacillus sp. bacterium is at least 98.2% identical to SEQ ID NO: 5. In embodiments, the nucleotide sequence of the 16S rRNA gene of the fetal Lactobacillus sp. bacterium is at least 98.3% identical to SEQ ID NO: 5. In embodiments, the nucleotide sequence of the 16S rRNA gene of the fetal Lactobacillus sp. bacterium is at least 98.4% identical to SEQ ID NO: 5. In embodiments, the nucleotide sequence of the 16S rRNA gene of the fetal Lactobacillus sp. bacterium is at least 98.5% identical to SEQ ID NO: 5. In embodiments, the nucleotide sequence of the 16S rRNA gene of the fetal Lactobacillus sp. bacterium is at least 98.6% identical to SEQ ID NO: 5. In embodiments, the nucleotide sequence of the 16S rRNA gene of the fetal Lactobacillus sp. bacterium is at least 98.7% identical to SEQ ID NO: 5. In embodiments, the nucleotide sequence of the 16S rRNA gene of the fetal Lactobacillus sp. bacterium is at least 98.8% identical to SEQ ID NO: 5. In embodiments, the nucleotide sequence of the 16S rRNA gene of the fetal Lactobacillus sp. bacterium is at least 98.9% identical to SEQ ID NO: 5. In embodiments, the nucleotide sequence of the 16S rRNA gene of the fetal Lactobacillus sp. bacterium is at least 99% identical to SEQ ID NO: 5. In embodiments, the nucleotide sequence of the 16S rRNA gene of the fetal Lactobacillus sp. bacterium is at least 99.1% identical to SEQ ID NO: 5. In embodiments, the nucleotide sequence of the 16S rRNA gene of the fetal Lactobacillus sp. bacterium is at least 99.2% identical to SEQ ID NO: 5. In embodiments, the nucleotide sequence of the 16S rRNA gene of the fetal Lactobacillus sp. bacterium is at least 99.3% identical to SEQ ID NO: 5. In embodiments, the nucleotide sequence of the 16S rRNA gene of the fetal Lactobacillus sp. bacterium is at least 99.4% identical to SEQ ID NO: 5. In embodiments, the nucleotide sequence of the 16S rRNA gene of the fetal Lactobacillus sp. bacterium is at least 99.5% identical to SEQ ID NO: 5. In embodiments, the nucleotide sequence of the 16S rRNA gene of the fetal Lactobacillus sp. bacterium is at least 99.6% identical to SEQ ID NO: 5. In embodiments, the nucleotide sequence of the 16S rRNA gene of the fetal Lactobacillus sp. bacterium is at least 99.7% identical to SEQ ID NO: 5. In embodiments, the nucleotide sequence of the 16S rRNA gene of the fetal Lactobacillus sp. bacterium is at least 99.8% identical to SEQ ID NO: 5. In embodiments, the nucleotide sequence of the 16S rRNA gene of the fetal Lactobacillus sp. bacterium is at least 99.9% identical to SEQ ID NO: 5. In embodiments, the nucleotide sequence of the 16S rRNA gene of the fetal Lactobacillus sp. bacterium is identical to SEQ ID NO: 5.

In embodiments, the nucleotide sequence of V4 region of the 16S rRNA gene of the fetal Lactobacillus sp. bacterium is at least 95, 96, 97, 98, 99, or 99.5% identical to SEQ ID NO: 6. In embodiments, the nucleotide sequence of V4 region of the 16S rRNA gene of the fetal Lactobacillus sp. bacterium is at least 95% identical to SEQ ID NO: 6. In embodiments, the nucleotide sequence of V4 region of the 16S rRNA gene of the fetal Lactobacillus sp. bacterium is at least 96% identical to SEQ ID NO: 6. In embodiments, the nucleotide sequence of V4 region of the 16S rRNA gene of the fetal Lactobacillus sp. bacterium is at least 97% identical to SEQ ID NO: 6. In embodiments, the nucleotide sequence of V4 region of the 16S rRNA gene of the fetal Lactobacillus sp. bacterium is at least 97.1% identical to SEQ ID NO: 6. In embodiments, the nucleotide sequence of V4 region of the 16S rRNA gene of the fetal Lactobacillus sp. bacterium is at least 97.2% identical to SEQ ID NO: 6. In embodiments, the nucleotide sequence of V4 region of the 16S rRNA gene of the fetal Lactobacillus sp. bacterium is at least 97.3% identical to SEQ ID NO: 6. In embodiments, the nucleotide sequence of V4 region of the 16S rRNA gene of the fetal Lactobacillus sp. bacterium is at least 97.4% identical to SEQ ID NO: 6. In embodiments, the nucleotide sequence of V4 region of the 16S rRNA gene of the fetal Lactobacillus sp. bacterium is at least 97.5% identical to SEQ ID NO: 6. In embodiments, the nucleotide sequence of V4 region of the 16S rRNA gene of the fetal Lactobacillus sp. bacterium is at least 97.6% identical to SEQ ID NO: 6. In embodiments, the nucleotide sequence of V4 region of the 16S rRNA gene of the fetal Lactobacillus sp. bacterium is at least 97.7% identical to SEQ ID NO: 6. In embodiments, the nucleotide sequence of V4 region of the 16S rRNA gene of the fetal Lactobacillus sp. bacterium is at least 97.8% identical to SEQ ID NO: 6. In embodiments, the nucleotide sequence of V4 region of the 16S rRNA gene of the fetal Lactobacillus sp. bacterium is at least 97.9% identical to SEQ ID NO: 6. In embodiments, the nucleotide sequence of V4 region of the 16S rRNA gene of the fetal Lactobacillus sp. bacterium is at least 98% identical to SEQ ID NO: 6. In embodiments, the nucleotide sequence of V4 region of the 16S rRNA gene of the fetal Lactobacillus sp. bacterium is at least 98.1% identical to SEQ ID NO: 6. In embodiments, the nucleotide sequence of V4 region of the 16S rRNA gene of the fetal Lactobacillus sp. bacterium is at least 98.2% identical to SEQ ID NO: 6. In embodiments, the nucleotide sequence of V4 region of the 16S rRNA gene of the fetal Lactobacillus sp. bacterium is at least 98.3% identical to SEQ ID NO: 6. In embodiments, the nucleotide sequence of V4 region of the 16S rRNA gene of the fetal Lactobacillus sp. bacterium is at least 98.4% identical to SEQ ID NO: 6. In embodiments, the nucleotide sequence of V4 region of the 16S rRNA gene of the fetal Lactobacillus sp. bacterium is at least 98.5% identical to SEQ ID NO: 6. In embodiments, the nucleotide sequence of V4 region of the 16S rRNA gene of the fetal Lactobacillus sp. bacterium is at least 98.6% identical to SEQ ID NO: 6. In embodiments, the nucleotide sequence of V4 region of the 16S rRNA gene of the fetal Lactobacillus sp. bacterium is at least 98.7% identical to SEQ ID NO: 6. In embodiments, the nucleotide sequence of V4 region of the 16S rRNA gene of the fetal Lactobacillus sp. bacterium is at least 98.8% identical to SEQ ID NO: 6. In embodiments, the nucleotide sequence of V4 region of the 16S rRNA gene of the fetal Lactobacillus sp. bacterium is at least 98.9% identical to SEQ ID NO: 6. In embodiments, the nucleotide sequence of V4 region of the 16S rRNA gene of the fetal Lactobacillus sp. bacterium is at least 99% identical to SEQ ID NO: 6. In embodiments, the nucleotide sequence of V4 region of the 16S rRNA gene of the fetal Lactobacillus sp. bacterium is at least 99.1% identical to SEQ ID NO: 6. In embodiments, the nucleotide sequence of V4 region of the 16S rRNA gene of the fetal Lactobacillus sp. bacterium is at least 99.2% identical to SEQ ID NO: 6. In embodiments, the nucleotide sequence of V4 region of the 16S rRNA gene of the fetal Lactobacillus sp. bacterium is at least 99.3% identical to SEQ ID NO: 6. In embodiments, the nucleotide sequence of V4 region of the 16S rRNA gene of the fetal Lactobacillus sp. bacterium is at least 99.4% identical to SEQ ID NO: 6. In embodiments, the nucleotide sequence of V4 region of the 16S rRNA gene of the fetal Lactobacillus sp. bacterium is at least 99.5% identical to SEQ ID NO: 6. In embodiments, the nucleotide sequence of V4 region of the 16S rRNA gene of the fetal Lactobacillus sp. bacterium is at least 99.6% identical to SEQ ID NO: 6. In embodiments, the nucleotide sequence of V4 region of the 16S rRNA gene of the fetal Lactobacillus sp. bacterium is at least 99.7% identical to SEQ ID NO: 6. In embodiments, the nucleotide sequence of V4 region of the 16S rRNA gene of the fetal Lactobacillus sp. bacterium is at least 99.8% identical to SEQ ID NO: 6. In embodiments, the nucleotide sequence of V4 region of the 16S rRNA gene of the fetal Lactobacillus sp. bacterium is at least 99.9% identical to SEQ ID NO: 6. In embodiments, the nucleotide sequence of the V4 region of the 16S rRNA gene of the fetal Lactobacillus sp. bacterium is identical to SEQ ID NO: 6.

In embodiments, the nucleotide sequence of V4 region of the 16S rRNA gene of the fetal Lactobacillus sp. bacterium is at least 95, 96, 97, 98, 99, or 99.5% identical to SEQ ID NO: 1. In embodiments, the nucleotide sequence of V4 region of the 16S rRNA gene of the fetal Lactobacillus sp. bacterium is at least 95% identical to SEQ ID NO: 1. In embodiments, the nucleotide sequence of V4 region of the 16S rRNA gene of the fetal Lactobacillus sp. bacterium is at least 96% identical to SEQ ID NO: 1. In embodiments, the nucleotide sequence of V4 region of the 16S rRNA gene of the fetal Lactobacillus sp. bacterium is at least 97% identical to SEQ ID NO: 1. In embodiments, the nucleotide sequence of V4 region of the 16S rRNA gene of the fetal Lactobacillus sp. bacterium is at least 97.1% identical to SEQ ID NO: 1. In embodiments, the nucleotide sequence of V4 region of the 16S rRNA gene of the fetal Lactobacillus sp. bacterium is at least 97.2% identical to SEQ ID NO: 1. In embodiments, the nucleotide sequence of V4 region of the 16S rRNA gene of the fetal Lactobacillus sp. bacterium is at least 97.3% identical to SEQ ID NO: 1. In embodiments, the nucleotide sequence of V4 region of the 16S rRNA gene of the fetal Lactobacillus sp. bacterium is at least 97.4% identical to SEQ ID NO: 1. In embodiments, the nucleotide sequence of V4 region of the 16S rRNA gene of the fetal Lactobacillus sp. bacterium is at least 97.5% identical to SEQ ID NO: 1. In embodiments, the nucleotide sequence of V4 region of the 16S rRNA gene of the fetal Lactobacillus sp. bacterium is at least 97.6% identical to SEQ ID NO: 1. In embodiments, the nucleotide sequence of V4 region of the 16S rRNA gene of the fetal Lactobacillus sp. bacterium is at least 97.7% identical to SEQ ID NO: 1. In embodiments, the nucleotide sequence of V4 region of the 16S rRNA gene of the fetal Lactobacillus sp. bacterium is at least 97.8% identical to SEQ ID NO: 1. In embodiments, the nucleotide sequence of V4 region of the 16S rRNA gene of the fetal Lactobacillus sp. bacterium is at least 97.9% identical to SEQ ID NO: 1. In embodiments, the nucleotide sequence of V4 region of the 16S rRNA gene of the fetal Lactobacillus sp. bacterium is at least 98% identical to SEQ ID NO: 1. In embodiments, the nucleotide sequence of V4 region of the 16S rRNA gene of the fetal Lactobacillus sp. bacterium is at least 98.1% identical to SEQ ID NO: 1. In embodiments, the nucleotide sequence of V4 region of the 16S rRNA gene of the fetal Lactobacillus sp. bacterium is at least 98.2% identical to SEQ ID NO: 1. In embodiments, the nucleotide sequence of V4 region of the 16S rRNA gene of the fetal Lactobacillus sp. bacterium is at least 98.3% identical to SEQ ID NO: 1. In embodiments, the nucleotide sequence of V4 region of the 16S rRNA gene of the fetal Lactobacillus sp. bacterium is at least 98.4% identical to SEQ ID NO: 1. In embodiments, the nucleotide sequence of V4 region of the 16S rRNA gene of the fetal Lactobacillus sp. bacterium is at least 98.5% identical to SEQ ID NO: 1. In embodiments, the nucleotide sequence of V4 region of the 16S rRNA gene of the fetal Lactobacillus sp. bacterium is at least 98.6% identical to SEQ ID NO: 1. In embodiments, the nucleotide sequence of V4 region of the 16S rRNA gene of the fetal Lactobacillus sp. bacterium is at least 98.7% identical to SEQ ID NO: 1. In embodiments, the nucleotide sequence of V4 region of the 16S rRNA gene of the fetal Lactobacillus sp. bacterium is at least 98.8% identical to SEQ ID NO: 1. In embodiments, the nucleotide sequence of V4 region of the 16S rRNA gene of the fetal Lactobacillus sp. bacterium is at least 98.9% identical to SEQ ID NO: 1. In embodiments, the nucleotide sequence of V4 region of the 16S rRNA gene of the fetal Lactobacillus sp. bacterium is at least 99% identical to SEQ ID NO: 1. In embodiments, the nucleotide sequence of V4 region of the 16S rRNA gene of the fetal Lactobacillus sp. bacterium is at least 99.1% identical to SEQ ID NO: 1. In embodiments, the nucleotide sequence of V4 region of the 16S rRNA gene of the fetal Lactobacillus sp. bacterium is at least 99.2% identical to SEQ ID NO: 1. In embodiments, the nucleotide sequence of V4 region of the 16S rRNA gene of the fetal Lactobacillus sp. bacterium is at least 99.3% identical to SEQ ID NO: 1. In embodiments, the nucleotide sequence of V4 region of the 16S rRNA gene of the fetal Lactobacillus sp. bacterium is at least 99.4% identical to SEQ ID NO: 1. In embodiments, the nucleotide sequence of V4 region of the 16S rRNA gene of the fetal Lactobacillus sp. bacterium is at least 99.5% identical to SEQ ID NO: 1. In embodiments, the nucleotide sequence of V4 region of the 16S rRNA gene of the fetal Lactobacillus sp. bacterium is at least 99.6% identical to SEQ ID NO: 1. In embodiments, the nucleotide sequence of V4 region of the 16S rRNA gene of the fetal Lactobacillus sp. bacterium is at least 99.7% identical to SEQ ID NO: 1. In embodiments, the nucleotide sequence of V4 region of the 16S rRNA gene of the fetal Lactobacillus sp. bacterium is at least 99.8% identical to SEQ ID NO: 1. In embodiments, the nucleotide sequence of V4 region of the 16S rRNA gene of the fetal Lactobacillus sp. bacterium is at least 99.9% identical to SEQ ID NO: 1. In embodiments, the nucleotide sequence of the V4 region of the 16S rRNA gene of the fetal Lactobacillus sp. bacterium is identical to SEQ ID NO: 1.

In embodiments, the nucleotide sequence of the 16S rRNA gene of the fetal Micrococcus sp. bacterium is at least 95, 96, 97, 98, 99, or 99.5% identical to SEQ ID NO: 4. In embodiments, the nucleotide sequence of the 16S rRNA gene of the fetal Micrococcus sp. bacterium is at least 95% identical to SEQ ID NO: 4. In embodiments, the nucleotide sequence of the 16S rRNA gene of the fetal Micrococcus sp. bacterium is at least 96% identical to SEQ ID NO: 4. In embodiments, the nucleotide sequence of the 16S rRNA gene of the fetal Micrococcus sp. bacterium is at least 97% identical to SEQ ID NO: 4. In embodiments, the nucleotide sequence of the 16S rRNA gene of the fetal Micrococcus sp. bacterium is at least 97.1% identical to SEQ ID NO: 4. In embodiments, the nucleotide sequence of the 16S rRNA gene of the fetal Micrococcus sp. bacterium is at least 97.2% identical to SEQ ID NO: 4. In embodiments, the nucleotide sequence of the 16S rRNA gene of the fetal Micrococcus sp. bacterium is at least 97.3% identical to SEQ ID NO: 4. In embodiments, the nucleotide sequence of the 16S rRNA gene of the fetal Micrococcus sp. bacterium is at least 97.4% identical to SEQ ID NO: 4. In embodiments, the nucleotide sequence of the 16S rRNA gene of the fetal Micrococcus sp. bacterium is at least 97.5% identical to SEQ ID NO: 4. In embodiments, the nucleotide sequence of the 16S rRNA gene of the fetal Micrococcus sp. bacterium is at least 97.6% identical to SEQ ID NO: 4. In embodiments, the nucleotide sequence of the 16S rRNA gene of the fetal Micrococcus sp. bacterium is at least 97.7% identical to SEQ ID NO: 4. In embodiments, the nucleotide sequence of the 16S rRNA gene of the fetal Micrococcus sp. bacterium is at least 97.8% identical to SEQ ID NO: 4. In embodiments, the nucleotide sequence of the 16S rRNA gene of the fetal Micrococcus sp. bacterium is at least 97.9% identical to SEQ ID NO: 4. In embodiments, the nucleotide sequence of the 16S rRNA gene of the fetal Micrococcus sp. bacterium is at least 98% identical to SEQ ID NO: 4. In embodiments, the nucleotide sequence of the 16S rRNA gene of the fetal Micrococcus sp. bacterium is at least 98.1% identical to SEQ ID NO: 4. In embodiments, the nucleotide sequence of the 16S rRNA gene of the fetal Micrococcus sp. bacterium is at least 98.2% identical to SEQ ID NO: 4. In embodiments, the nucleotide sequence of the 16S rRNA gene of the fetal Micrococcus sp. bacterium is at least 98.3% identical to SEQ ID NO: 4. In embodiments, the nucleotide sequence of the 16S rRNA gene of the fetal Micrococcus sp. bacterium is at least 98.4% identical to SEQ ID NO: 4. In embodiments, the nucleotide sequence of the 16S rRNA gene of the fetal Micrococcus sp. bacterium is at least 98.5% identical to SEQ ID NO: 4. In embodiments, the nucleotide sequence of the 16S rRNA gene of the fetal Micrococcus sp. bacterium is at least 98.6% identical to SEQ ID NO: 4. In embodiments, the nucleotide sequence of the 16S rRNA gene of the fetal Micrococcus sp. bacterium is at least 98.7% identical to SEQ ID NO: 4. In embodiments, the nucleotide sequence of the 16S rRNA gene of the fetal Micrococcus sp. bacterium is at least 98.8% identical to SEQ ID NO: 4. In embodiments, the nucleotide sequence of the 16S rRNA gene of the fetal Micrococcus sp. bacterium is at least 98.9% identical to SEQ ID NO: 4. In embodiments, the nucleotide sequence of the 16S rRNA gene of the fetal Micrococcus sp. bacterium is at least 99% identical to SEQ ID NO: 4. In embodiments, the nucleotide sequence of the 16S rRNA gene of the fetal Micrococcus sp. bacterium is at least 99.1% identical to SEQ ID NO: 4. In embodiments, the nucleotide sequence of the 16S rRNA gene of the fetal Micrococcus sp. bacterium is at least 99.2% identical to SEQ ID NO: 4. In embodiments, the nucleotide sequence of the 16S rRNA gene of the fetal Micrococcus sp. bacterium is at least 99.3% identical to SEQ ID NO: 4. In embodiments, the nucleotide sequence of the 16S rRNA gene of the fetal Micrococcus sp. bacterium is at least 99.4% identical to SEQ ID NO: 4. In embodiments, the nucleotide sequence of the 16S rRNA gene of the fetal Micrococcus sp. bacterium is at least 99.5% identical to SEQ ID NO: 4. In embodiments, the nucleotide sequence of the 16S rRNA gene of the fetal Micrococcus sp. bacterium is at least 99.6% identical to SEQ ID NO: 4. In embodiments, the nucleotide sequence of the 16S rRNA gene of the fetal Micrococcus sp. bacterium is at least 99.7% identical to SEQ ID NO: 4. In embodiments, the nucleotide sequence of the 16S rRNA gene of the fetal Micrococcus sp. bacterium is at least 99.8% identical to SEQ ID NO: 4. In embodiments, the nucleotide sequence of the 16S rRNA gene of the fetal Micrococcus sp. bacterium is at least 99.9% identical to SEQ ID NO: 4. In embodiments, the nucleotide sequence of the 16S rRNA gene of the fetal Micrococcus sp. bacterium is identical to SEQ ID NO: 4.

In embodiments, the nucleotide sequence of the V4 region of the 16S rRNA gene of the fetal Micrococcus sp. bacterium is at least 95, 96, 97, 98, 99, or 99.5% identical to SEQ ID NO: 2. In embodiments, the nucleotide sequence of the V4 region of the 16S rRNA gene of the fetal Micrococcus sp. bacterium is at least 95% identical to SEQ ID NO: 2. In embodiments, the nucleotide sequence of the V4 region of the 16S rRNA gene of the fetal Micrococcus sp. bacterium is at least 96% identical to SEQ ID NO: 2. In embodiments, the nucleotide sequence of the V4 region of the 16S rRNA gene of the fetal Micrococcus sp. bacterium is at least 97% identical to SEQ ID NO: 2. In embodiments, the nucleotide sequence of the V4 region of the 16S rRNA gene of the fetal Micrococcus sp. bacterium is at least 97.1% identical to SEQ ID NO: 2. In embodiments, the nucleotide sequence of the V4 region of the 16S rRNA gene of the fetal Micrococcus sp. bacterium is at least 97.2% identical to SEQ ID NO: 2. In embodiments, the nucleotide sequence of the V4 region of the 16S rRNA gene of the fetal Micrococcus sp. bacterium is at least 97.3% identical to SEQ ID NO: 2. In embodiments, the nucleotide sequence of the V4 region of the 16S rRNA gene of the fetal Micrococcus sp. bacterium is at least 97.4% identical to SEQ ID NO: 2. In embodiments, the nucleotide sequence of the V4 region of the 16S rRNA gene of the fetal Micrococcus sp. bacterium is at least 97.5% identical to SEQ ID NO: 2. In embodiments, the nucleotide sequence of the V4 region of the 16S rRNA gene of the fetal Micrococcus sp. bacterium is at least 97.6% identical to SEQ ID NO: 2. In embodiments, the nucleotide sequence of the V4 region of the 16S rRNA gene of the fetal Micrococcus sp. bacterium is at least 97.7% identical to SEQ ID NO: 2. In embodiments, the nucleotide sequence of the V4 region of the 16S rRNA gene of the fetal Micrococcus sp. bacterium is at least 97.8% identical to SEQ ID NO: 2. In embodiments, the nucleotide sequence of the V4 region of the 16S rRNA gene of the fetal Micrococcus sp. bacterium is at least 97.9% identical to SEQ ID NO: 2. In embodiments, the nucleotide sequence of the V4 region of the 16S rRNA gene of the fetal Micrococcus sp. bacterium is at least 98% identical to SEQ ID NO: 2. In embodiments, the nucleotide sequence of the V4 region of the 16S rRNA gene of the fetal Micrococcus sp. bacterium is at least 98.1% identical to SEQ ID NO: 2. In embodiments, the nucleotide sequence of the V4 region of the 16S rRNA gene of the fetal Micrococcus sp. bacterium is at least 98.2% identical to SEQ ID NO: 2. In embodiments, the nucleotide sequence of the V4 region of the 16S rRNA gene of the fetal Micrococcus sp. bacterium is at least 98.3% identical to SEQ ID NO: 2. In embodiments, the nucleotide sequence of the V4 region of the 16S rRNA gene of the fetal Micrococcus sp. bacterium is at least 98.4% identical to SEQ ID NO: 2. In embodiments, the nucleotide sequence of the V4 region of the 16S rRNA gene of the fetal Micrococcus sp. bacterium is at least 98.5% identical to SEQ ID NO: 2. In embodiments, the nucleotide sequence of the V4 region of the 16S rRNA gene of the fetal Micrococcus sp. bacterium is at least 98.6% identical to SEQ ID NO: 2. In embodiments, the nucleotide sequence of the V4 region of the 16S rRNA gene of the fetal Micrococcus sp. bacterium is at least 98.7% identical to SEQ ID NO: 2. In embodiments, the nucleotide sequence of the V4 region of the 16S rRNA gene of the fetal Micrococcus sp. bacterium is at least 98.8% identical to SEQ ID NO: 2. In embodiments, the nucleotide sequence of the V4 region of the 16S rRNA gene of the fetal Micrococcus sp. bacterium is at least 98.9% identical to SEQ ID NO: 2. In embodiments, the nucleotide sequence of the V4 region of the 16S rRNA gene of the fetal Micrococcus sp. bacterium is at least 99% identical to SEQ ID NO: 2. In embodiments, the nucleotide sequence of the V4 region of the 16S rRNA gene of the fetal Micrococcus sp. bacterium is at least 99.1% identical to SEQ ID NO: 2. In embodiments, the nucleotide sequence of the V4 region of the 16S rRNA gene of the fetal Micrococcus sp. bacterium is at least 99.2% identical to SEQ ID NO: 2. In embodiments, the nucleotide sequence of the V4 region of the 16S rRNA gene of the fetal Micrococcus sp. bacterium is at least 99.3% identical to SEQ ID NO: 2. In embodiments, the nucleotide sequence of the V4 region of the 16S rRNA gene of the fetal Micrococcus sp. bacterium is at least 99.4% identical to SEQ ID NO: 2. In embodiments, the nucleotide sequence of the V4 region of the 16S rRNA gene of the fetal Micrococcus sp. bacterium is at least 99.5% identical to SEQ ID NO: 2. In embodiments, the nucleotide sequence of the V4 region of the 16S rRNA gene of the fetal Micrococcus sp. bacterium is at least 99.6% identical to SEQ ID NO: 2. In embodiments, the nucleotide sequence of the V4 region of the 16S rRNA gene of the fetal Micrococcus sp. bacterium is at least 99.7% identical to SEQ ID NO: 2. In embodiments, the nucleotide sequence of the V4 region of the 16S rRNA gene of the fetal Micrococcus sp. bacterium is at least 99.8% identical to SEQ ID NO: 2. In embodiments, the nucleotide sequence of the V4 region of the 16S rRNA gene of the fetal Micrococcus sp. bacterium is at least 99.9% identical to SEQ ID NO: 2. In embodiments, the nucleotide sequence of the V4 region of the 16S rRNA gene of the fetal Micrococcus sp. bacterium is identical to SEQ ID NO: 2.

In embodiments, the nucleotide sequence of the V4 region of the 16S rRNA gene of the fetal Micrococcus sp. bacterium is at least 95, 96, 97, 98, 99, or 99.5% identical to SEQ ID NO: 7. In embodiments, the nucleotide sequence of the V4 region of the 16S rRNA gene of the fetal Micrococcus sp. bacterium is at least 95% identical to SEQ ID NO: 7. In embodiments, the nucleotide sequence of the V4 region of the 16S rRNA gene of the fetal Micrococcus sp. bacterium is at least 96% identical to SEQ ID NO: 7. In embodiments, the nucleotide sequence of the V4 region of the 16S rRNA gene of the fetal Micrococcus sp. bacterium is at least 97% identical to SEQ ID NO: 7. In embodiments, the nucleotide sequence of the V4 region of the 16S rRNA gene of the fetal Micrococcus sp. bacterium is at least 97.1% identical to SEQ ID NO: 7. In embodiments, the nucleotide sequence of the V4 region of the 16S rRNA gene of the fetal Micrococcus sp. bacterium is at least 97.2% identical to SEQ ID NO: 7. In embodiments, the nucleotide sequence of the V4 region of the 16S rRNA gene of the fetal Micrococcus sp. bacterium is at least 97.3% identical to SEQ ID NO: 7. In embodiments, the nucleotide sequence of the V4 region of the 16S rRNA gene of the fetal Micrococcus sp. bacterium is at least 97.4% identical to SEQ ID NO: 7. In embodiments, the nucleotide sequence of the V4 region of the 16S rRNA gene of the fetal Micrococcus sp. bacterium is at least 97.5% identical to SEQ ID NO: 7. In embodiments, the nucleotide sequence of the V4 region of the 16S rRNA gene of the fetal Micrococcus sp. bacterium is at least 97.6% identical to SEQ ID NO: 7. In embodiments, the nucleotide sequence of the V4 region of the 16S rRNA gene of the fetal Micrococcus sp. bacterium is at least 97.7% identical to SEQ ID NO: 7. In embodiments, the nucleotide sequence of the V4 region of the 16S rRNA gene of the fetal Micrococcus sp. bacterium is at least 97.8% identical to SEQ ID NO: 7. In embodiments, the nucleotide sequence of the V4 region of the 16S rRNA gene of the fetal Micrococcus sp. bacterium is at least 97.9% identical to SEQ ID NO: 7. In embodiments, the nucleotide sequence of the V4 region of the 16S rRNA gene of the fetal Micrococcus sp. bacterium is at least 98% identical to SEQ ID NO: 7. In embodiments, the nucleotide sequence of the V4 region of the 16S rRNA gene of the fetal Micrococcus sp. bacterium is at least 98.1% identical to SEQ ID NO: 7. In embodiments, the nucleotide sequence of the V4 region of the 16S rRNA gene of the fetal Micrococcus sp. bacterium is at least 98.2% identical to SEQ ID NO: 7. In embodiments, the nucleotide sequence of the V4 region of the 16S rRNA gene of the fetal Micrococcus sp. bacterium is at least 98.3% identical to SEQ ID NO: 7. In embodiments, the nucleotide sequence of the V4 region of the 16S rRNA gene of the fetal Micrococcus sp. bacterium is at least 98.4% identical to SEQ ID NO: 7. In embodiments, the nucleotide sequence of the V4 region of the 16S rRNA gene of the fetal Micrococcus sp. bacterium is at least 98.5% identical to SEQ ID NO: 7. In embodiments, the nucleotide sequence of the V4 region of the 16S rRNA gene of the fetal Micrococcus sp. bacterium is at least 98.6% identical to SEQ ID NO: 7. In embodiments, the nucleotide sequence of the V4 region of the 16S rRNA gene of the fetal Micrococcus sp. bacterium is at least 98.7% identical to SEQ ID NO: 7. In embodiments, the nucleotide sequence of the V4 region of the 16S rRNA gene of the fetal Micrococcus sp. bacterium is at least 98.8% identical to SEQ ID NO: 7. In embodiments, the nucleotide sequence of the V4 region of the 16S rRNA gene of the fetal Micrococcus sp. bacterium is at least 98.9% identical to SEQ ID NO: 7. In embodiments, the nucleotide sequence of the V4 region of the 16S rRNA gene of the fetal Micrococcus sp. bacterium is at least 99% identical to SEQ ID NO: 7. In embodiments, the nucleotide sequence of the V4 region of the 16S rRNA gene of the fetal Micrococcus sp. bacterium is at least 99.1% identical to SEQ ID NO: 7. In embodiments, the nucleotide sequence of the V4 region of the 16S rRNA gene of the fetal Micrococcus sp. bacterium is at least 99.2% identical to SEQ ID NO: 7. In embodiments, the nucleotide sequence of the V4 region of the 16S rRNA gene of the fetal Micrococcus sp. bacterium is at least 99.3% identical to SEQ ID NO: 7. In embodiments, the nucleotide sequence of the V4 region of the 16S rRNA gene of the fetal Micrococcus sp. bacterium is at least 99.4% identical to SEQ ID NO: 7. In embodiments, the nucleotide sequence of the V4 region of the 16S rRNA gene of the fetal Micrococcus sp. bacterium is at least 99.5% identical to SEQ ID NO: 7. In embodiments, the nucleotide sequence of the V4 region of the 16S rRNA gene of the fetal Micrococcus sp. bacterium is at least 99.6% identical to SEQ ID NO: 7. In embodiments, the nucleotide sequence of the V4 region of the 16S rRNA gene of the fetal Micrococcus sp. bacterium is at least 99.7% identical to SEQ ID NO: 7. In embodiments, the nucleotide sequence of the V4 region of the 16S rRNA gene of the fetal Micrococcus sp. bacterium is at least 99.8% identical to SEQ ID NO: 7. In embodiments, the nucleotide sequence of the V4 region of the 16S rRNA gene of the fetal Micrococcus sp. bacterium is at least 99.9% identical to SEQ ID NO: 7. In embodiments, the nucleotide sequence of the V4 region of the 16S rRNA gene of the fetal Micrococcus sp. bacterium is identical to SEQ ID NO: 7.

In embodiments, the Lactobacillus sp. (a) reduces activation of antigen presenting cells; (b) reduces the expression of CD86 and/or CD83 on antigen presenting cells; (c) induces expression of the tolerogenic integrin CD103 on dendritic cells; (d) induces expression of the tolerogenic integrin CD103 on CD11c+ dendritic cells; and/or promotes regulatory T cell accumulation (e.g., compared to a standard control).

In embodiments, the Micrococcus sp. reduces IFNγ production by memory promyelocytic leukemia zinc finger protein (PLZF)+ T cells (e.g., compared to a standard control).

In embodiments the level of PLZF+ CD161+ T cells increases in the subject after administration.

In embodiments, the fetal Lactobacillus sp. bacterium is Lacto166. In embodiments, the fetal Lactobacillus sp. bacterium is Lacto167. In embodiments, the Micrococcus sp. bacterium is Micro36.

Current probiotic therapies have not been evaluated for impact on the developing human intestine. Disclosed herein are bacterial strains identified in the human fetal intestine. In embodiments, these species or strains shape lifelong immunity through generation of T cell memory. In embodiments, these fetal intestinal bacteria, isolated from fetal meconium, are distinct from their phylogenetic relatives, several of which are used in current probiotic on the market. In embodiments, strains disclosed herein exhibit an even greater protective as live biotherapeutics. In embodiments, methods and compositions provided herein are effective for teating a co-morbidities of premature birth, such as such as neonatal sepsis, necrotizing enterocolitis, cerebral palsy, and respiratory illnesses. In embodiments, the Micrococcus and/or Lactobacillus strain that is administered is associated with a decreased inflammatory state of the fetal intestine. In embodiments, strains disclosed herein are useful for decreasing inflammation in the fetus to prevent premature birth and its co-morbidities. In embodiments, provided herein is a medical treatment to promote lifelong immune tolerance and reduce disease severity for fetuses or neonates at high risk of chronic inflammatory diseases, such as asthma by supplementation with Micrococcus sp. and Lactobacillus sp. Also provided is an interventional care for pregnant women undergoing or at high-risk for preterm labor. Without being bound by any scientific theory, the neonatal period has been identified as a high-risk window for developing chronic inflammatory diseases such as asthma. In embodiments, during this period bacteria and fungi begin to colonize the infant intestine and shape lifelong immunity. Included herein are two fetal intestinal bacteria belonging to the Micrococcus and Lactobacillus genera, which are highly correlated with intestinal immune cell profiles. Without being bound by any scientific theory, a bacterial presence in the human intestine occurs earlier than previously appreciated. In embodiments, these fetal intestinal bacterial strains promote immune tolerance development through immune tolerance in humans. In embodiments, fetal isolates of Lactobacillus sp. and Micrococcus sp. exert significantly different effects on fetal immunity than currently publically available strains. Provided herein is therapy for asthma newborns and infants at high risk of chronic inflammatory diseases by vaginal/oral supplementation with these Micrococcus and/or Lactobacillus strains to increase immune system maturation and/or Treg function. Also provided is therapy for pregnant women to avoid pre-term labor. In embodiments, therapeutic oral supplementation with Micrococcus and/or Lactobacillus strains provided herein in high-risk for asthma newborns and infants increases immune system maturation and/or Treg function. In embodiments, therapeutic vaginal supplementation with Micrococcus and/or Lactobacillus strains provided herein in pregnant women increases immune system maturation and/or Treg function in the fetus. In embodiments, therapeutic vaginal/oral supplementation with Micrococcus and/or Lactobacillus strains provided herein in pregnant women decreases inflammation in the fetus to prevent premature birth. In embodiments, therapeutic vaginal/oral supplementation with Micrococcus and/or Lactobacillus strains provided herein in pregnant women decreases inflammation in the fetus to prevent childhood obesity. In embodiments, this inflammation is associated with gut microbiome perturbation in the earliest phases of post-natal life. In embodiments, therapeutic oral supplementation with Micrococcus and/or Lactobacillus strains provided herein in patients with chronic inflammatory disease down-regulates inflammation. In embodiments, non-limiting examples of methods and compositions provided herein include the ability to treat fetuses or neonates at high risk of chronic inflammatory diseases, the provision of interventional care for women undergoing or at high-risk for preterm labor, therapies that are biologically relevant than other treatments, and greater efficiency with respect to fetal immunity compared to other strains.

### III. METHODS OF DETECTING, CULTURING, AND ISOLATING BACTERIA

In an aspect, provided herein is a method of detecting a polynucleotide in a fetal intestine (e.g., tissue such as an intestine biopsy or section). In embodiments, the method comprises detecting whether a polynucleotide having a sequence that is at least 95%, 96%, 97%, 98%, 99%, or 100% identical to SEQ ID NO: 1, SEQ ID NO: 2, SEQ ID NO: 3, SEQ ID NO: 4, SEQ ID NO: 5, SEQ ID NO: 6, or SEQ ID NO: 7 is present in a bacterium or biological sample obtained from the fetal intestine.

In an aspect, provided herein is a method of detecting a polynucleotide in a meconium. In embodiments, the method comprises detecting whether a polynucleotide having a sequence that is at least 95%, 96%, 97%, 98%, 99%, or 100% identical to SEQ ID NO: 1, SEQ ID NO: 2, SEQ ID NO: 3, SEQ ID NO: 4, SEQ ID NO: 5, SEQ ID NO: 6, or SEQ ID NO: 7 is present in a bacterium or biological sample obtained from the meconium.

In an aspect, provided herein is a method of detecting a polynucleotide in amniotic fluid. In embodiments, the method comprises detecting whether a polynucleotide having a sequence that is at least 95%, 96%, 97%, 98%, 99%, or 100% identical to SEQ ID NO: 1, SEQ ID NO: 2, SEQ ID NO: 3, SEQ ID NO: 4, SEQ ID NO: 5, SEQ ID NO: 6, or SEQ ID NO: 7 is present in a bacterium or biological sample obtained from the amniotic fluid.

In an aspect, provided herein is a method of detecting a polynucleotide in a placenta. In embodiments, the method comprises detecting whether a polynucleotide having a sequence that is at least 95%, 96%, 97%, 98%, 99%, or 100% identical to SEQ ID NO: 1, SEQ ID NO: 2, SEQ ID NO: 3, SEQ ID NO: 4, SEQ ID NO: 5, SEQ ID NO: 6, or SEQ ID NO: 7 is present in a bacterium or biological sample obtained from the placenta.

In an aspect, provided herein is a method of detecting a polynucleotide in a bacterium, comprising detecting whether a polynucleotide having a sequence that is at least 95%, 96%, 97%, 98%, 99%, or 100% identical to SEQ ID NO: 1, SEQ ID NO: 2, SEQ ID NO: 3, SEQ ID NO: 4, SEQ ID NO: 5, SEQ ID NO: 6, or SEQ ID NO: 7 is present in a bacterium obtained from a fetal intestine, amniotic fluid, meconium, or a placenta.

In embodiments, a method herein comprises detecting a polynucleotide comprises a sequence that is at least about 95%, 96%, 97%, 98%, 99%, or 100% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, a method herein comprises detecting a polynucleotide comprises a sequence that is at least about 95%, 96%, 97%, 98%, 99%, or 100% identical to SEQ ID NO: 1. In embodiments, a method herein comprises detecting a polynucleotide comprises a sequence that is at least about 95%, 96%, 97%, 98%, 99%, or 100% identical to SEQ ID NO: 2. In embodiments, a method herein comprises detecting a polynucleotide comprises a sequence that is at least about 95%, 96%, 97%, 98%, 99%, or 100% identical to SEQ ID NO: 3. In embodiments, a method herein comprises detecting a polynucleotide comprises a sequence that is at least about 95%, 96%, 97%, 98%, 99%, or 100% identical to SEQ ID NO: 4. In embodiments, a method herein comprises detecting a polynucleotide comprises a sequence that is at least about 95%, 96%, 97%, 98%, 99%, or 100% identical to SEQ ID NO: 5. In embodiments, a method herein comprises detecting a polynucleotide comprises a sequence that is at least about 95%, 96%, 97%, 98%, 99%, or 100% identical to SEQ ID NO: 6. In embodiments, the nucleotide sequence of the polynucleotide is at least 95% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 96% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 97% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 97.1% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 97.2% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 97.3% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 97.4% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 97.5% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 97.6% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 97.7% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 97.8% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 97.9% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 98% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 98.1% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 98.2% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 98.3% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 98.4% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 98.5% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 98.6% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 98.7% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 98.8% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 98.9% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 99% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 99.1% identical SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 99.2% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 99.3% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 99.4% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 99.5% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 99.6% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 99.7% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 99.8% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is at least 99.9% identical to SEQ ID NO: 1, SEQ ID NO: 2, SEQ ID NO: 3, SEQ ID NO: 4, SEQ ID NO: 5, SEQ ID NO: 6, or SEQ ID NO: 7. In embodiments, the nucleotide sequence of the polynucleotide is identical to SEQ ID NO: 1, SEQ ID NO: 2, SEQ ID NO: 3, SEQ ID NO: 4, SEQ ID NO: 5, SEQ ID NO: 6, or SEQ ID NO: 7.

In an aspect, provided herein is a method of culturing an isolated bacterium, the method comprising obtaining a bacterium comprising a 16S rRNA gene V4 region comprising a sequence that is at least about identical to SEQ ID NO: 1 or SEQ ID NO: 2, wherein the bacterium has been isolated from amniotic fluid or meconium, and culturing the bacterium.

In an aspect, provided herein is a method of culturing an isolated bacterium, the method comprising obtaining a bacterium comprising a 16S rRNA gene comprising a sequence that is at least 95%, 96%, 97%, 98%, 99%, or 100% identical to SEQ ID NO: 3, SEQ ID NO: 4, or SEQ ID NO: 5 wherein the bacterium has been isolated from a fetal intestine, amniotic fluid, meconium, or a placenta, and culturing the bacterium.

In an aspect, provided herein is a method of culturing an isolated bacterium, the method comprising obtaining a bacterium comprising a 16S rRNA gene comprising a sequence that is at least 95%, 96%, 97%, 98%, 99%, or 100% identical to SEQ ID NO: 6 or SEQ ID NO: 7, wherein the bacterium has been isolated from a fetal intestine, amniotic fluid, meconium, or a placenta, and culturing the bacterium.

In an aspect, provided herein is a method of culturing a fetal Micrococcus sp. bacterium and/or a fetal Lactobacillus sp. bacterium, the method comprising incubating the bacterium in or on a medium comprising a eukaryotic cell, and/or a placental hormone.

In an aspect, provided herein is a method of culturing a fetal Micrococcus sp. bacterium and/or a fetal Lactobacillus sp. bacterium, the method comprising incubating the bacterium in or on a medium comprising an epithelial cell and/or a placental hormone.

In an aspect, provided herein is a method of culturing a fetal Micrococcus sp. bacterium and/or a fetal Lactobacillus sp. bacterium, the method comprising incubating the bacterium in or on a medium comprising a monocyte or a macrophage, and/or a placental hormone.

In an aspect, provided herein is a method of isolating a fetal Micrococcus sp. bacterium and/or a fetal Lactobacillus sp. bacterium. In embodiments, the method comprises (i) incubating a culture medium comprising (a) a biological sample suspected of containing the bacterium and (b) a eukaryotic cell, and/or a placental hormone, thereby producing a pre-isolate culture; (ii) selecting the fetal Micrococcus sp. bacterium and/or a fetal Lactobacillus sp. bacterium. In embodiments, selecting the fetal Micrococcus sp. bacterium and/or a fetal Lactobacillus sp. bacterium comprises streaking a portion of the pre-isolate culture onto a selection plate (e.g., an plate comprising medium that comprises a gel-like or solid state such as a medium comprising agarose), and selecting a single colony of the fetal Micrococcus sp. bacterium and/or a fetal Lactobacillus sp. bacterium from the plate.

In an aspect, provided herein is a method of isolating a fetal Micrococcus sp. bacterium and/or a fetal Lactobacillus sp. bacterium. In embodiments, the method comprises (i) incubating a culture medium comprising (a) a biological sample suspected of containing the bacterium and (b) an epithelial cell, and/or a placental hormone, thereby producing a pre-isolate culture; (ii) selecting the fetal Micrococcus sp. bacterium and/or a fetal Lactobacillus sp. bacterium. In embodiments, selecting the fetal Micrococcus sp. bacterium and/or a fetal Lactobacillus sp. bacterium comprises streaking a portion of the pre-isolate culture onto a selection plate (e.g., an plate comprising medium that comprises a gel-like or solid state such as a medium comprising agarose), and selecting a single colony of the fetal Micrococcus sp. bacterium and/or a fetal Lactobacillus sp. bacterium from the plate.

In an aspect, provided herein is a method of isolating a fetal Micrococcus sp. bacterium and/or a fetal Lactobacillus sp. bacterium. In embodiments, the method comprises (i) incubating a culture medium comprising (a) a biological sample suspected of containing the bacterium and (b) a monocyte or a macrophage, and/or a placental hormone, thereby producing a pre-isolate culture; (ii) selecting the fetal Micrococcus sp. bacterium and/or a fetal Lactobacillus sp. bacterium. In embodiments, selecting the fetal Micrococcus sp. bacterium and/or a fetal Lactobacillus sp. bacterium comprises streaking a portion of the pre-isolate culture onto a selection plate (e.g., an plate comprising medium that comprises a gel-like or solid state such as a medium comprising agarose), and selecting a single colony of the fetal Micrococcus sp. bacterium and/or a fetal Lactobacillus sp. bacterium from the plate.

In embodiments, the biological sample is a fetal intestine biopsy, meconium, amniotic fluid, placenta tissue, or a bodily fluid obtained from a placenta.

In embodiments, the medium comprises a placental hormone.

In embodiments, the placental hormone is the only source of carbon in the medium.

In embodiments, the placental hormone is progesterone, estradiol, human placental lactogen, human chorionic gonadotropin, relaxin, estriol (E3), sterol (E4), pregnenolone, pregnenolone sulfate, or dehydroepiandrosterone (DHEA). In embodiments, the placental hormone is progesterone. In embodiments, the placental hormone is estradiol. In embodiments, the placental hormone is human placental lactogen. In embodiments, the placental hormone is human chorionic gonadotropin. In embodiments, the placental hormone is relaxin. In embodiments, the placental hormone is progesterone or estradiol. In embodiments, the placental hormone is an analogue or derivative of a naturally occurring placental hormone. In embodiments, the placental hormone is estriol (E3). In embodiments, the placental hormone is sterol (E4). In embodiments, the placental hormone is pregnenolone. In embodiments, the placental hormone is pregnenolone sulfate. In embodiments, the placental hormone is dehydroepiandrosterone (DHEA).

In embodiments, the estradiol is β-estradiol.

In embodiments, the β-estradiol is 17 β-estradiol.

In embodiments, the medium comprises a eukaryotic cell.

In embodiments, the medium comprises an epithelial cell.

In embodiments, the medium comprises a monocyte.

In embodiments, the medium comprises a macrophage.

In embodiments, the monocyte is a primary monocyte or the macrophage is a primary macrophage.

In embodiments, the monocyte or macrophage is a cell line.

In embodiments, the cell line is a THP-1 human monocytic cell line or RAW264.7.

In embodiments, the epithelial cell is a primary epithelial cell.

In embodiments, the epithelial cell is a cell line.

In embodiments, the cell line is a CACO2 cell line.

Non-limiting examples of media include chopped meat carbohydrate broth (e.g., CMC from Anaerobe Systems), brain heart infusion (e.g., BHI from TekNova) agar plate, tryptic soy broth (BD), luria broth, tryptic soy broth supplemented with 5% defibrinated horse blood (e.g., TSBB from Fisher Scientific). In embodiments, the medium is chopped meat carbohydrate broth (e.g., CMC from Anaerobe Systems). In embodiments, the medium is brain heart infusion (e.g., BHI from TekNova). In embodiments, the medium is tryptic soy broth. In embodiments, the medium is luria broth. In embodiments, the medium is tryptic soy broth. In embodiments, the medium is luria broth and tryptic soy broth. In embodiments, the medium is luria broth and tryptic soy broth without blood. In embodiments, the medium comprises blood. In embodiments, the medium does not comprise blood. In embodiments, the medium is tryptic soy broth. In embodiments, the medium is tryptic soy broth supplemented with about 5% defibrinated horse blood. In embodiments, a medium is in a liquid, hydrogel, gel, semi-solid, or solid form. In embodiments, medium is mixed with agarose. In embodiments, the medium comprises 0.5-2, 0.7-2.5, 2.5-5, 1-5, 5-10, 10-15, or 15-25 agarose by weight. In embodiments, the medium is Roswell Park Memorial Institute (RPMI, GIBCO). In embodiments, the medium (e.g., RPMI) does not comprise an antibiotic. In embodiments, the medium (e.g., RPMI) is supplemented with fetal bovine serum (e.g., 5, 6, 7, 8, 9, 10, 11, 12, 5-10, 10-12, or 9-11% fetal bovine serum). In embodiments, the medium is supplemented with sodium pyruvate (e.g., 0.5, 0.75, 1, 1.25, 1.5, 0.5-1.5, or 0.75-1.25 mM sodium pyruvate). In embodiments, the medium is supplemented with L-glutamine (e.g., 1.5, 1.75, 2, 2.25, 2.5, or 1.75-2.25 mM L-glutamine). In embodiments, the medium is supplemented non-essential amino acids. In embodiments, the medium is supplemented with 4-(2-hydroxyethyl)-1-piperazineethanesulfonic acid (HEPES) (e.g., 5, 6, 7, 8, 9, 10, 11, 12, 5-10, 10-12, or 9-11 mM HEPES). In embodiments, the medium is RPMI without antibiotics and supplemented with 10% fetal bovine serum, 1 mM sodium pyruvate, 2 mM L-glutamine, 1× non-essential amino acids, and 10 mM HEPES (cRPMI). In embodiments, the medium (e.g., cRPMI) comprises monocytes of macrophages.

In embodiments, Micrococcus sp. and/or Lactobacillus sp. is cultured together with a eukaryotic cell. In embodiments, the eukaryotic cell is a monocyte, a macrophage, or an epithelial cell. In embodiments, the eukaryotic cell is a primary cell. In embodiments, the eukaryotic cell is a cell line. In embodiments, the cell line is a THP-1 human monocytic cell line, RAW264.7, or CACO2.

In embodiments, eukaryotic cells (such as monocytes, macrophages, or epithelial cells) are in the medium in an amount of from 1×10⁶ to 1×10⁸, from 1×10⁶ to 1×10⁷, from 1×10⁷ to 1×10⁸, from 2×10⁶ to 1×10⁸, from 1×10⁶ to 3×10⁶, from 1.5×10⁶ to 2.5×10⁷, or about 1×10⁶, 1.5×10⁶, 2×10⁶, 2.5×10⁶, 3×10⁶, 3.5×10⁶, 4×10⁶, 4.5×10⁶, or 5×10⁶ cells per 20 mL of medium. In embodiments, eukaryotic cells (such as monocytes, macrophages, or epithelial cells) are in the medium in an amount of from 1×10⁴ to 1×10⁶, from 1×10⁴ to 1×10⁵, from 1×10⁵ to 1×10⁶, from 2×10⁴ to 1×10⁶, from 1×10⁴ to 3×10⁴, from 1.5×10⁴ to 2.5×10⁵, or about 1×10⁴, 1.5×10⁴, 2×10⁴, 2.5×10⁴, 3×10⁴, 3.5×10⁴, 4×10⁴, 4.5×10⁴, or 5×10⁴ cells per mL of medium.

In embodiments, detecting a polynucleotide comprises isolating the polynucleotide and contacting the polynucleotide with a probe or a primer (e.g., a single primer or a pair of primers that flank a whole or a part of a gene of interest). In embodiments, a probe or a primer hybridizes with a polynucleotide under stringent hybridization conditions. In embodiments, detecting a polynucleotide comprising a sequence that is at least about 95%, 96%, 97%, 98%, 99%, or 100% identical to SEQ ID NO: 1, SEQ ID NO: 2, SEQ ID NO: 3, SEQ ID NO: 4, SEQ ID NO: 5, SEQ ID NO: 6, or SEQ ID NO: 7 comprises contacting a biological sample or nucleic acids obtained from a biological sample with a probe or a primer that binds to SEQ ID NO: 1, SEQ ID NO: 2, SEQ ID NO: 3, SEQ ID NO: 4, SEQ ID NO: 5, SEQ ID NO: 6, or SEQ ID NO: 7 under stringent conditions. In embodiments, detecting a polynucleotide comprises sequencing.

In embodiments, detecting a polynucleotide comprises a microarray. In embodiments, detecting a polynucleotide does not comprise a microarray. In embodiments, detecting a polynucleotide comprises a polymerase chain reaction.

### IV. ISOLATED BACTERIA AND COMPOSITIONS

In an aspect, provided herein is an isolated fetal Micrococcus sp. bacterium and/or an isolated fetal Lactobacillus sp. bacterium.

In embodiments, the bacterium is lyophilized.

In embodiments, the nucleotide sequence of the 16S rRNA gene of the fetal Lactobacillus sp. bacterium is at least 95, 96, 97, 98, 99, or 99.5% identical to SEQ ID NO: 3. In embodiments, the nucleotide sequence of the 16S rRNA gene of the fetal Lactobacillus sp. bacterium is at least 95% identical to SEQ ID NO: 3. In embodiments, the nucleotide sequence of the 16S rRNA gene of the fetal Lactobacillus sp. bacterium is at least 96% identical to SEQ ID NO: 3. In embodiments, the nucleotide sequence of the 16S rRNA gene of the fetal Lactobacillus sp. bacterium is at least 97% identical to SEQ ID NO: 3. In embodiments, the nucleotide sequence of the 16S rRNA gene of the fetal Lactobacillus sp. bacterium is at least 97.1% identical to SEQ ID NO: 3. In embodiments, the nucleotide sequence of the 16S rRNA gene of the fetal Lactobacillus sp. bacterium is at least 97.2% identical to SEQ ID NO: 3. In embodiments, the nucleotide sequence of the 16S rRNA gene of the fetal Lactobacillus sp. bacterium is at least 97.3% identical to SEQ ID NO: 3. In embodiments, the nucleotide sequence of the 16S rRNA gene of the fetal Lactobacillus sp. bacterium is at least 97.4% identical to SEQ ID NO: 3. In embodiments, the nucleotide sequence of the 16S rRNA gene of the fetal Lactobacillus sp. bacterium is at least 97.5% identical to SEQ ID NO: 3. In embodiments, the nucleotide sequence of the 16S rRNA gene of the fetal Lactobacillus sp. bacterium is at least 97.6% identical to SEQ ID NO: 3. In embodiments, the nucleotide sequence of the 16S rRNA gene of the fetal Lactobacillus sp. bacterium is at least 97.7% identical to SEQ ID NO: 3. In embodiments, the nucleotide sequence of the 16S rRNA gene of the fetal Lactobacillus sp. bacterium is at least 97.8% identical to SEQ ID NO: 3. In embodiments, the nucleotide sequence of the 16S rRNA gene of the fetal Lactobacillus sp. bacterium is at least 97.9% identical to SEQ ID NO: 3. In embodiments, the nucleotide sequence of the 16S rRNA gene of the fetal Lactobacillus sp. bacterium is at least 98% identical to SEQ ID NO: 3. In embodiments, the nucleotide sequence of the 16S rRNA gene of the fetal Lactobacillus sp. bacterium is at least 98.1% identical to SEQ ID NO: 3. In embodiments, the nucleotide sequence of the 16S rRNA gene of the fetal Lactobacillus sp. bacterium is at least 98.2% identical to SEQ ID NO: 3. In embodiments, the nucleotide sequence of the 16S rRNA gene of the fetal Lactobacillus sp. bacterium is at least 98.3% identical to SEQ ID NO: 3. In embodiments, the nucleotide sequence of the 16S rRNA gene of the fetal Lactobacillus sp. bacterium is at least 98.4% identical to SEQ ID NO: 3. In embodiments, the nucleotide sequence of the 16S rRNA gene of the fetal Lactobacillus sp. bacterium is at least 98.5% identical to SEQ ID NO: 3. In embodiments, the nucleotide sequence of the 16S rRNA gene of the fetal Lactobacillus sp. bacterium is at least 98.6% identical to SEQ ID NO: 3. In embodiments, the nucleotide sequence of the 16S rRNA gene of the fetal Lactobacillus sp. bacterium is at least 98.7% identical to SEQ ID NO: 3. In embodiments, the nucleotide sequence of the 16S rRNA gene of the fetal Lactobacillus sp. bacterium is at least 98.8% identical to SEQ ID NO: 3. In embodiments, the nucleotide sequence of the 16S rRNA gene of the fetal Lactobacillus sp. bacterium is at least 98.9% identical to SEQ ID NO: 3. In embodiments, the nucleotide sequence of the 16S rRNA gene of the fetal Lactobacillus sp. bacterium is at least 99% identical to SEQ ID NO: 3. In embodiments, the nucleotide sequence of the 16S rRNA gene of the fetal Lactobacillus sp. bacterium is at least 99.1% identical to SEQ ID NO: 3. In embodiments, the nucleotide sequence of the 16S rRNA gene of the fetal Lactobacillus sp. bacterium is at least 99.2% identical to SEQ ID NO: 3. In embodiments, the nucleotide sequence of the 16S rRNA gene of the fetal Lactobacillus sp. bacterium is at least 99.3% identical to SEQ ID NO: 3. In embodiments, the nucleotide sequence of the 16S rRNA gene of the fetal Lactobacillus sp. bacterium is at least 99.4% identical to SEQ ID NO: 3. In embodiments, the nucleotide sequence of the 16S rRNA gene of the fetal Lactobacillus sp. bacterium is at least 99.5% identical to SEQ ID NO: 3. In embodiments, the nucleotide sequence of the 16S rRNA gene of the fetal Lactobacillus sp. bacterium is at least 99.6% identical to SEQ ID NO: 3. In embodiments, the nucleotide sequence of the 16S rRNA gene of the fetal Lactobacillus sp. bacterium is at least 99.7% identical to SEQ ID NO: 3. In embodiments, the nucleotide sequence of the 16S rRNA gene of the fetal Lactobacillus sp. bacterium is at least 99.8% identical to SEQ ID NO: 3. In embodiments, the nucleotide sequence of the 16S rRNA gene of the fetal Lactobacillus sp. bacterium is at least 99.9% identical to SEQ ID NO: 3. In embodiments, the nucleotide sequence of the 16S rRNA gene of the fetal Lactobacillus sp. bacterium is identical to SEQ ID NO: 3.

In embodiments, the nucleotide sequence of the 16S rRNA gene of the fetal Lactobacillus sp. bacterium is at least 95, 96, 97, 98, 99, or 99.5% identical to SEQ ID NO: 5. In embodiments, the nucleotide sequence of the 16S rRNA gene of the fetal Lactobacillus sp. bacterium is at least 95% identical to SEQ ID NO: 5. In embodiments, the nucleotide sequence of the 16S rRNA gene of the fetal Lactobacillus sp. bacterium is at least 96% identical to SEQ ID NO: 5. In embodiments, the nucleotide sequence of the 16S rRNA gene of the fetal Lactobacillus sp. bacterium is at least 97% identical to SEQ ID NO: 5. In embodiments, the nucleotide sequence of the 16S rRNA gene of the fetal Lactobacillus sp. bacterium is at least 97.1% identical to SEQ ID NO: 5. In embodiments, the nucleotide sequence of the 16S rRNA gene of the fetal Lactobacillus sp. bacterium is at least 97.2% identical to SEQ ID NO: 5. In embodiments, the nucleotide sequence of the 16S rRNA gene of the fetal Lactobacillus sp. bacterium is at least 97.3% identical to SEQ ID NO: 5. In embodiments, the nucleotide sequence of the 16S rRNA gene of the fetal Lactobacillus sp. bacterium is at least 97.4% identical to SEQ ID NO: 5. In embodiments, the nucleotide sequence of the 16S rRNA gene of the fetal Lactobacillus sp. bacterium is at least 97.5% identical to SEQ ID NO: 5. In embodiments, the nucleotide sequence of the 16S rRNA gene of the fetal Lactobacillus sp. bacterium is at least 97.6% identical to SEQ ID NO: 5. In embodiments, the nucleotide sequence of the 16S rRNA gene of the fetal Lactobacillus sp. bacterium is at least 97.7% identical to SEQ ID NO: 5. In embodiments, the nucleotide sequence of the 16S rRNA gene of the fetal Lactobacillus sp. bacterium is at least 97.8% identical to SEQ ID NO: 5. In embodiments, the nucleotide sequence of the 16S rRNA gene of the fetal Lactobacillus sp. bacterium is at least 97.9% identical to SEQ ID NO: 5. In embodiments, the nucleotide sequence of the 16S rRNA gene of the fetal Lactobacillus sp. bacterium is at least 98% identical to SEQ ID NO: 5. In embodiments, the nucleotide sequence of the 16S rRNA gene of the fetal Lactobacillus sp. bacterium is at least 98.1% identical to SEQ ID NO: 5. In embodiments, the nucleotide sequence of the 16S rRNA gene of the fetal Lactobacillus sp. bacterium is at least 98.2% identical to SEQ ID NO: 5. In embodiments, the nucleotide sequence of the 16S rRNA gene of the fetal Lactobacillus sp. bacterium is at least 98.3% identical to SEQ ID NO: 5. In embodiments, the nucleotide sequence of the 16S rRNA gene of the fetal Lactobacillus sp. bacterium is at least 98.4% identical to SEQ ID NO: 5. In embodiments, the nucleotide sequence of the 16S rRNA gene of the fetal Lactobacillus sp. bacterium is at least 98.5% identical to SEQ ID NO: 5. In embodiments, the nucleotide sequence of the 16S rRNA gene of the fetal Lactobacillus sp. bacterium is at least 98.6% identical to SEQ ID NO: 5. In embodiments, the nucleotide sequence of the 16S rRNA gene of the fetal Lactobacillus sp. bacterium is at least 98.7% identical to SEQ ID NO: 5. In embodiments, the nucleotide sequence of the 16S rRNA gene of the fetal Lactobacillus sp. bacterium is at least 98.8% identical to SEQ ID NO: 5. In embodiments, the nucleotide sequence of the 16S rRNA gene of the fetal Lactobacillus sp. bacterium is at least 98.9% identical to SEQ ID NO: 5. In embodiments, the nucleotide sequence of the 16S rRNA gene of the fetal Lactobacillus sp. bacterium is at least 99% identical to SEQ ID NO: 5. In embodiments, the nucleotide sequence of the 16S rRNA gene of the fetal Lactobacillus sp. bacterium is at least 99.1% identical to SEQ ID NO: 5. In embodiments, the nucleotide sequence of the 16S rRNA gene of the fetal Lactobacillus sp. bacterium is at least 99.2% identical to SEQ ID NO: 5. In embodiments, the nucleotide sequence of the 16S rRNA gene of the fetal Lactobacillus sp. bacterium is at least 99.3% identical to SEQ ID NO: 5. In embodiments, the nucleotide sequence of the 16S rRNA gene of the fetal Lactobacillus sp. bacterium is at least 99.4% identical to SEQ ID NO: 5. In embodiments, the nucleotide sequence of the 16S rRNA gene of the fetal Lactobacillus sp. bacterium is at least 99.5% identical to SEQ ID NO: 5. In embodiments, the nucleotide sequence of the 16S rRNA gene of the fetal Lactobacillus sp. bacterium is at least 99.6% identical to SEQ ID NO: 5. In embodiments, the nucleotide sequence of the 16S rRNA gene of the fetal Lactobacillus sp. bacterium is at least 99.7% identical to SEQ ID NO: 5. In embodiments, the nucleotide sequence of the 16S rRNA gene of the fetal Lactobacillus sp. bacterium is at least 99.8% identical to SEQ ID NO: 5. In embodiments, the nucleotide sequence of the 16S rRNA gene of the fetal Lactobacillus sp. bacterium is at least 99.9% identical to SEQ ID NO: 5. In embodiments, the nucleotide sequence of the 16S rRNA gene of the fetal Lactobacillus sp. bacterium is identical to SEQ ID NO: 5.

In embodiments, the nucleotide sequence of V4 region of the 16S rRNA gene of the fetal Lactobacillus sp. bacterium is at least 95, 96, 97, 98, 99, or 99.5% identical to SEQ ID NO: 6. In embodiments, the nucleotide sequence of V4 region of the 16S rRNA gene of the fetal Lactobacillus sp. bacterium is at least 95% identical to SEQ ID NO: 6. In embodiments, the nucleotide sequence of V4 region of the 16S rRNA gene of the fetal Lactobacillus sp. bacterium is at least 96% identical to SEQ ID NO: 6. In embodiments, the nucleotide sequence of V4 region of the 16S rRNA gene of the fetal Lactobacillus sp. bacterium is at least 97% identical to SEQ ID NO: 6. In embodiments, the nucleotide sequence of V4 region of the 16S rRNA gene of the fetal Lactobacillus sp. bacterium is at least 97.1% identical to SEQ ID NO: 6. In embodiments, the nucleotide sequence of V4 region of the 16S rRNA gene of the fetal Lactobacillus sp. bacterium is at least 97.2% identical to SEQ ID NO: 6. In embodiments, the nucleotide sequence of V4 region of the 16S rRNA gene of the fetal Lactobacillus sp. bacterium is at least 97.3% identical to SEQ ID NO: 6. In embodiments, the nucleotide sequence of V4 region of the 16S rRNA gene of the fetal Lactobacillus sp. bacterium is at least 97.4% identical to SEQ ID NO: 6. In embodiments, the nucleotide sequence of V4 region of the 16S rRNA gene of the fetal Lactobacillus sp. bacterium is at least 97.5% identical to SEQ ID NO: 6. In embodiments, the nucleotide sequence of V4 region of the 16S rRNA gene of the fetal Lactobacillus sp. bacterium is at least 97.6% identical to SEQ ID NO: 6. In embodiments, the nucleotide sequence of V4 region of the 16S rRNA gene of the fetal Lactobacillus sp. bacterium is at least 97.7% identical to SEQ ID NO: 6. In embodiments, the nucleotide sequence of V4 region of the 16S rRNA gene of the fetal Lactobacillus sp. bacterium is at least 97.8% identical to SEQ ID NO: 6. In embodiments, the nucleotide sequence of V4 region of the 16S rRNA gene of the fetal Lactobacillus sp. bacterium is at least 97.9% identical to SEQ ID NO: 6. In embodiments, the nucleotide sequence of V4 region of the 16S rRNA gene of the fetal Lactobacillus sp. bacterium is at least 98% identical to SEQ ID NO: 6. In embodiments, the nucleotide sequence of V4 region of the 16S rRNA gene of the fetal Lactobacillus sp. bacterium is at least 98.1% identical to SEQ ID NO: 6. In embodiments, the nucleotide sequence of V4 region of the 16S rRNA gene of the fetal Lactobacillus sp. bacterium is at least 98.2% identical to SEQ ID NO: 6. In embodiments, the nucleotide sequence of V4 region of the 16S rRNA gene of the fetal Lactobacillus sp. bacterium is at least 98.3% identical to SEQ ID NO: 6. In embodiments, the nucleotide sequence of V4 region of the 16S rRNA gene of the fetal Lactobacillus sp. bacterium is at least 98.4% identical to SEQ ID NO: 6. In embodiments, the nucleotide sequence of V4 region of the 16S rRNA gene of the fetal Lactobacillus sp. bacterium is at least 98.5% identical to SEQ ID NO: 6. In embodiments, the nucleotide sequence of V4 region of the 16S rRNA gene of the fetal Lactobacillus sp. bacterium is at least 98.6% identical to SEQ ID NO: 6. In embodiments, the nucleotide sequence of V4 region of the 16S rRNA gene of the fetal Lactobacillus sp. bacterium is at least 98.7% identical to SEQ ID NO: 6. In embodiments, the nucleotide sequence of V4 region of the 16S rRNA gene of the fetal Lactobacillus sp. bacterium is at least 98.8% identical to SEQ ID NO: 6. In embodiments, the nucleotide sequence of V4 region of the 16S rRNA gene of the fetal Lactobacillus sp. bacterium is at least 98.9% identical to SEQ ID NO: 6. In embodiments, the nucleotide sequence of V4 region of the 16S rRNA gene of the fetal Lactobacillus sp. bacterium is at least 99% identical to SEQ ID NO: 6. In embodiments, the nucleotide sequence of V4 region of the 16S rRNA gene of the fetal Lactobacillus sp. bacterium is at least 99.1% identical to SEQ ID NO: 6. In embodiments, the nucleotide sequence of V4 region of the 16S rRNA gene of the fetal Lactobacillus sp. bacterium is at least 99.2% identical to SEQ ID NO: 6. In embodiments, the nucleotide sequence of V4 region of the 16S rRNA gene of the fetal Lactobacillus sp. bacterium is at least 99.3% identical to SEQ ID NO: 6. In embodiments, the nucleotide sequence of V4 region of the 16S rRNA gene of the fetal Lactobacillus sp. bacterium is at least 99.4% identical to SEQ ID NO: 6. In embodiments, the nucleotide sequence of V4 region of the 16S rRNA gene of the fetal Lactobacillus sp. bacterium is at least 99.5% identical to SEQ ID NO: 6. In embodiments, the nucleotide sequence of V4 region of the 16S rRNA gene of the fetal Lactobacillus sp. bacterium is at least 99.6% identical to SEQ ID NO: 6. In embodiments, the nucleotide sequence of V4 region of the 16S rRNA gene of the fetal Lactobacillus sp. bacterium is at least 99.7% identical to SEQ ID NO: 6. In embodiments, the nucleotide sequence of V4 region of the 16S rRNA gene of the fetal Lactobacillus sp. bacterium is at least 99.8% identical to SEQ ID NO: 6. In embodiments, the nucleotide sequence of V4 region of the 16S rRNA gene of the fetal Lactobacillus sp. bacterium is at least 99.9% identical to SEQ ID NO: 6. In embodiments, the nucleotide sequence of the V4 region of the 16S rRNA gene of the fetal Lactobacillus sp. bacterium is identical to SEQ ID NO: 6.

In embodiments, the nucleotide sequence of V4 region of the 16S rRNA gene of the fetal Lactobacillus sp. bacterium is at least 95, 96, 97, 98, 99, or 99.5% identical to SEQ ID NO: 1. In embodiments, the nucleotide sequence of V4 region of the 16S rRNA gene of the fetal Lactobacillus sp. bacterium is at least 95% identical to SEQ ID NO: 1. In embodiments, the nucleotide sequence of V4 region of the 16S rRNA gene of the fetal Lactobacillus sp. bacterium is at least 96% identical to SEQ ID NO: 1. In embodiments, the nucleotide sequence of V4 region of the 16S rRNA gene of the fetal Lactobacillus sp. bacterium is at least 97% identical to SEQ ID NO: 1. In embodiments, the nucleotide sequence of V4 region of the 16S rRNA gene of the fetal Lactobacillus sp. bacterium is at least 97.1% identical to SEQ ID NO: 1. In embodiments, the nucleotide sequence of V4 region of the 16S rRNA gene of the fetal Lactobacillus sp. bacterium is at least 97.2% identical to SEQ ID NO: 1. In embodiments, the nucleotide sequence of V4 region of the 16S rRNA gene of the fetal Lactobacillus sp. bacterium is at least 97.3% identical to SEQ ID NO: 1. In embodiments, the nucleotide sequence of V4 region of the 16S rRNA gene of the fetal Lactobacillus sp. bacterium is at least 97.4% identical to SEQ ID NO: 1. In embodiments, the nucleotide sequence of V4 region of the 16S rRNA gene of the fetal Lactobacillus sp. bacterium is at least 97.5% identical to SEQ ID NO: 1. In embodiments, the nucleotide sequence of V4 region of the 16S rRNA gene of the fetal Lactobacillus sp. bacterium is at least 97.6% identical to SEQ ID NO: 1. In embodiments, the nucleotide sequence of V4 region of the 16S rRNA gene of the fetal Lactobacillus sp. bacterium is at least 97.7% identical to SEQ ID NO: 1. In embodiments, the nucleotide sequence of V4 region of the 16S rRNA gene of the fetal Lactobacillus sp. bacterium is at least 97.8% identical to SEQ ID NO: 1. In embodiments, the nucleotide sequence of V4 region of the 16S rRNA gene of the fetal Lactobacillus sp. bacterium is at least 97.9% identical to SEQ ID NO: 1. In embodiments, the nucleotide sequence of V4 region of the 16S rRNA gene of the fetal Lactobacillus sp. bacterium is at least 98% identical to SEQ ID NO: 1. In embodiments, the nucleotide sequence of V4 region of the 16S rRNA gene of the fetal Lactobacillus sp. bacterium is at least 98.1% identical to SEQ ID NO: 1. In embodiments, the nucleotide sequence of V4 region of the 16S rRNA gene of the fetal Lactobacillus sp. bacterium is at least 98.2% identical to SEQ ID NO: 1. In embodiments, the nucleotide sequence of V4 region of the 16S rRNA gene of the fetal Lactobacillus sp. bacterium is at least 98.3% identical to SEQ ID NO: 1. In embodiments, the nucleotide sequence of V4 region of the 16S rRNA gene of the fetal Lactobacillus sp. bacterium is at least 98.4% identical to SEQ ID NO: 1. In embodiments, the nucleotide sequence of V4 region of the 16S rRNA gene of the fetal Lactobacillus sp. bacterium is at least 98.5% identical to SEQ ID NO: 1. In embodiments, the nucleotide sequence of V4 region of the 16S rRNA gene of the fetal Lactobacillus sp. bacterium is at least 98.6% identical to SEQ ID NO: 1. In embodiments, the nucleotide sequence of V4 region of the 16S rRNA gene of the fetal Lactobacillus sp. bacterium is at least 98.7% identical to SEQ ID NO: 1. In embodiments, the nucleotide sequence of V4 region of the 16S rRNA gene of the fetal Lactobacillus sp. bacterium is at least 98.8% identical to SEQ ID NO: 1. In embodiments, the nucleotide sequence of V4 region of the 16S rRNA gene of the fetal Lactobacillus sp. bacterium is at least 98.9% identical to SEQ ID NO: 1. In embodiments, the nucleotide sequence of V4 region of the 16S rRNA gene of the fetal Lactobacillus sp. bacterium is at least 99% identical to SEQ ID NO: 1. In embodiments, the nucleotide sequence of V4 region of the 16S rRNA gene of the fetal Lactobacillus sp. bacterium is at least 99.1% identical to SEQ ID NO: 1. In embodiments, the nucleotide sequence of V4 region of the 16S rRNA gene of the fetal Lactobacillus sp. bacterium is at least 99.2% identical to SEQ ID NO: 1. In embodiments, the nucleotide sequence of V4 region of the 16S rRNA gene of the fetal Lactobacillus sp. bacterium is at least 99.3% identical to SEQ ID NO: 1. In embodiments, the nucleotide sequence of V4 region of the 16S rRNA gene of the fetal Lactobacillus sp. bacterium is at least 99.4% identical to SEQ ID NO: 1. In embodiments, the nucleotide sequence of V4 region of the 16S rRNA gene of the fetal Lactobacillus sp. bacterium is at least 99.5% identical to SEQ ID NO: 1. In embodiments, the nucleotide sequence of V4 region of the 16S rRNA gene of the fetal Lactobacillus sp. bacterium is at least 99.6% identical to SEQ ID NO: 1. In embodiments, the nucleotide sequence of V4 region of the 16S rRNA gene of the fetal Lactobacillus sp. bacterium is at least 99.7% identical to SEQ ID NO: 1. In embodiments, the nucleotide sequence of V4 region of the 16S rRNA gene of the fetal Lactobacillus sp. bacterium is at least 99.8% identical to SEQ ID NO: 1. In embodiments, the nucleotide sequence of V4 region of the 16S rRNA gene of the fetal Lactobacillus sp. bacterium is at least 99.9% identical to SEQ ID NO: 1. In embodiments, the nucleotide sequence of the V4 region of the 16S rRNA gene of the fetal Lactobacillus sp. bacterium is identical to SEQ ID NO: 1.

In embodiments, the Lactobacillus sp. reduces activation of antigen presenting cells (e. g., compared to a standard control).

In embodiments, the Lactobacillus sp. reduces the expression of CD86 and/or CD83 on antigen presenting cells (e.g., compared to a standard control).

In embodiments, the Lactobacillus sp. induces expression of the tolerogenic integrin CD103 on dendritic cells (e.g., compared to a standard control).

In embodiments, the Lactobacillus sp. induces expression of the tolerogenic integrin CD103 on CD11c+ dendritic cells; and/or promotes regulatory T cell accumulation (e.g., compared to a standard control).

In embodiments, the nucleotide sequence of the 16S rRNA gene of the fetal Micrococcus sp. bacterium is at least 95, 96, 97, 98, 99, or 99.5% identical to SEQ ID NO: 4. In embodiments, the nucleotide sequence of the 16S rRNA gene of the fetal Micrococcus sp. bacterium is at least 95% identical to SEQ ID NO: 4. In embodiments, the nucleotide sequence of the 16S rRNA gene of the fetal Micrococcus sp. bacterium is at least 96% identical to SEQ ID NO: 4. In embodiments, the nucleotide sequence of the 16S rRNA gene of the fetal Micrococcus sp. bacterium is at least 97% identical to SEQ ID NO: 4. In embodiments, the nucleotide sequence of the 16S rRNA gene of the fetal Micrococcus sp. bacterium is at least 97.1% identical to SEQ ID NO: 4. In embodiments, the nucleotide sequence of the 16S rRNA gene of the fetal Micrococcus sp. bacterium is at least 97.2% identical to SEQ ID NO: 4. In embodiments, the nucleotide sequence of the 16S rRNA gene of the fetal Micrococcus sp. bacterium is at least 97.3% identical to SEQ ID NO: 4. In embodiments, the nucleotide sequence of the 16S rRNA gene of the fetal Micrococcus sp. bacterium is at least 97.4% identical to SEQ ID NO: 4. In embodiments, the nucleotide sequence of the 16S rRNA gene of the fetal Micrococcus sp. bacterium is at least 97.5% identical to SEQ ID NO: 4. In embodiments, the nucleotide sequence of the 16S rRNA gene of the fetal Micrococcus sp. bacterium is at least 97.6% identical to SEQ ID NO: 4. In embodiments, the nucleotide sequence of the 16S rRNA gene of the fetal Micrococcus sp. bacterium is at least 97.7% identical to SEQ ID NO: 4. In embodiments, the nucleotide sequence of the 16S rRNA gene of the fetal Micrococcus sp. bacterium is at least 97.8% identical to SEQ ID NO: 4. In embodiments, the nucleotide sequence of the 16S rRNA gene of the fetal Micrococcus sp. bacterium is at least 97.9% identical to SEQ ID NO: 4. In embodiments, the nucleotide sequence of the 16S rRNA gene of the fetal Micrococcus sp. bacterium is at least 98% identical to SEQ ID NO: 4. In embodiments, the nucleotide sequence of the 16S rRNA gene of the fetal Micrococcus sp. bacterium is at least 98.1% identical to SEQ ID NO: 4. In embodiments, the nucleotide sequence of the 16S rRNA gene of the fetal Micrococcus sp. bacterium is at least 98.2% identical to SEQ ID NO: 4. In embodiments, the nucleotide sequence of the 16S rRNA gene of the fetal Micrococcus sp. bacterium is at least 98.3% identical to SEQ ID NO: 4. In embodiments, the nucleotide sequence of the 16S rRNA gene of the fetal Micrococcus sp. bacterium is at least 98.4% identical to SEQ ID NO: 4. In embodiments, the nucleotide sequence of the 16S rRNA gene of the fetal Micrococcus sp. bacterium is at least 98.5% identical to SEQ ID NO: 4. In embodiments, the nucleotide sequence of the 16S rRNA gene of the fetal Micrococcus sp. bacterium is at least 98.6% identical to SEQ ID NO: 4. In embodiments, the nucleotide sequence of the 16S rRNA gene of the fetal Micrococcus sp. bacterium is at least 98.7% identical to SEQ ID NO: 4. In embodiments, the nucleotide sequence of the 16S rRNA gene of the fetal Micrococcus sp. bacterium is at least 98.8% identical to SEQ ID NO: 4. In embodiments, the nucleotide sequence of the 16S rRNA gene of the fetal Micrococcus sp. bacterium is at least 98.9% identical to SEQ ID NO: 4. In embodiments, the nucleotide sequence of the 16S rRNA gene of the fetal Micrococcus sp. bacterium is at least 99% identical to SEQ ID NO: 4. In embodiments, the nucleotide sequence of the 16S rRNA gene of the fetal Micrococcus sp. bacterium is at least 99.1% identical to SEQ ID NO: 4. In embodiments, the nucleotide sequence of the 16S rRNA gene of the fetal Micrococcus sp. bacterium is at least 99.2% identical to SEQ ID NO: 4. In embodiments, the nucleotide sequence of the 16S rRNA gene of the fetal Micrococcus sp. bacterium is at least 99.3% identical to SEQ ID NO: 4. In embodiments, the nucleotide sequence of the 16S rRNA gene of the fetal Micrococcus sp. bacterium is at least 99.4% identical to SEQ ID NO: 4. In embodiments, the nucleotide sequence of the 16S rRNA gene of the fetal Micrococcus sp. bacterium is at least 99.5% identical to SEQ ID NO: 4. In embodiments, the nucleotide sequence of the 16S rRNA gene of the fetal Micrococcus sp. bacterium is at least 99.6% identical to SEQ ID NO: 4. In embodiments, the nucleotide sequence of the 16S rRNA gene of the fetal Micrococcus sp. bacterium is at least 99.7% identical to SEQ ID NO: 4. In embodiments, the nucleotide sequence of the 16S rRNA gene of the fetal Micrococcus sp. bacterium is at least 99.8% identical to SEQ ID NO: 4. In embodiments, the nucleotide sequence of the 16S rRNA gene of the fetal Micrococcus sp. bacterium is at least 99.9% identical to SEQ ID NO: 4. In embodiments, the nucleotide sequence of the 16S rRNA gene of the fetal Micrococcus sp. bacterium is identical to SEQ ID NO: 4.

In embodiments, the nucleotide sequence of the V4 region of the 16S rRNA gene of the fetal Micrococcus sp. bacterium is at least 95, 96, 97, 98, 99, or 99.5% identical to SEQ ID NO: 2. In embodiments, the nucleotide sequence of the V4 region of the 16S rRNA gene of the fetal Micrococcus sp. bacterium is at least 95% identical to SEQ ID NO: 2. In embodiments, the nucleotide sequence of the V4 region of the 16S rRNA gene of the fetal Micrococcus sp. bacterium is at least 96% identical to SEQ ID NO: 2. In embodiments, the nucleotide sequence of the V4 region of the 16S rRNA gene of the fetal Micrococcus sp. bacterium is at least 97% identical to SEQ ID NO: 2. In embodiments, the nucleotide sequence of the V4 region of the 16S rRNA gene of the fetal Micrococcus sp. bacterium is at least 97.1% identical to SEQ ID NO: 2. In embodiments, the nucleotide sequence of the V4 region of the 16S rRNA gene of the fetal Micrococcus sp. bacterium is at least 97.2% identical to SEQ ID NO: 2. In embodiments, the nucleotide sequence of the V4 region of the 16S rRNA gene of the fetal Micrococcus sp. bacterium is at least 97.3% identical to SEQ ID NO: 2. In embodiments, the nucleotide sequence of the V4 region of the 16S rRNA gene of the fetal Micrococcus sp. bacterium is at least 97.4% identical to SEQ ID NO: 2. In embodiments, the nucleotide sequence of the V4 region of the 16S rRNA gene of the fetal Micrococcus sp. bacterium is at least 97.5% identical to SEQ ID NO: 2. In embodiments, the nucleotide sequence of the V4 region of the 16S rRNA gene of the fetal Micrococcus sp. bacterium is at least 97.6% identical to SEQ ID NO: 2. In embodiments, the nucleotide sequence of the V4 region of the 16S rRNA gene of the fetal Micrococcus sp. bacterium is at least 97.7% identical to SEQ ID NO: 2. In embodiments, the nucleotide sequence of the V4 region of the 16S rRNA gene of the fetal Micrococcus sp. bacterium is at least 97.8% identical to SEQ ID NO: 2. In embodiments, the nucleotide sequence of the V4 region of the 16S rRNA gene of the fetal Micrococcus sp. bacterium is at least 97.9% identical to SEQ ID NO: 2. In embodiments, the nucleotide sequence of the V4 region of the 16S rRNA gene of the fetal Micrococcus sp. bacterium is at least 98% identical to SEQ ID NO: 2. In embodiments, the nucleotide sequence of the V4 region of the 16S rRNA gene of the fetal Micrococcus sp. bacterium is at least 98.1% identical to SEQ ID NO: 2. In embodiments, the nucleotide sequence of the V4 region of the 16S rRNA gene of the fetal Micrococcus sp. bacterium is at least 98.2% identical to SEQ ID NO: 2. In embodiments, the nucleotide sequence of the V4 region of the 16S rRNA gene of the fetal Micrococcus sp. bacterium is at least 98.3% identical to SEQ ID NO: 2. In embodiments, the nucleotide sequence of the V4 region of the 16S rRNA gene of the fetal Micrococcus sp. bacterium is at least 98.4% identical to SEQ ID NO: 2. In embodiments, the nucleotide sequence of the V4 region of the 16S rRNA gene of the fetal Micrococcus sp. bacterium is at least 98.5% identical to SEQ ID NO: 2. In embodiments, the nucleotide sequence of the V4 region of the 16S rRNA gene of the fetal Micrococcus sp. bacterium is at least 98.6% identical to SEQ ID NO: 2. In embodiments, the nucleotide sequence of the V4 region of the 16S rRNA gene of the fetal Micrococcus sp. bacterium is at least 98.7% identical to SEQ ID NO: 2. In embodiments, the nucleotide sequence of the V4 region of the 16S rRNA gene of the fetal Micrococcus sp. bacterium is at least 98.8% identical to SEQ ID NO: 2. In embodiments, the nucleotide sequence of the V4 region of the 16S rRNA gene of the fetal Micrococcus sp. bacterium is at least 98.9% identical to SEQ ID NO: 2. In embodiments, the nucleotide sequence of the V4 region of the 16S rRNA gene of the fetal Micrococcus sp. bacterium is at least 99% identical to SEQ ID NO: 2. In embodiments, the nucleotide sequence of the V4 region of the 16S rRNA gene of the fetal Micrococcus sp. bacterium is at least 99.1% identical to SEQ ID NO: 2. In embodiments, the nucleotide sequence of the V4 region of the 16S rRNA gene of the fetal Micrococcus sp. bacterium is at least 99.2% identical to SEQ ID NO: 2. In embodiments, the nucleotide sequence of the V4 region of the 16S rRNA gene of the fetal Micrococcus sp. bacterium is at least 99.3% identical to SEQ ID NO: 2. In embodiments, the nucleotide sequence of the V4 region of the 16S rRNA gene of the fetal Micrococcus sp. bacterium is at least 99.4% identical to SEQ ID NO: 2. In embodiments, the nucleotide sequence of the V4 region of the 16S rRNA gene of the fetal Micrococcus sp. bacterium is at least 99.5% identical to SEQ ID NO: 2. In embodiments, the nucleotide sequence of the V4 region of the 16S rRNA gene of the fetal Micrococcus sp. bacterium is at least 99.6% identical to SEQ ID NO: 2. In embodiments, the nucleotide sequence of the V4 region of the 16S rRNA gene of the fetal Micrococcus sp. bacterium is at least 99.7% identical to SEQ ID NO: 2. In embodiments, the nucleotide sequence of the V4 region of the 16S rRNA gene of the fetal Micrococcus sp. bacterium is at least 99.8% identical to SEQ ID NO: 2. In embodiments, the nucleotide sequence of the V4 region of the 16S rRNA gene of the fetal Micrococcus sp. bacterium is at least 99.9% identical to SEQ ID NO: 2. In embodiments, the nucleotide sequence of the V4 region of the 16S rRNA gene of the fetal Micrococcus sp. bacterium is identical to SEQ ID NO: 2.

In embodiments, the nucleotide sequence of the V4 region of the 16S rRNA gene of the fetal Micrococcus sp. bacterium is at least 95, 96, 97, 98, 99, or 99.5% identical to SEQ ID NO: 7. In embodiments, the nucleotide sequence of the V4 region of the 16S rRNA gene of the fetal Micrococcus sp. bacterium is at least 95% identical to SEQ ID NO: 7. In embodiments, the nucleotide sequence of the V4 region of the 16S rRNA gene of the fetal Micrococcus sp. bacterium is at least 96% identical to SEQ ID NO: 7. In embodiments, the nucleotide sequence of the V4 region of the 16S rRNA gene of the fetal Micrococcus sp. bacterium is at least 97% identical to SEQ ID NO: 7. In embodiments, the nucleotide sequence of the V4 region of the 16S rRNA gene of the fetal Micrococcus sp. bacterium is at least 97.1% identical to SEQ ID NO: 7. In embodiments, the nucleotide sequence of the V4 region of the 16S rRNA gene of the fetal Micrococcus sp. bacterium is at least 97.2% identical to SEQ ID NO: 7. In embodiments, the nucleotide sequence of the V4 region of the 16S rRNA gene of the fetal Micrococcus sp. bacterium is at least 97.3% identical to SEQ ID NO: 7. In embodiments, the nucleotide sequence of the V4 region of the 16S rRNA gene of the fetal Micrococcus sp. bacterium is at least 97.4% identical to SEQ ID NO: 7. In embodiments, the nucleotide sequence of the V4 region of the 16S rRNA gene of the fetal Micrococcus sp. bacterium is at least 97.5% identical to SEQ ID NO: 7. In embodiments, the nucleotide sequence of the V4 region of the 16S rRNA gene of the fetal Micrococcus sp. bacterium is at least 97.6% identical to SEQ ID NO: 7. In embodiments, the nucleotide sequence of the V4 region of the 16S rRNA gene of the fetal Micrococcus sp. bacterium is at least 97.7% identical to SEQ ID NO: 7. In embodiments, the nucleotide sequence of the V4 region of the 16S rRNA gene of the fetal Micrococcus sp. bacterium is at least 97.8% identical to SEQ ID NO: 7. In embodiments, the nucleotide sequence of the V4 region of the 16S rRNA gene of the fetal Micrococcus sp. bacterium is at least 97.9% identical to SEQ ID NO: 7. In embodiments, the nucleotide sequence of the V4 region of the 16S rRNA gene of the fetal Micrococcus sp. bacterium is at least 98% identical to SEQ ID NO: 7. In embodiments, the nucleotide sequence of the V4 region of the 16S rRNA gene of the fetal Micrococcus sp. bacterium is at least 98.1% identical to SEQ ID NO: 7. In embodiments, the nucleotide sequence of the V4 region of the 16S rRNA gene of the fetal Micrococcus sp. bacterium is at least 98.2% identical to SEQ ID NO: 7. In embodiments, the nucleotide sequence of the V4 region of the 16S rRNA gene of the fetal Micrococcus sp. bacterium is at least 98.3% identical to SEQ ID NO: 7. In embodiments, the nucleotide sequence of the V4 region of the 16S rRNA gene of the fetal Micrococcus sp. bacterium is at least 98.4% identical to SEQ ID NO: 7. In embodiments, the nucleotide sequence of the V4 region of the 16S rRNA gene of the fetal Micrococcus sp. bacterium is at least 98.5% identical to SEQ ID NO: 7. In embodiments, the nucleotide sequence of the V4 region of the 16S rRNA gene of the fetal Micrococcus sp. bacterium is at least 98.6% identical to SEQ ID NO: 7. In embodiments, the nucleotide sequence of the V4 region of the 16S rRNA gene of the fetal Micrococcus sp. bacterium is at least 98.7% identical to SEQ ID NO: 7. In embodiments, the nucleotide sequence of the V4 region of the 16S rRNA gene of the fetal Micrococcus sp. bacterium is at least 98.8% identical to SEQ ID NO: 7. In embodiments, the nucleotide sequence of the V4 region of the 16S rRNA gene of the fetal Micrococcus sp. bacterium is at least 98.9% identical to SEQ ID NO: 7. In embodiments, the nucleotide sequence of the V4 region of the 16S rRNA gene of the fetal Micrococcus sp. bacterium is at least 99% identical to SEQ ID NO: 7. In embodiments, the nucleotide sequence of the V4 region of the 16S rRNA gene of the fetal Micrococcus sp. bacterium is at least 99.1% identical to SEQ ID NO: 7. In embodiments, the nucleotide sequence of the V4 region of the 16S rRNA gene of the fetal Micrococcus sp. bacterium is at least 99.2% identical to SEQ ID NO: 7. In embodiments, the nucleotide sequence of the V4 region of the 16S rRNA gene of the fetal Micrococcus sp. bacterium is at least 99.3% identical to SEQ ID NO: 7. In embodiments, the nucleotide sequence of the V4 region of the 16S rRNA gene of the fetal Micrococcus sp. bacterium is at least 99.4% identical to SEQ ID NO: 7. In embodiments, the nucleotide sequence of the V4 region of the 16S rRNA gene of the fetal Micrococcus sp. bacterium is at least 99.5% identical to SEQ ID NO: 7. In embodiments, the nucleotide sequence of the V4 region of the 16S rRNA gene of the fetal Micrococcus sp. bacterium is at least 99.6% identical to SEQ ID NO: 7. In embodiments, the nucleotide sequence of the V4 region of the 16S rRNA gene of the fetal Micrococcus sp. bacterium is at least 99.7% identical to SEQ ID NO: 7. In embodiments, the nucleotide sequence of the V4 region of the 16S rRNA gene of the fetal Micrococcus sp. bacterium is at least 99.8% identical to SEQ ID NO: 7. In embodiments, the nucleotide sequence of the V4 region of the 16S rRNA gene of the fetal Micrococcus sp. bacterium is at least 99.9% identical to SEQ ID NO: 7. In embodiments, the nucleotide sequence of the V4 region of the 16S rRNA gene of the fetal Micrococcus sp. bacterium is identical to SEQ ID NO: 7.

In embodiments, the Micrococcus sp. reduces IFNγ production by memory promyelocytic leukemia zinc finger protein (PLZF)+ T cells.

In embodiments, the fetal Lactobacillus sp. bacterium is Lacto166. In embodiments, the fetal Lactobacillus sp. bacterium is Lacto167. In embodiments, the Micrococcus sp. bacterium is Micro36.

In an aspect, provided herein is a composition comprising an isolated fetal Micrococcus sp. bacterium and/or an isolated fetal Lactobacillus sp. bacterium and a carrier that is suitable for oral or vaginal administration.

In embodiments, the composition comprises less than about 10, 9, 8, 7, 6, 5, 4, 3, or 2 different species of bacteria. In embodiments, the composition comprises less than about 10 different species of bacteria. In embodiments, the composition comprises less than about 9 different species of bacteria. In embodiments, the composition comprises less than about 8 different species of bacteria. In embodiments, the composition comprises less than about 7 different species of bacteria. In embodiments, the composition comprises less than about 6 different species of bacteria. In embodiments, the composition comprises less than about 5 different species of bacteria. In embodiments, the composition comprises less than about 4 different species of bacteria. In embodiments, the composition comprises less than about 3 different species of bacteria. In embodiments, the composition comprises less than about 2 different species of bacteria.

In embodiments, the composition is a capsule, a tablet, a suspension, a suppository, a powder, a solid, a semi-solid, a liquid, a cream, an oil, an oil-in-water emulsion, a water-in-oil emulsion, or an aqueous solution. In embodiments, the composition is a capsule. In embodiments, the composition is a tablet. In embodiments, the composition a suspension. In embodiments, the composition is a suppository. In embodiments, the composition is a powder. In embodiments, the composition is a solid. In embodiments, the composition is a semi-solid. In embodiments, the composition is a liquid. In embodiments, the composition is a cream. In embodiments, the composition is an oil. In embodiments, the composition is an oil-in-water emulsion. In embodiments, the composition is a water-in-oil emulsion. In embodiments, the composition is an aqueous solution.

In embodiments, the composition has a water activity (a_{w}) less than about 0.9, 0.8, 0.7, 0.6, 0.5, 0.4, 0.3, 0.2, or 0.1 at 20° C. In embodiments, the composition has a water activity (a_{w}) less than about 0.9 at 20° C. In embodiments, the composition has a water activity (a_{w}) less than about 0.8 at 20° C. In embodiments, the composition has a water activity (a_{w}) less than about 0.7 at 20° C. In embodiments, the composition has a water activity (a_{w}) less than about 0.6 at 20° C. In embodiments, the composition has a water activity (a_{w}) less than about 0.5 at 20° C. In embodiments, the composition has a water activity (a_{w}) less than about 0.4 at 20° C. In embodiments, the composition has a water activity (a_{w}) less than about 0.3 at 20° C. In embodiments, the composition has a water activity (a_{w}) less than about 0.2 at 20° C. In embodiments, the composition has a water activity (a_{w}) less than about 0.1 at 20° C.

In embodiments, the composition is a food or a beverage. In embodiments, the composition is a substitute for breast milk (e.g., infant formula). In embodiments, the composition is liquid or dry (e.g., powdered) infant formula.

In embodiments, a carrier that is suitable for oral or vaginal administration is a pharmaceutically acceptable carrier.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

In an aspect, provided herein is an artificial culture comprising an isolated fetal Micrococcus sp. bacterium and/or an isolated fetal Lactobacillus sp. bacterium and a medium.

In embodiments, the artificial culture comprises a placental hormone.

In embodiments, the placental hormone is the only source of carbon in the medium.

In embodiments, the placental hormone is progesterone, estradiol, human placental lactogen, human chorionic gonadotropin, relaxin, estriol (E3), sterol (E4), pregnenolone, pregnenolone sulfate, or dehydroepiandrosterone (DHEA). In embodiments, the placental hormone is progesterone. In embodiments, the placental hormone is estradiol. In embodiments, the placental hormone is human placental lactogen. In embodiments, the placental hormone is human chorionic gonadotropin. In embodiments, the placental hormone is relaxin. In embodiments, the placental hormone is progesterone or estradiol. In embodiments, the placental hormone is an analogue or derivative of a naturally occurring placental hormone. In embodiments, the placental hormone is estriol (E3). In embodiments, the placental hormone is sterol (E4). In embodiments, the placental hormone is pregnenolone. In embodiments, the placental hormone is pregnenolone sulfate. In embodiments, the placental hormone is dehydroepiandrosterone (DHEA).

In embodiments, the estradiol is β-estradiol.

In embodiments, the β-estradiol is 17β-estradiol.

In embodiments, the artificial culture further comprises a monocyte.

In embodiments, the artificial culture further comprises a macrophage.

In embodiments, the monocyte is a primary monocyte or the macrophage is a primary macrophage.

In embodiments, the monocyte is a monocyte a cell line or the macrophage is a macrophage cell line.

In embodiments, the cell line is a THP-1 human monocytic cell line or RAW264.7.

In embodiments, the epithelial cell is a primary epithelial cell.

In embodiments, the epithelial cell is a cell line.

In embodiments, the cell line is a CACO2 cell line.

In embodiments, the artificial culture is in a cell culture plate, a flask, or a biofermentor. In embodiments, the artificial culture is in a cell culture plate. In embodiments, the artificial culture is in a flask. In embodiments, the artificial culture is in or a biofermentor.

In embodiments, the cell culture plate is an agar plate.

In embodiments, Micrococcus sp. is cultured alone in cRPMI prepared as described in Example 1, brain heart infusion (BHI), or tryptone soya agar (TSA).

In embodiments, Lactobacillus sp. can be cultured alone in cRPMI prepared as described in Example 1 or TSA+blood (e.g., about 1, 2, 3, 4, 5, 6, 7, 8, 9, 10, 2-7, 4-6% blood, such as horse blood).

In embodiments, Micrococcus sp. and/or Lactobacillus sp. is cultured together with a eukaryotic cell. In embodiments, the eukaryotic cell is a monocyte, a macrophage, or an epithelial cell. In embodiments, the eukaryotic cell is a primary cell. In embodiments, the eukaryotic cell is a cell line. In embodiments, the cell line is a THP-1 human monocytic cell line, RAW264.7, or CACO2.

In embodiments, Micrococcus sp. and/or Lactobacillus sp. is cultured together with monocytes, the cells can be cultured in cRPMI as described in Example 1.

In embodiments, Micrococcus sp. and/or Lactobacillus sp. cells are cultured under hypoxic conditions. In embodiments, the hypoxic conditions mimick the conditions in the fetal intestine. In embodiments, bacterial culture methods are enhanced at 37° C., 4% 02, 5% CO₂ to mimick hypoxic conditions in the fetal intestine. In embodiments, Micrococcus sp. and/or Lactobacillus sp. cells are cultured at ambient oxygen levels. In embodiments, the Lactobacillus sp. cells, but not the Micrococcus sp. cells grow in completely anaerobic conditions (0% 02). In embodiments, the culture temperature is about 37° C.

In embodiments, Micrococcus sp. and/or Lactobacillus sp. cells are cultured at about 1%, 2%, 3%, 4%, 5%, 6%, 7%, 8%, 9%, 10%, 1-5%, 2-5%, 3-5%, 4-5%, or 5-10% 02. In embodiments, Micrococcus sp. and/or Lactobacillus sp. cells are cultured at about 1%, 2%, 3%, 4%, 5%, 6%, 7%, 8%, 9%, 10%, 1-5%, 2-5%, 3-5%, 4-5%, or 5-10% CO₂.

### V. EXAMPLES

### Example 1: Viable Bacteria are Present in Human Intestine in Utero

Mucosal immunity develops in the human fetal intestine by 11-14 weeks gestation, yet whether microbes exist in utero and interact with intestinal immunity is unknown. Of 50 human fetal meconium samples profiled, Lactobacillus- (n=6) or Micrococcaceae-enriched (n=9) meconium were most commonly detected and associated with distinct intestinal epithelial transcriptomes and proportions of lamina propria PLZF+ CD161+ CD4+ T cells. Fetal intestinal bacterial isolates, identified by whole genome sequencing as Lactobacillus jensenii or Micrococcus luteus, grew on placental hormones, remained viable within fetal antigen presenting cells, and exhibited species-specific immunomodulatory capacity mirroring features observed ex vivo. Thus, intestinal bacteria with distinct immunomodulatory capacities are variably present during human gestation.

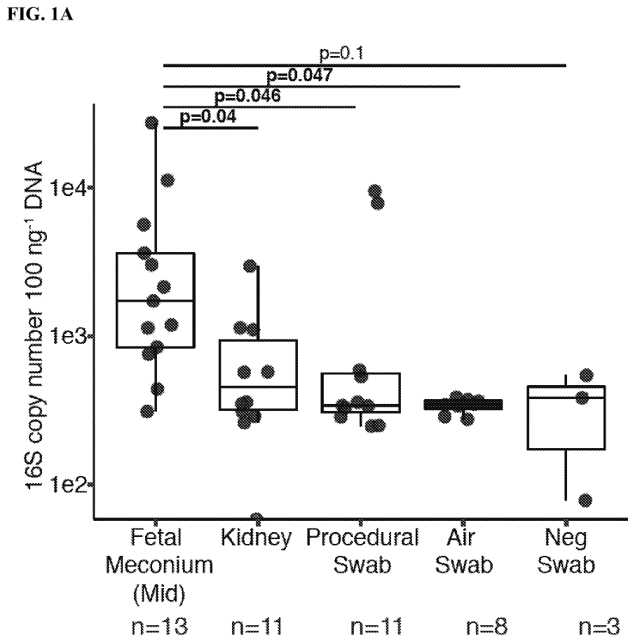

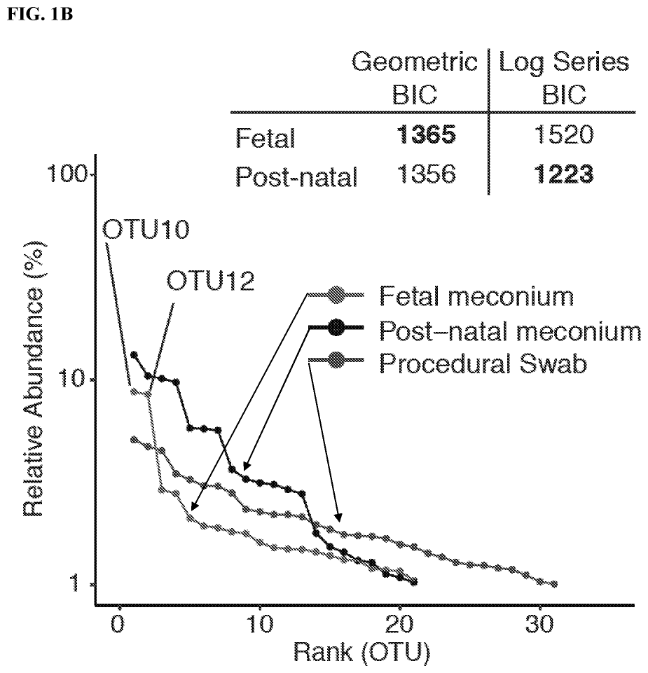

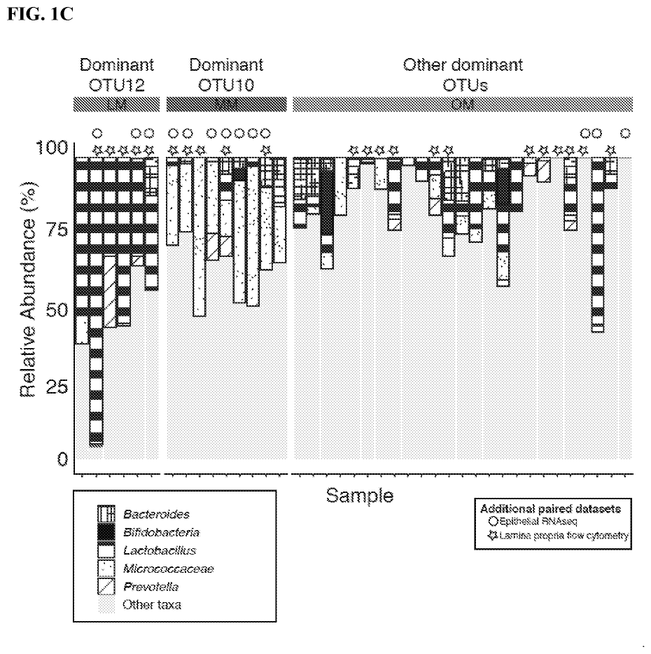

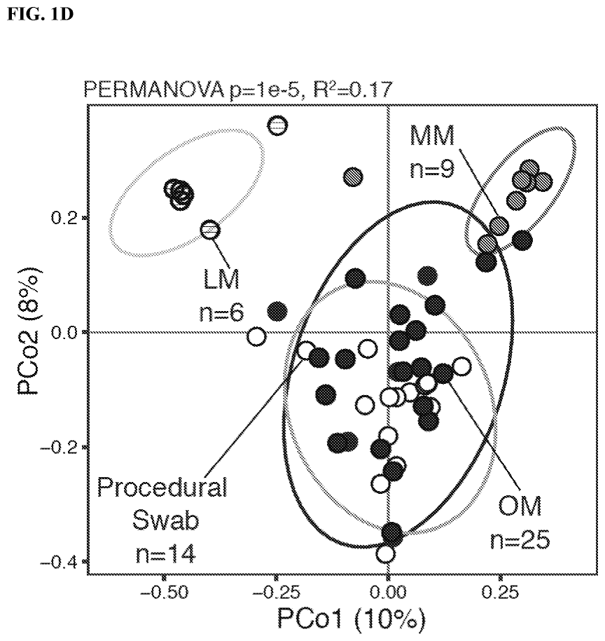

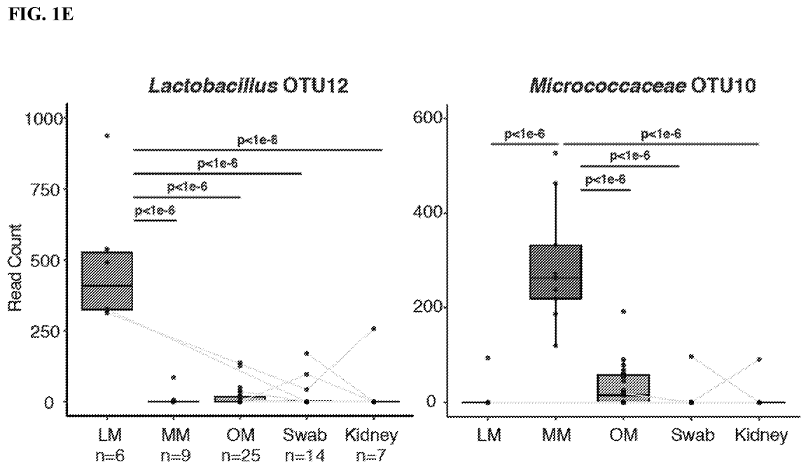

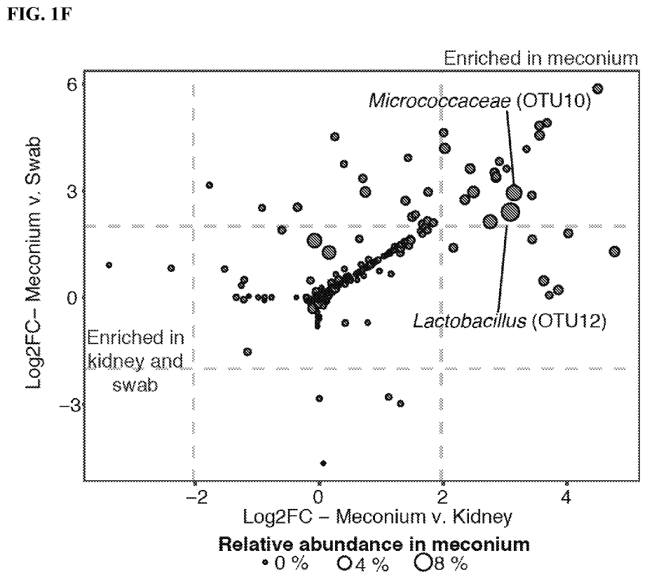

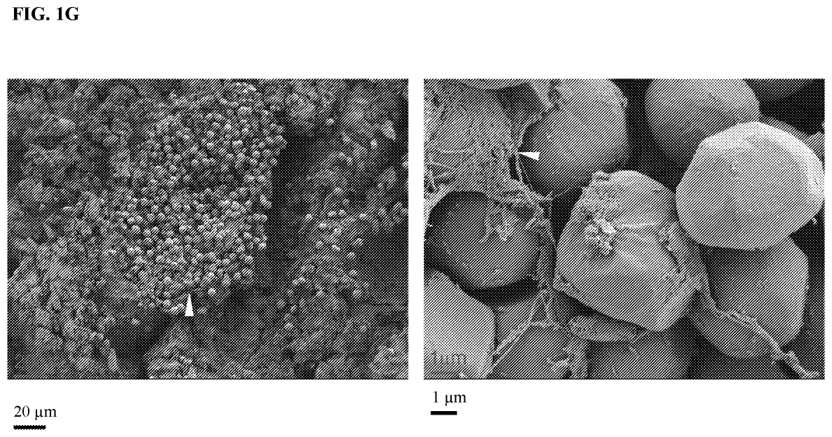

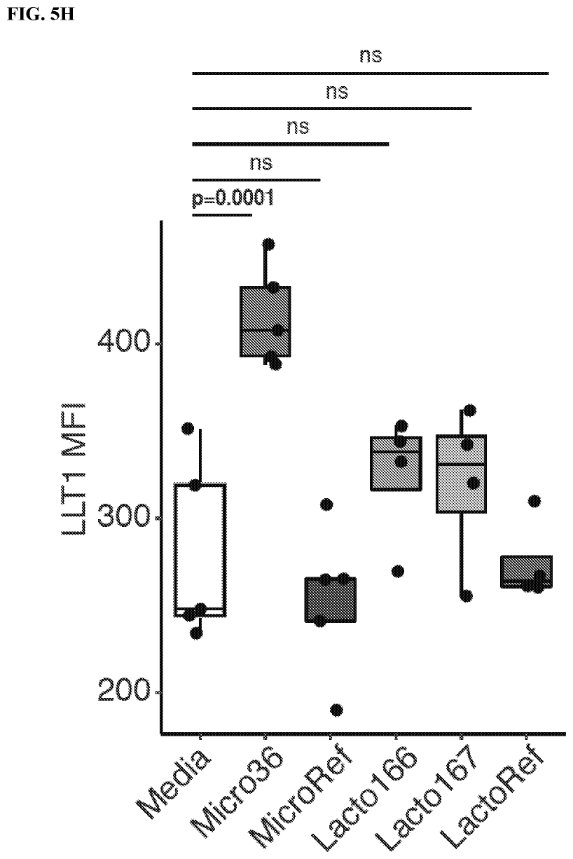

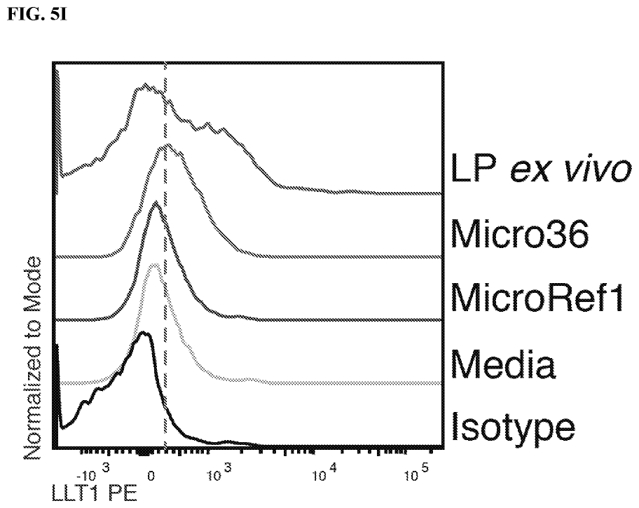

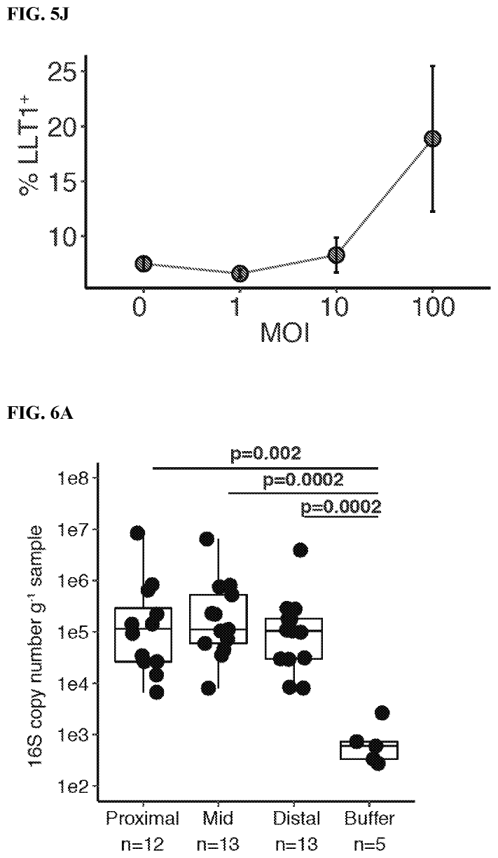

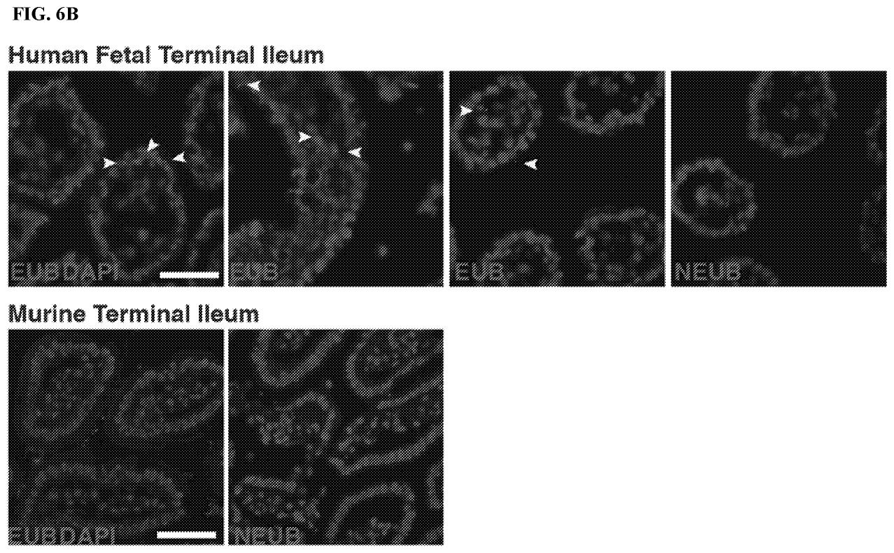

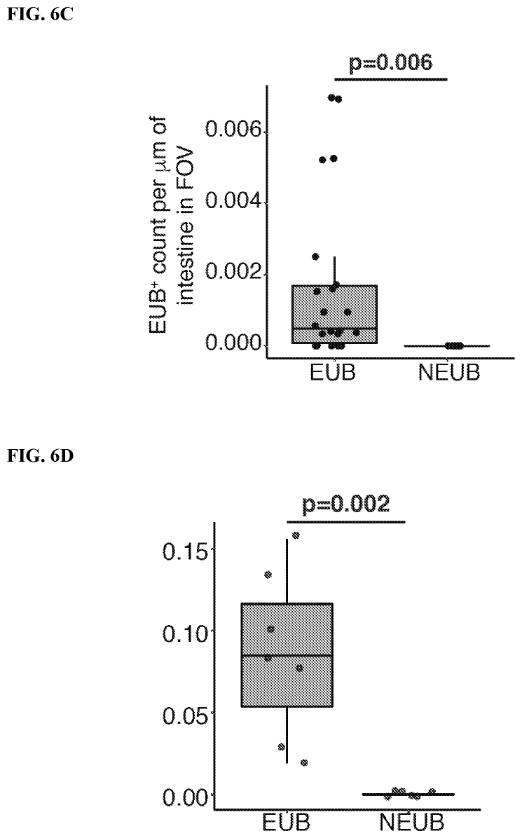

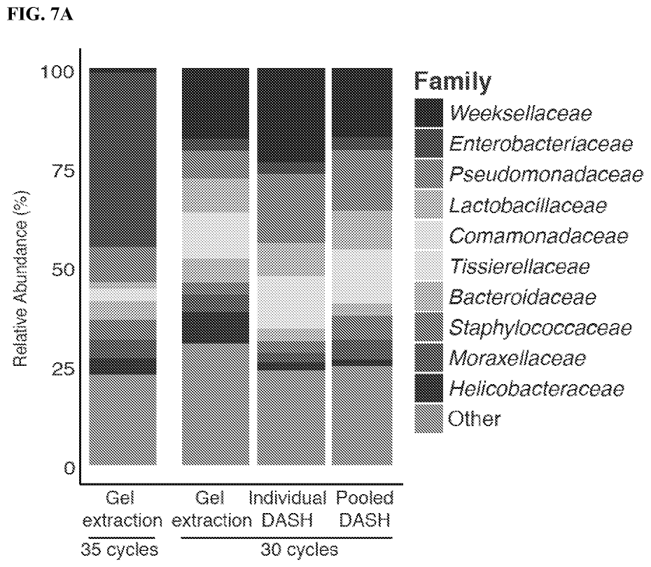

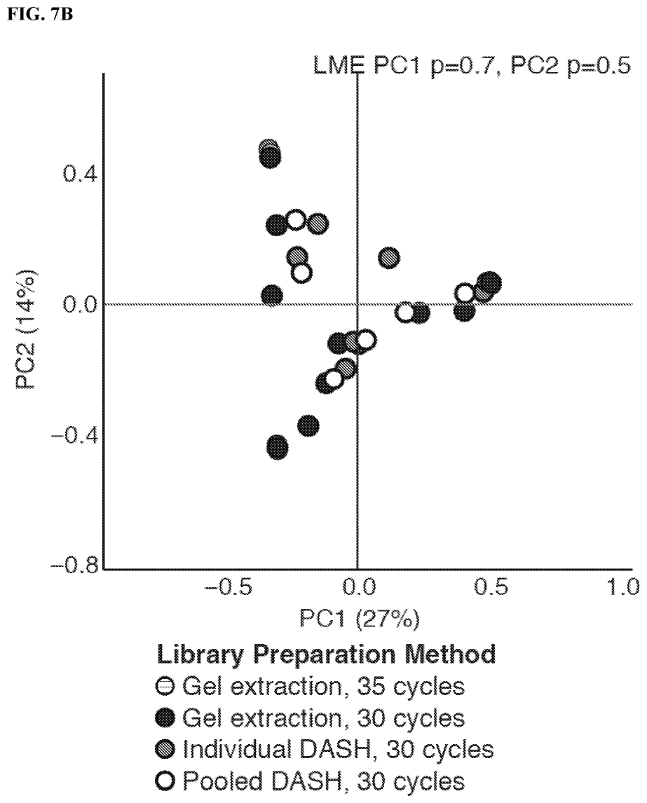

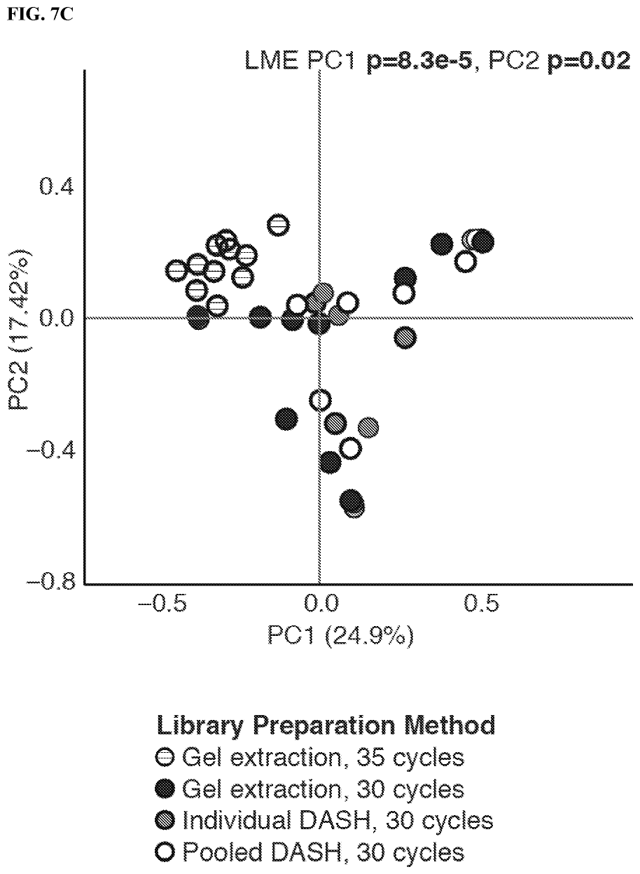

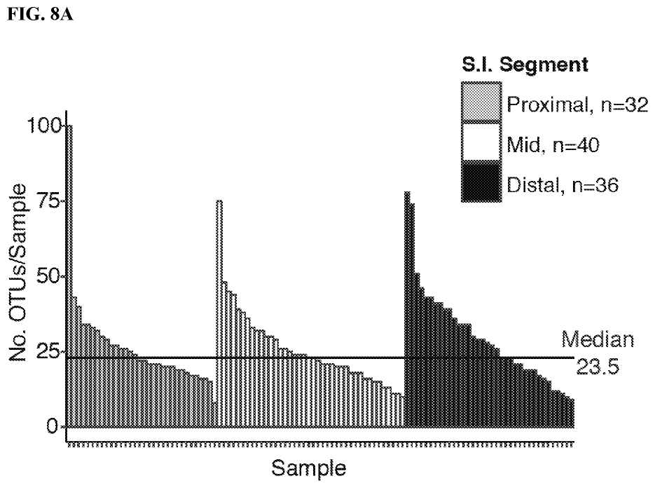

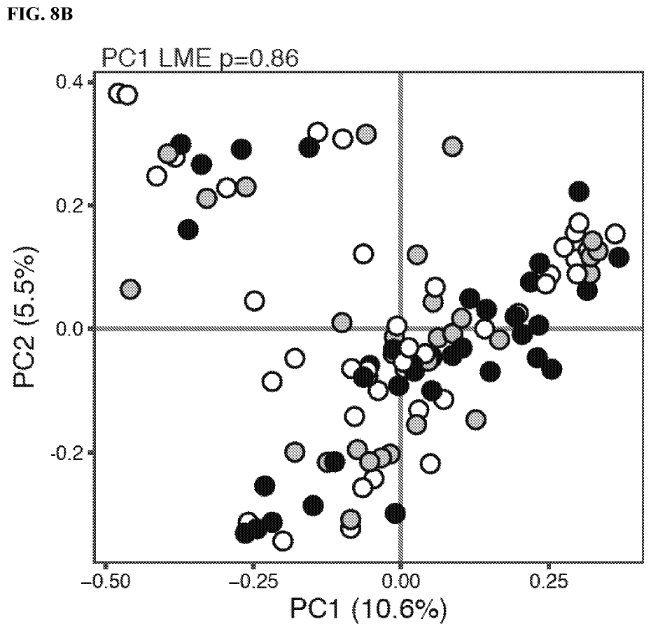

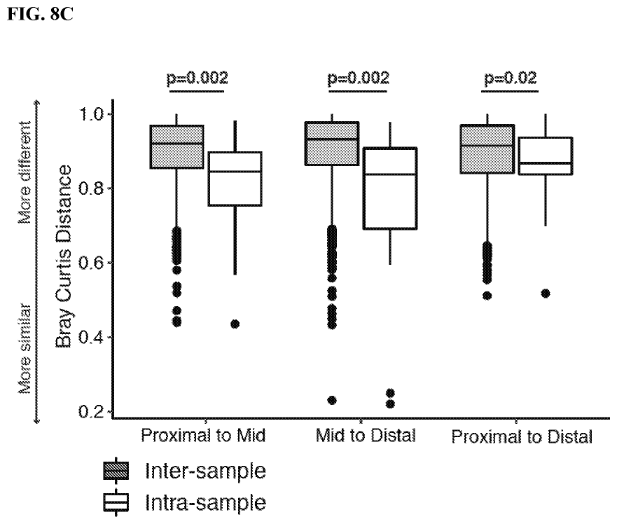

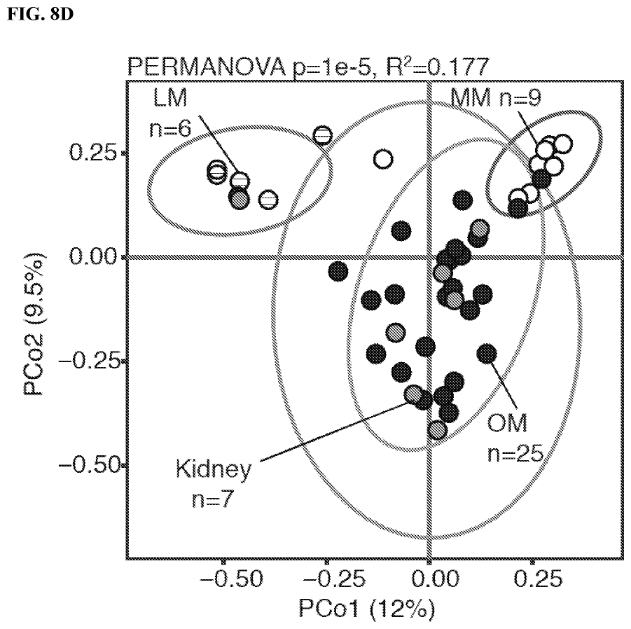

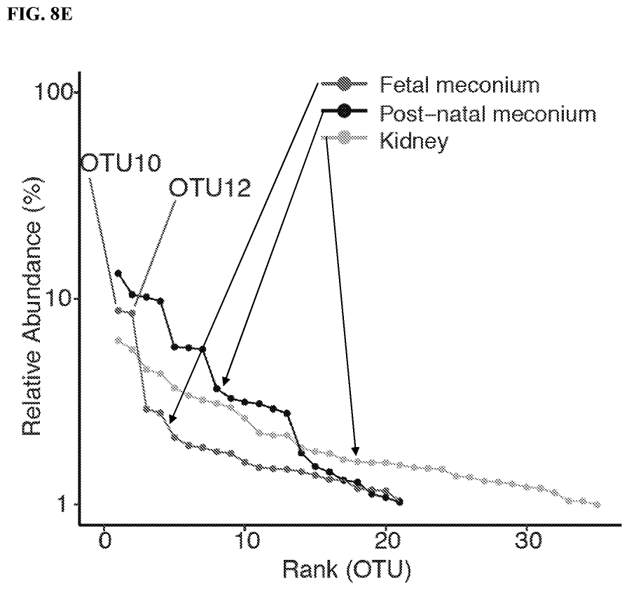

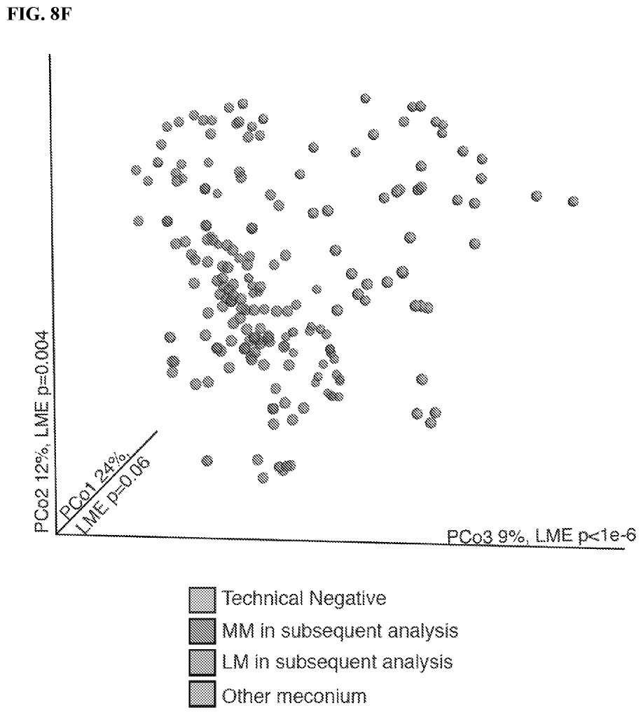

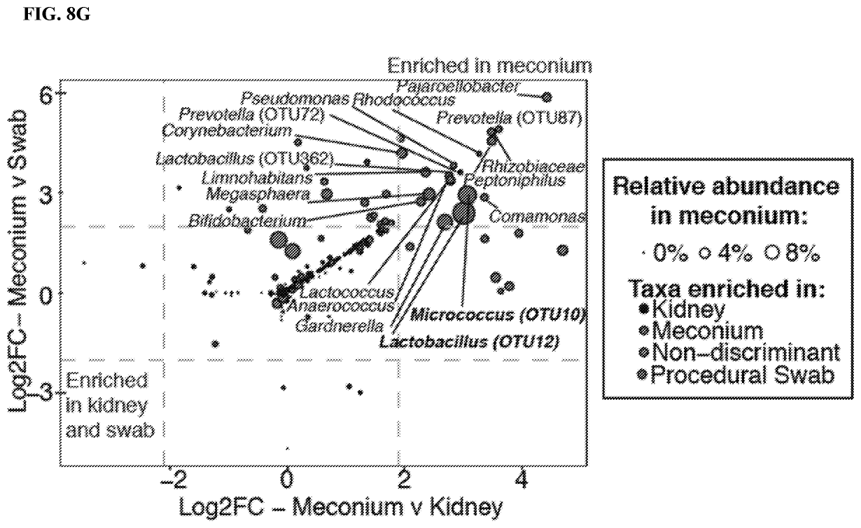

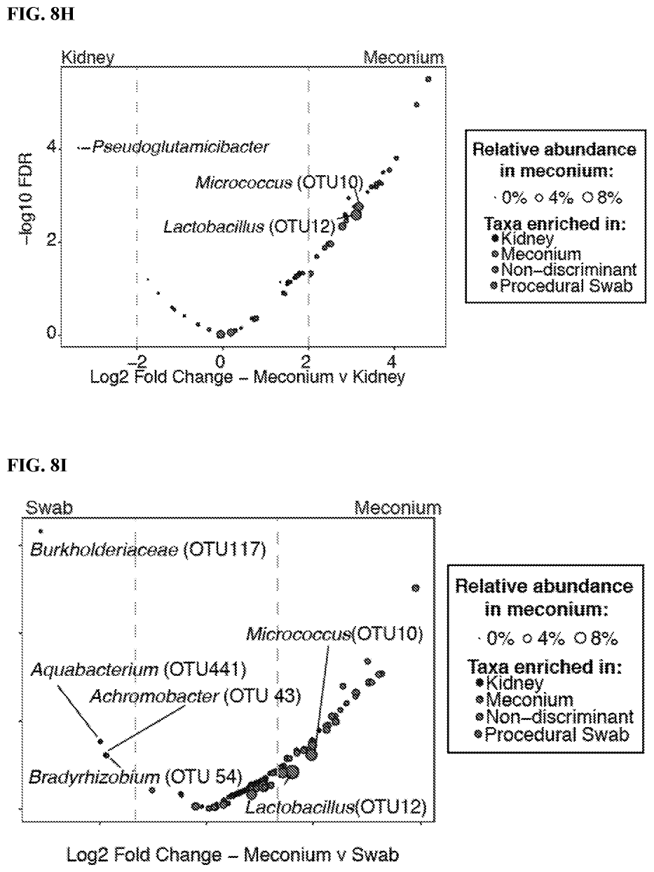

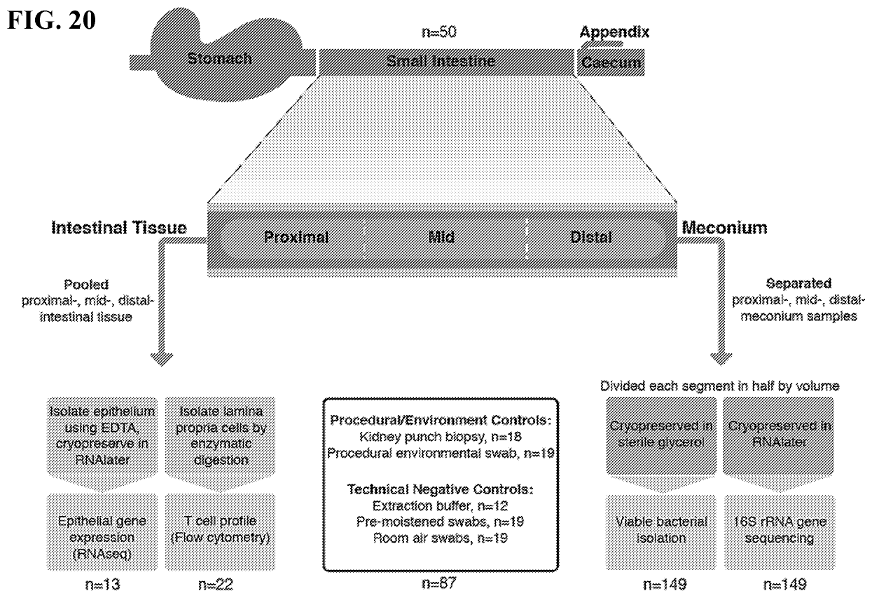

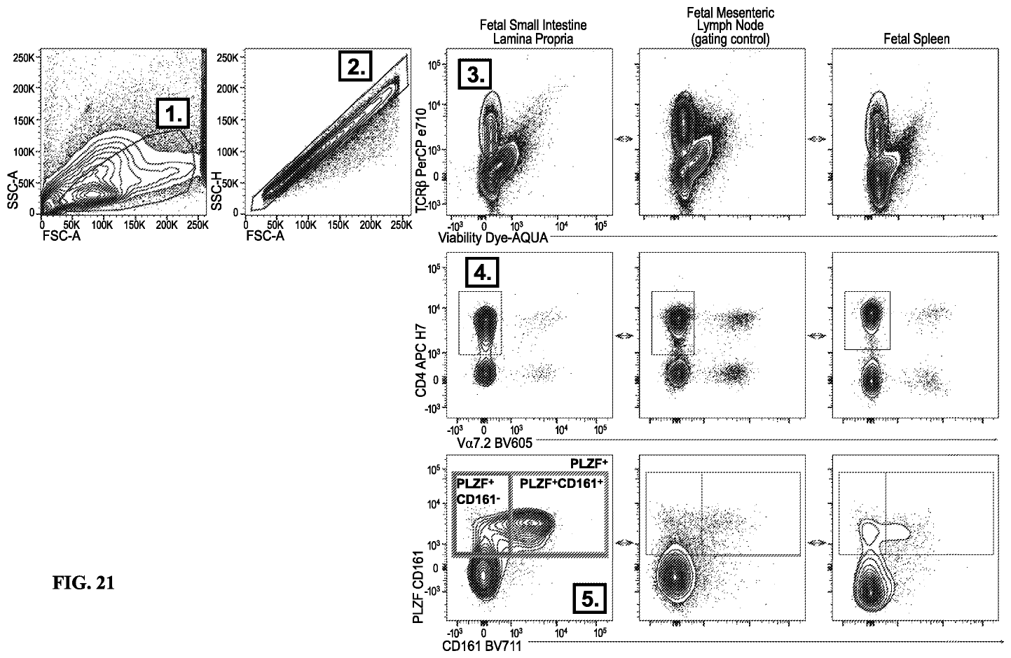

We established a bank of human fetal small intestine meconium samples (n=50 subjects; n=149 samples, n=87 technical and procedural controls; FIG. 20; Tables 1A-B; Example 2) to determine whether a bacterial signal is evident during the second trimester of gestation. Irrespective of the small intestine segment sampled, total bacterial burden by 16S rRNA copy number was low in fetal meconium, but consistently greater than that of extraction buffer, procedural swab, hospital room air swab, blank cotton swab, or fetal kidney controls (FIG. 1A, FIG. 6A). Fluorescent in situ hybridization for eubacteria of fetal ileal sections reaffirmed this observation (FIGS. 6B-D). To enhance bacterial signal prior to V4 16S rRNA gene amplification, human mitochondrial 16S DNA (mtDNA) was depleted using Cas9 targeting (Depletion of Abundant Sequences by Hybridization, DASH; Example 2) [17]; this did not alter the profile of detected bacteria as compared to gel extraction (FIGS. 7A-C). In 16S rRNA datasets stringently controlled for environmental and procedural contamination (Tables 1-2, Example 2), a simple bacterial profile was identified in 40 subjects comprising a median of 23.5 operational taxonomic units (OTUs) with ≥5 sequence read counts per sample (n=10 samples did not yield sufficient sequence reads following filtering; Tables 1-2, FIG. 8A). Bacterial profiles did not significantly differ in technical replicates sampled along the length of the intestine within subjects (n=108 samples; LME p=0.78; FIG. 8B) and inter-sample distances were greater than intra-sample distances (FIG. 8C). Thus, subsequent analyses focused on the mid-segment (n=40) of the small intestine.

Fetal and post-natal meconium samples (the latter from an independent study of healthy, term neonates [15]) exhibited distinct taxon distributions compared with procedural swab controls (FIG. 1B). Post-natal meconium microbiota best fit a log-series model (indicating expansion of rarer taxa), while fetal meconium best fit a geometric series model, indicative of dominance by a small number of taxa and consistent with early stages of ecological primary succession [18] (FIG. 1B). Lactobacillus OTU12 and Micrococcaceae OTU10 represented the two highest ranked fetal meconium taxa by relative abundance (FIG. 1B) and the dominant taxon within distinct subsets of samples (Lactobacillus-meconium, LM; n=6, or Micrococcaceae-meconium, MM; n=9; FIG. 1C). The remaining samples were variably dominated by other bacterial taxa (Other-meconium, OM; n=25), including distinct taxa within Lactobacillus and Micrococcaceae, as well as Bacteroides, Bifidobacteria, and Prevotella (FIG. 1C). Though OM samples represented the majority of meconium studied, their 16S rRNA profiles were similar to that of biological controls (FIG. 1D). In contrast, LM and MM exhibited significantly distinct bacterial profiles from OM samples, procedural and kidney controls (PERMANOVA, R²=0.167, p=1 e−5; FIG. 1D, FIGS. 8D-E), and from a variety of technical controls (n=48, FIG. 8F). Lactobacillus OTU12 and Micrococcaceae OTU10 were not identified as contaminants using stringent thresholds (decontam R package; p threshold=0.6) and were either undetected or found at extremely low levels in controls and OM samples (FIG. 1E). These two taxa were also amongst a number of OTUs both significantly enriched and highest in abundance in fetal meconium (DESEQ2; L2FC FDR<0.05) compared to procedural swabs and kidney controls (FIG. 1F, FIGS. 8G-I) and were the dominant taxa in this subset of samples. Thus, we considered Lactobacillus OTU12 and Micrococcaceae OTU10 to represent a bona fide bacterial signal in the human fetal intestine.

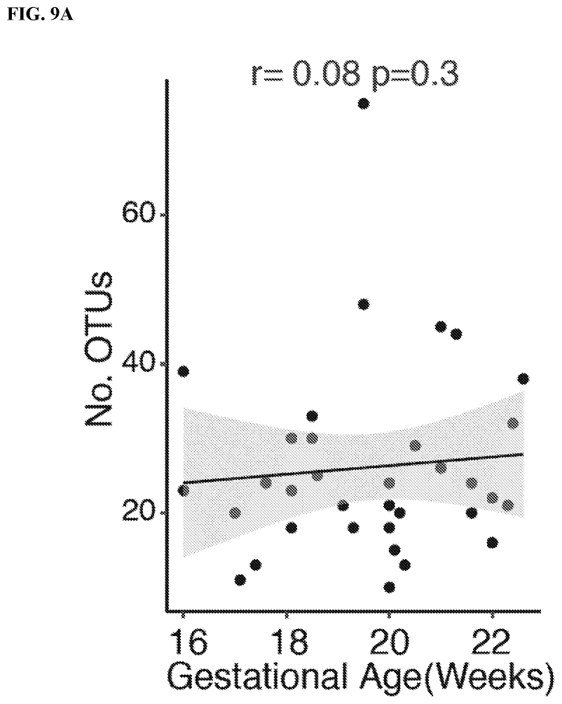

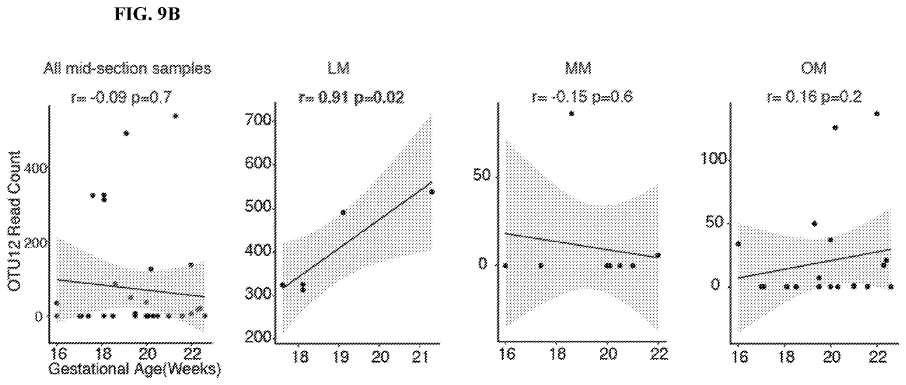

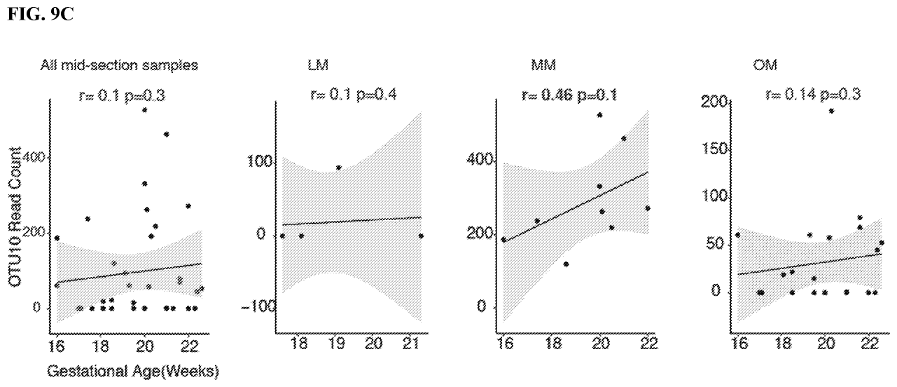

The number of detected OTUs per sample and the relative abundance of OTU12 or OTU10 was not significantly correlated with gestational age when all samples were considered (FIGS. 9A-C). However, within the LM group a positive correlation between OTU12 read counts and gestational age was observed (Pearson's r=0.91, p=0.02) and a similar trend for OTU10 was observed within the MM group (Pearson's r=0.5, p=0.1; FIGS. 9B-C). These taxa did not exhibit a significant correlation with gestational age within the OM group (FIGS. 9B-C).

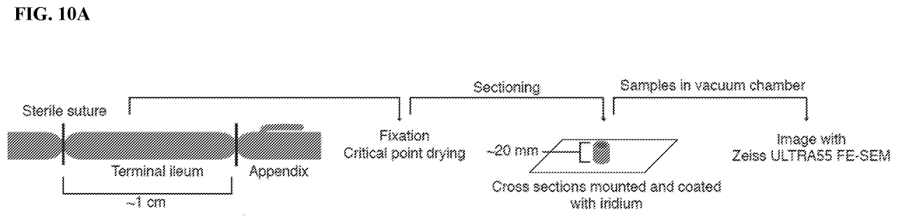

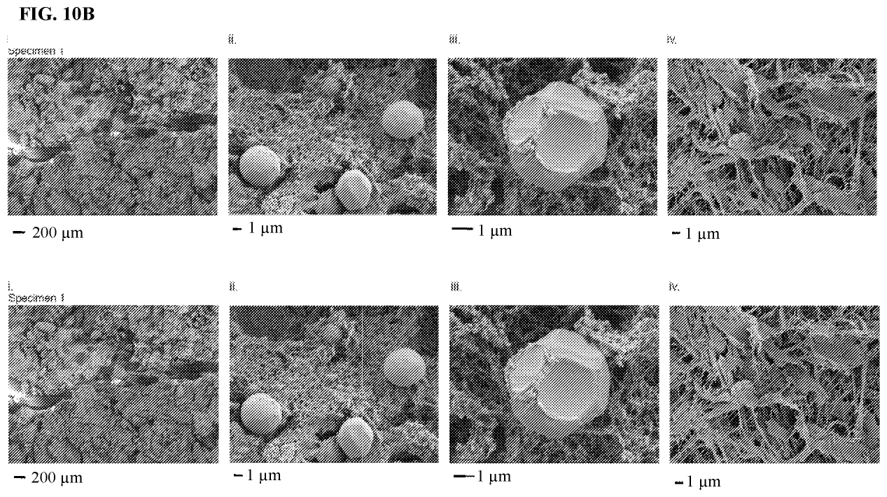

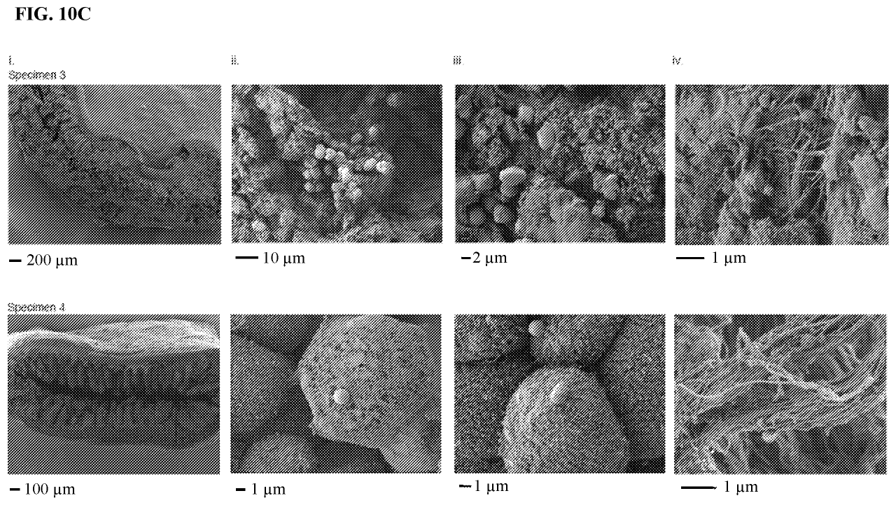

To further validate the presence of bacteria in the fetal intestine, we performed scanning electron microscopy (SEM) of four additional fetal terminal ileum specimens, minimizing intestinal lumen exposure to the environment (FIG. 10A, Example 2). In three of four independent fetal specimens (FIG. 10B-C; Specimens 1-3), clusters of tightly packed cellular structures morphologically and proportionally consistent with bacterial cocci were observed in discrete, isolated pockets of meconium, deeply embedded within existing mucin structures (FIG. 1G, FIG. 10B-C). Confirming their localization to meconium, these cocci structures were not observed in sub-epithelial regions such as the lamina propria or muscularis, where collagen tendrils were evident (FIG. 10B-C(panel iv)). Thus, consistent with our molecular and FISH-based analyses (FIG. 6B-C), we observed discrete cellular structures consistent with coccoid bacterial morphology, embedded within isolated pockets of fetal intestinal meconium during the second trimester of human gestation.

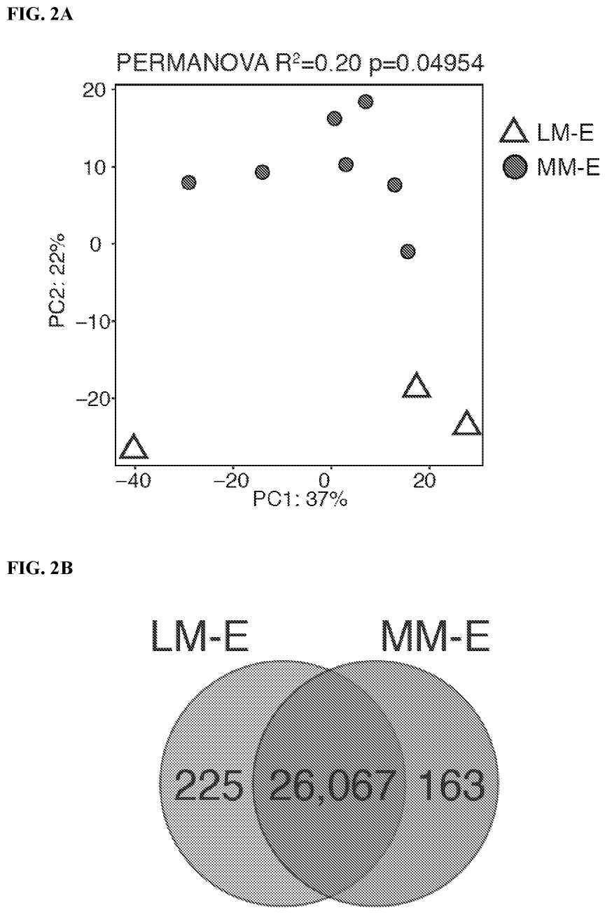

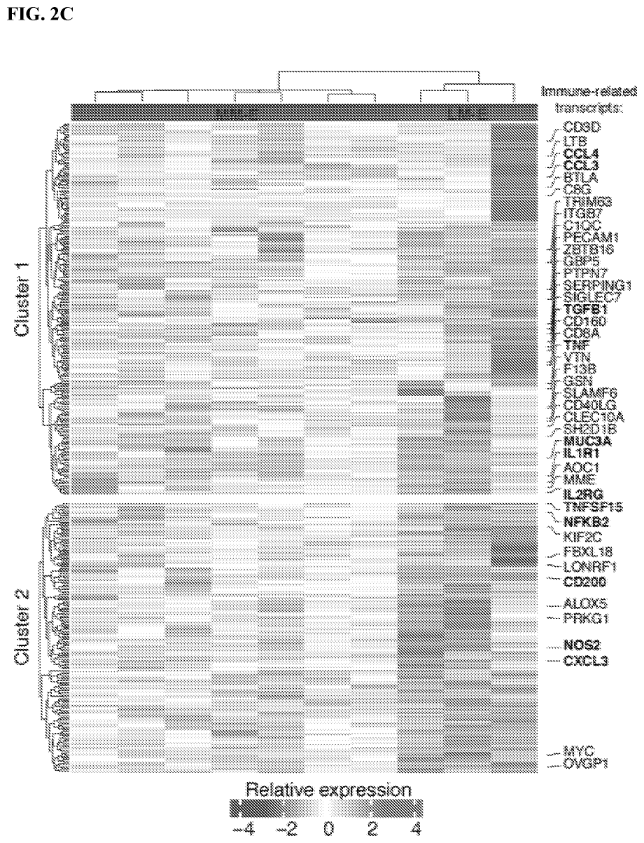

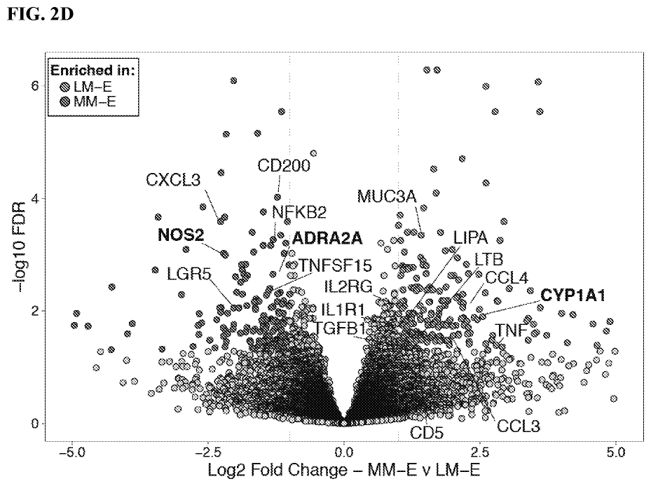

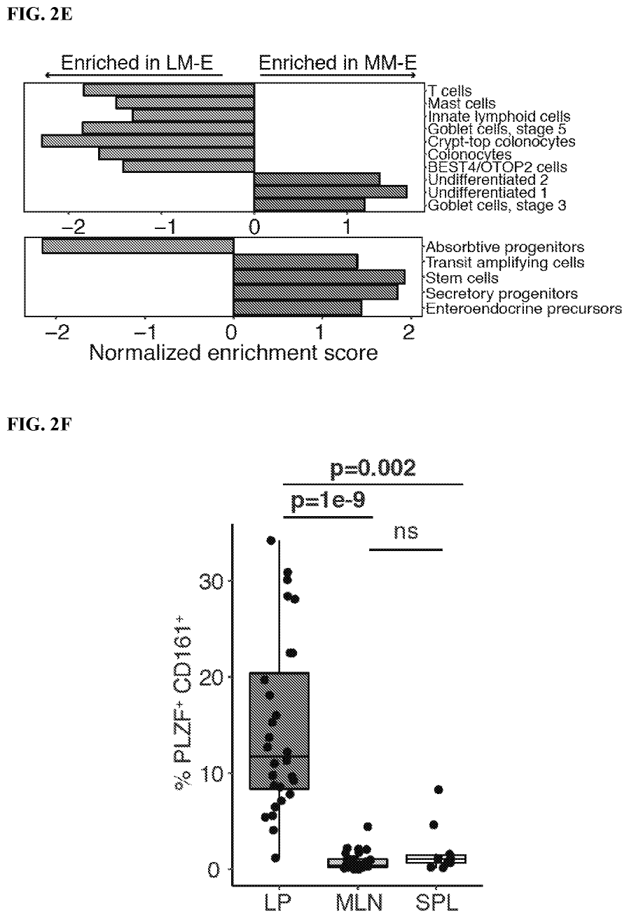

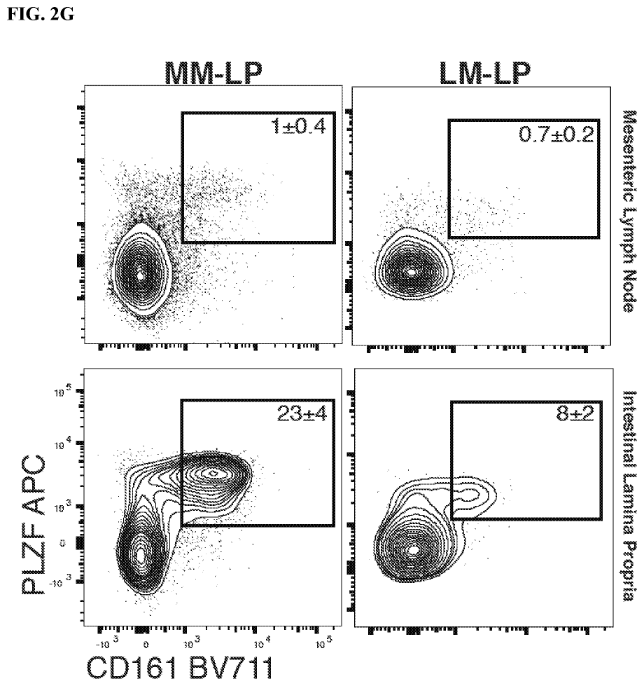

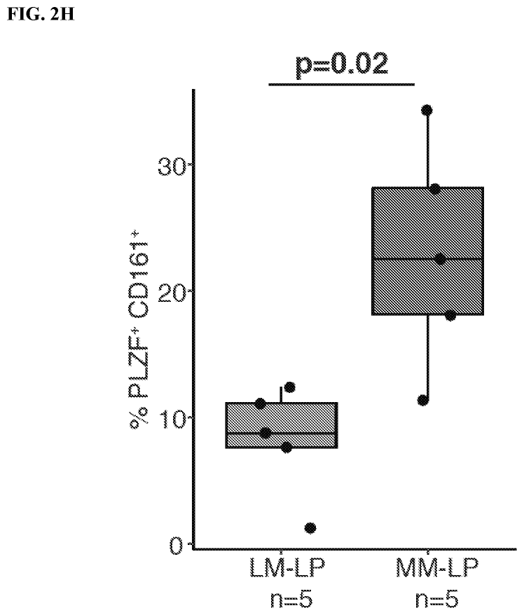

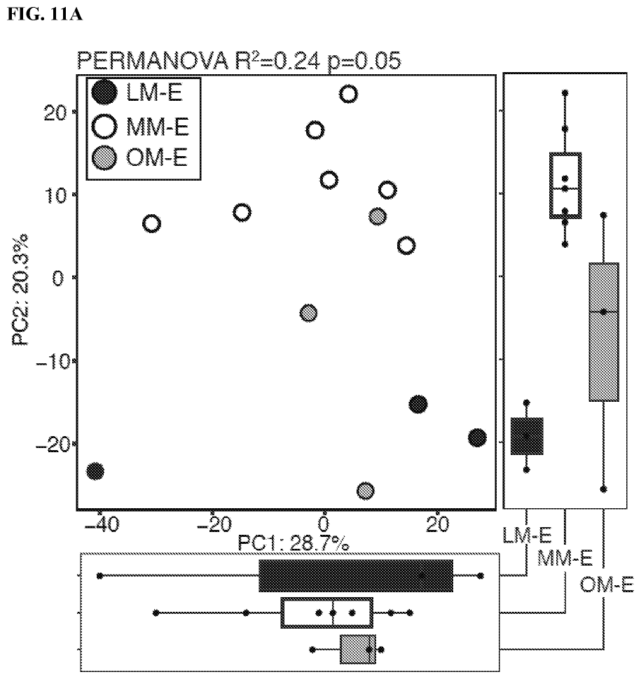

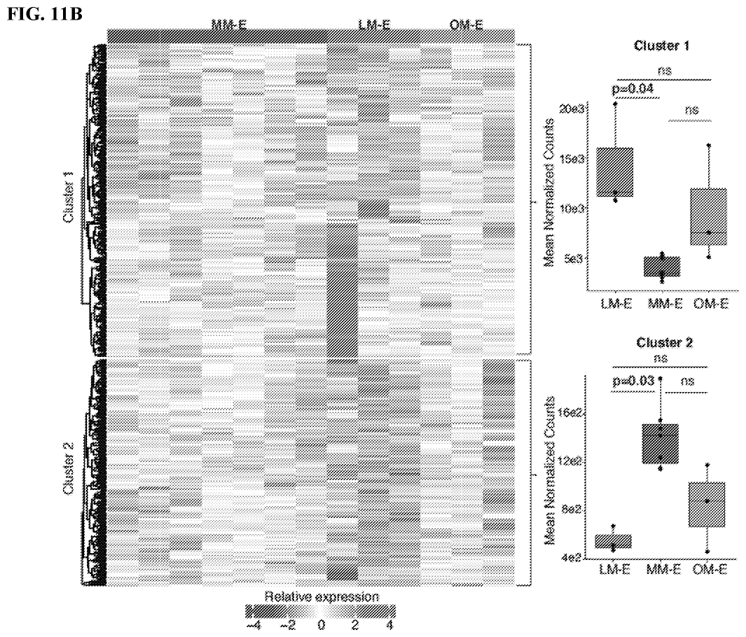

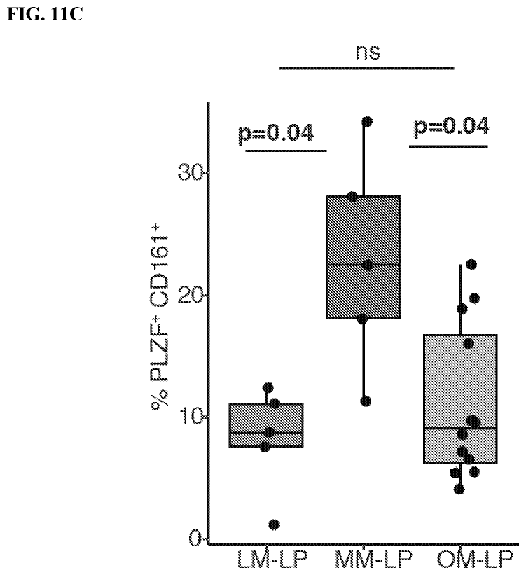

To investigate host response to the presence of specific Lactobacillus and Micrococcaceae in fetal meconium, we performed intestinal epithelial cell layer RNA sequencing (RNASeq) of specimens that were classified as LM, MM, and OM by 16S rRNA profiling (n=3, n=7, n=3, respectively). LM-associated epithelium (LM-E) and MM-associated epithelium (MM-E) exhibited distinct transcriptional profiles (PERMANOVA p=0.04954 R²=0.20, FIG. 2A); the OM-associated epithelium (OM-E) group was interspersed between LM-E and MM-E (FIG. 11A) and exhibited intermediate expression of differentially expressed genes associated with LM-E (Cluster 1) and MM-E (Cluster 2; FIG. 11B). Focusing our epithelial transcriptome analysis on LM-E and MM-E, we noted that LM-E was significantly enriched for 225 and MM-E for 163 transcripts (FDR <0.05, L2FC|1|; FIG. 2B-C, Table 3). LM-E exhibited differentially expressed genes associated with metabolism (e.g. CYP1A1; FIG. 2D) and gene set enrichment analysis (GSEA) identified genes associated with absorption and mature goblet cells [19] (e.g. MUC3A; FIG. 2E, Table 3), consistent with the development of epithelial barrier integrity and function. In contrast, MM-E exhibited upregulation of transcripts associated with undifferentiated and precursor cell populations such as immature goblet cells, stem cells, transit amplifying cells, and enteroendocrine precursors [19] (e.g. LGR5; FIG. 2E, Table 3). Divergent expression of transcripts associated with TLR-signaling (NFKB2, TNFSF15, TNF, LTB) and phagolysosome function (LIPA, NOS2) were also observed (FIGS. 2C-D, Table 3). MM-E upregulated the innate immune cell chemoattractant CXCL3 and the macrophage inhibitory protein CD200, while LM-E was enriched for chemokines CCL3 and CCL4 (FIG. 2C), indicating distinct programs of immune cell activation and recruitment in the presence of these bacteria. The LM-E transcriptome was also enriched for genes associated with activation of immune cells including T cells, mast cells, and innate lymphoid cells by GSEA [19] (e.g. TGFB1, TNF, IL1R1, IL2RG and CD5; FIG. 2E).

To assess adaptive immune cell phenotypes, a subset of fetal lamina propria (LP) samples paired with meconium (n=22) were profiled by flow cytometry at the time of sample collection. This confirmed recent findings [6] that PLZF⁺ CD161⁺ CD4⁺ Vα7.2⁻TCRαβ⁺ T cells were highly abundant in the fetal lamina propria in contrast to mesenteric lymph node and spleen (FIG. 2I, FIGS. 2F-G). Compared with LM-associated samples (LM-LP; n=5), MM lamina propria (MM-LP; n=5) exhibited significantly greater proportions of PLZF⁺ CD161⁺ T cells (FIG. 2H). OM-associated LP (OM-LP) had similar proportions of these T cells to LM-LP, but significantly lower than MM-LP (FIG. 11C). Thus, the presence of Micrococcaceae and Lactobacillus in fetal meconium is associated with distinct mucosal immunity which may contribute to their selective enrichment in utero.

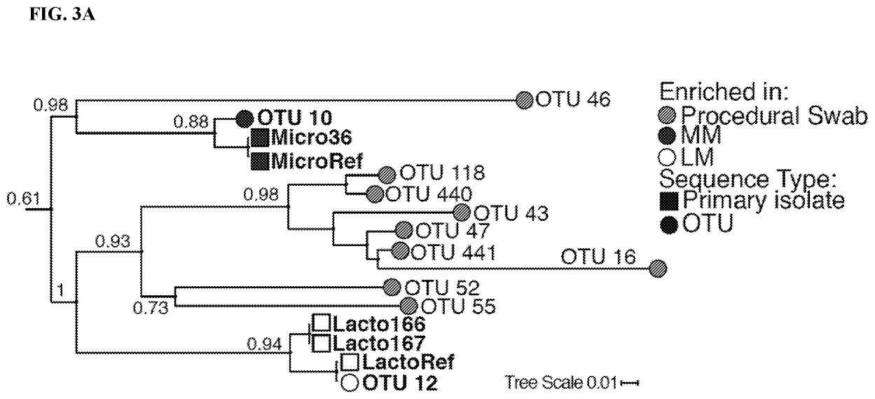

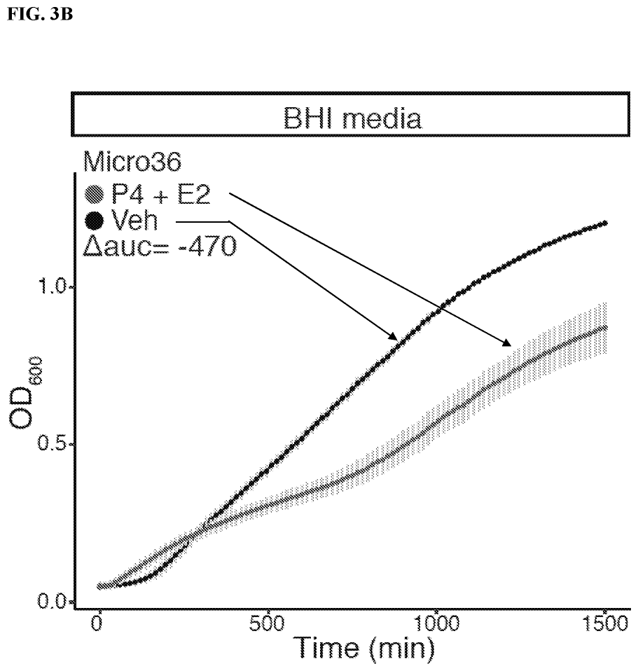

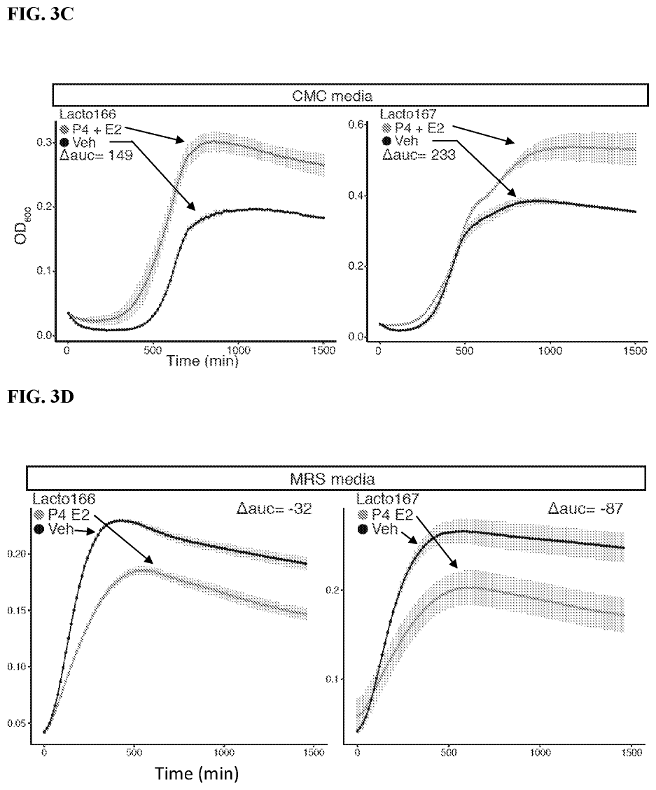

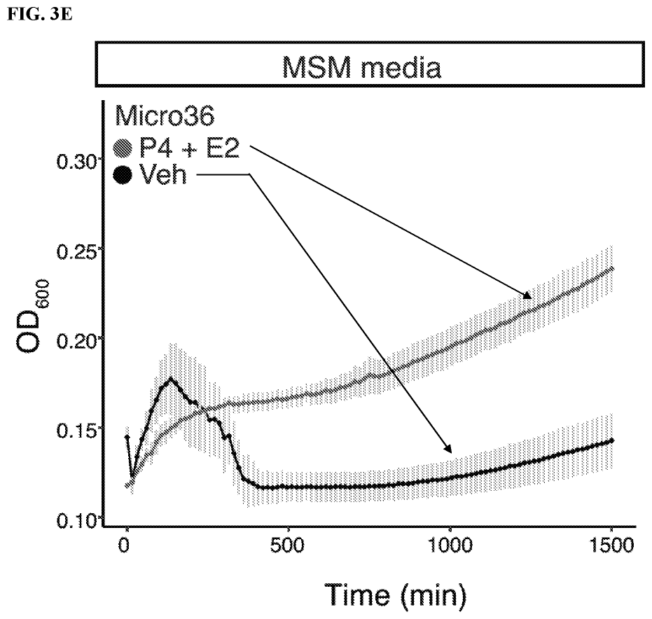

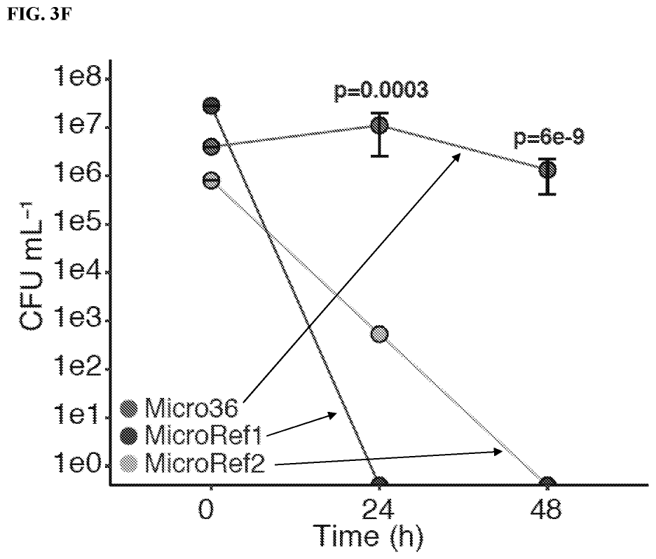

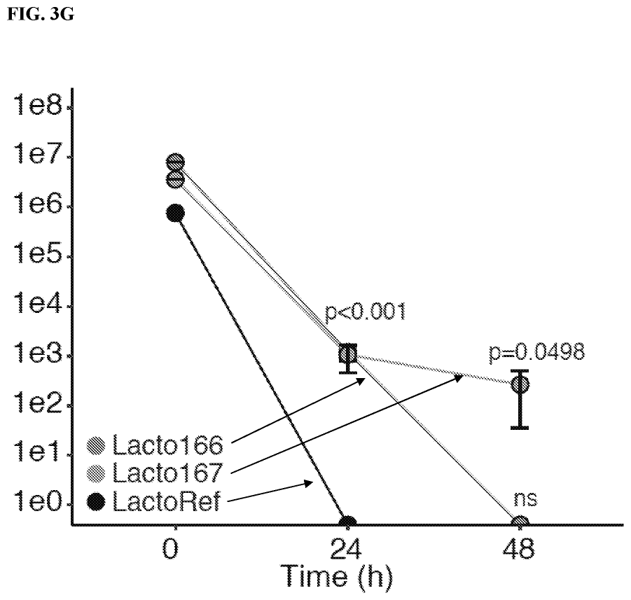

To determine whether intestinal Lactobacillus and Micrococaceae were viable, we attempted isolation from cryopreserved LM and MM fetal meconium samples with the highest read counts for each taxon, respectively. Isolates could not be recovered using traditional selective media for these genera and were only obtained under culture conditions that mimicked the fetal intestinal environment (Tables 4A-B), including the addition of placental steroid hormones or THP1 human monocyte cells to isolation media. Using the SILVA database to classify full-length 16S rRNA gene sequences of fetal isolates, two isolates from two independent specimens were classified as Lactobacillus (Lacto166 and Lacto167) and the third as Micrococcus (Micro36; Tables 4A-B). The V4 region of the Lactobacillus isolates exhibited high homology with OTU12 (96% for each) and the Micrococcus isolate with OTU10 (97%; FIG. 3A, FIGS. 12A-B, Tables 4A-B).

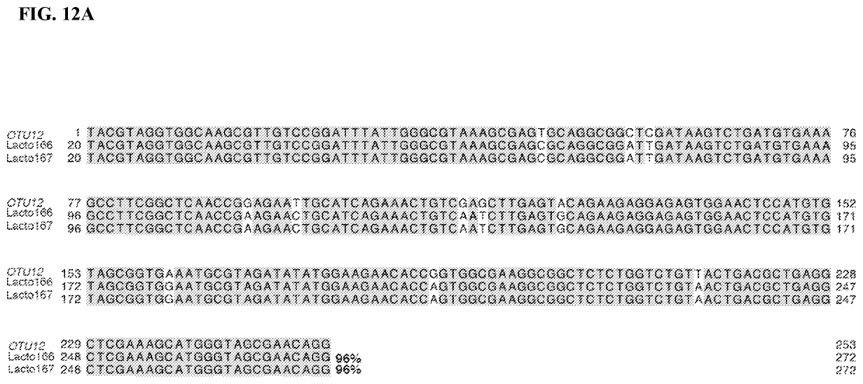

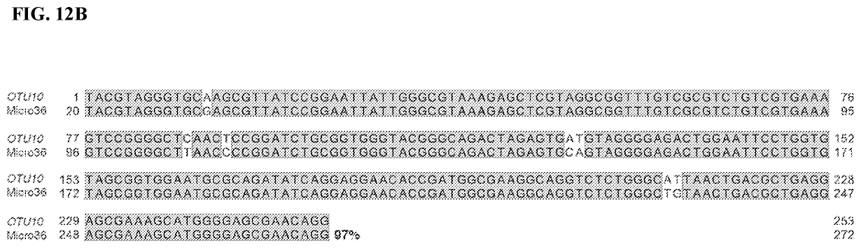

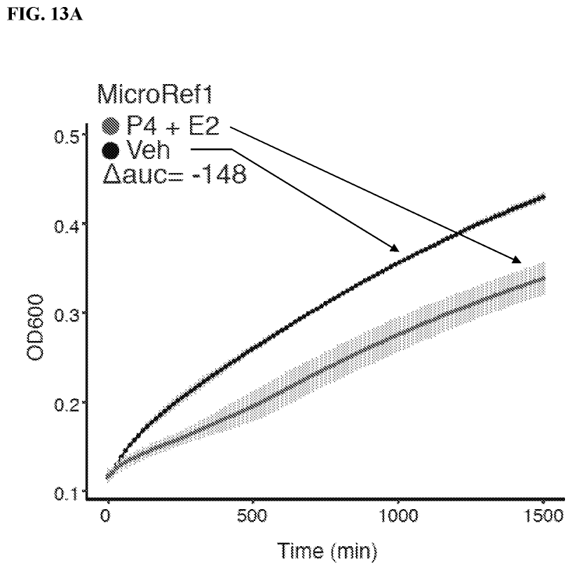

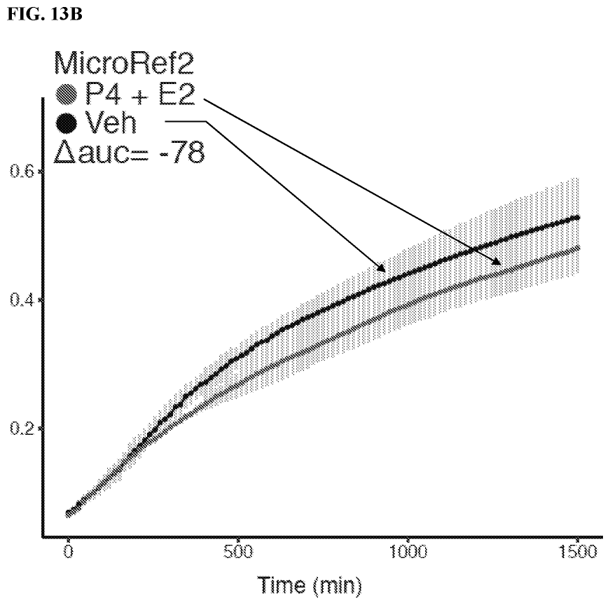

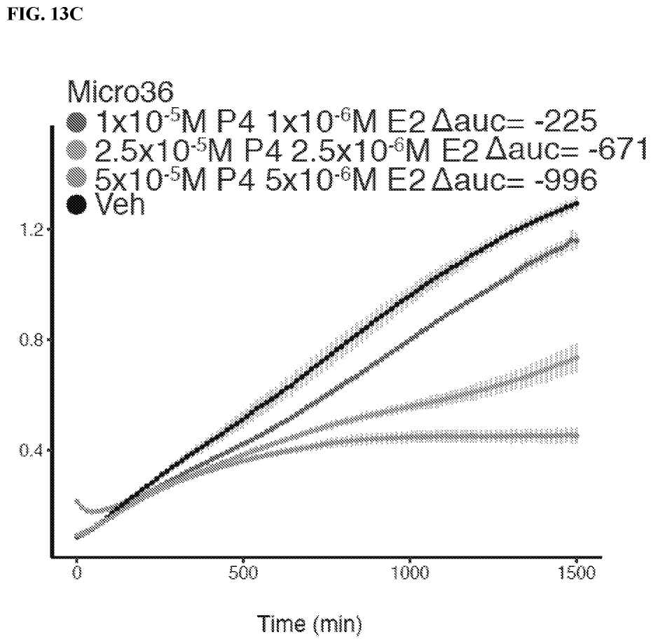

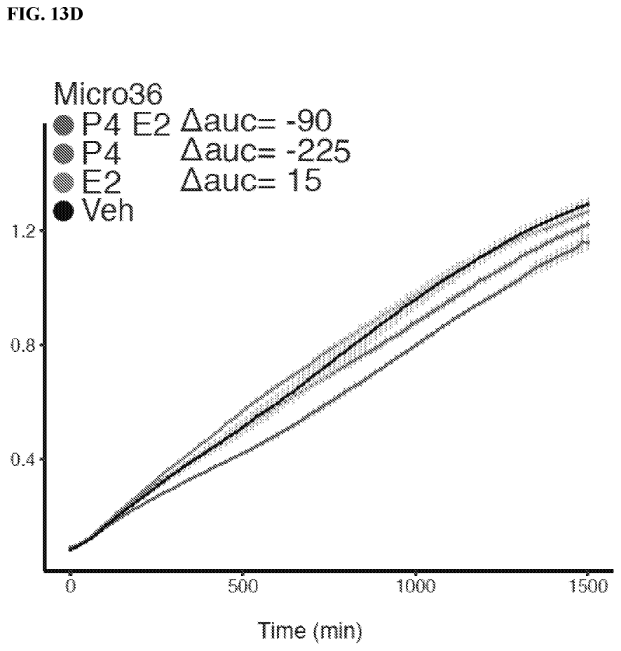

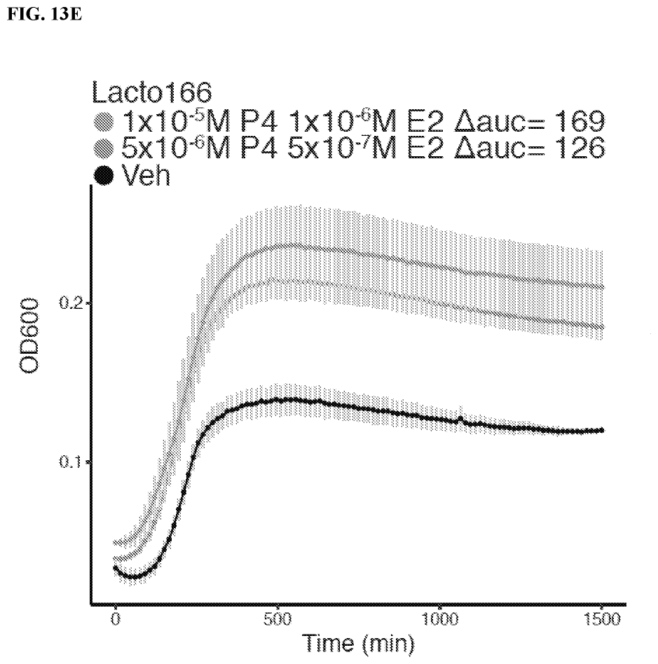

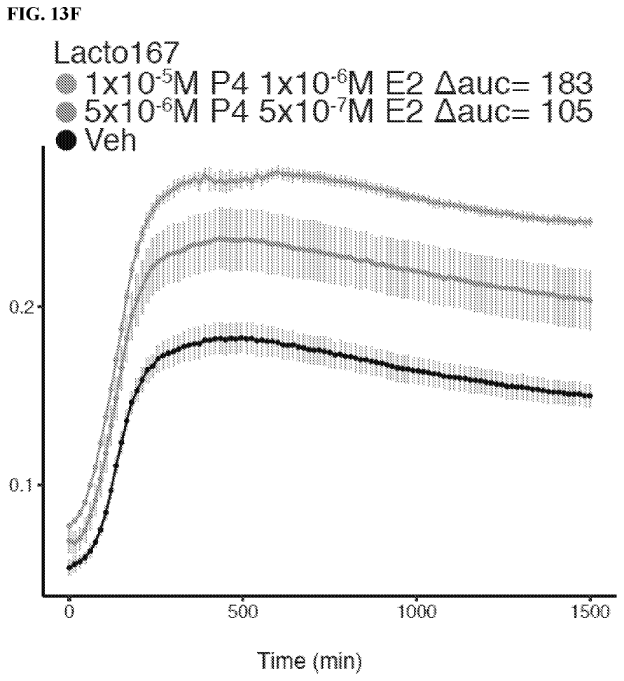

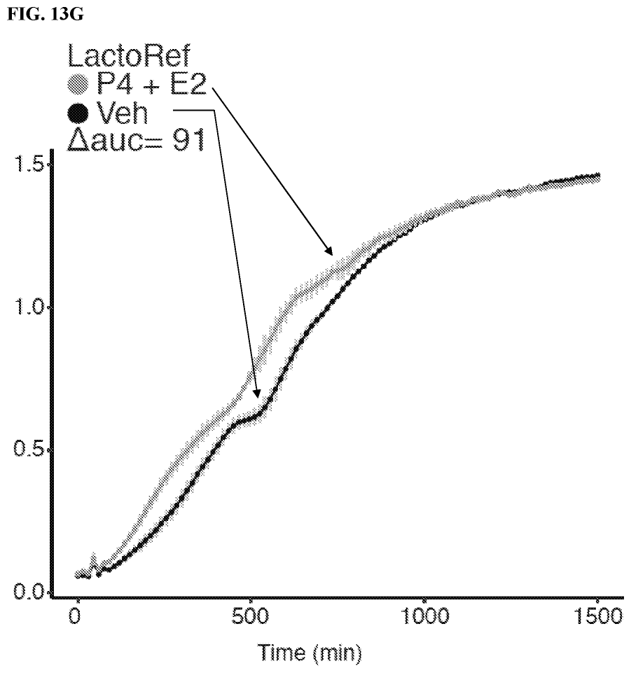

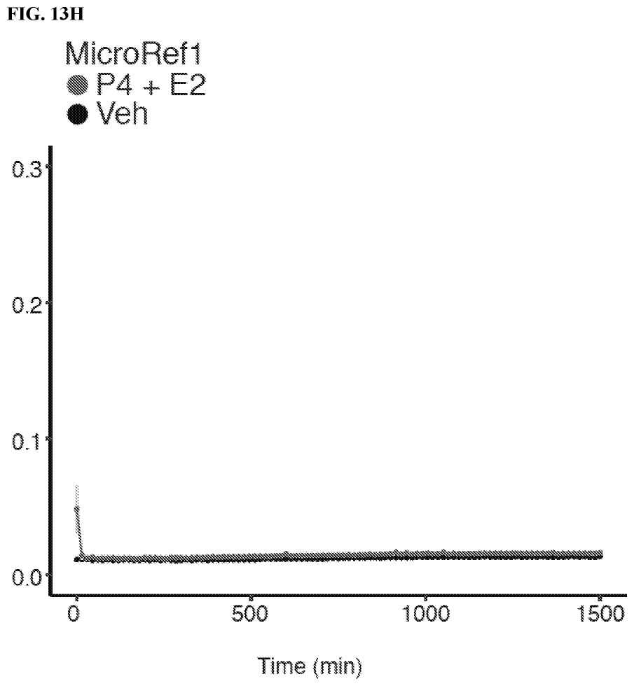

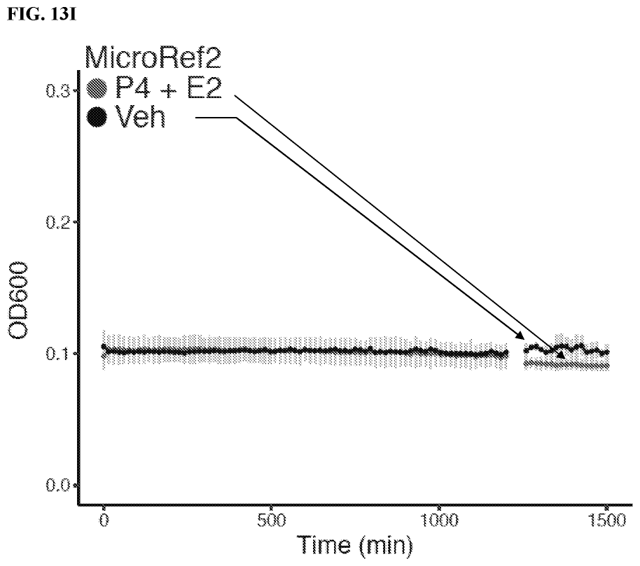

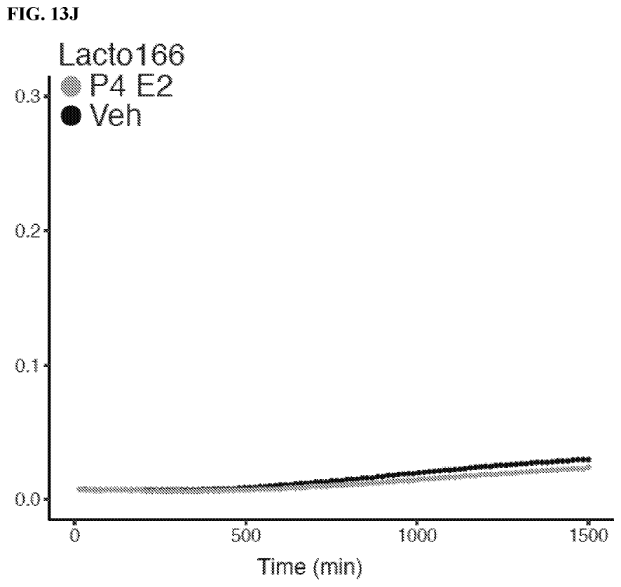

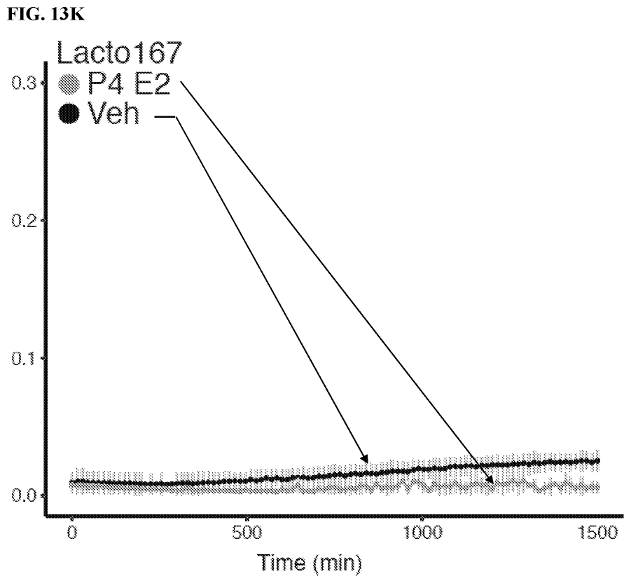

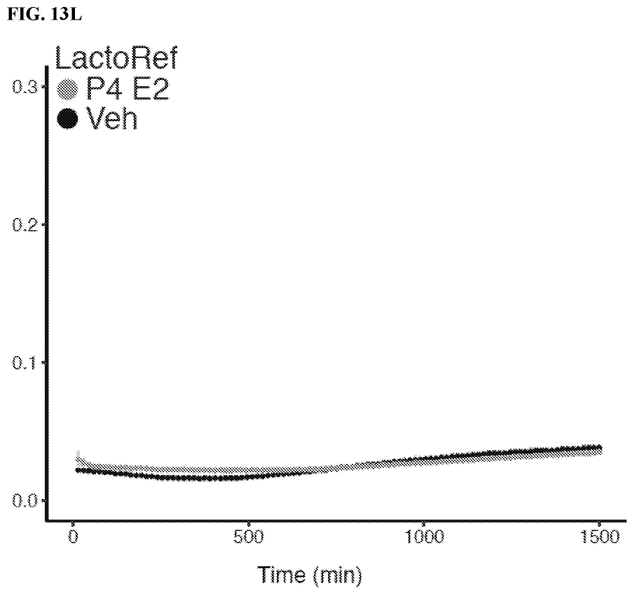

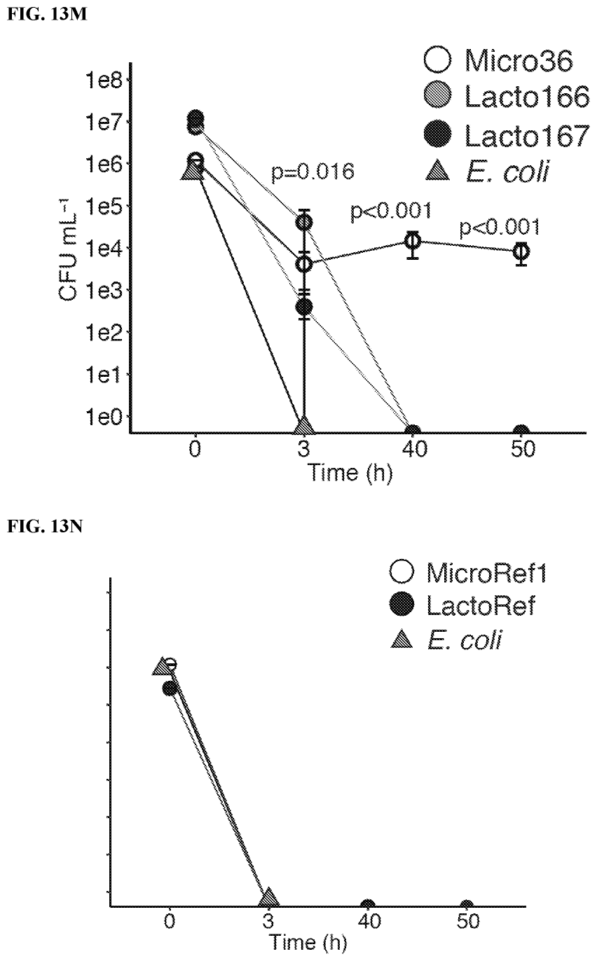

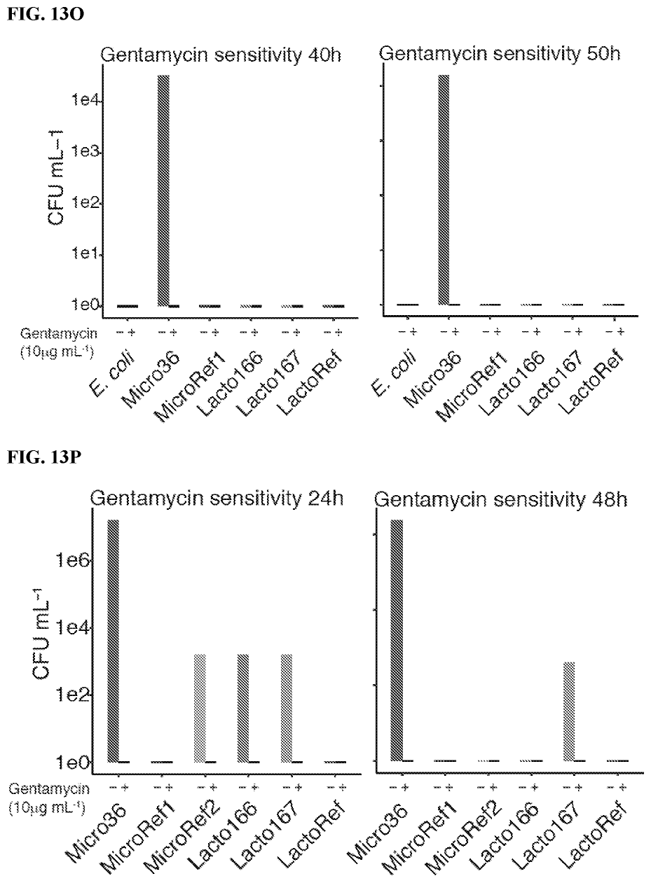

The requirement of placental steroid hormones for the initial isolation of fetal Lactobacillus and Micrococcus strains, led us to hypothesize that these isolates are specifically adapted to survival in the presence of these hormones. In carbon-rich media, peak third trimester cord blood concentrations [20] of progesterone alone or in combination with β-estradiol (but not β-estradiol alone), inhibited the growth of Micro36 and two reference M. luteus strains (MicroRef1 ATCC12693 and MicroRef2 ATCC12698; FIG. 3B, FIGS. 13A-D), consistent with reported bacteriostatic effects of steroid hormones [21]. Nutritional conditions influenced growth of fetal Lacto166 and Lacto167 in the presence of placental hormones; growth was enhanced in nutrient-rich chopped-meat carbohydrate (CMC) media, but inhibited in De Man, Rogosa and Sharpe (MRS) media (FIG. 3C-D, FIG. 13E-F). However, growth of phylogenetically related L. iners reference strain (LactoRef; ATCC55195) was unaffected by hormone addition in CMC media (FIG. 13G), suggesting species-specific adaptations to growth in the presence of pregnancy hormones. Micro36 exhibited the unique ability to grow on progesterone and β-estradiol in carbon limiting media (FIG. 3E), culture conditions in which MicroRef1 and 2 and all Lactobacillus strains were incapable of growth (FIG. 13H-L). These data suggest that placental hormones in concert with nutritional substrate availability may act as a selective pressure for fetal-adapted bacterial strain survival and growth.

The necessity of monocytes for initial Micrococcus isolation (Tables 4A-B) suggested the capacity for survival within phagocytic cells. Isolated fetal intestinal HLA-DR⁺ antigen presenting cells (APCs) were cleared of intracellular bacteria (See Example 2), incubated with fetal Lactobacillus and Micrococcus isolates to permit phagocytosis, and followed by gentamycin protection assays. At 24h, 1×10³ CFU mL⁻¹ of Lacto166 and Lacto167 and 1×10⁷ CFU mL⁻¹ of Micro 36 were recovered. Both Micro36 and Lacto167 remained viable in APCs at 48h at 1×10⁶ CFU mL⁻¹ or 1×10³, respectively (FIGS. 3F-G), indicating a capacity for prolonged intracellular survival. Control reference strains LactoRef, MicroRef1 and to a lesser extent MicroRef2 were non-viable under comparable conditions (FIGS. 3F-G). Similar results were obtained using a RAW264.7 macrophage cell line with an additional E. coli control, an extracellular bacterium (FIGS. 13M-N). Gentamycin resistance did not develop in the time course of either of these experiments (FIGS. 13O-P). The ability of fetal isolates to persist inside phagocytes offers a potential mechanism of entry of viable microbes into the fetal intestine.

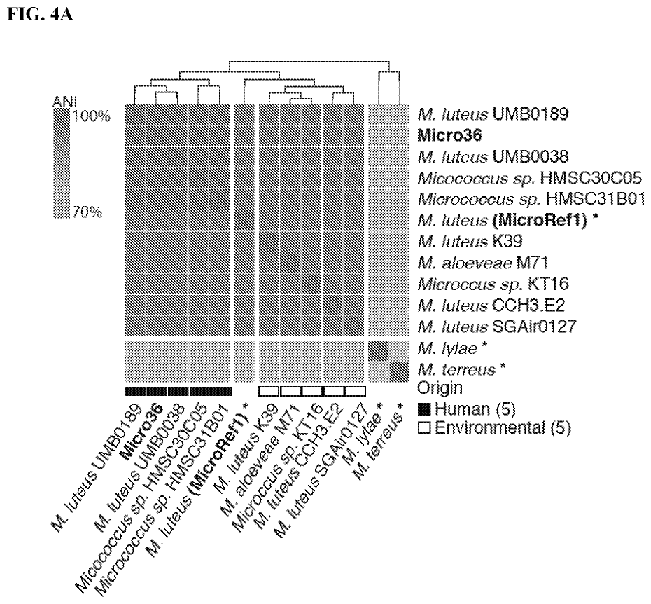

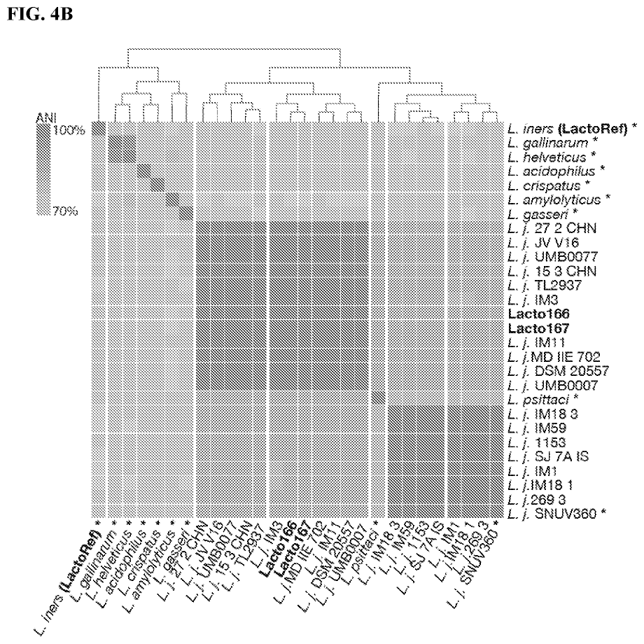

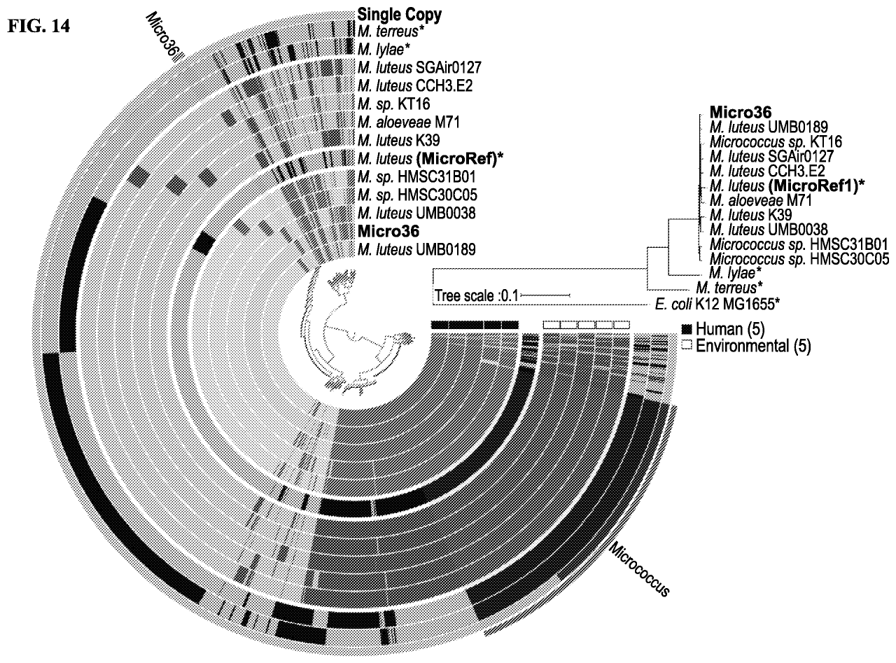

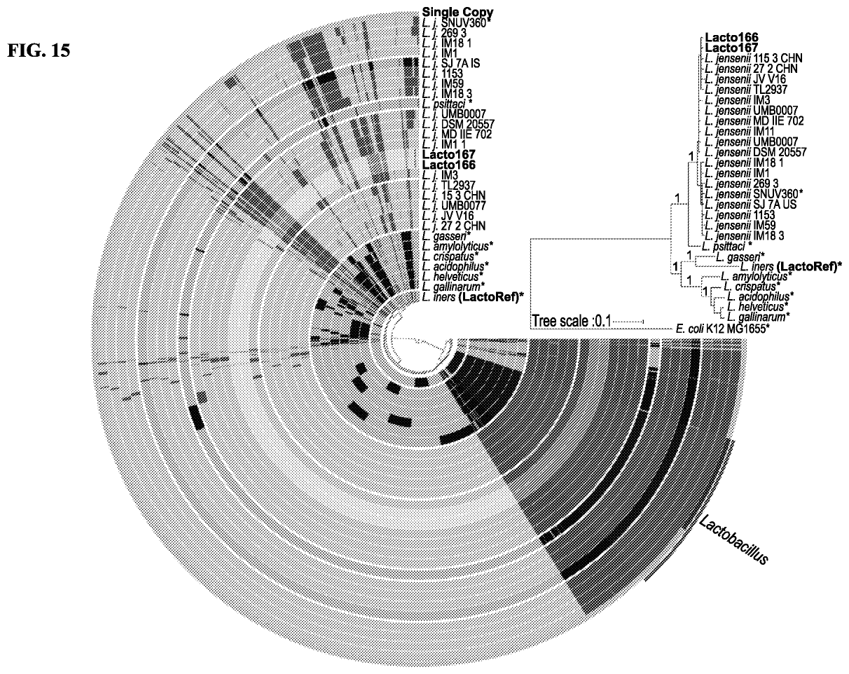

Whole genome sequencing of Lacto166, Lacto167, and Micro36 (Tables 5A-B) permitted high resolution taxonomy of fetal isolates and identified shared and unique genomic features when compared to phylogenetically related bacteria. Micro36 exhibited 96.9% whole genome average nucleotide identity (ANI) to a reference genome of M. luteus and clustered by whole genome ANI with other human, but not environmental M. luteus isolates (FIG. 4A, Tables 6A-D). Pan-genomic analysis of our fetal Micrococcus and all available Micrococcus genomes identified shared single-copy genes (FIG. 14) used to build highly resolved phylogeny (bootstrap value=1 for relevant clade, FIG. 14, inset). Using a 96.5% ANI speciation cut-off [22], Micro36 was classified as a strain of M. luteus. Lacto166 and Lacto167 exhibited >99.9% whole genome ANI to each other. Comparison with all publicly available reference or representative genomes within Lactobacillus indicated that Lacto166 and Lacto167 exhibited greatest similarity to L. jensenii (99.86% ANI for both, FIG. 4B, FIG. 15, Tables 7A-J) and shared single-copy genes (bootstrap value=1 for relevant clade, FIG. 15, inset) confirmed them as L. jensenii strains.

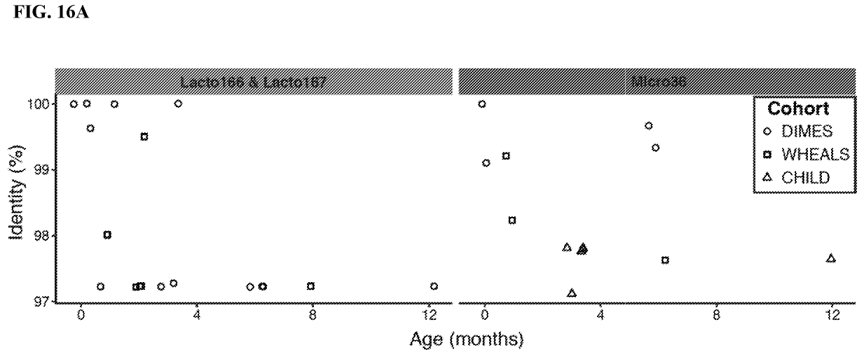

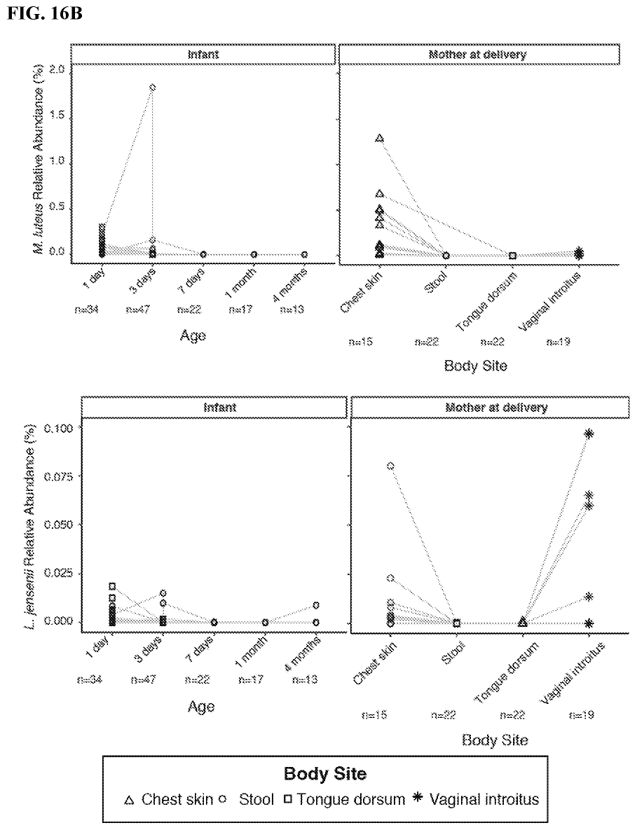

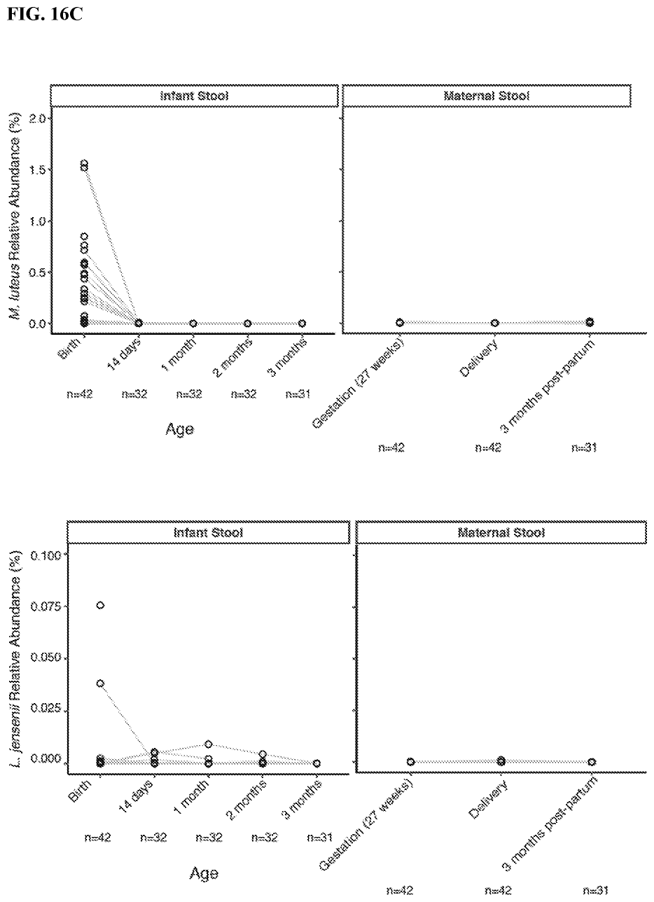

To determine whether these isolates were found in post-natal infant samples, we utilized publicly available 16S rRNA data from three independent early-life cohorts [15,16,23]. Sequences exhibiting ≥97% homology to our fetal isolates were detected throughout early life (up to 12 months; Tables 8A-B); however, sequences with the highest homology (≥99%, Tables 8A-B) were primarily found in infant meconium (first stool) samples (FIG. 16A). M. luteus and L. jensenii were low in abundance in infant samples but were highest in meconium in two independent metagenomic cohorts [24,25] (FIG. 16B-C). These species were detected on maternal chest and in vaginal introitus at delivery and were not highly abundant in maternal stool (FIG. 16B-C). This suggests that Micro36, Lacto166, and Lacto167 or highly related strains may persist at least until birth in the intestine and may be succeeded by phylogenetically related species in infancy.

Comparative genomics of Lacto166 and Lacto167 to the reference genome of L. jensenii identified 304 genes unique to fetal isolates; 123 were successfully annotated using NCBI clusters of orthologous groups (COG) database. Lacto166 and Lacto167 genomes encoded a type IV secretion system component VirD4, utilized by H. pylori for epithelial invasion [26] and consistent with our observed enrichment of bacterial invasion-associated transcripts in LM-E (FIG. 2C). Compared to M. luteus (MicroRef1), Micro36 exhibited 425 unique genes 256 of which were annotated. Genomic features of Micro36 included two sterol carrier proteins, reactive oxygen and nitrogen radical reducing enzymes, and genes in the catechol pathway. While the prevalence of these genes is yet to be determined, these data offer plausible mechanisms by which Micro36 may grow on placental hormones [27] (FIG. 3E), remain viable in phagocytes [28] (FIG. 3F), and under conditions of elevated NOS2 [29] (FIG. 2C).

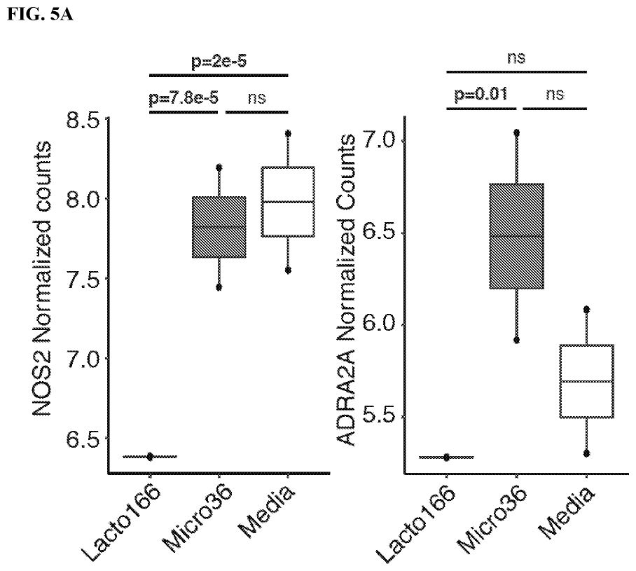

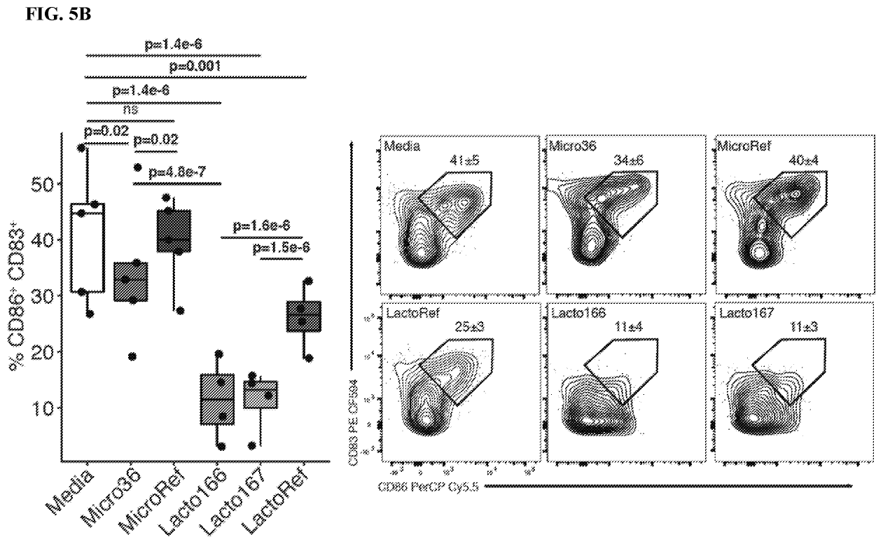

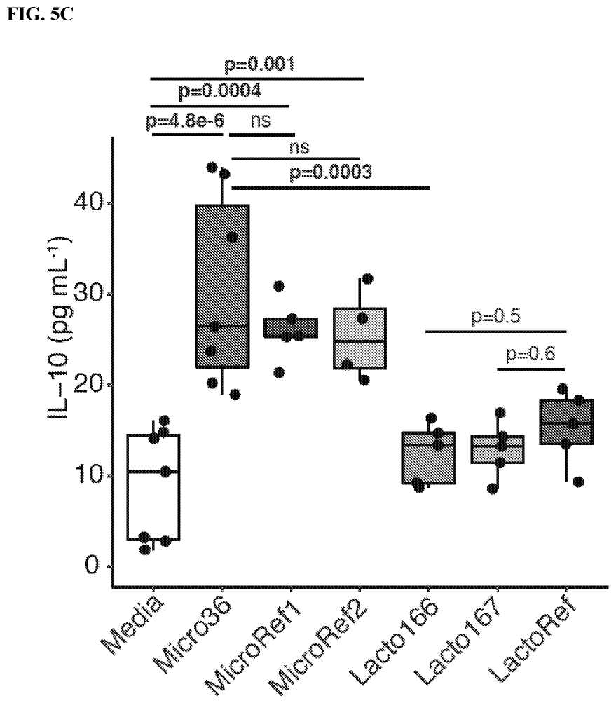

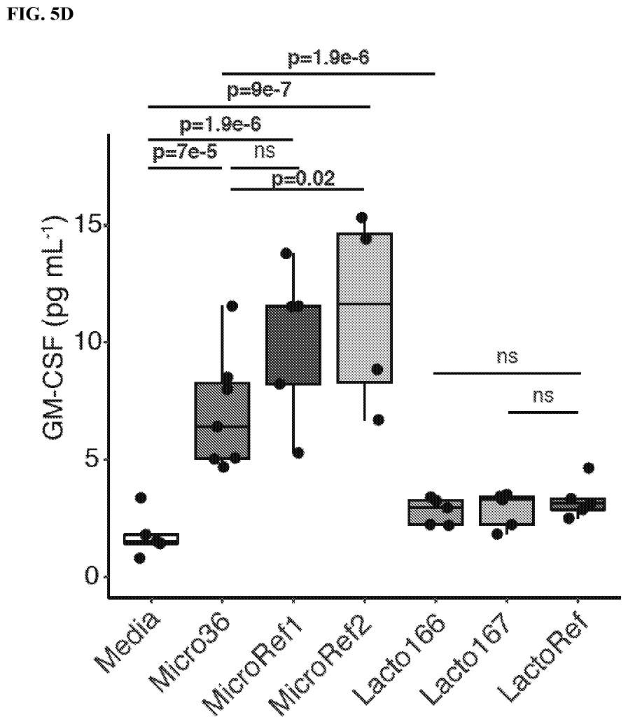

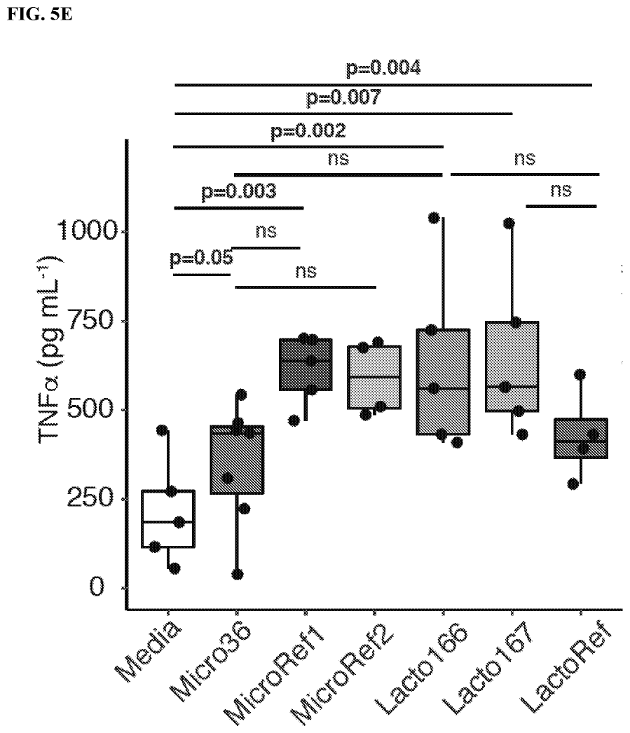

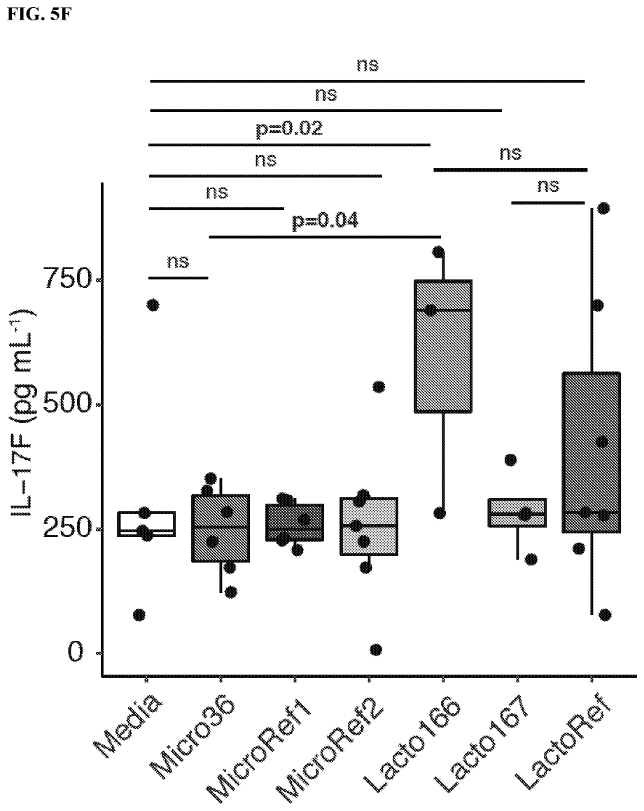

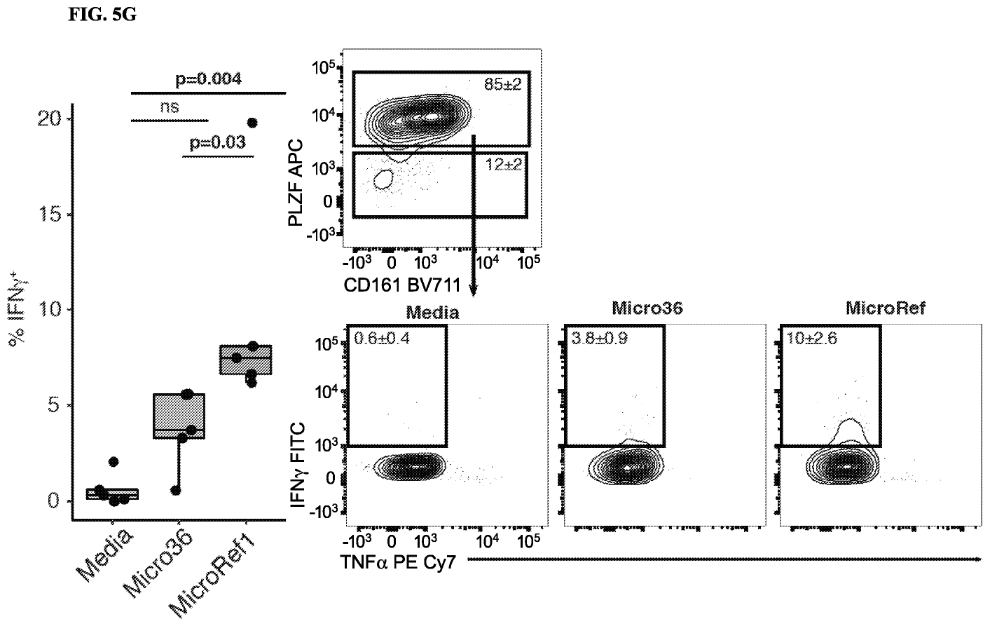

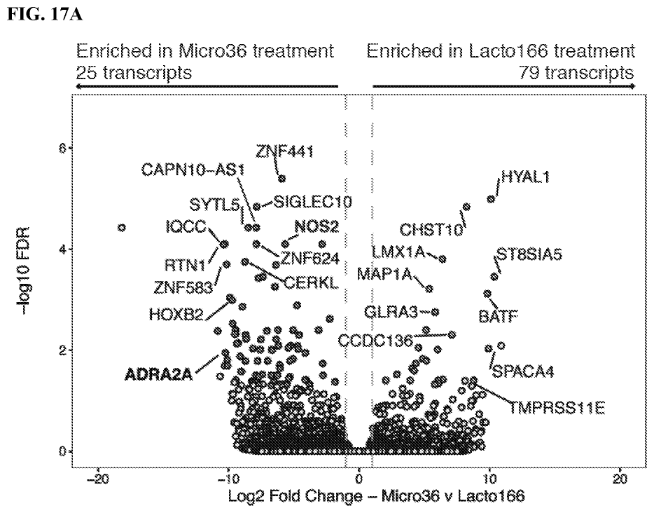

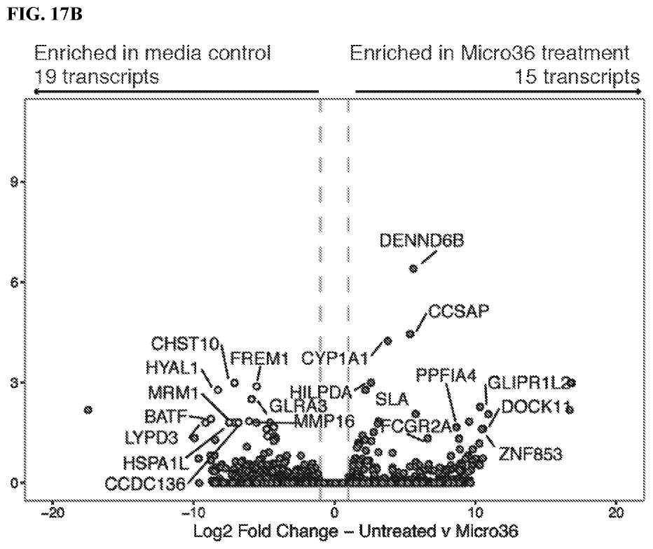

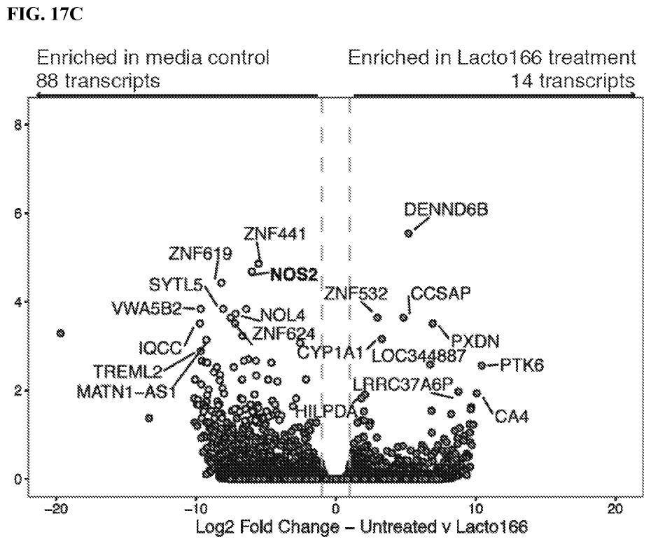

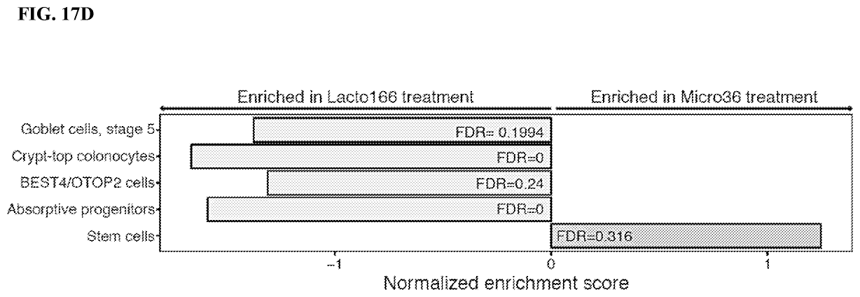

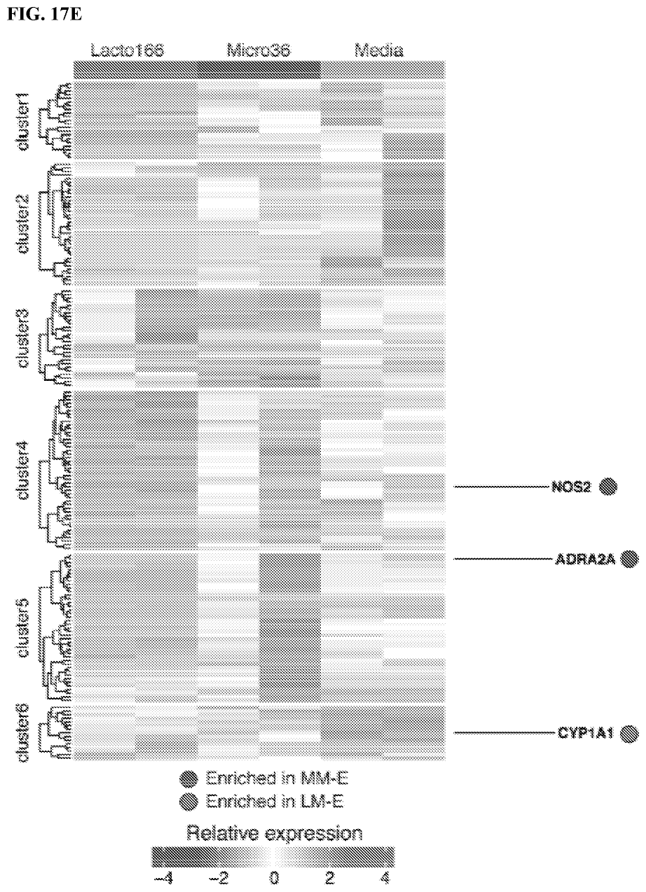

Fetal intestinal immune profiling indicated that the Lactobacillus and Micrococcus associated with distinct programs of immune function (FIGS. 2A-H). We thus examined the capacity of fetal isolates to induce transcriptional features observed ex vivo, by profiling the transcriptome of primary human fetal intestinal epithelial cells (n=2) exposed to Lacto166 or Micro36 for four hours in vitro. Transcriptional changes were observed when bacterial exposed epithelia were compared to media controls and with respect to each other (FIGS. 17A-C). As expected, short-term exposure to bacterial isolates in vitro did not fully recapitulate the global fetal intestinal transcriptome patterns observed in LM-E and MM-E (FIGS. 17A-C). Nonetheless differentially expressed genes consistent with ex vivo findings were identified (FIG. 5A; FIGS. 17D-E). Lacto166 treatment elicited genes associated with crypt-top colonyctes and absorptive progenitors (FIG. 17D) as observed in LM-E (FIG. 2E), while Micro36 exposure exhibited a trend toward significance of genes associated with stem cells (FIG. 17D) mirroring MM-E (FIG. 2E) by gene set enrichment analysis of epithelial cell states. Lactobacillus exposure specifically decreased NOS2 expression (FIG. 5A), consistent with its down regulation in LM-E (FIG. 2C). Micrococcus exposure induced the expression of ADRA2A, the alpha2A adrenoreceptor expressed on epithelial stem cells in intestinal crypts [30], which was also enriched in MM-E (FIG. 5A; FIG. 17E). These data suggest that even following short-term fetal bacterial exposure, fetal intestinal epithelial cells exhibit transcriptional responses that partially recapitulate transcriptional features associated with the presence of these bacteria ex vivo

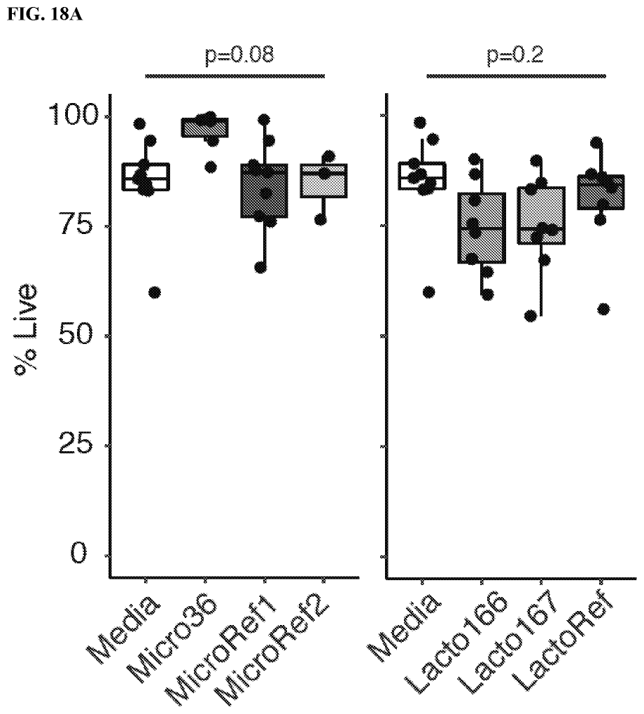

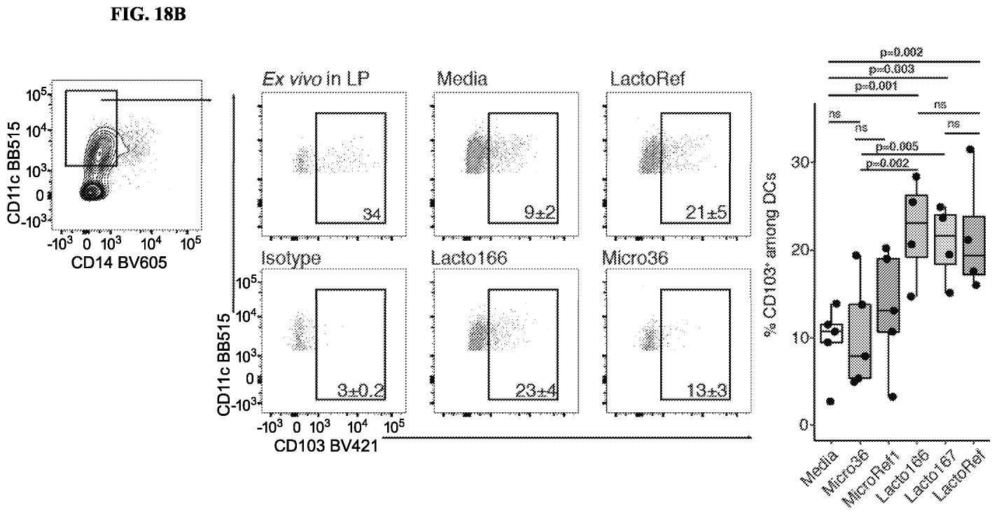

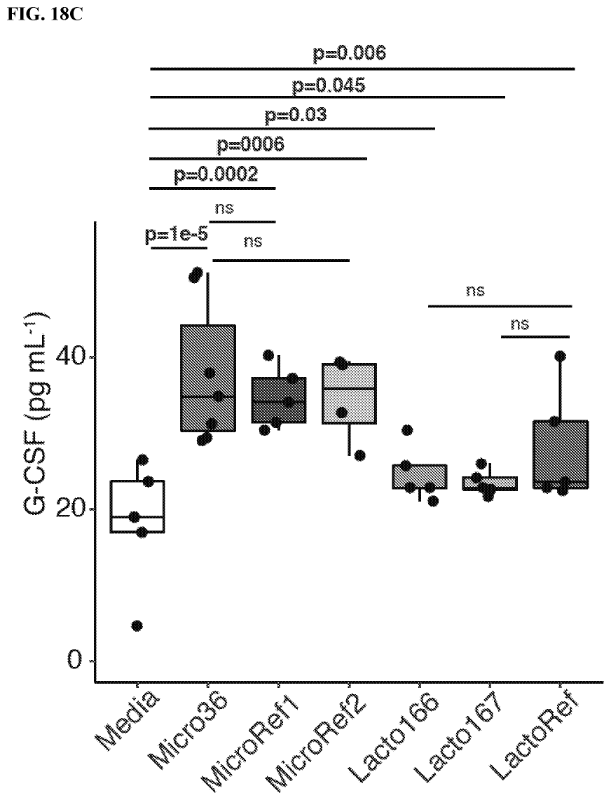

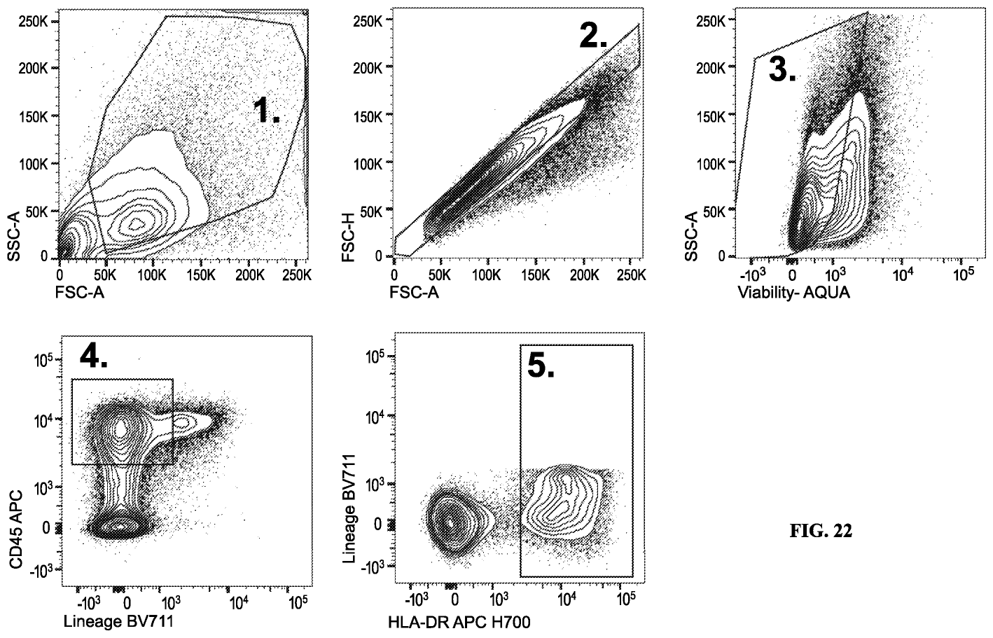

We next assessed the capacity of live fetal bacterial isolates or respective reference species to activate primary fetal splenic HLA-DR⁺ antigen presenting cells (APCs) (FIG. 22). Without decreasing cell viability (FIG. 18A), exposure to Lacto166 and Lacto167 significantly reduced the co-expression of the APC co-stimulatory molecules CD86 and CD83, required for efficient human T cell priming [31] (FIG. 5B), suggestive of a tolerance-promoting mechanism. In parallel, all Lactobacillus strains increased expression of the tolerogenic integrin CD103 [32] on splenic CD11c⁺ dendritic cells (DCs) in comparison with media controls (FIG. 18B). In contrast, Micrococcus strains did not reduce APC activation (FIG. 5B) or increase CD103 expression (FIG. 18B). However, all Micrococcus strains induced fetal APC production of cytokines associated with maturation of intestinal macrophages (GM-CSF and G-CSF) as well as IL-10 (FIG. 5C-D, FIG. 18C), which promote a tolerogenic environment [33-35]. These in vitro findings are also consistent with our observation that MM-E transcriptomes are enriched for macrophage recruitment and inhibitory transcripts (FIG. 2C). Lactobacillus induced greater TNFα in APC culture supernatants compared with Micro36 (FIG. 5E), which has been implicated in promoting epithelial development [36] and these transcriptional features were also observed in LM-E datasets (FIG. 2C).

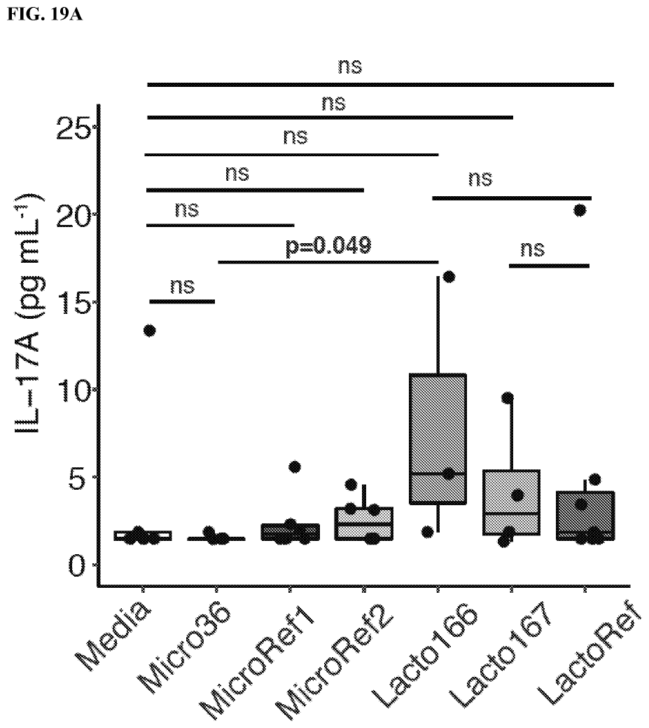

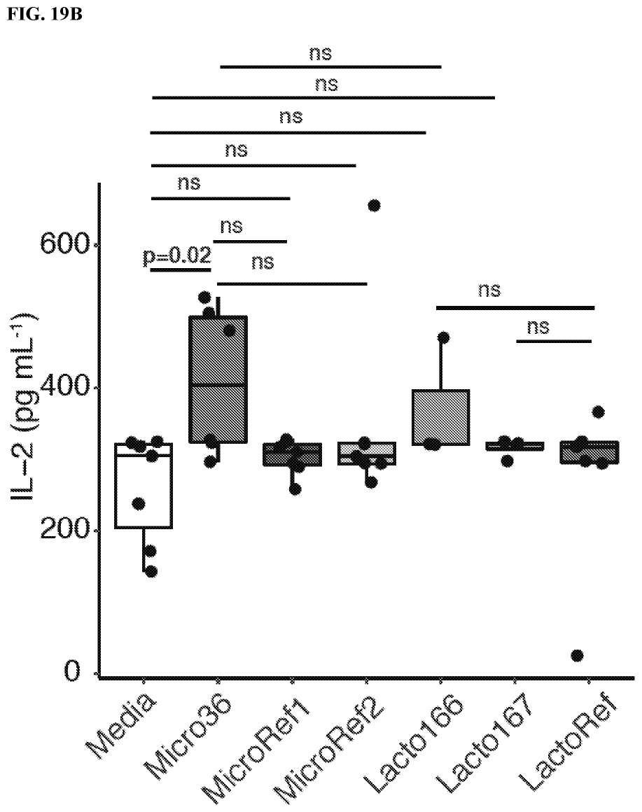

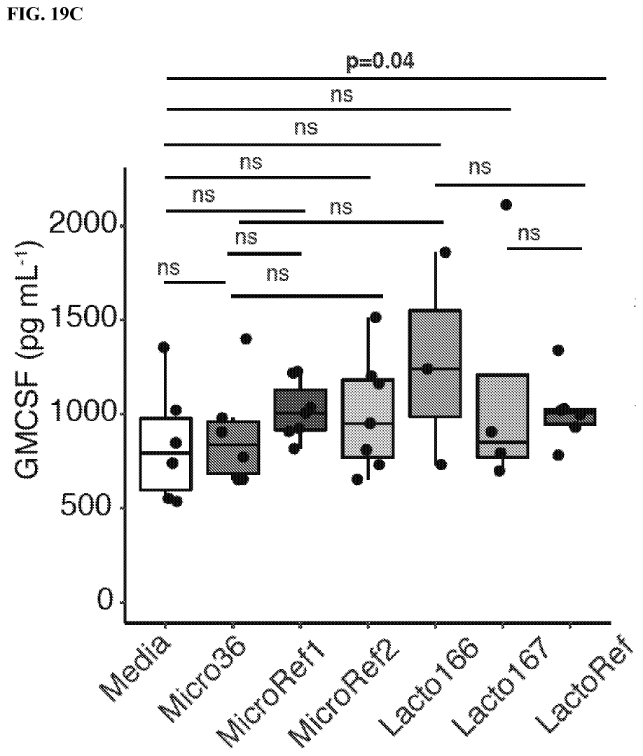

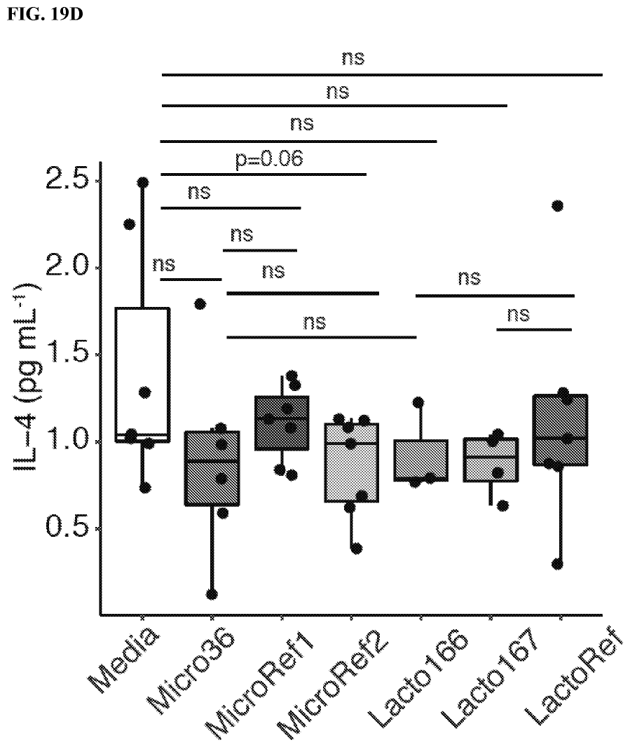

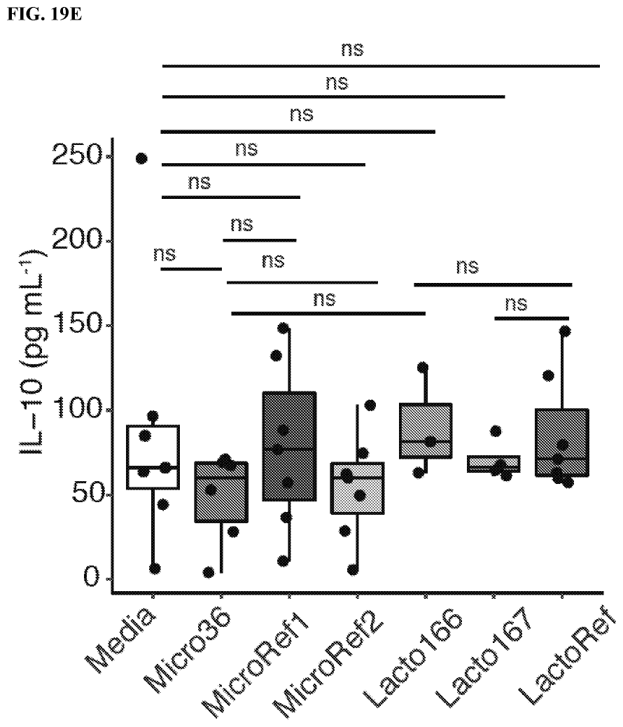

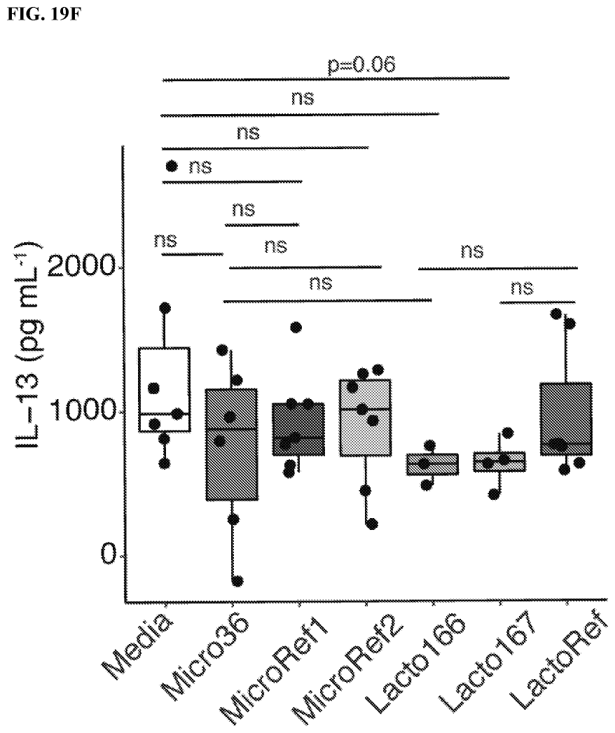

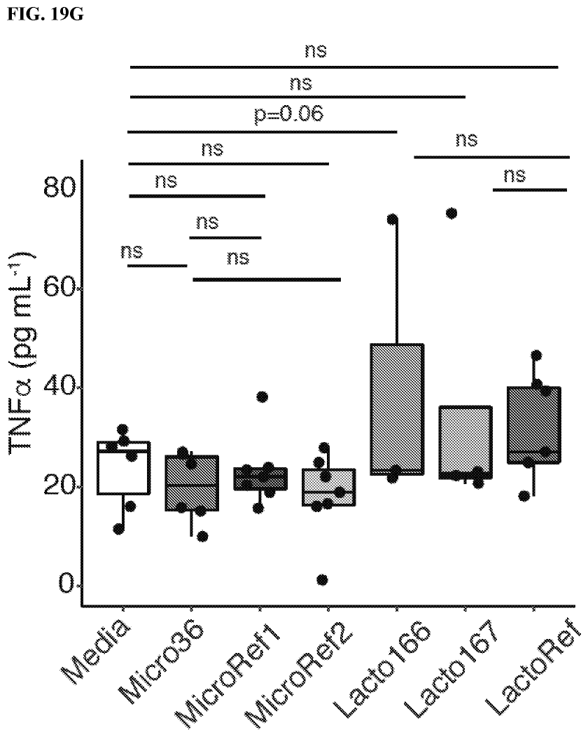

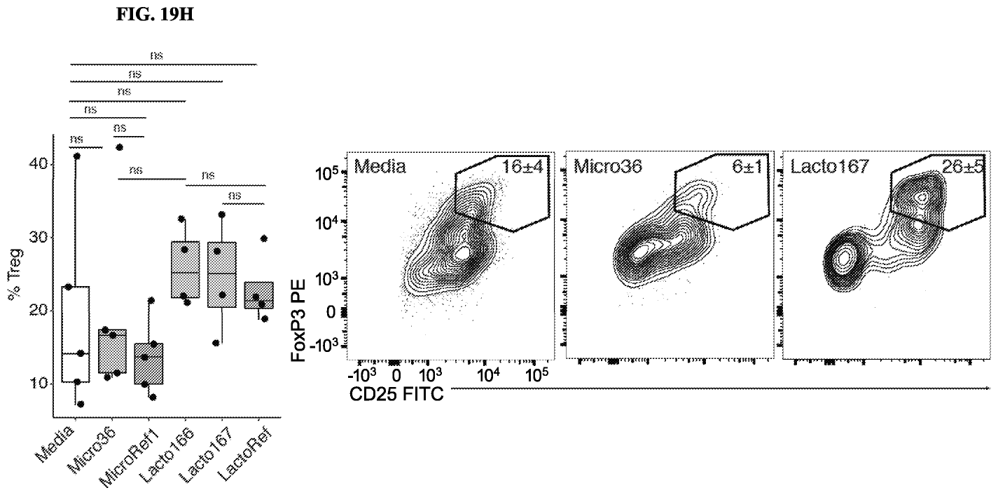

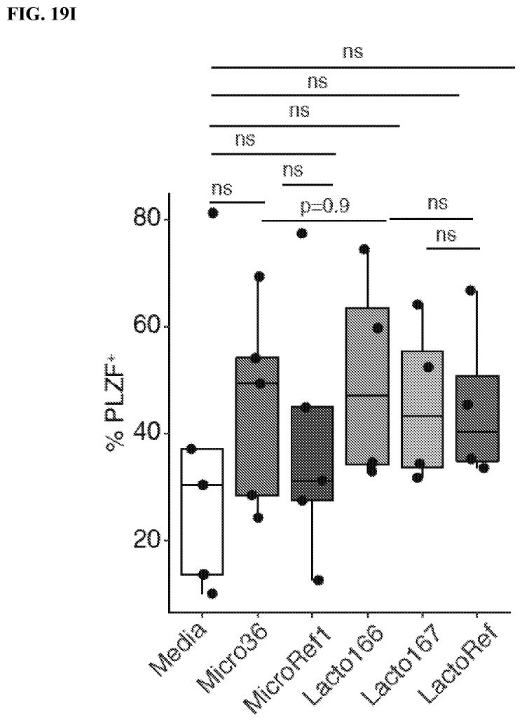

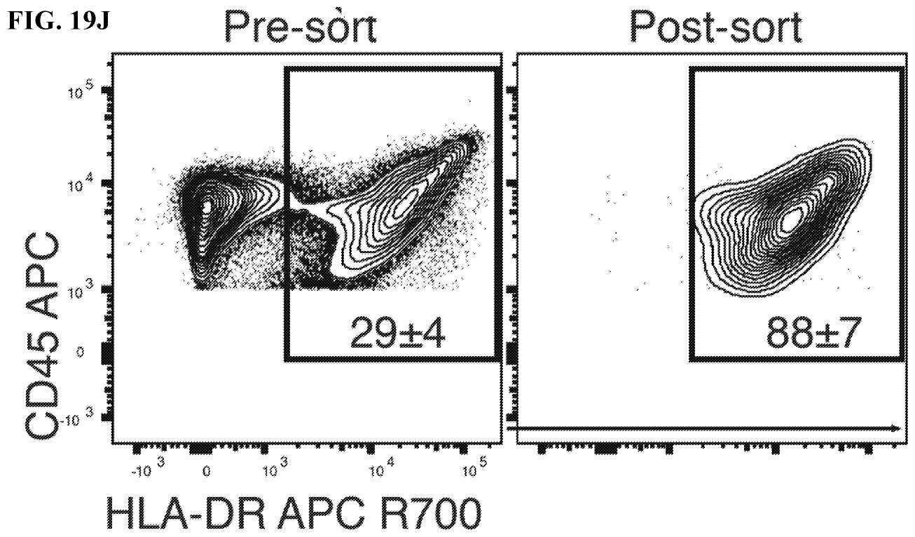

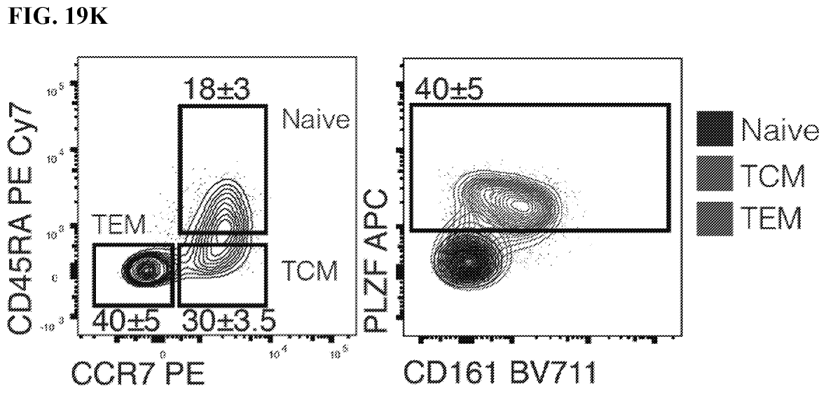

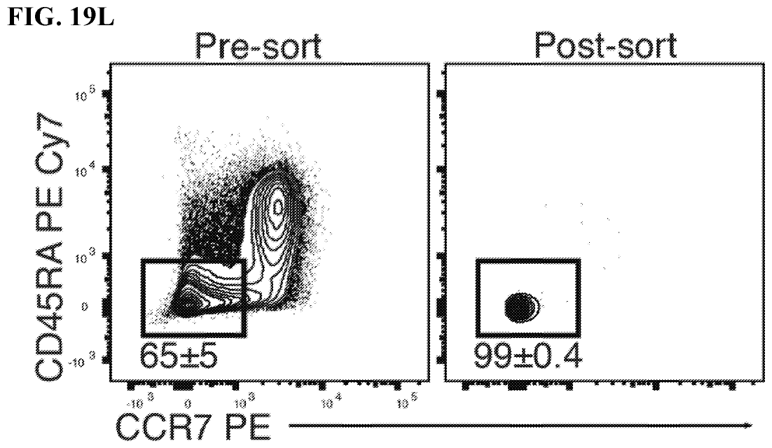

When autologous intestinal T cells were added to APC co-cultures, Lacto166 induced the production of IL-17F and IL17A in culture supernatants (FIG. 5F, FIG. 19A), cytokines known to promote epithelial barrier integrity [37] and consistent with more mature epithelial function observed in LM-E (FIG. 2E). Micro36 pre-treatment of APCs induced IL-2 production in APC-T cell co-cultures (FIG. 19B); significant changes in GMCSF, IL-4, IL-10, IL-13, or TNFA were not observed (FIGS. 19C-G). Despite the strain-specific cytokine response, proportional accumulation of regulatory T (Treg) and PLZF+ T cells was unrelated to Micrococcus or Lactobacillus exposure under the in vitro conditions tested (FIGS. 19H-I). However, Micro36 impacted PLZF+ T cell function through modulation of APC phenotype. Sorted splenic APCs (FIG. 19J) pre-conditioned with Micro36 or MicroRef1 were co-incubated with pure (>99%), autologous, fetal intestinal effector memory T cells, the majority of which expressed PLZF and c-type lectin CD161 [6] (FIGS. 19K-L). Micro36 exposure resulted in a significant reduction of IFNγ production by these T cells as compared to MicroRef (FIG. 5G). Ligation of CD161 inhibits IFNγ production by fetal intestinal PLZF⁺ CD161⁺ T cells [6]. LLT1, the natural ligand for CD161, is expressed on fetal intestinal macrophages [6] and can be induced upon TLR activation of APCs [38]. Exposure to Micro36 exclusively induced LLT1 expression on splenic APCs in proportion with multiplicity of infection (FIGS. 5H-J), albeit to lower levels than observed in lamina propria APCs ex vivo.

These data suggest that fetal Lactobacillus promoted intestinal epithelial maturation and immune tolerance by limiting APC activation. In contrast, fetal Micrococcus induced tolerogenic APCs and inhibited IFNγ production by fetal memory T cells, indicating strain-specific immunomodulatory mechanisms.

Through molecular bacterial detection, immune profiling, microscopy, strain isolation and ex vivo studies, this study provides evidence for viable bacteria in the human fetal intestine during mid-gestation. Consistent with features of early ecosystem development, the enrichment of Lactobacillus (LM) or Micrococcus (MM) in subsets of fetal meconium was detected. In the context of low bacterial burden, 16S rRNA analysis is noisy [39,40] and may necessitate both molecular enrichment of the bacterial DNA and stringent filtering, the latter of which may have reduced true signal in a majority of meconium specimens (OM samples). This led us to focus our efforts on LM and MM fetal meconium and to apply a variety of approaches to confirm the presence and effect of viable Lactobacillus and Micrococcus in utero.

Fetal Lactobacillus or Micrococcus most likely arise from maternal cervico-vaginal microbiomes, which commonly house both genera [41,42]. While our fetal Lactobacillus and Micrococcus isolates exhibited genome similarity to vaginal strains, they also encoded strain-specific genes not found in genomes of these closely related strains, which may provide them with a survival advantage under the strong selective conditions of the fetal intestine. The prevalence of these strains and genes necessitates further study as strains of other genera in the human microbiome may also exhibit similar capacities. It is also plausible that genes that permit survival in the fetal intestine are also useful for vaginal survival during pregnancy. Placental hormones (progesterone and β-estradiol) can be detected in maternal circulation [43], plausibly selecting for bacteria that exhibit enhanced survival in this hormonal environment. The combination of progesterone, β-estradiol, and nutrient availability influenced growth capacities of fetal strains in vitro. Thus, steroid hormone concentrations, coupled with nutrient availability, may influence the presence of Lactobacillus or Micrococcus in utero. Hormone levels are highly variable between pregnant women [43], as is nutrition, offering a plausible explanation for enrichment of Lactobacillus or Micrococcus in subsets of meconium samples. However, we acknowledge that additional maternal factors unaccounted for in this study, such as host genetics, race, and health status also contribute to the inherent variability within pregnant mothers that may influence the presence of fetal bacteria.

Lactobacillus and Micrococcus in the fetal intestine modulates mucosal immunity and reciprocally, the immune system influences which microbes are tolerated by the host [44]. By investigating epithelial and lamina propria immunity of paired samples, we found numerous immune correlates specific to the presence of Lactobacillus or Micrococcus in fetal meconium. A number of ex vivo observations could be recapitulated by fetal Lactobacillus and Micrococcus isolates in vitro. However, other developmental factors such as stem cell niche [45], the predisposition for fetal T cells to develop into regulatory T cells [46], and antigens from swallowed amniotic fluid [47] also shape prenatal immunity.

Recent studies of fetal immunity have led to the hypothesis that bacterial signals in utero initiate an adaptive immune response [31], including T cell activation [6-8,48]. Fetal T cells respond to non-inherited maternal- and self-antigens [46,48] and are capable of memory formation in the intestine [6-8]. The presence of bacteria in the fetal intestine suggests that bacterial antigens may also contribute to T cell activation. Fetal intestinal T cells do not exclusively exhibit a tolerogenic phenotype [6-8]. Their ability to produce inflammatory cytokines in the absence of systemic inflammation indicates intestinal compartmentalization of immune response in utero [6], which may be essential for tolerance or clearance of fetal intestinal bacteria. Micrococcus enrichment in the fetal gut associated with increased proportions of IFNF-producing mucosal memory PLZF⁺ CD161⁺ T cells [6] and only the fetal Micrococcus isolate reduced IFNγ production by these T cells. While fetal Micrococcus likely elicits a number of responses, the specific induction of LLT1 on antigen presenting cells identifies a potential bacterial mechanism of immune regulation that is unique to fetal adaptive immunity [6]. Thus, by suppressing inflammation, Micrococcus may foster a tolerant environment that permits its survival in utero.

How fetal bacteria access and persist in the fetal compartment remains underexplored, though the ability of fetal bacterial isolates to grow on pregnancy hormones and survive within phagocytes offer plausible mechanisms. The impact of viable bacterial presence in the fetal intestine on lifelong immunity are unknown.

However, these findings indicate that human bacterial-immune interactions may variably occur in utero and that these bacteria exhibit distinct immunomodulatory capacities.

### Tables

TABLE 1A
Fetal meconium bank

| | | | Median 16S |
|---|---|---|---|
| | | | rRNA V4 |
| | | | Sequencing Read |
| | | No. samples | Count Per Sample |
| | No. samples | successfully | prior to technical |
| Sample Type | banked | sequenced * | negative filtering |
| Technical negative | 50 | 48 | 35,906 |
| controls | | | |
| Extraction Buffer | 12 | 12 | 12,957 |
| Room Air swab | 19 | 19 | 39,651 |
| Pre-moistened swab | 19 | 17 | 40,805 |
| Technical positive | 3 | 3 | 281,284 |
| controls | | | |
| Mock community | 3 | 3 | 281,284 |
| Procedural | 37 | 35 | 23,300 |
| Environment | | | |
| controls | | | |
| Procedural swab | 19 | 17 | 27,534 |
| Kidney | 18 | 18 | 12,510 |
| Fetal meconium | 150 | 107 | 30,508 |
| Proximal | 50 | 47 | 33,391 |
| Mid | 50 | 46 | 30,247.5 |
| Distal | 50 | 45 | 29,826 |

TABLE 1B
Fetal meconium bank

| | | | No. samples with |
|---|---|---|---|
| | Median 16S rRNA V4 | | sequences |
| | Sequencing Read Count | | remaining after |
| | Per Sample after | Reads | technical negative |
| | technical negative | remaining after | filtering and |
| Sample Type | filtering | filtering (%) | rareifying |
| Technical negative controls | | | |
| Extraction Buffer | | | |
| Room Air swab | | | |
| Pre-moistened swab | | | |
| Technical positive controls | 13,766 | 4.89 | 3 |
| Mock co nnnnunity | 13,766 | 4.89 | 3 |
| Procedural Environment controls | 1,822 | 7.82 | 21 |
| Procedural swab | 2,847 | 10.34 | 14 |
| Kidney | 645 | 5.16 | 7 |
| Fetal meconium | 4,424 | 14.50 | 108 |
| Proximal | 3,301 | 9.89 | 32 |
| Mid | 4,786 | 15.82 | 40 |
| Distal | 4,488 | 15.05 | 36 |

Sample characteristics in Tables 1A and 1B were as follows: Median (SD) weeks gestational age was 20 (±2.2);
No. paired epithelial cell layer transcirptomes was 13;
No. paired lamina propria T cell profiles was 22.
* Sequences were obtained after de-multiplexing.

TABLE 2A
Contaminant OTUs filtered with respect to technical negative controls

| OTU ID | Kingdom | Phylum | Class | Order |
|---|---|---|---|---|
| OTU_1 | Bacteria | Proteobacteria | Gammaproteobacteria | Enterobacteriales |
| OTU_1098 | Bacteria | Proteobacteria | Gammaproteobacteria | Betaproteobacteriales |
| OTU_1121 | Bacteria | Proteobacteria | Gammaproteobacteria | Enterobacteriales |
| OTU_1136 | Bacteria | Proteobacteria | Gammaproteobacteria | Pseudomonadales |
| OTU_1167 | Bacteria | Proteobacteria | Gammaproteobacteria | Betaproteobacteriales |
| OTU_1174 | Bacteria | Proteobacteria | Gammaproteobacteria | Betaproteobacteriales |
| OTU_1182 | Bacteria | Proteobacteria | Gammaproteobacteria | Pseudomonadales |
| OTU_1199 | Bacteria | Proteobacteria | Gammaproteobacteria | Betaproteobacteriales |
| OTU_15 | Bacteria | Proteobacteria | Gammaproteobacteria | Xanthomonadales |
| OTU _17 | Bacteria | Proteobacteria | Gammaproteobacteria | Oceanospirillales |
| OTU_2 | Bacteria | Proteobacteria | Gammaproteobacteria | Betaproteobacteriales |
| OTU_24 | Bacteria | Proteobacteria | Gammaproteobacteria | Betaproteobacteriales |
| OTU_263 | Bacteria | Proteobacteria | Gammaproteobacteria | Pseudomonadales |
| OTU_3 | Bacteria | Proteobacteria | Gammaproteobacteria | Pseudomonadales |
| OTU_4 | Bacteria | Proteobacteria | Deltaproteobacteria | Myxococcales |
| OTU_498 | Bacteria | Proteobacteria | Gammaproteobacteria | Betaproteobacteriales |
| OTU_5 | Bacteria | Proteobacteria | Gammaproteobacteria | Betaproteobacteriales |
| OTU_6 | Bacteria | Verrucomicrobia | Verrucomicrobiae | Verrucomicrobiales |
| OTU_63 | Bacteria | Actinobacteria | Actinobacteria | Propionibacteriales |
| OTU_7 | Bacteria | Firmicutes | Bacilli | Bacillales |
| OTU_9 | Bacteria | Proteobacteria | Gammaproteobacteria | Betaproteobacteriales |
| OTU_933 | Bacteria | Proteobacteria | Gammaproteobacteria | Pseudomonadales |

TABLE 2B
Contaminant OTUs filtered with respect to
technical negative controls

| OTU ID | Family | Genus |
|---|---|---|
| OTU_1 | Enterobacteriaceae | Escherichia/Shigella |
| OTU_1098 | Burkholderiaceae | Burkholderia-Caballeronia- |
| | | Paraburkholderia |
| OTU _1121 | Enterobacteriaceae | NA |
| OTU_1136 | Pseudomonadaceae | Pseudomonas |
| OTU_1167 | Burkholderiaceae | NA |
| OTU_1174 | Burkholderiaceae | NA |
| OTU_1182 | Pseudomonadaceae | NA |
| OTU_1199 | Burkholderiaceae | NA |
| OTU_15 | Xanthomonadaceae | Stenotrophomonas |
| OTU _17 | Halomonadaceae | Halomonas |
| OTU_2 | Burkholderiaceae | Burkholderia-Caballeronia- |
| | | Paraburkholderia |
| OTU_24 | Burkholderiaceae | Ralstonia |
| OTU_263 | Pseudomonadaceae | Pseudomonas |
| OTU_3 | Pseudomonadaceae | Pseudomonas |
| OTU_4 | Myxococcaceae | Myxococcus |
| OTU_498 | Burkholderiaceae | Burkholderia-Caballeronia- |
| | | Paraburkholderia |
| OTU_5 | Burkholderiaceae | Delftia |
| OTU_6 | Akkermansiaceae | Akkermansia |
| OTU_63 | Propionibacteriaceae | Cutibacterium |
| OTU_7 | Staph ylococcaceae | Staphylococcus |
| OTU_9 | Burkholderiaceae | Sphaerotilus |
| OTU_933 | Pseudomonadaceae | Pseudomonas |

TABLE 3
Significantly and differentially expressed genes in LM-E and MM-E

| | | | False | |
|---|---|---|---|---|
| | | Log2 Fold | discovery | |
| Gene | Base Mean | Change | rate (FDR) | Enriched in |
| PCAT7 | 8.651486949 | −4.959006057 | 0.018117153 | MM-E |
| HOXB6 | 14.74952205 | −4.919177633 | 0.011041066 | MM-E |
| WNT10A | 9.1413152 | −4.704686014 | 0.018541571 | MM-E |
| HOXB-AS3 | 6.669746998 | −4.281481957 | 0.048923609 | MM-E |
| LINC00668 | 25.90886452 | −4.268498542 | 0.003762794 | MM-E |
| CDH7 | 12.06245867 | −3.98078514 | 0.02547227 | MM-E |
| NANOS3 | 10.17677543 | −3.892175375 | 0.016735483 | MM-E |
| ADRA2C | 27.10304841 | −3.473047235 | 0.001881037 | MM-E |
| EPHB1 | 214.5986656 | −3.417129974 | 0.00021404 | MM-E |
| CPZ | 27.52385286 | −3.348038149 | 0.047561786 | MM-E |
| FZD9 | 57.65251263 | −2.988740234 | 0.005143097 | MM-E |
| ALOXE3 | 81.88892643 | −2.905913618 | 0.000810088 | MM-E |
| RAB40A | 18.59326274 | −2.775526909 | 0.042765357 | MM-E |
| CES5AP1 | 29.86863134 | −2.682323475 | 0.018117153 | MM-E |
| ACTN2 | 30.85197838 | −2.67114205 | 0.026226824 | MM-E |
| WSCD2 | 79.20988249 | −2.662819847 | 0.01123804 | MM-E |
| L1TD1 | 54.63412587 | −2.619721451 | 0.016366901 | MM-E |
| HS3ST1 | 216.4038216 | −2.59556415 | 0.000141402 | MM-E |
| RASL10A | 20.15935923 | −2.469665924 | 0.033060003 | MM-E |
| HFM1 | 17.72540362 | −2.372200552 | 0.045329791 | MM-E |
| C1orf95 | 47.03475841 | −2.341516573 | 0.014289478 | MM-E |
| NMUR2 | 84.02048635 | −2.285211128 | 0.008858331 | MM-E |
| NPR2 | 161.6257821 | −2.269721039 | 0.000257312 | MM-E |
| SLC34A2 | 135.8512812 | −2.268945944 | 0.010593459 | MM-E |
| LAMA2 | 211.6001534 | −2.259879996 | 3.49E-05 | MM-E |
| FOXQ1 | 1002.484627 | −2.25929226 | 0.008717552 | MM-E |
| NXF3 | 21.97492878 | −2.220811869 | 0.019859992 | MM-E |
| NPPC | 28.28336026 | −2.216658734 | 0.02793787 | MM-E |
| CA12 | 297.7260425 | −2.214492856 | 0.000947901 | MM-E |
| CXCL3 | 2006.38609 | −2.194242939 | 0.00021411 | MM-E |
| LOC101927630 | 36.48668577 | −2.188230512 | 0.043317358 | MM-E |
| NOS2 | 188.2398668 | −2.183843359 | 0.001022799 | MM-E |
| NMNAT2 | 49.90680546 | −2.177550352 | 0.022653918 | MM-E |
| LOC642846 | 269.3201154 | −2.167481038 | 7.21E-06 | MM-E |
| RNU12 | 26.92945062 | −2.164853314 | 0.035257504 | MM-E |
| SERPINA7 | 34.77073341 | −2.143400303 | 0.025369741 | MM-E |
| MIR378D2 | 31.95163204 | −2.140638202 | 0.010968635 | MM-E |
| SLC14A2 | 179.0450388 | −2.041282078 | 0.02092633 | MM-E |
| OVGP1 | 146.1512368 | −2.031036106 | 8.03E-07 | MM-E |
| VASN | 150.74635 | −2.030707991 | 0.008858331 | MM-E |
| LYPD8 | 106.2198186 | −2.029235933 | 0.017546438 | MM-E |
| FBXO16 | 62.70234755 | −1.995065159 | 0.002471512 | MM-E |
| BEND7 | 66.5589585 | −1.974430122 | 0.00466868 | MM-E |
| LGR5 | 6770.669082 | −1.93541127 | 0.008858331 | MM-E |
| POU3F1 | 53.26705622 | −1.905470706 | 0.02471569 | MM-E |
| RGMB-AS1 | 170.1776329 | −1.902397061 | 0.003103339 | MM-E |
| EGOT | 83.04091814 | −1.886597891 | 0.001515563 | MM-E |
| C7orf61 | 44.69240109 | −1.872632851 | 0.001865361 | MM-E |
| AXIN2 | 673.8127907 | −1.87028248 | 0.005042477 | MM-E |
| C19orf18 | 35.73593204 | −1.83873857 | 0.002233296 | MM-E |
| KCNJ14 | 84.14111071 | −1.816806693 | 0.002651879 | MM-E |
| RNVU1-14 | 335.5012872 | −1.809469813 | 0.001485219 | MM-E |
| GPC4 | 288.2755406 | −1.806396453 | 0.023697105 | MM-E |
| SCUBE2 | 281.6602748 | −1.787242397 | 0.008057009 | MM-E |
| AIF1L | 107.2165181 | −1.784799005 | 0.019599574 | MM-E |
| LOC101928100 | 203.1875517 | −1.776817252 | 0.002357699 | MM-E |
| KCNS3 | 248.0183629 | −1.733919554 | 0.033702364 | MM-E |
| LURAP1L-AS1 | 30.94944414 | −1.720097124 | 0.035826395 | MM-E |
| NOTCH3 | 416.3416182 | −1.689731542 | 0.045329791 | MM-E |
| MYC | 5121.974183 | −1.68897923 | 0.000399714 | MM-E |
| PABPC4L | 42.95792415 | −1.675088088 | 0.022880244 | MM-E |
| FAM171A1 | 1833.402124 | −1.663585337 | 0.000897665 | MM-E |
| GYLL1B | 233.4110046 | −1.662310433 | 0.047461188 | MM-E |
| PLCB4 | 1299.753251 | −1.650394064 | 0.006900096 | MM-E |
| LOXL1-AS1 | 76.79971859 | −1.640968095 | 0.02109749 | MM-E |
| MEX3A | 100.9009908 | −1.633362135 | 0.008858331 | MM-E |
| ARC | 1047.557695 | −1.625045869 | 0.009425752 | MM-E |
| PRKG1 | 139.4706533 | −1.619843691 | 0.006091691 | MM-E |
| TCAP | 26.69233405 | −1.593753464 | 0.044097489 | MM-E |
| CHRNA10 | 59.95220581 | −1.591580678 | 0.0499932 | MM-E |
| ZNRF3 | 1158.894619 | −1.589820903 | 6.94E-06 | MM-E |
| MGC50722 | 118.796859 | −1.589386229 | 0.006600696 | MM-E |
| ALOX5 | 1259.284063 | −1.567110833 | 0.00678467 | MM-E |
| FLJ31104 | 27.56430353 | −1.56660304 | 0.038549322 | MM-E |
| GAS2 | 239.3206859 | −1.559831287 | 0.033967335 | MM-E |
| NKILA | 70.94079094 | −1.546755967 | 0.02341088 | MM-E |
| MTSS1L | 64.81303817 | −1.518054447 | 0.014768634 | MM-E |
| NR4A1 | 16002.10353 | −1.489926139 | 0.018884899 | MM-E |
| LRRTM2 | 72.0991238 | −1.488809257 | 0.034897747 | MM-E |
| ADAMTS19 | 125.632282 | −1.487619471 | 0.000685222 | MM-E |
| SMTNL2 | 1218.865402 | −1.483810743 | 0.000173434 | MM-E |
| GPR161 | 284.9051551 | −1.476757056 | 0.012114938 | MM-E |
| ZSCAN12P1 | 310.7459748 | −1.470948045 | 0.034170359 | MM-E |
| CEP112 | 47.85624303 | −1.451606713 | 0.031907826 | MM-E |
| MND1 | 173.0958045 | −1.444569751 | 0.031511474 | MM-E |
| MEGF6 | 246.6363433 | −1.444364183 | 0.045329791 | MM-E |
| RHOV | 329.4369846 | −1.443922599 | 0.042765357 | MM-E |
| ELN | 107.7633429 | −1.440863428 | 0.020663186 | MM-E |
| DFNB59 | 66.93994714 | −1.439257373 | 0.014289478 | MM-E |
| MACROD2 | 104.2406382 | −1.430500491 | 0.017791313 | MM-E |
| MSI1 | 235.9239227 | −1.427921268 | 0.018606773 | MM-E |
| MNS1 | 145.3310743 | −1.42725988 | 0.027127275 | MM-E |
| SEMA4A | 199.7966861 | −1.426210613 | 0.005229378 | MM-E |
| DNMT3B | 227.4045048 | −1.424680805 | 0.034231221 | MM-E |
| RPGRIP1L | 181.1331442 | −1.421262073 | 0.014183607 | MM-E |
| TRPV4 | 107.1513861 | −1.412591379 | 0.045755668 | MM-E |
| ARHGAP39 | 467.3039477 | −1.400310338 | 0.034231221 | MM-E |
| TNFSF15 | 2348.942639 | −1.391340629 | 0.004448957 | MM-E |
| C5orf34 | 75.26522578 | −1.39111586 | 0.027974026 | MM-E |
| HBEGF | 6004.766903 | −1.383286445 | 0.041972168 | MM-E |
| RASL10B | 142.9873434 | −1.380171337 | 0.028065767 | MM-E |
| SETBP1 | 242.4009315 | −1.365148212 | 0.003996363 | MM-E |
| NFKB2 | 1730.570338 | −1.349489539 | 0.000685222 | MM-E |
| GNA14 | 89.80482731 | −1.343618965 | 0.013890125 | MM-E |
| EDN1 | 36686.0727 | −1.338724371 | 0.008717552 | MM-E |
| C18orf54 | 69.14691846 | −1.323400482 | 0.045755668 | MM-E |
| PVT1 | 178.2650316 | −1.312214055 | 0.039883206 | MM-E |
| RIMS2 | 141.5886217 | −1.311210448 | 0.023285283 | MM-E |
| SLC12A2 | 6906.985631 | −1.309058542 | 0.008411394 | MM-E |
| ADRA2A | 528.7215772 | −1.308678084 | 0.002233296 | MM-E |
| CHRM3 | 830.6272421 | −1.308175661 | 0.034231221 | MM-E |
| DFNB31 | 239.8696049 | −1.306232795 | 0.014436729 | MM-E |
| KCTD15 | 441.5458703 | −1.303725051 | 0.000539443 | MM-E |
| CTD-3080P12.3 | 2899.193152 | −1.302406658 | 0.015425302 | MM-E |
| CDR2L | 550.7081739 | −1.294482207 | 0.038030994 | MM-E |
| ROBO2 | 478.6162243 | −1.279111022 | 0.038384684 | MM-E |
| POLE2 | 263.3313471 | −1.274837929 | 0.034185359 | MM-E |
| RGMB | 2554.416581 | −1.272741751 | 0.020663186 | MM-E |
| FAM83D | 549.1358288 | −1.265260493 | 0.014446282 | MM-E |
| SALL4 | 974.2348226 | −1.247159949 | 0.040401208 | MM-E |
| EPHB2 | 3917.736439 | −1.24218703 | 0.032039798 | MM-E |
| LINC00342 | 152.4281474 | −1.229489631 | 0.00678467 | MM-E |
| PMP22 | 2223.506244 | −1.228511325 | 0.033018035 | MM-E |
| CD200 | 258.6104938 | −1.226367833 | 9.51E-05 | MM-E |
| NEIL3 | 211.4945095 | −1.224152546 | 0.034231221 | MM-E |
| ARHGEF38 | 1508.13545 | −1.22058815 | 0.004986866 | MM-E |
| FBXL18 | 476.5367421 | −1.214439061 | 0.034185359 | MM-E |
| CDCA7 | 3959.965977 | −1.212393144 | 0.01587915 | MM-E |
| FAM86FP | 133.985802 | −1.2110227 | 0.033133011 | MM-E |
| FILIP1L | 1143.784253 | −1.207937292 | 0.019062533 | MM-E |
| LINC00641 | 232.6891202 | −1.204879846 | 0.010299889 | MM-E |
| KIAA0319 | 113.7961184 | −1.201275736 | 0.043014725 | MM-E |
| ME3 | 839.4763575 | −1.193922616 | 0.011018776 | MM-E |
| OR51E1 | 266.5929011 | −1.18673081 | 0.035134194 | MM-E |
| SHCBP1 | 183.4394538 | −1.174924186 | 0.03706488 | MM-E |
| CEP128 | 139.4354378 | −1.174442385 | 0.004722316 | MM-E |
| ARHGAP8 | 962.7128601 | −1.172633465 | 0.027328562 | MM-E |
| C2orf72 | 2673.990963 | −1.166059567 | 0.000450259 | MM-E |
| SIX5 | 209.0972063 | −1.152112676 | 0.022019539 | MM-E |
| LONRF1 | 1510.533589 | −1.151365024 | 0.044808477 | MM-E |
| CSPP1 | 815.1859019 | −1.149836249 | 2.86E-06 | MM-E |
| PLK4 | 387.9237023 | −1.142434073 | 0.016366901 | MM-E |
| CEP131 | 522.0410444 | −1.139671998 | 0.007616271 | MM-E |
| KIF2C | 842.8401865 | −1.136116891 | 0.028117293 | MM-E |
| ZNF367 | 527.8607884 | −1.121264871 | 0.010968635 | MM-E |
| CYR61 | 14952.94096 | −1.119960245 | 0.047070201 | MM-E |
| EMC3-AS1 | 116.8879563 | −1.117938543 | 0.017144684 | MM-E |
| EZH2 | 1543.575815 | −1.112532758 | 0.006590811 | MM-E |
| ZNF618 | 602.9929501 | −1.102039738 | 0.000947901 | MM-E |
| PNPLA3 | 105.7239956 | −1.1005022 | 0.045755668 | MM-E |
| TAF1A | 336.8721961 | −1.09633272 | 0.014289478 | MM-E |
| RAD54B | 352.1899718 | −1.083962197 | 0.032039798 | MM-E |
| TSEN2 | 634.2458491 | −1.078259688 | 0.026252763 | MM-E |
| DZANK1 | 104.8204618 | −1.073786273 | 0.034185359 | MM-E |
| GSTM2 | 1188.91397 | −1.072215859 | 0.000623013 | MM-E |
| CHAF1B | 268.0062196 | −1.061673684 | 0.035869826 | MM-E |
| PAPD7 | 604.9957128 | −1.042866526 | 0.000257312 | MM-E |
| CENPF | 1408.530771 | −1.024015404 | 0.014689971 | MM-E |
| ENC1 | 9365.297897 | −1.017260496 | 0.007973377 | MM-E |
| TTC21A | 186.852232 | −1.015808321 | 0.028101018 | MM-E |
| CAMK1D | 403.664879 | −1.014268903 | 0.001531482 | MM-E |
| DACH1 | 1794.060966 | −1.008518999 | 0.00825271 | MM-E |
| SMTN | 3564.060253 | −1.001057577 | 0.00504639 | MM-E |
| AGXT2 | 3385.387486 | 1.000680967 | 0.000300639 | LM-E |
| LSP1 | 562.0696811 | 1.004609152 | 0.027592876 | LM-E |
| PIPDX | 2408.559655 | 1.005174551 | 0.011197424 | LM-E |
| NAAA | 3194.229583 | 1.006934446 | 0.047642246 | LM-E |
| AMDHD1 | 250.3134026 | 1.018146131 | 0.034170359 | LM-E |
| TNFSF10 | 4210.51754 | 1.021338958 | 0.008057009 | LM-E |
| SLC43A2 | 6836.316022 | 1.021433492 | 0.043512619 | LM-E |
| SIRT4 | 164.5498508 | 1.024191733 | 0.045755668 | LM-E |
| PNMA1 | 1299.461174 | 1.029170775 | 0.000197861 | LM-E |
| SMLR1 | 4858.75881 | 1.029683231 | 0.008057009 | LM-E |
| GPAT3 | 1425.442552 | 1.039993272 | 0.000557432 | LM-E |
| TREH | 7184.254013 | 1.042547839 | 0.002357699 | LM-E |
| MSRB1 | 3460.349402 | 1.044098343 | 0.037357224 | LM-E |
| IL1R1 | 160.6189988 | 1.044667026 | 0.017144684 | LM-E |
| GSN | 11555.56023 | 1.062750933 | 0.045165215 | LM-E |
| IL2RG | 3997.300125 | 1.068682104 | 0.009074611 | LM-E |
| GLMP | 6041.957401 | 1.071598873 | 0.001590383 | LM-E |
| AKAP12 | 116.4173565 | 1.073239658 | 0.017854124 | LM-E |
| MFI2 | 30411.04906 | 1.073668411 | 0.006772401 | LM-E |
| C2orf88 | 1413.396813 | 1.08074704 | 0.001865361 | LM-E |
| DOK1 | 142.7150926 | 1.083787028 | 0.014183607 | LM-E |
| ABHD6 | 4485.106932 | 1.084092213 | 0.004235326 | LM-E |
| LOC100270746 | 143.388674 | 1.085271775 | 0.035134194 | LM-E |
| MFI2-AS1 | 2670.219203 | 1.090701346 | 0.018117153 | LM-E |
| C1QC | 427.6448367 | 1.09440214 | 0.02994981 | LM-E |
| SLC22A17 | 1595.725184 | 1.100634971 | 0.006684394 | LM-E |
| ITGB7 | 397.3010281 | 1.102370504 | 0.001676486 | LM-E |
| PFKFB4 | 1222.511201 | 1.106223789 | 0.017144684 | LM-E |
| P4HA2-AS1 | 248.8460005 | 1.109226403 | 0.000897665 | LM-E |
| RENBP | 1022.258291 | 1.110818947 | 0.00825271 | LM-E |
| SLC16A4 | 2750.49756 | 1.112486758 | 0.025348081 | LM-E |
| RGS14 | 769.1305071 | 1.11273627 | 0.038018641 | LM-E |
| NOP9 | 7552.573924 | 1.12413866 | 0.001679136 | LM-E |
| TMIGD1 | 2413.829351 | 1.13662545 | 0.023076116 | LM-E |
| LIPA | 14098.8539 | 1.138327431 | 0.015263477 | LM-E |
| STK32C | 687.208622 | 1.145691044 | 0.037180604 | LM-E |
| FABP2 | 18839.01677 | 1.153734419 | 0.028337659 | LM-E |
| AMN | 69661.57922 | 1.156469731 | 0.019062533 | LM-E |
| PDZK1 | 2671.534262 | 1.15651815 | 0.013859792 | LM-E |
| AOC1 | 15203.18186 | 1.15821901 | 0.039354991 | LM-E |
| HNF4A-AS1 | 526.6237608 | 1.164666663 | 0.000399714 | LM-E |
| TARP | 85.64730167 | 1.165884381 | 0.039878808 | LM-E |
| TGFB1 | 184.1587069 | 1.168773252 | 0.038954837 | LM-E |
| SLC16A3 | 666.3207457 | 1.172165157 | 0.048425203 | LM-E |
| CIDEB | 7222.819692 | 1.172590594 | 0.001807622 | LM-E |
| KAZALD1 | 459.9894203 | 1.18313306 | 0.001688002 | LM-E |
| PCP4L1 | 241.509445 | 1.18683344 | 0.045329791 | LM-E |
| MYL3 | 339.9312772 | 1.189715236 | 0.028095907 | LM-E |
| ENPP3 | 1874.751914 | 1.195254633 | 0.011022593 | LM-E |
| ZBTB16 | 237.6180754 | 1.198455331 | 0.041405961 | LM-E |
| REEP1 | 2556.959636 | 1.201635381 | 0.013030521 | LM-E |
| KCNG1 | 2140.092515 | 1.206233494 | 0.001730778 | LM-E |
| MME | 16681.54852 | 1.210028373 | 0.027127275 | LM-E |
| TRIM63 | 284.1529105 | 1.214513887 | 0.032302404 | LM-E |
| GGN | 254.5687388 | 1.223823798 | 0.045329791 | LM-E |
| CPVL | 20111.03679 | 1.226007455 | 0.017080088 | LM-E |
| M1R192 | 131.4441235 | 1.226835254 | 0.014183607 | LM-E |
| TM6SF2 | 2490.528727 | 1.228605126 | 0.023484891 | LM-E |
| SLC15A1 | 13988.87574 | 1.235796305 | 0.010968635 | LM-E |
| TRPM4 | 336.2205185 | 1.236182841 | 0.003848639 | LM-E |
| MYO15A | 101.7183771 | 1.237612955 | 0.013069266 | LM-E |
| EFEMP2 | 120.120935 | 1.239462744 | 0.014183607 | LM-E |
| FAM132A | 2761.672598 | 1.245428993 | 0.02471569 | LM-E |
| CLEC10A | 52.75953571 | 1.262823732 | 0.011243881 | LM-E |
| UNC93A | 2410.486932 | 1.267480806 | 0.001679136 | LM-E |
| SASH3 | 342.2037765 | 1.271974492 | 0.016736053 | LM-E |
| ALOX5AP | 162.2037035 | 1.272042591 | 0.031097187 | LM-E |
| FLJ22763 | 3589.136583 | 1.272873045 | 0.011022593 | LM-E |
| MUC3A | 4563.341101 | 1.282863754 | 0.000399714 | LM-E |
| SLC28A1 | 2495.342817 | 1.286359064 | 0.003986406 | LM-E |
| PLCB2 | 163.3669478 | 1.30250326 | 0.029959857 | LM-E |
| CD5 | 64.57680706 | 1.314666903 | 0.044787445 | LM-E |
| ABCG5 | 1823.894132 | 1.315575137 | 0.003762794 | LM-E |
| LTB | 407.842644 | 1.320031367 | 0.034185359 | LM-E |
| LINC00616 | 28.7663487 | 1.329646899 | 0.022916509 | LM-E |
| REEP2 | 570.4384779 | 1.330537741 | 0.004374546 | LM-E |
| LINC00675 | 364.2931336 | 1.331404198 | 0.002624756 | LM-E |
| TUBA3FP | 61.09122808 | 1.332160766 | 0.045755668 | LM-E |
| NHLRC4 | 107.9600683 | 1.349698782 | 0.047251574 | LM-E |
| NPL | 1805.225372 | 1.357788852 | 0.026700902 | LM-E |
| CAPN3 | 11155.38665 | 1.362875817 | 0.003762794 | LM-E |
| LOC100133286 | 3725.864492 | 1.367851882 | 0.025126393 | LM-E |
| CYTH4 | 74.69667444 | 1.370071381 | 0.026249577 | LM-E |
| RAB42 | 1910.144097 | 1.390140269 | 0.012114938 | LM-E |
| DGKA | 287.6504932 | 1.395414502 | 0.008057009 | LM-E |
| SHBG | 1696.898935 | 1.396786195 | 0.003986406 | LM-E |
| LOC102467214 | 591.6898506 | 1.398397682 | 0.028581138 | LM-E |
| THSD7A | 288.7923303 | 1.401578846 | 0.030301279 | LM-E |
| TMEM151A | 57.64585109 | 1.405329059 | 0.014289478 | LM-E |
| CBR1 | 7333.545061 | 1.40583648 | 0.02711296 | LM-E |
| APOBEC1 | 1737.124109 | 1.408856297 | 0.001122434 | LM-E |
| MPP1 | 3207.997032 | 1.41720761 | 0.000450259 | LM-E |
| DUSP9 | 96.88920722 | 1.420442086 | 0.032889662 | LM-E |
| SLC34A3 | 3487.169128 | 1.449537839 | 0.001531176 | LM-E |
| TGM1 | 47.63354355 | 1.450420088 | 0.001298645 | LM-E |
| PAPL | 176.4468344 | 1.458116801 | 0.009644134 | LM-E |
| MAMDC4 | 58789.85242 | 1.462226222 | 0.000147606 | LM-E |
| SPIRE1 | 108.9921683 | 1.4644036 | 0.025804517 | LM-E |
| CD8A | 108.944692 | 1.468871589 | 0.029890319 | LM-E |
| PTPN7 | 153.560904 | 1.507909082 | 0.002175997 | LM-E |
| FRMD1 | 516.3820233 | 1.510816285 | 0.001588209 | LM-E |
| ENPP7 | 26477.35996 | 1.520721745 | 5.19E-07 | LM-E |
| PITPNM3 | 67.34060121 | 1.522859859 | 0.031875434 | LM-E |
| SECTM1 | 184.8290217 | 1.525112615 | 0.004261603 | LM-E |
| GDPD2 | 1370.518852 | 1.526569987 | 0.035885536 | LM-E |
| GFOD1 | 108.0575581 | 1.531593878 | 0.015021414 | LM-E |
| CD244 | 120.7936264 | 1.544555743 | 0.017930649 | LM-E |
| TM4SF5 | 2057.943428 | 1.562684067 | 0.006945573 | LM-E |
| STAR | 40.92258061 | 1.566842695 | 0.030301279 | LM-E |
| SH2D1B | 77.6739858 | 1.57302953 | 0.006900096 | LM-E |
| A2M | 113.8245729 | 1.57407166 | 0.003762794 | LM-E |
| SPANXN2 | 57.48240259 | 1.599754835 | 0.011658634 | LM-E |
| SLC2A7 | 147.6506746 | 1.601897947 | 0.004018553 | LM-E |
| TAGAP | 269.0685658 | 1.605696298 | 0.010968635 | LM-E |
| LOC388242 | 37.92691256 | 1.618754883 | 0.026605415 | LM-E |
| LOC613038 | 37.92691256 | 1.618754883 | 0.026605415 | LM-E |
| IKZF3 | 127.211745 | 1.626779639 | 0.004261603 | LM-E |
| A4GALT | 202.2729765 | 1.629306809 | 0.017483215 | LM-E |
| PRF1 | 132.2266665 | 1.647933134 | 3.00E-05 | LM-E |
| GPX3 | 656.031556 | 1.661358496 | 0.015899425 | LM-E |
| TTBK1 | 78.07689154 | 1.681876168 | 0.034340079 | LM-E |
| CIDEC | 2895.15844 | 1.691297258 | 7.96E-05 | LM-E |
| DGAT2 | 3215.740069 | 1.709850603 | 5.19E-07 | LM-E |
| ASIC3 | 40.36794931 | 1.714239419 | 0.014289478 | LM-E |
| SERPINE2 | 2506.302309 | 1.723513723 | 0.03256876 | LM-E |
| LOC101926963 | 105.4331048 | 1.724204362 | 0.014183607 | LM-E |
| FZD4 | 143.9315225 | 1.734234121 | 0.019278829 | LM-E |
| SH2D3C | 72.11715161 | 1.738919725 | 0.028483993 | LM-E |
| SERPING1 | 41.92840082 | 1.742341508 | 0.011288854 | LM-E |
| Cl11orf86 | 2882.237789 | 1.755304041 | 0.004448957 | LM-E |
| APOA1-AS | 262632.168 | 1.75699033 | 0.009212094 | LM-E |
| GIMAP7 | 81.72257311 | 1.761027091 | 0.034340079 | LM-E |
| CD3D | 154.0437705 | 1.763059079 | 0.027974026 | LM-E |
| BEGAIN | 195.4977337 | 1.773326287 | 0.000402194 | LM-E |
| APOA1 | 563298.695 | 1.796866065 | 0.006590811 | LM-E |
| F13B | 351.5148538 | 1.822618865 | 0.017209805 | LM-E |
| GBPS | 32.77839989 | 1.826824372 | 0.017546438 | LM-E |
| PECAM1 | 59.93169942 | 1.829490403 | 0.006600696 | LM-E |
| PLXDC1 | 22.67437759 | 1.853231819 | 0.008057009 | LM-E |
| VTN | 1997.731018 | 1.856597291 | 0.033018035 | LM-E |
| OTOP3 | 2425.97822 | 1.861205677 | 0.00084974 | LM-E |
| SLC38A3 | 80.79145313 | 1.871910659 | 0.003762794 | LM-E |
| ZG16 | 552.6271651 | 1.875831338 | 0.018117153 | LM-E |
| RARRE S3 | 307.8405168 | 1.881073436 | 0.016735483 | LM-E |
| NMUR1 | 47.96616816 | 1.89687475 | 0.033634341 | LM-E |
| CD69 | 801.4795788 | 1.903990599 | 0.006600696 | LM-E |
| PLEKHN1 | 106.0857972 | 1.932360672 | 0.033133011 | LM-E |
| DKK1 | 76.57517991 | 1.935847482 | 0.040563415 | LM-E |
| P2RX2 | 700.3330788 | 1.946832177 | 0.014289478 | LM-E |
| PHLDA3 | 358.1944413 | 1.960296892 | 0.002357699 | LM-E |
| FSTL1 | 107.918045 | 1.989682101 | 0.000810088 | LM-E |
| PCDH7 | 130.4058889 | 1.994054669 | 0.018606773 | LM-E |
| CYSLTR1 | 16.36262195 | 2.00380374 | 0.027402265 | LM-E |
| PNCK | 2558.444023 | 2.014527341 | 0.002357699 | LM-E |
| SLAMF6 | 35.80522583 | 2.054144792 | 0.018128577 | LM-E |
| C8G | 3297.63904 | 2.068220395 | 0.005885872 | LM-E |
| CYP2B7P | 2298.309465 | 2.081531515 | 0.001137341 | LM-E |
| FBLN2 | 99.39215515 | 2.08545985 | 0.005655911 | LM-E |
| CCL3 | 223.060639 | 2.100582853 | 0.02711296 | LM-E |
| MRGPRF | 17.61455412 | 2.102939426 | 0.025804517 | LM-E |
| LING01 | 504.6214903 | 2.10635471 | 0.002830454 | LM-E |
| CD160 | 117.1330868 | 2.144779667 | 0.006900096 | LM-E |
| LOC400043 | 68.75223492 | 2.154867092 | 0.013758221 | LM-E |
| CYP3A4 | 1239.396816 | 2.171844511 | 1.97E-05 | LM-E |
| CCL4L2 | 718.1477853 | 2.180277761 | 0.013409603 | LM-E |
| CCL4 | 328.8918663 | 2.180790089 | 0.008704583 | LM-E |
| APOC3 | 61341.46476 | 2.183494763 | 0.005072149 | LM-E |
| ALDOB | 12761.6415 | 2.191797875 | 0.007263063 | LM-E |
| CCL4L1 | 707.3127006 | 2.197670226 | 0.013198956 | LM-E |
| ALPI | 5839.143349 | 2.208321901 | 0.017854124 | LM-E |
| SLC22A7 | 51.25961483 | 2.248424439 | 0.001485219 | LM-E |
| HBA2 | 346.6239428 | 2.257660623 | 0.015263477 | LM-E |
| LINC00671 | 58.73732319 | 2.269080019 | 0.025631517 | LM-E |
| SIGLEC7 | 23.95039501 | 2.281349356 | 0.019565021 | LM-E |
| TREML3P | 36.04101379 | 2.285115261 | 0.002175997 | LM-E |
| DCLK2 | 16.44792496 | 2.298167334 | 0.035134194 | LM-E |
| CD4OLG | 11.62832448 | 2.299476059 | 0.038614976 | LM-E |
| XCL2 | 33.74716796 | 2.320177898 | 0.02471569 | LM-E |
| PM20D1 | 110.9959034 | 2.389640199 | 0.013004478 | LM-E |
| ADGRF1 | 36.21198055 | 2.446570021 | 0.014289478 | LM-E |
| GIMAP6 | 108.0298791 | 2.475689398 | 0.002233296 | LM-E |
| TMEM132B | 36.33778204 | 2.487370499 | 0.009425752 | LM-E |
| CYP1A1 | 69.64980339 | 2.547605576 | 0.012642972 | LM-E |
| 4-Sep | 1078.319906 | 2.595002993 | 0.00478362 | LM-E |
| EFEMP1 | 36.12365776 | 2.604558942 | 0.038273656 | LM-E |
| ASPDH | 103.4840262 | 2.607496108 | 1.02E-06 | LM-E |
| ACSM2B | 61.51831137 | 2.60900445 | 5.32E-05 | LM-E |
| ANKRD2 | 12.37918238 | 2.643749112 | 0.049533875 | LM-E |
| TNF | 34.19477787 | 2.716698036 | 0.033133011 | LM-E |
| HPR | 48.67123183 | 2.732516536 | 0.02711296 | LM-E |
| APOA4 | 313587.2217 | 2.775884458 | 2.86E-06 | LM-E |
| PART1 | 13.98596608 | 2.795454936 | 0.040591295 | LM-E |
| APOA5 | 43.25455871 | 2.811745767 | 0.006600696 | LM-E |
| M1R198 | 7.823320286 | 2.824536122 | 0.006772401 | LM-E |
| SEPT4-AS1 | 202.4789386 | 2.865275195 | 0.000557432 | LM-E |
| IMPG2 | 13.45492424 | 2.905949143 | 0.043396042 | LM-E |
| G6PC | 116.3431418 | 2.939244399 | 0.000257312 | LM-E |
| DLGAP1 | 47.69190466 | 3.035091998 | 0.004003618 | LM-E |
| BTLA | 10.25947077 | 3.36203134 | 0.014894532 | LM-E |
| FAM205C | 10.90260561 | 3.384038339 | 0.033011385 | LM-E |
| DPPA4 | 11.29030897 | 3.384534523 | 0.01354762 | LM-E |
| FGF12 | 22.8626664 | 3.425932791 | 0.004374546 | LM-E |
| FABP6 | 2713.079447 | 3.481603799 | 0.017209805 | LM-E |
| DUOXA2 | 7.989387148 | 3.497135899 | 0.025355462 | LM-E |
| LOC101929696 | 9.703210649 | 3.533053893 | 0.024074715 | LM-E |
| LPAR3 | 31.29012013 | 3.570664465 | 8.46E-07 | LM-E |
| TPO | 41.21778229 | 3.598594618 | 2.86E-06 | LM-E |
| TDRD10 | 11.13826867 | 3.60525867 | 0.00875909 | LM-E |
| 5T851A2 | 14.38741819 | 3.724254437 | 0.02711296 | LM-E |
| UPK3B | 9.080948707 | 3.99773204 | 0.010999563 | LM-E |
| Cl7orf105 | 3.870217375 | 4.102219679 | 0.036791762 | LM-E |
| LOC101929532 | 12.25884812 | 4.207871983 | 0.011658634 | LM-E |
| M1R3193 | 9.4223208 | 4.574911055 | 0.017080088 | LM-E |
| HCFC1-AS1 | 9.086731018 | 4.649473603 | 0.04062786 | LM-E |
| LINC01272 | 8.199589596 | 4.820699777 | 0.022744108 | LM-E |
| LOC200772 | 8.986438185 | 4.886272818 | 0.015425302 | LM-E |
| LINC01529 | 9.670297171 | 5.129805856 | 0.018845779 | LM-E |
| VEGFC | 3.729305979 | 5.254170297 | 0.028101018 | LM-E |
| EPB42 | 6.638885006 | 5.393394241 | 0.000399714 | LM-E |
| PRDM8 | 5.253867942 | 5.52373613 | 0.038030994 | LM-E |
| ADAD2 | 5.886364013 | 5.638920982 | 0.032039798 | LM-E |
| LOC102723544 | 5.736052635 | 5.650484475 | 0.029890319 | LM-E |
| LOC400558 | 4.780263845 | 6.248385513 | 0.031511474 | LM-E |
| GPR31 | 8.124129378 | 6.692767034 | 0.005887074 | LM-E |
| LOC100506159 | 83.38815143 | 7.582717454 | 0.032039798 | LM-E |

TABLE 4A
Fetal Meconium Isolates

| | | Meconium subset | | |
|---|---|---|---|---|
| Isolate | Meconium | by 16S V4 rRNA | | |
| Identifier | SampleID | Sequencing | Isolation Media | Isolation Conditions |
| M45 | 15391 | MM | cRPMI + THP1 monocytes | 48h 37C stationary conditions |
| M46 | 15391 | MM | cRPMI + THP1 monocytes | 48h 37C stationary conditions |
| Micro36 | 1543J | MM | cRPMI + THP1 monocytes | 48h 37C stationary conditions |
| M44 | 1539J | MM | cRPMI + THP1 monocytes | 48h 37C stationary conditions |
| M47 | 1539J | MM | cRPMI + THP1 monocytes | 48h 37C stationary conditions |
| M52 | 1539J | MM | cRPMI + THP1 monocytes | 48h 37C stationary conditions |
| M155 | 1539J | MM | cRPMI + THP1 monocytes | 48h 37C stationary conditions |
| M49 | 1539J | MM | cRPMI + THP1 monocytes | 48h 37C stationary conditions |
| M53 | 1544J | MM | cRPMI + THP1 monocytes | 48h 37C stationary conditions |
| M37 | 1543J | MM | cRPMI + THP1 monocytes | 48h 37C stationary conditons |
| M38 | 1543J | MM | cRPMI + THP1 monocytes | 48h 37C stationary conditons |
| M39 | 1543J | MM | cRPMI + THP1 monocytes | 48h 37C stationary conditions |
| MicroRef | NA | NA | NA | Obtained from ATCC, Cat. No, 4698 |
| LactoRef | NA | NA | NA | Obtained from ATCC. Cat. No. 55195 |
| Lacto166 | 1652J | LM | TSA + 5% Horse Blood + P4 and E2 | 48h 37C 5% CO2 stationary conditions |
| Lacto167 | 1611J | LM | TSA + 5% Horse Blood + P4 and E2 | 48h 37C 5% CO2 stationary conditions |

TABLE 4B
Fetal Meconium Isolates

| Isolate | | | % Taxonomy | % Identity | % Identity |
|---|---|---|---|---|---|
| Identifier | Subcutture Coditions | SILVA Taxonomy | Identity | to OTU10 | to OTU12 |
| M45 | BHI, 48h 37C stationaiy conditions | Bacteria;Proteobacteria: | 99.075 | 75 | 79.835 |
| | | Gammaproteobacteria ; | | | |
| | | Pseudomonadales; | | | |
| | | Pseudomonadaceae; | | | |
| | | Pseudomonas; | | | |
| M46 | BHI, 48h 37C stationary conditions | Bacteria;Proteobacteria; | 98.4536 | 74.699 | 79.508 |
| | | Gammaproteobacteria; | | | |
| | | Pseudomonadales; | | | |
| | | Pseudomonadaceae; | | | |
| | | Pseudomonas; | | | |
| Micro36 | BHI, 48h 37C stationary conditions | Bacteria;Actinobacteria; | 99.8304 | 97.233 | 76.285 |
| | | Actinobacteria; | | | |
| | | Micrococcales; | | | |
| | | Micrococcaceae; | | | |
| | | Micrococcus; | | | |
| M44 | BHI, 48h 37C stationary conditions | Bacteria;Proteobacteria; | 99.7927 | 79.051 | 76.078 |
| | | Gammaproteobacteria; | | | |
| | | Xanthorrionadales; | | | |
| | | Xanthomonadaceae; | | | |
| | | Stenotrophomonas; | | | |
| M47 | BHI, 48h 37C stationary conditions | Bacteria;Proteobacteria; | 99.793 | 79.051 | 76.078 |
| | | Gammaproteobacteria; | | | |
| | | Xanthomonadales; | | | |
| | | Xanthomonadaceae; | | | |
| | | Stenotrophomonas; | | | |
| M52 | BHI, 48h 37C stationary conditions | Bacteria:Proteobacteria: | 99.5889 | 79.051 | 76.078 |
| | | Gammaproteobacteria; | | | |
| | | Xanthomonadales; | | | |
| | | Xanthomonadaceae; | | | |
| | | Stenotrophmonas; | | | |
| M155 | BHI, 48h 37C stationary conditions | Bacteria;Proteobacteria; | 997923 | 79.051 | 76.078 |
| | | Gammaproteobacteria; | | | |
| | | Xanthomonadales; | | | |
| | | Xanthomonadaceae; | | | |
| | | Stenotrophomonas; | | | |
| M49 | BHI, 48h 37C stationary conditions | Bacteria;Proteobactena; | 98.9765 | 78.656 | 75.686 |
| | | Gammaproteobacteria; | | | |
| | | Xanthomonadales; | | | |
| | | Xanthomonadaceae; | | | |
| | | Stenotrophomonas; | | | |
| M53 | BHI, 48h 37C stationary conditions | Bacteria;Proteobacteria; | 99,0702 | 78.656 | 75.686 |
| | | Gammaproteobacteria; | | | |
| | | Xanthornonadales; | | | |
| | | Xanthomonadaceae; | | | |
| | | Stenotrophomonas; | | | |
| M37 | BHI, 48h 37C stationary conditions | Bacteria:Actinobacteria; | 99.5767 | 92.095 | 75.494 |
| | | Actinobacteria; | | | |
| | | Micrococcales; | | | |
| | | Microbacteriaceae; | | | |
| | | Microbacterium; | | | |
| M38 | BHI, 48h 37C stationary conditions | Bacteria;Actinobacteria; | 99.7901 | 92.095 | 75.494 |
| | | Actinobacteria; | | | |
| | | Micrococcales; | | | |
| | | Microbacteriaceae; | | | |
| | | Microbacterium; | | | |
| M39 | BHI, 48h 37C stationary conditions | Bacteria;Actinobacteria; | 99.894 | 92.095 | 75.494 |
| | | Actinobateria; | | | |
| | | Micrococcales; | | | |
| | | Microbacteriaceae; | | | |
| | | Microbacterium; | | | |
| MicroRef | BHI, 48h 37C stationary conditions | Bacteria;Actinobacteria; | 100 | 97.233 | 76.285 |
| | | Actinobacteria; | | | |
| | | Micrococcales; | | | |
| | | Micrococcacsae; | | | |
| | | Micrococcus; | | | |
| LactoRef | TSA + 5% Horse Blood, 37C 5% | Bacteria;Firmicutes;Bacilli; | 100 | 75.099 | 100 |
| | CO2 stationary conditions | Lactobacillales; | | | |
| | | Lactobacillaceae; | | | |
| | | Lactobacilius; | | | |
| Lacto166 | TSA + 5% Horse Blood, 37C 5% | Bacteria;Firmicutes;Bacilli; | 95.2 | 77.47 | 95.652 |
| | CO2 stationary conditions | Lactobacillales; | | | |
| | | Lactobacillaceae; | | | |
| | | Lactobacilius; | | | |
| Lacto167 | TSA + 5% Horse Blood, 37C 5% | Bacteria;Firmicutes;Bacilli; | 97.384 | 77.47 | 95.652 |
| | CO2 stationary conditions | Lactobacillales; | | | |
| | | Lactobacillaceae; | | | |
| | | Lactobacilius; | | | |

TABLE 5
AFetal Meconium Isolate Whole Genome Sequencing Statistics

| | | Meconium subset by | | | |
|---|---|---|---|---|---|
| Isolate | Meconium | 16S V4 rRNA | No. | Total genome | |
| Identifier | SampleID | Sequencing | contigs | length | N50 |
| Micro36 | 1543J | MM | 101 | 2584920 | 59431 |
| Lacto166 | 1652J | LM | 44 | 1669151 | 69019 |
| Lacto167 | 1611J | LM | 41 | 1633781 | 65981 |

TABLE 5B
Fetal Meconium Isolate Whole Genome Sequencing Statistics

| | | | | | | Reads | | |
|---|---|---|---|---|---|---|---|---|
| | | | | | | mapped | x Mean | |
| | | | | | | to contig | Coverage | |
| Isolate | | | | Largest | GC | assembly | (Standard | NCBI |
| Identifier | N75 | L50 | L75 | Contig | (%) | (%) | Deviation) | Accession |
| Micro36 | 27520 | 14 | 31 | 205052 | 72.79 | 99.17 | 511.3 | PRJNA498337 |
| | | | | | | | (332.3) | |
| Lacto166 | 53049 | 7 | 14 | 251276 | 34.14 | 99.5 | 1280.1 | PRJNA498338 |
| | | | | | | | (836.7) | |
| Lacto167 | 52618 | 8 | 14 | 250289 | 34.18 | 99.2 | 768.3 | PRJNA498340 |
| | | | | | | | (400.6) | |

TABLE 6A
Average nucleotide identity and coverage of
Micro36 against all available genomes in Micrococcus

| Average nucleotide | | M. aloveae | M. luteus | M. luteus | M. luteus | M. luteus |
|---|---|---|---|---|---|---|
| identity (%) | Micro36 | M.71 | CCH3 E2 | K39 | NCTC 2665 | SGAir0127 |
| Micro36 | 100 | 97.9974632 | 98.0426837 | 98.0584686 | 96.8318581 | 98.0360053 |
| M. aloeveae M.71 | 98.0583597 | 100 | 98.2177074 | 98.2522981 | 96.951068 | 98.1041768 |
| M. luteus CCH3 E2 | 98.1339428 | 98.1773169 | 100 | 98.0411098 | 97.0526662 | 98.2727932 |
| M. luteus K39 | 98.0735709 | 98.2583714 | 98.0613886 | 100 | 96.9305351 | 98.0665795 |
| M. luteus NCTC 2665 | 96.8906281 | 96.9782001 | 96.9734035 | 96.9312467 | 100 | 97.1005789 |
| M. luteus SGAir0127 | 98.1351183 | 98.0976999 | 98.2995674 | 98.0946873 | 97.1321742 | 100 |
| M. luteus UMB0038 | 98.3404044 | 97.9101996 | 97.9579452 | 98.2649475 | 96.720887 | 98.1521142 |
| M. luteus UMB0189 | 98.701493 | 97.9904329 | 98.0826111 | 98.0003536 | 96.7558408 | 98.0725415 |
| M. lylae NBRC 15355 | 79.5906967 | 79.596312 | 80.0220329 | 79.8794871 | 79.2570298 | 79.6977507 |
| M. terreus CG M.CC 1.7054 | 76.4594776 | 76.3096553 | 76.8020678 | 76.4750488 | 76.538867 | 76.5018682 |
| Micococcus sp HMSC30C05 | 98.7962828 | 97.9767778 | 98.1086493 | 98.0463374 | 96.7658293 | 98.145107 |
| Microccus sp KT16 | 98.1580842 | 98.2533806 | 98.1960056 | 98.1892312 | 97.0347042 | 98.1321247 |
| Micrococcus sp HMSC31601 | 98.6799031 | 98.063257 | 98.0897519 | 98.1964479 | 96.7697774 | 98.1642525 |

TABLE 6B
Average nucleotide identity and coverage of
Micro36 against all available genomes in Micrococcus

| | | | | M. terreus | | | |
|---|---|---|---|---|---|---|---|
| Average nucleotide | M. luteus | M. luteus | M. lylae | CG M.CC | Micococcus sp | Microccus sp | Micrococcus sp |
| identity (%) | UMB0038 | UMB0189 | NBRC 15355 | 1.7054 | HMSC30C05 | KT16 | HMSC31B01 |
| Micro36 | 98.4259949 | 98.7903333 | 79.5441421 | 76.3321721 | 98.844429 | 98.0907957 | 98.7530279 |
| M. aloeveae M.71 | 98.0323145 | 98.0554157 | 79.5200384 | 76.4159475 | 98.044476 | 98.1644046 | 98.1657169 |
| M. luteus CCH3 E2 | 98.0219056 | 98.1267268 | 79.8534474 | 76.861865 | 98.1046368 | 98.0996323 | 98.1242562 |
| M. luteus K39 | 98.3225642 | 98.1132818 | 79.9813266 | 76.5418922 | 98.1832695 | 98.0729653 | 98.3366949 |
| M. luteus NCTC 2665 | 96.8776828 | 96.8099104 | 79.4501095 | 76.5501732 | 96.8119252 | 96.9880309 | 96.9651932 |
| M. luteus SGAir0127 | 98.1772406 | 98.1140785 | 79.7867218 | 76.6759268 | 98.3413836 | 98.0944176 | 98.3678172 |
| M. luteus UMB0038 | 100 | 98.4231542 | 79.5841112 | 76.4308441 | 98.5050303 | 97.962887 | 98.3978776 |
| M. luteus UMB0189 | 98.4038791 | 100 | 79.8542671 | 76.66552 | 98.8244134 | 98.0234393 | 98.6148494 |
| M. lylae NBRC 15355 | 79.5073341 | 79.6598372 | 100 | 76.7967064 | 81.1839407 | 79.5266632 | 80.9103028 |
| M. terreus CG M.CC 1.7054 | 76.5411418 | 76.4877541 | 76.8581604 | 100 | 77.6194206 | 76.3994446 | 77.4471057 |
| Micococcus sp HMSC30C05 | 98.4663876 | 98.8571791 | 80.4421481 | 77.1884204 | 100 | 97.9964088 | 98.7269168 |
| Microccus sp KT16 | 98.0689642 | 98.1618233 | 79.4923301 | 76.5689704 | 98.0163331 | 100 | 98.2473815 |
| Micrococcus sp HMSC31B01 | 98.386403 | 98.6634489 | 80.2363515 | 77.2879698 | 98.7856975 | 98.0992954 | 100 |

TABLE 6C
Average nucleotide identity and coverage of
Micro36 against all available genomes in Micrococcus

| | | M. aloeveae | M. luteus | M. luteus | M. luteus | M. luteus |
|---|---|---|---|---|---|---|
| Coverage (%) | Micro36 | M.71 | CCH3 E2 | K39 | NCTC 2665 | SGAir0127 |
| Micro36 | 100 | 83.3640731 | 74.9213867 | 83.7818504 | 80.3114326 | 86.8532244 |
| M. aloeveae M.71 | 87.8231357 | 100 | 80.0527094 | 88.6762747 | 84.1765452 | 90.0758525 |
| M. luteus CCH3 E2 | 87.5038776 | 88.994926 | 100 | 87.1331219 | 85.0703047 | 92.7274746 |
| M. luteus K39 | 86.8862843 | 86.5966554 | 76.2960685 | 100 | 80.8739082 | 88.5367991 |
| M. luteus NCTC 2665 | 81.5400602 | 80.9222113 | 74.2990376 | 79.7961055 | 100 | 84.4308318 |
| M. luteus SGAir0127 | 83.3635211 | 82.1925109 | 76.7605113 | 82.5782167 | 79.7155606 | 100 |
| M. luteus UMB0038 | 86.8692747 | 82.1332369 | 73.4637293 | 84.4213601 | 78.6036088 | 86.5571399 |
| M. luteus UMB0189 | 88.6925171 | 83.8478119 | 76.9701168 | 82.7154535 | 80.5015732 | 86.3060112 |
| M. lylae NBRC 15355 | 46.0601326 | 46.7478751 | 42.6779808 | 45.3530648 | 45.8833005 | 46.8621886 |
| M. terreus CG M.CC 1.7054 | 30.601324 | 30.4688877 | 28.9420635 | 30.498264 | 30.7417928 | 31.2462429 |
| Micococcus sp HMSC30C05 | 89.2157763 | 82.4377237 | 77.1510441 | 82.9948935 | 79.0718493 | 85.1177171 |
| Microccus sp KT16 | 84.965775 | 86.8352255 | 78.2585247 | 85.7340894 | 81.7601638 | 88.0130252 |
| Micrococcus sp HMSC31601 | 88.8081621 | 84.7208394 | 78.1902894 | 86.1459013 | 80.7045115 | 86.7864923 |

TABLE 6D
Average nucleotide identity and coverage of
Micro36 against all available genomes in Micrococcus

| | | | | M. terreus | | | |
|---|---|---|---|---|---|---|---|
| | M. luteus | M. luteus | M. lylae | CG M.CC | Micococcus sp | Microccus sp | Micrococcus sp |
| Coverage (%) | UMB0038 | UMB0189 | NBRC 15355 | 1.7054 | HMSC30C05 | KT16 | HMSC31B01 |
| Micro36 | 90.1085734 | 88.8951806 | 49.678459 | 36.9744903 | 67.282659 | 84.118552 | 71.0484488 |
| M. aloeveae M.71 | 89.9614393 | 89.1684003 | 53.2920046 | 38.781243 | 65.5513721 | 90.5592566 | 71.4985857 |
| M. luteus CCH3 E2 | 89.7042105 | 89.9263894 | 53.9978493 | 41.3096542 | 68.1703148 | 90.8781455 | 72.4821171 |
| M. luteus K39 | 90.4752317 | 85.9361472 | 49.0657787 | 37.600628 | 63.8213349 | 87.3585876 | 70.4646556 |
| M. luteus NCTC 2665 | 83.7452126 | 82.2078072 | 49.4105986 | 38.7595123 | 59.8459396 | 82.1081709 | 64.2425704 |
| M. luteus SGAir0127 | 86.6515815 | 83.5910681 | 47.2193923 | 35.7847143 | 61.7214926 | 83.346118 | 66.4710004 |
| M. luteus UMB0038 | 100 | 87.4870367 | 47.9415392 | 36.7571612 | 64.91809 | 83.3389318 | 68.2697585 |
| M. luteus UMB0189 | 90.515027 | 100 | 49.6052184 | 38.3238554 | 67.4427281 | 86.7944543 | 71.531826 |
| M. lylae NBRC 15355 | 46.6416418 | 46.8189952 | 100 | 33.3913341 | 35.5187932 | 46.7879407 | 37.6656894 |
| M. terreus CG M.CC 1.7054 | 30.9336944 | 31.1177579 | 29.0543219 | 100 | 25.4541185 | 30.9467793 | 26.3797805 |
| Micococcus sp HMSC30C05 | 88.5848336 | 89.0556175 | 50.0670221 | 39.8682838 | 100 | 85.1102256 | 86.3697408 |
| Microccus sp KT16 | 87.8992882 | 88.1194914 | 50.1889625 | 38.4822355 | 64.7602893 | 100 | 69.2750951 |
| Micrococcus sp HMSC31B01 | 89.1743942 | 89.9032875 | 50.761072 | 40.0620385 | 82.2534571 | 85.9590465 | 100 |

TABLE 7A
Average nucleotide identity and genome coverage (%) of
Lacto166 and Lacto167 against select genomes in Lactobacillus

| | | | | | L. gasseri | |
|---|---|---|---|---|---|---|
| Average nucleotide | L. acidophilus | | L. crispatus | | ATCC 33323 | |
| identity (%) | NCFM | L. amylolyticus | ST1 | L. gallinarum | JCM 1131 | L. helveticus |
| L. acidophilus NCFM | 100 | 76.8136653 | 80.2160797 | 81.0835843 | 74.7548482 | 80.9441115 |
| L. amylolyticus | 77.0339467 | 100 | 77.0280152 | 77.4410259 | 74.5350894 | 77.476334 |
| L. crispatus ST1 | 80.2120639 | 76.8555016 | 100 | 80.5921279 | 74.557128 | 80.5181746 |
| L. gallinarum | 81.1264058 | 78.4402909 | 81.0102368 | 100 | 74.1202228 | 96.9981251 |
| L. gasseri ATCC 33323 JCM 1131 | 75.0460225 | 74.7181725 | 74.8327152 | 74.8131266 | 100 | 74.8377105 |
| L. helveticus | 80.9911766 | 77.8549325 | 80.7615433 | 97.4487066 | 74.5852155 | 100 |
| L. iners DSM 13335 | 72.2595444 | 71.8760655 | 71.9495243 | 72.048145 | 73.6642057 | 72.0603843 |
| L. jensenii 1153 | 73.7351403 | 73.3792088 | 73.5055877 | 73.8355363 | 73.6414655 | 73.7697194 |
| L. jensenii 115 3 CHN | 74.0043779 | 73.6077216 | 73.6693774 | 73.9063925 | 73.6169352 | 73.8289504 |
| L. jensenii 269 3 | 73.9447771 | 73.4844514 | 73.7394221 | 74.1040845 | 73.7436511 | 73.9440239 |
| L. jensenii 27 2 CHN | 73.9373617 | 73.703925 | 73.6466032 | 73.9880265 | 73.5525782 | 73.8609432 |
| L. jensenii DSM 20557 | 74.0216991 | 73.6807389 | 73.638506 | 73.994551 | 73.563393 | 73.859041 |
| L. jensenii IM1 | 74.0147525 | 73.6684432 | 73.8000731 | 74.2207532 | 73.8172777 | 74.1861149 |
| L. jensenii IM11 | 73.9456063 | 73.7610494 | 73.7314026 | 74.047523 | 73.6455309 | 73.8634865 |
| L. jensenii IM18 1 | 73.7085873 | 73.1200577 | 73.3759149 | 73.6204728 | 73.2597528 | 73.6106498 |
| L. jensenii IM18 3 | 73.9300964 | 73.2898823 | 73.5576376 | 74.0035845 | 73.6511306 | 73.8426753 |
| L. jensenii IM3 | 73.909933 | 73.6742442 | 73.7988927 | 74.2379227 | 73.6180247 | 74.0789464 |
| L. jensenii IM59 | 73.7588059 | 73.0361354 | 73.3314439 | 73.8162468 | 73.5512391 | 73.563467 |
| L. jensenii JV V16 | 73.9233197 | 73.7463665 | 73.8635056 | 74.4109787 | 73.6007018 | 74.224 |
| L. jensenii MD IIE 702 | 73.903762 | 73.8644062 | 73.7226943 | 74.1763016 | 73.429458 | 74.0390342 |
| L. jensenii SJ 7A US | 73.8718968 | 73.4223115 | 73.561261 | 73.8987218 | 73.6871333 | 73.8499201 |
| L. jensenii SNUV360 RefSeq | 73.9325983 | 73.7856961 | 73.8763904 | 74.6547607 | 73.5289793 | 74.6364776 |
| L. jensenii T L.2937 | 74.1417373 | 74.1930139 | 74.0604089 | 74.8419284 | 73.8422052 | 74.6341272 |
| L. jensenii UMB0007 | 73.9670664 | 73.8417896 | 73.6631786 | 74.1753049 | 73.5639857 | 73.9976849 |
| L. jensenii UMB0077 | 73.8699137 | 73.7896508 | 73.6862471 | 74.2347512 | 73.5666998 | 74.0085074 |
| L. psittaci DSM 15354 | 73.8507744 | 73.4142115 | 73.678441 | 73.8602228 | 73.5792533 | 73.7820654 |
| Lacto166 | 73.9234719 | 73.624228 | 73.6295 | 74.2105279 | 73.7141944 | 74.0197473 |
| Lacto167 | 73.8537613 | 73.5932304 | 73.6110465 | 74.0443571 | 73.7138364 | 73.8847963 |

TABLE 7B
Average nucleotide identity and genome coverage (%) of
Lacto166 and Lacto167 against select genomes in Lactobacillus

| | L. iners | | | | | |
|---|---|---|---|---|---|---|
| Average nucleotide | DSM | L. jensenii | L. jensenii | L. jensenii | L. jensenii | L. jensenii |
| identity (%) | 13335 | 1153 | 115 3 CHN | 269 3 | 27 2 CH N | DSM 20557 |
| L. acidophilus NCFM | 72.7281623 | 74.08018 | 74.01565 | 74.0918567 | 74.0584832 | 74.0815105 |
| L. amylolyticus | 72.7574042 | 73.9381411 | 74.024254 | 73.8820748 | 74.0075641 | 74.1635224 |
| L. crispatus ST1 | 72.5783489 | 73.8204021 | 73.9094432 | 73.7642669 | 73.9264263 | 73.9636006 |
| L. gallinarum | 72.5059168 | 74.75035 | 74.6280927 | 74.1870275 | 74.697189 | 74.3941638 |
| L. gasseri ATCC 33323 JCM 1131 | 74.4463814 | 74.3626094 | 74.1578777 | 74.3655997 | 74.164431 | 74.2547028 |
| L. helveticus | 72.6551627 | 74.530534 | 74.3901839 | 74.0000431 | 74.412552 | 74.4060214 |
| L. iners DSM 13335 | 100 | 72.3524717 | 72.2213574 | 72.305189 | 72.3417922 | 72.3049467 |
| L. jensenii 1153 | 72.3301237 | 100 | 88.0652078 | 99.888598 | 88.0514643 | 88.2479477 |
| L. jensenii 115 3 CHN | 72.4073932 | 88.0794945 | 100 | 88.0533873 | 99.9274972 | 99.9564542 |
| L. jensenii 269 3 | 72.4563086 | 99.9060998 | 88.0696445 | 100 | 88.0791759 | 88.2459404 |
| L. jensenii 27 2 CH N | 72.3702962 | 88.0808379 | 99.8632107 | 88.0709668 | 100 | 99.5982118 |
| L. jensenii DSM 20557 | 72.4957125 | 88.1934362 | 99.8906655 | 88.1399968 | 99.6184959 | 100 |
| L. jensenii IM1 | 72.6597169 | 99.6807889 | 87.8275503 | 99.6512171 | 87.8287905 | 88.0593232 |
| L. jensenii IM11 | 72.4856512 | 88.0547954 | 99.8940765 | 88.2553545 | 99.4883971 | 99.7410423 |
| L. jensenii IM18 1 | 72.1338699 | 99.7390607 | 88.01205 | 99.7383814 | 88.0211264 | 88.0908075 |
| L. jensenii IM18 3 | 72.3074862 | 99.7880672 | 88.0658446 | 99.8104097 | 88.0256563 | 88.4294618 |
| L. jensenii IM3 | 72.3417265 | 88.0521129 | 99.810147 | 88.071588 | 99.7595197 | 99.6572364 |
| L. jensenii IM59 | 72.1472247 | 99.7098303 | 87.9547206 | 99.7354513 | 87.9401775 | 88.0560795 |
| L. jensenii JV V16 | 72.3340424 | 88.1481418 | 99.8403424 | 87.9433314 | 99.9381811 | 99.530879 |
| L. jensenii MD IIE 702 | 72.409653 | 88.1561621 | 99.8725271 | 88.383362 | 99.4866153 | 99.6568017 |
| L. jensenii Si 7A US | 72.4254856 | 99.980457 | 88.0457952 | 99.8915083 | 88.0820684 | 88.2411955 |
| L. jensenii SNUV360 RefSeq | 72.5040904 | 99.8634828 | 88.0786436 | 99.877382 | 88.117053 | 88.3101961 |
| L. jensenii T L.2937 | 72.6782282 | 88.4238463 | 99.8769307 | 88.2087474 | 99.7562166 | 99.7844973 |
| L. jensenii UMB0007 | 72.2018855 | 88.181962 | 99.9299786 | 88.3005132 | 99.543981 | 99.588329 |
| L. jensenii UMB0077 | 72.1851623 | 88.3022103 | 99.9153266 | 88.2801205 | 99.927984 | 99.9584876 |
| L. psittaci DSM 15354 | 72.1415833 | 80.2890103 | 83.0475765 | 80.298923 | 83.0452381 | 83.0812444 |
| Lacto166 | 72.1542306 | 88.1400279 | 99.8768568 | 88.2997145 | 99.8182185 | 99.7640934 |
| Lacto167 | 72.1200618 | 88.1536409 | 99.8705142 | 88.332849 | 99.829002 | 99.7813781 |

TABLE 7C
Average nucleotide identity and genome coverage (%) of
Lacto 166 and Lacto 167 against select genomes in Lactobacillus

| Average nucleotide | L. jensenii | L. jensenii | L. jensenii | L. jensenii | L. jensenii | L. jensenii |
|---|---|---|---|---|---|---|
| identity (%) | IM1 | IM11 | IM18 1 | IM18 3 | IM3 | IM59 |
| L. acidophilus NCFM | 74.1408959 | 74.0593925 | 73.5892704 | 74.0836248 | 74.0870474 | 73.6859301 |
| L. amylolyticus | 74.0081177 | 74.0432263 | 73.0645206 | 73.9394434 | 73.9987987 | 73.1130264 |
| L. crispatus ST1 | 73.8565051 | 73.8838341 | 73.2390336 | 73.8231114 | 73.8825615 | 73.2847504 |
| L. gallinarum | 74.7085599 | 73.972087 | 73.5692393 | 74.8247386 | 74.2972793 | 73.4496792 |
| L. gasseri ATCC 33323 JCM 1131 | 74.3842554 | 74.2654789 | 73.4072176 | 74.3497066 | 74.0868022 | 73.6415132 |
| L. helveticus | 74.4788701 | 74.0203429 | 73.592463 | 74.4899098 | 74.3146003 | 73.4185419 |
| L. iners DSM 13335 | 72.3618016 | 72.263058 | 72.0495816 | 72.3387515 | 72.3608942 | 72.1925445 |
| L. jensenii 1153 | 99.6485106 | 88.1105772 | 99.6971898 | 99.8593166 | 87.9609709 | 99.7949308 |
| L. jensenii 115 3 CHN | 87.8811122 | 99.9298862 | 87.9008174 | 88.0256115 | 99.8083585 | 87.9696683 |
| L. jensenii 269 3 | 99.6070573 | 88.2602873 | 99.6495874 | 99.8556375 | 88.0578252 | 99.7760395 |
| L. jensenii 27 2 CH N | 87.9058693 | 99.5118276 | 87.965406 | 88.0839246 | 99.8413385 | 88.0102098 |
| L. jensenii DSM 20557 | 88.0266484 | 99.7274617 | 87.9841693 | 88.4541853 | 99.7633492 | 88.1473979 |
| L. jensenii IM1 | 100 | 88.078229 | 99.6368884 | 99.7125155 | 87.7954263 | 99.5899784 |
| L. jensenii IM11 | 88.0265586 | 100 | 87.9635795 | 88.3491712 | 99.5806224 | 88.0495944 |
| L. jensenii IM18 1 | 99.6833764 | 88.0857808 | 100 | 99.7717449 | 87.9593873 | 99.6521888 |
| L. jensenii IM18 3 | 99.6557225 | 88.3478942 | 99.6956412 | 100 | 87.9166471 | 99.6439074 |
| L. jensenii IM3 | 87.8357819 | 99.5411303 | 87.8700099 | 88.0460982 | 100 | 87.9191382 |
| L. jensenii IM59 | 99.5171426 | 87.9511779 | 99.5561765 | 99.6767729 | 87.8510924 | 100 |
| L. jensenii JV V16 | 87.6999689 | 99.4642349 | 87.7858688 | 88.1819303 | 99.7849624 | 87.9407578 |
| L. jensenii MD IIE 702 | 88.0864154 | 99.8950534 | 88.0025109 | 88.4662583 | 99.554428 | 88.0700878 |
| L. jensenii SJ 7A US | 99.657614 | 88.1125396 | 99.7172192 | 99.8489617 | 87.9743625 | 99.7290878 |
| L. jensenii SNUV360 RefSeq | 99.5126251 | 88.4759123 | 99.5454709 | 99.8456926 | 88.1524385 | 99.7916025 |
| L. jensenii T L.2937 | 88.0445071 | 99.7470391 | 87.897357 | 88.5850637 | 99.8245877 | 88.0500794 |
| L. jensenii UMB0007 | 88.0468507 | 99.5398912 | 87.9709976 | 88.4907797 | 99.7403862 | 88.1919804 |
| L. jensenii UMB0077 | 88.2399018 | 99.9119048 | 88.1335618 | 88.3462902 | 99.8117249 | 88.2245235 |
| L. psittaci DSM 15354 | 80.4300689 | 83.0011477 | 80.105919 | 80.3020942 | 83.1053806 | 80.0843073 |
| Lacto166 | 88.1058755 | 99.7117554 | 87.9866538 | 88.1523357 | 99.7410384 | 88.1554834 |
| Lacto167 | 88.1365872 | 99.7153979 | 88.024672 | 88.1648867 | 99.7442346 | 88.1702695 |

TABLE 7D
Average nucleotide identity and genome coverage (%) of
Lacto166 and Lacto 167 against select genomes in Lactobacillus

| | | | | L. jensenii | | |
|---|---|---|---|---|---|---|
| Average nucleotide | L. jensenii | L. jensenii | L. jensenii | SNUV360 | L. jensenii | L. jensenii |
| identity (%) | JV V16 | MD IIE 702 | SJ 7A US | RefSeq | T L.2937 | UMB0007 |
| L. acidophilus NCFM | 74.0931433 | 74.0706941 | 74.0885407 | 74.0851895 | 74.1325821 | 74.109831 |
| L. amylolyticus | 74.0235246 | 74.1091342 | 73.9814169 | 73.9151216 | 74.1927188 | 74.1692967 |
| L. crispatus ST1 | 73.8978208 | 73.9096686 | 73.8264387 | 73.8403029 | 74.0145541 | 73.9741901 |
| L. gallinarum | 74.3286499 | 74.4896643 | 74.8782347 | 74.9017489 | 74.4359539 | 74.4157348 |
| L. gasseri ATCC 33323 JCM 1131 | 74.1280516 | 74.2486599 | 74.3973744 | 74.3550637 | 74.2965372 | 74.2657368 |
| L. helveticus | 74.3219023 | 74.3667544 | 74.6244618 | 74.5793524 | 74.4685099 | 74.4216643 |
| L. iners DSM 13335 | 72.376767 | 72.3013794 | 72.3974483 | 72.3318071 | 72.3379795 | 72.2900205 |
| L. jensenii 1153 | 87.9904843 | 88.1364855 | 99.9983357 | 99.9252168 | 88.1008088 | 88.221688 |
| L. jensenii 115 3 CHN | 99.8836093 | 99.9221907 | 88.0510188 | 87.9899737 | 99.9283656 | 99.9550479 |
| L. jensenii 269 3 | 88.0124044 | 88.3539531 | 99.9115214 | 99.8763194 | 88.0755725 | 88.3511316 |
| L. jensenii 27 2 CHN | 99.9369607 | 99.5012198 | 88.072184 | 87.9972568 | 99.7457484 | 99.6980623 |
| L. jensenii DSM 20557 | 99.5518878 | 99.7326637 | 88.1946427 | 88.1436216 | 99.753591 | 99.7934459 |
| L. jensenii IM1 | 87.784928 | 88.0844551 | 99.6963226 | 99.6144184 | 87.9696782 | 88.0309992 |
| L. jensenii IM11 | 99.4327664 | 99.9798721 | 88.0560912 | 88.425897 | 99.6707971 | 99.8024805 |
| L. jensenii IM18 1 | 87.9837757 | 88.0770283 | 99.7391392 | 99.7001928 | 88.0305149 | 88.0679274 |
| L. jensenii IM18 3 | 87.9423251 | 88.3743527 | 99.8128151 | 99.8194001 | 88.2414746 | 88.5109782 |
| L. jensenii IM3 | 99.6963016 | 99.5265323 | 88.0586563 | 88.173469 | 99.8217169 | 99.5981195 |
| L. jensenii IM59 | 87.8691379 | 87.956849 | 99.7387099 | 99.7343255 | 87.9173888 | 88.1351375 |
| L. jensenii JV V16 | 100 | 99.4502371 | 88.1320347 | 88.0752558 | 99.6681785 | 99.6652744 |
| L. jensenii MD IIE 702 | 99.4030131 | 100 | 88.1487849 | 88.5723836 | 99.6358014 | 99.721926 |
| L. jensenii SJ 7A US | 88.0225874 | 88.1285029 | 100 | 99.9223089 | 88.1199638 | 88.2174541 |
| L. jensenii SNUV360 RefSeq | 88.0253148 | 88.5766972 | 99.9133146 | 100 | 88.1216415 | 88.4087415 |
| L. jensenii T L.2937 | 99.6791902 | 99.7001868 | 88.410272 | 88.3291719 | 100 | 99.7976149 |
| L. jensenii UMB0007 | 99.4694563 | 99.5470106 | 88.1899799 | 88.2521899 | 99.5151417 | 100 |
| L. jensenii UMB0077 | 99.833941 | 99.9278207 | 88.3296474 | 88.2801872 | 99.8951316 | 99.9503338 |
| L. psittaci DSM 15354 | 83.0812189 | 83.0203253 | 80.3029236 | 80.1901413 | 83.0745874 | 83.0404885 |
| Lacto166 | 99.7449299 | 99.7105436 | 88.1533437 | 88.2120316 | 99.8465397 | 99.7773159 |
| Lacto167 | 99.7472413 | 99.7115416 | 88.1640631 | 88.2161547 | 99.8501201 | 99.7918705 |

TABLE 7E
Average nucleotide identity and genome coverage (%) of
Lacto166 and Lacto167 against select genomes in Lactobacillus

| Average nucleotide | L. jensenii | L. psittaei | | |
|---|---|---|---|---|
| identity (%) | UMB0077 | DSM 15354 | Lacto166 | Lacto167 |
| L. acidophilus NCFM | 74.0788153 | 74.1072284 | 74.0729014 | 74.0443441 |
| L. amylolyticus | 74.1736592 | 74.1130974 | 74.1228949 | 74.0918444 |
| L. crispatus ST1 | 73.986327 | 73.9840257 | 73.9195612 | 73.8907188 |
| L. gallinarum | 74.6398143 | 74.0440547 | 74.6436874 | 74.6116939 |
| L. gasseri ATCC 33323 JCM 1131 | 74.2633173 | 74.1636286 | 74.2100952 | 74.1544297 |
| L. helveticus | 74.5546265 | 74.1671492 | 74.395695 | 74.385131 |
| L. iners DSM 13335 | 72.3144447 | 72.1234863 | 72.2748735 | 72.2168586 |
| L. jensenii 1153 | 88.2080916 | 80.1501245 | 88.087158 | 88.1049531 |
| L. jensenii 115 3 CH N | 99.9176234 | 83.0258683 | 99.9203217 | 99.8730438 |
| L. jensenii 269 3 | 88.2290024 | 80.3042135 | 88.3213116 | 88.326569 |
| L. jensenii 27 2 CH N | 99.8801675 | 83.0671929 | 99.7923414 | 99.7695918 |
| L. jensenii DSM 20557 | 99.9299887 | 83.0735979 | 99.7878799 | 99.7548861 |
| L. jensenii IM1 | 88.1286779 | 80.4953181 | 88.0563619 | 88.0476705 |
| L. jensenii IM11 | 99.8654245 | 83.1766516 | 99.6804496 | 99.650817 |
| L. jensenii IM18 1 | 88.1528096 | 80.1263616 | 88.0733331 | 88.0742858 |
| L. jensenii IM18 3 | 88.2202233 | 80.1915823 | 88.0745174 | 88.0934577 |
| L. jensenii I M3 | 99.797482 | 83.048246 | 99.6793416 | 99.6583646 |
| L. jensenii IM59 | 88.0805719 | 80.2496188 | 88.0648286 | 88.0648715 |
| L. jensenii JV V16 | 99.8331097 | 83.2423571 | 99.7269769 | 99.6931014 |
| L. jensenii MD IIE 702 | 99.830664 | 83.0830294 | 99.6404091 | 99.5869019 |
| L. jensenii Si 7A US | 88.1990911 | 80.3247792 | 88.0999622 | 88.0996405 |
| L. jensenii SNUV360 RefSeq | 88.3006618 | 80.3006393 | 88.255679 | 88.2646732 |
| L. jensenii T L.2937 | 99.8914966 | 83.1371239 | 99.8606899 | 99.8272422 |
| L. jensenii UMB0007 | 99.9240041 | 83.0855194 | 99.8064471 | 99.7762743 |
| L. jensenii UMB0077 | 100 | 83.2098252 | 99.8931557 | 99.8753058 |
| L. psittaci DSM 15354 | 83.0494622 | 100 | 83.0482228 | 83.0062044 |
| Lacto166 | 99.844139 | 83.0790113 | 100 | 99.9600327 |
| Lacto167 | 99.8863209 | 83.0338526 | 99.9983867 | 100 |

TABLE 7F
Average nucleotide identity and genome coverage (%) of
Lacto166 and Lacto167 against select genomes in Lactobacillus

| | | | | | L. gasseri | |
|---|---|---|---|---|---|---|
| | L. acidophilus | | L. crispatus | | ATCC 33323 | |
| Coverage (%) | NCFM | L. amylolyticus | ST1 | L. gallinarum | JCM 1131 | L. helveticus |
| L. acidophilus NCFM | 100 | 48.4738358 | 64.7643412 | 57.4599209 | 39.6140573 | 62.3458035 |
| L. amylolyticus | 58.5749589 | 100 | 57.8905612 | 54.3279889 | 41.7525398 | 56.7395481 |
| L. crispatus ST1 | 62.337525 | 45.3145885 | 100 | 54.9409469 | 36.8862268 | 59.5977507 |
| L. gallinarum | 48.3681449 | 41.9182898 | 52.691447 | 100 | 31.1552808 | 69.5987253 |
| L. gasseri ATCC 33323 JCM 1131 | 41.8752507 | 35.7051458 | 40.5756562 | 37.4949323 | 100 | 39.5561034 |
| L. helveticus | 57.3163355 | 44.7214942 | 58.3995153 | 71.1312178 | 34.6105195 | 100 |
| L. iners DSM 13335 | 36.2278685 | 32.1806693 | 34.1825494 | 33.9654318 | 41.172419 | 35.2213323 |
| L. jensenii 1153 | 38.4226584 | 33.6014703 | 36.7046571 | 34.9863526 | 34.3746436 | 36.4745915 |
| L. jensenii 115 3 CHN | 40.1372881 | 34.6290606 | 39.4183756 | 36.4216544 | 36.0172157 | 38.0079932 |
| L. jensenii 269 3 | 39.9276207 | 35.8001198 | 39.0851775 | 37.1014356 | 35.7112558 | 38.108912 |
| L. jensenii 27 2 CHN | 38.8567621 | 32.8555478 | 37.5088182 | 35.376168 | 34.6631325 | 36.867335 |
| L. jensenii DSM 20557 | 36.4564583 | 31.7712775 | 36.006074 | 33.0690274 | 32.7532534 | 34.5323845 |
| L. jensenii IM1 | 40.2382602 | 36.3679647 | 39.1116468 | 36.9557631 | 36.2246297 | 38.1792835 |
| L. jensenii IM11 | 38.3969717 | 33.0494165 | 37.4050994 | 34.3140078 | 34.4865382 | 36.3039143 |
| L. jensenii IM18 1 | 40.7573777 | 36.3948016 | 39.9385068 | 37.0909457 | 37.3763569 | 38.6474749 |
| L. jensenii IM18 3 | 37.8128405 | 34.4938639 | 36.9646882 | 34.4939838 | 34.5746317 | 36.4473423 |
| L. jensenii IM3 | 36.5426052 | 31.9500193 | 35.3227493 | 32.6767734 | 32.2434484 | 34.4732595 |
| L. jensenii IM59 | 39.0247385 | 34.9616619 | 37.7046618 | 35.3248553 | 35.1908043 | 36.9801213 |
| L. jensenii JV V16 | 37.9266862 | 34.4268388 | 37.8185669 | 35.1763439 | 33.4508444 | 36.6777592 |
| L. jensenii MD IIE 702 | 37.772363 | 32.8727014 | 37.1015251 | 34.4069164 | 34.2576459 | 36.073458 |
| L. jensenii SJ 7A US | 39.2826885 | 34.670668 | 37.438417 | 34.7968075 | 34.9297785 | 36.3806875 |
| L. jensenii SNUV360 RefSeq | 37.9579808 | 34.8491017 | 37.128425 | 35.4403027 | 34.5117757 | 36.2538607 |
| L. jensenii T L.2937 | 39.9642189 | 36.4024247 | 40.3410983 | 37.599891 | 36.2994927 | 38.8114043 |
| L. jensenii UMB0007 | 36.1346198 | 31.2318609 | 35.4725879 | 33.2122635 | 33.2435177 | 34.4782592 |
| L. jensenii UMB0077 | 43.2949857 | 37.3182073 | 42.3532246 | 39.0853822 | 38.4827456 | 41.3138862 |
| L. psittaci DSM 15354 | 39.7782159 | 35.8554806 | 39.4702149 | 36.6993075 | 36.6131683 | 37.9317653 |
| Lacto166 | 38.7825109 | 34.0426997 | 37.1730643 | 34.8213077 | 34.0035867 | 36.0278835 |
| Lacto167 | 39.1752457 | 34.731484 | 37.9821811 | 35.8819566 | 34.3723896 | 36.6793321 |

TABLE 7G
Average nucleotide identity and genome coverage (%) of
Lacto166 and Lacto167 against select genomes in Lactobacillus

| | L. iners | L. jensenii | L. jensenii | L. jensenii | L. jensenii | L. jensenii |
|---|---|---|---|---|---|---|
| Coverage (%) | DSM 13335 | 1153 | 115 3 CHN | 269 3 | 27 2 CHN | DSM 20557 |
| L. acidophilus NCFM | 24.838831 | 32.6974859 | 33.6463412 | 32.6043861 | 33.4896366 | 33.5268063 |
| L. amylolyticus | 27.0526889 | 36.8326022 | 35.7207692 | 36.622827 | 35.6132532 | 35.7189966 |
| L. crispatus ST1 | 22.4356279 | 30.963786 | 31.835034 | 30.6554892 | 31.4413793 | 31.4706477 |
| L. gallinarum | 19.2852228 | 26.7631724 | 28.3096722 | 25.8628792 | 27.2976214 | 29.4359651 |
| L. gasseri ATCC 33323 JCM 1131 | 29.8294411 | 31.3144281 | 31.4115585 | 31.3964611 | 31.4155704 | 31.3259887 |
| L. helveticus | 21.6078716 | 29.1979526 | 30.1038192 | 28.0855676 | 29.625291 | 30.317095 |
| L. iners DSM 13335 | 100 | 35.7935552 | 35.1550387 | 35.7205304 | 35.2467697 | 35.4023679 |
| L. jensenii 1153 | 27.5173777 | 100 | 74.3685175 | 92.7918431 | 74.6158699 | 75.2109592 |
| L. jensenii 115 3 CHN | 28.3879508 | 77.4223951 | 100 | 77.3864464 | 97.3217263 | 90.4749517 |
| L. jensenii 269 3 | 29.0999593 | 96.4876733 | 77.3524347 | 100 | 77.3449416 | 77.5589583 |
| L. jensenii 27 2 CHN | 27.4357554 | 74.4241311 | 92.2578964 | 71.1667679 | 100 | 88.6373962 |
| L. jensenii DSM 20557 | 26.094601 | 72.1605734 | 83.2659508 | 71.5777266 | 85.7004872 | 100 |
| L. jensenii IM1 | 28.2638636 | 93.1221613 | 78.8873711 | 92.5684518 | 78.9992347 | 76.0678461 |
| L. jensenii IM11 | 26.8937765 | 73.4605864 | 87.3876212 | 74.678431 | 88.5264288 | 94.2064251 |
| L. jensenii IM18 1 | 29.1662423 | 97.1486766 | 79.2298436 | 97.0913348 | 79.3150778 | 78.4991259 |
| L. jensenii IM18 3 | 28.0725507 | 93.8964217 | 73.7897266 | 91.6415179 | 76.1418876 | 78.0945865 |
| L. jensenii IM3 | 25.123247 | 70.5951542 | 88.0645271 | 70.9105166 | 91.5801646 | 86.414301 |
| L. jensenii IM59 | 28.422395 | 96.9386135 | 75.8429892 | 95.4722992 | 75.8569605 | 76.8916402 |
| L. jensenii JV V16 | 26.1295405 | 72.0526463 | 89.6891512 | 70.9409874 | 95.9822336 | 86.1617586 |
| L. jensenii MD IIE 702 | 26.2505071 | 72.6890037 | 86.7795909 | 73.7488479 | 88.0304498 | 93.9531363 |
| L. jensenii SJ 7A US | 27.5678198 | 97.0815301 | 75.5311369 | 93.5827455 | 75.7279706 | 76.2877908 |
| L. jensenii SNUV360 RefSeq | 27.1944099 | 91.1342535 | 73.6387872 | 91.6059488 | 73.9240608 | 74.4610604 |
| L. jensenii T L.2937 | 28.0819678 | 76.1332234 | 92.3229578 | 74.7493199 | 94.1976115 | 90.9484987 |
| L. jensenii UMB0007 | 25.7944211 | 72.5374908 | 82.7317795 | 72.262465 | 86.9013448 | 92.7230579 |
| L. jensenii UMB0077 | 30.6607836 | 81.4666952 | 96.1536158 | 81.2974325 | 95.6239292 | 96.0695501 |
| L. psittaci DSM 15354 | 28.6821419 | 66.6797593 | 67.0932144 | 66.7794448 | 67.4686879 | 65.2029488 |
| Lacto166 | 27.8543867 | 74.661549 | 90.8208836 | 76.0652476 | 90.6953384 | 92.9008852 |
| Lacto167 | 28.4503317 | 74.8752686 | 90.686312 | 76.4807899 | 90.6517727 | 93.0002919 |

TABLE 7H
Average nucleotide identity and genome coverage (%) of
Lacto166 and Lacto167 against select genomes in Lactobacillus

| | L. jensenii | L. jensenii | L. jensenii | L. jensenii | L. jensenii | L. jensenii |
|---|---|---|---|---|---|---|
| Coverage (%) | IM1 | IM11 | IM18 1 | IM18 3 | IM3 | IM59 |
| L. acidophilus NCFM | 32.7672606 | 33.4784506 | 31.3960453 | 32.3865848 | 33.4846205 | 31.3795923 |
| L. amylolyticus | 37.3369918 | 35.5610539 | 34.507777 | 36.8189106 | 35.71569 | 34.5113222 |
| L. crispatus ST1 | 31.3704108 | 32.2348856 | 29.9000911 | 30.9121503 | 31.6650034 | 29.4038992 |
| L. gallinarum | 27.2781249 | 27.0591574 | 25.5559458 | 26.6661108 | 29.5190464 | 24.2499312 |
| L. gasseri ATCC 33323 JCM 1131 | 31.8167613 | 31.4531029 | 29.5834477 | 31.2590532 | 31.5965814 | 29.7093477 |
| L. helveticus | 29.658569 | 28.8151855 | 27.9183905 | 29.2032289 | 30.2475767 | 26.7575927 |
| L. iners DSM 13335 | 35.7705442 | 35.075048 | 34.5669272 | 35.8788681 | 34.9524009 | 34.9604625 |
| L. jensenii 1153 | 90.0576226 | 73.0423495 | 88.6185144 | 94.1462764 | 75.0190458 | 93.475695 |
| L. jensenii 115 3 CHN | 79.1290597 | 90.5331899 | 75.6327815 | 77.0609831 | 97.5317066 | 76.5749612 |
| L. jensenii 269 3 | 93.0077959 | 77.3520012 | 91.82383 | 95.6034919 | 78.4035689 | 96.2478334 |
| L. jensenii 27 2 CHN | 75.8212969 | 87.5570627 | 72.3068221 | 75.3427611 | 95.8142483 | 73.1756379 |
| L. jensenii DSM 20557 | 70.6014392 | 90.2035756 | 69.1650738 | 73.9154411 | 87.1455016 | 70.9655661 |
| L. jensenii IM1 | 100 | 75.8548377 | 90.4368728 | 93.6144237 | 79.6530918 | 90.7570708 |
| L. jensenii IM11 | 73.2177285 | 100 | 71.7151375 | 75.1748115 | 92.1734452 | 72.4393862 |
| L. jensenii IM18 1 | 96.2721396 | 78.5130073 | 100 | 96.6909807 | 79.613138 | 96.290885 |
| L. jensenii IM18 3 | 90.6400447 | 75.1487831 | 87.8248027 | 109 | 74.6942425 | 92.6616991 |
| L. jensenii IM3 | 72.4259609 | 86.7928495 | 68.6938882 | 70.2673478 | 100 | 69.3148396 |
| L. jensenii IM59 | 91.1153319 | 75.2139709 | 90.5815896 | 95.6091815 | 76.181525 | 100 |
| L. jensenii JV V16 | 73.840051 | 84.5754169 | 70.3469038 | 73.3046084 | 94.4785276 | 69.9424872 |
| L. jensenii MD IIE 702 | 72.8176953 | 98.6518162 | 70.8244119 | 74.4298874 | 91.7249834 | 71.4438318 |
| L. jensenii SJ 7A US | 91.2400767 | 74.6018646 | 89.3762866 | 95.3050861 | 76.01825 | 94.95253 |
| L. jensenii SNUV360 RefSeq | 88.971869 | 75.8340029 | 86.4535939 | 90.8182379 | 77.3745638 | 89.4444597 |
| L. jensenii T L.2937 | 75.8730242 | 89.6724885 | 73.1091623 | 77.7459444 | 93.9334074 | 72.3373516 |
| L. jensenii UMB0007 | 70.8543166 | 89.7796377 | 69.110057 | 75.6892156 | 86.4984555 | 71.8405613 |
| L. jensenii UMB0077 | 81.5926938 | 95.906411 | 79.7795924 | 81.0926266 | 96.9494102 | 79.8345046 |
| L. psittaci DSM 15354 | 68.198181 | 65.2651713 | 65.0748095 | 67.125946 | 67.5967624 | 65.8345065 |
| Lacto166 | 74.2601158 | 92.2293141 | 72.3928703 | 74.3443317 | 94.0140807 | 73.8719199 |
| Lacto167 | 75.0662593 | 92.7059423 | 72.6825167 | 75.1260471 | 93.901883 | 74.2203559 |

TABLE 7I
Average nucleotide identity and genome coverage (%) of
Lacto166 and Lacto167 against select genomes in Lactobacillus

| | | | | L. jensenii | | |
|---|---|---|---|---|---|---|
| | L. jensenii | L. jensenii | L. jensenii | SNUV360 | L. jensenii | L. jensenii |
| Coverage (%) | JV V16 | MD IIE 702 | SJ 7A US | RefSeq | T L.2937 | UMB0007 |
| L. acidophilus NCFM | 33.620508 | 33.484169 | 32.7622444 | 32.035404 | 33.9419932 | 33.7626658 |
| L. amylolyticus | 35.8665356 | 35.7075054 | 37.1264831 | 36.0993669 | 36.2044379 | 35.8238842 |
| L. crispatus ST1 | 31.7217292 | 31.5261499 | 31.1163437 | 30.178728 | 31.8134498 | 31.5996145 |
| L. gallinarum | 29.7363295 | 28.0675868 | 26.991867 | 26.5914935 | 29.8800479 | 29.7526257 |
| L. gasseri ATCC 33323 JCM 1131 | 31.4622353 | 31.4098693 | 31.6180663 | 30.8922802 | 31.864218 | 31.6296797 |
| L. helveticus | 30.5779042 | 29.7567832 | 29.3761452 | 28.6466662 | 30.6869951 | 30.4540025 |
| L. iners DSM 13335 | 35.126314 | 35.2009824 | 35.6693427 | 34.8459553 | 35.3446056 | 35.3804527 |
| L. jensenii 1153 | 75.0608373 | 73.298315 | 96.4155132 | 91.156333 | 74.5154246 | 75.742967 |
| L. jensenii 115 3 CHN | 97.8638133 | 91.034237 | 77.7161317 | 76.1037782 | 95.1235761 | 91.1312178 |
| L. jensenii 269 3 | 77.9920078 | 77.6270152 | 97.1378358 | 96.0874447 | 77.6725309 | 78.759025 |
| L. jensenii 27 2 CHN | 99.659238 | 88.2162562 | 74.5120315 | 73.2075639 | 92.804549 | 89.3616265 |
| L. jensenii DSM 20557 | 86.1628291 | 90.8213665 | 72.3872457 | 71.124556 | 86.3198454 | 92.5814584 |
| L. jensenii IM1 | 79.7948689 | 76.4359056 | 94.0391317 | 91.8219632 | 77.5119778 | 76.737657 |
| L. jensenii IM11 | 89.451647 | 99.9239847 | 73.6702654 | 75.7137322 | 90.1151072 | 93.1123498 |
| L. jensenii IM18 1 | 79.9205389 | 78.6761458 | 97.5990426 | 95.3280121 | 79.4136096 | 79.1669234 |
| L. jensenii IM18 3 | 76.8421753 | 76.5105895 | 95.0594906 | 90.8322206 | 76.977328 | 78.5732574 |
| L. jensenii IM3 | 93.7163898 | 87.6531354 | 71.0401277 | 72.6455781 | 88.2511308 | 86.8862082 |
| L. jensenii IM59 | 76.4816089 | 75.3357124 | 97.3104852 | 93.0966165 | 76.0556983 | 77.8590482 |
| L. jensenii JV V16 | 100 | 86.1460676 | 72.715278 | 71.4194497 | 91.9736871 | 87.3317722 |
| L. jensenii MD IIE 702 | 88.9657065 | 100 | 72.9693696 | 75.0514766 | 89.7026198 | 92.9008553 |
| L. jensenii SJ 7A US | 76.3598269 | 74.7278211 | 100 | 92.8628721 | 75.8031175 | 76.9847907 |
| L. jensenii SNUV360 RefSeq | 74.790865 | 76.6381294 | 93.0526124 | 100 | 74.4291245 | 75.2449509 |
| L. jensenii T L.2937 | 96.8208834 | 91.3527549 | 76.6491387 | 75.1670852 | 100 | 91.6493569 |
| L. jensenii UMB0007 | 87.9084826 | 90.5379372 | 72.8728619 | 71.2011304 | 88.0131188 | 100 |
| L. jensenii UMB0077 | 97.1823711 | 95.6858316 | 81.8351728 | 80.0721913 | 97.8182878 | 97.1510878 |
| L. psittaci DSM 15354 | 67.9308838 | 65.4237736 | 67.2196685 | 65.0611335 | 65.9877292 | 65.76496 |
| Lacto166 | 91.9397095 | 93.0264304 | 75.0084755 | 75.139112 | 92.5155045 | 93.1097478 |
| Lacto167 | 91.7733251 | 93.1039568 | 75.2083549 | 75.223103 | 92.2676611 | 93.0376823 |

TABLE 7.1
Average nucleotide identity and genome coverage (%) of
Lacto166 and Lacto167 against select genomes in Lactobacillus

| | L. jensenii | L. psittaei | | |
|---|---|---|---|---|
| Coverage (%) | UMB0077 | DSM 15354 | Lacto166 | Lacto167 |
| L. acidophilus NCFM | 33.6662553 | 31.4073316 | 33.6812536 | 33.4298943 |
| L. amylolyticus | 35.8335417 | 35.2118565 | 35.8301188 | 35.9252878 |
| L. crispatus ST1 | 31.5569845 | 30.3364248 | 31.4637466 | 31.6176748 |
| L. gallinarium | 27.8030994 | 24.4979539 | 27.1872955 | 28.2776272 |
| L. gassen ATCC 33323 JCM 1131 | 31.6940814 | 30.9316075 | 31.3920944 | 31.3348719 |
| L. helveticus | 29.7710387 | 27.250469 | 29.5291132 | 29.7774721 |
| L. iners DSM 13335 | 35.3661295 | 34.3893354 | 35.5515482 | 35.4515207 |
| L. jensenii 1153 | 72.8396395 | 62.4845026 | 74.8976138 | 73.7175039 |
| L. jensenii 115 3 CHN | 90.0037066 | 65.9252577 | 94.7765684 | 92.813482 |
| L. jensenii 269 3 | 76.1232008 | 65.0673416 | 79.3799583 | 78.1987792 |
| L. jensenii 27 2 CHN | 85.3052968 | 62.6570908 | 90.1513675 | 88.2430884 |
| L. jensenii DSM 20557 | 82.9927831 | 58.5840322 | 89.8008476 | 87.9670945 |
| L. jensenii IM1 | 76.0069599 | 65.2324022 | 77.1725854 | 76.742331 |
| L jensenii IM11 | 86.4389407 | 60.5285641 | 92.8323433 | 91.27951 |
| L. jensenii IM1.8 1 | 78.1075936 | 67.1.825043 | 79.5533962 | 78.3678367 |
| L. jensenii IM18 3 | 73.2230925 | 63.2605264 | 74.7959967 | 74.1789352 |
| L. jensenii IM3 | 82.324704 | 59.442462 | 89.6109962 | 87.6548969 |
| L. jensenii IM59 | 74.5073408 | 63.4454816 | 76.974.8528 | 75.8077838 |
| L. jensenii JV V16 | 83.5594504 | 60.4601141 | 87.930551.7 | 86.445171 |
| L. jensenii MD IIE 702 | 86.2809475 | 59.7300818 | 92.4618967 | 90.6734059 |
| L. jensenii SJ 7A US | 74.2624226 | 63.6296818 | 75.8976001 | 74.6985431 |
| L. jensenii SNUV360 RefSeg | 72.4487445 | 60.7433947 | 75.7224961 | 74.7383351 |
| L jensenli T L.2937 | 88.7929638 | 61.8755862 | 93.0553771 | 91.402607 |
| L. fensenii UMB0007 | 82.8468907 | 58.5330891 | 88.4548909 | 86.4605405 |
| L, ienseriii UMB0077 | 100 | 67.61171.14 | 97.3960295 | 95.6679921 |
| L. psittaci DSM 15354 | 64.9540591 | 100 | 65.9251179 | 65.7120061 |
| Lacto166 | 87.5156782 | 61.8963677 | 100 | 97.2276964 |
| Lacto167 | 87.5328543 | 63.1548897 | 99.2580268 | 100 |

TABLE 8A
Lacto166 and Micro36 sequences in post-natal infant cohorts

| | | | | | | | | Age of | | |
|---|---|---|---|---|---|---|---|---|---|---|
| | Query | | Similiarity | Length | | | | Stool | | |
| SampleID | Sequence | Count | (%) | (bp) | Mismached | Gaps | Participant | (months) | Sex | Cohort |
| A87 | Lacto166 | 1 | 98.02 | 253 | 5 | 0 | A87 | 1 | Male | WHEALS |
| A219 | Micro36 | 1 | 99.21 | 253 | 2 | 0 | A219 | 1 | Male | WHEALS |
| A233 | Micro37 | 86 | 98.23488 | 253 | 4.441860465 | 0.023256 | A233 | 1 | Male | WHEALS |
| A146 | Lacto166 | 4 | 99.505 | 253 | 1.25 | 0 | A146 | 2 | Male | WHEALS |
| A178 | Lacto166 | 1 | 97.23 | 253 | 7 | 0 | A178 | 2 | Male | WHEALS |
| A308 | Lacto166 | 1 | 97.23 | 253 | 7 | 0 | A308 | 2 | Male | WHEALS |
| A36 | Lacto166 | 1 | 97.23 | 253 | 7 | 0 | A36 | 2 | Male | WHEALS |
| A295 | Micro38 | 4 | 97.6275 | 253 | 6 | 0 | A295 | 6 | Male | WHEALS |
| A125 | Lacto166 | 1 | 97.23 | 253 | 7 | 0 | A125 | 8 | Male | WHEALS |
| B02M00 | Lacto166 | 5 | 100 | 253 | 0 | 0 | PB02 | 0 | Female | DIMES |
| B07M00 | Lacto166 | 299 | 99.62334 | 252.99 | 0.949832776 | 0 | PB07 | 0 | Female | DIMES |
| B12M00 | Lacto166 | 1 | 100 | 253 | 0 | 0 | PB12 | 0 | Female | DIMES |
| DB23M00 | Micro36 | 37 | 99.09865 | 253 | 2.27027027 | 0 | DB23 | 0 | Male | DIMES |
| DB29M00 | Micro36 | 1 | 100 | 253 | 0 | 0 | DB29 | 0 | Male | DIMES |
| B02M01 | Lacto166 | 1 | 100 | 253 | 0 | 0 | PB02 | 1 | Female | DIMES |
| DB10M01 | Lacto166 | 1 | 97.23 | 253 | 7 | 0 | DB10 | 1 | Female | DIMES |
| B02M03 | Lacto166 | 1 | 97.23 | 253 | 7 | 0 | PB02 | 3 | Female | DIMES |
| B13M03 | Lacto166 | 7 | 97.28714 | 253 | 6.857142857 | 0 | PB13 | 3 | Female | DIMES |
| B11M03 | Lacto166 | 1 | 100 | 253 | 0 | 0 | PB11 | 3 | Female | DIMES |
| DB10M06 | Lacto166 | 1 | 97.23 | 253 | 7 | 0 | DB10 | 6 | Female | DIMES |
| DB16M06 | Lacto166 | 1 | 97.23 | 253 | 7 | 0 | DB16 | 6 | Male | DIMES |
| B10M06 | Micro36 | 3 | 99.34 | 253 | 1.666666667 | 0 | PB10 | 6 | Female | DIMES |
| DB27M06 | Lacto166 | 1 | 97.23 | 253 | 7 | 0 | DB27 | 6 | Male | DIMES |
| DB11M06 | Micro36 | 23 | 99.67261 | 253 | 0.826086957 | 0 | DB11 | 6 | Female | DIMES |
| DB10M12 | Lacto166 | 4 | 97.23 | 253 | 7 | 0 | DB10 | 12 | Female | DIMES |
| ERR756407 | Micro39 | 3 | 97.81 | 137 | 3 | 0 | 7-048261-1-4_2 | 3 | Male | CHILD |
| ERR756501 | Micro40 | 1 | 97.76 | 134 | 3 | 0 | 7-096066-1-4_2 | 3 | Female | CHILD |
| ERR756508 | Micro41 | 1 | 97.79 | 136 | 3 | 0 | 7-108700-1-4_2 | 3 | Female | CHILD |
| ERR756922 | Micro43 | 1 | 97.12 | 139 | 2 | 1 | 7-208786-1-4_2 | 3 | Female | CHILD |
| ERR756996 | Micro44 | 1 | 97.81 | 137 | 3 | 0 | 7-236436-1-3_2 | 3 | Male | CHILD |
| ERR756880 | Micro42 | 5 | 97.644 | 135.8 | 3.2 | 0 | 7-193789_2 | 12 | Male | CHILD |

TABLE 8B
Lacto166 and Micro36 sequences in post-natal infant cohorts

| | | | | Fisher's exact p-value of odds |
|---|---|---|---|---|
| | | Number of | Percent of | ratio that younger samples (< 6 |
| | Number of | participants with | participants with | months) are enriched for |
| | participants in | >97% similarity to | >97% similarity to | samples with highest identity to |
| Cohort | cohort | OTU10 or OTU12 | OTU10 or OTU12 | OTU10 or OTU12 (>99% identity) |
| DIMES | 54 | 12 | 22.2 | 0.3 |
| WHEALS | 298 | 9 | 3.0 | 1 |
| CHILD | 319 | 6 | 1.9 | 1 |

### Example 2: Materials and Methods

### Human Samples and Consent

Human fetal tissue (small intestine, mesenteric lymph node, spleen) was obtained under the auspices of UCSF Committee on Human Research (CHR) approved protocols at 18-23 gestational weeks from the Department of Obstetrics, Gynecology and Reproductive Science at San Francisco General Hospital from terminated pregnancies. Samples were excluded in the case of: (1) known maternal or intrauterine infection, (2) intrauterine fetal demise, and/or (3) known or suspected chromosomal abnormality. No Human Patient Information (HPI) is associated with the data presented. Samples were transported in media on ice and processed within 2 hours after collection. All sample collection methods comply with the Helsinki Declaration.

### Sample Collection for Fetal Meconium Cohort

Uninterrupted stomach to caecum sections (fetal intestine), kidneys, spleens, and mesenteric lymph nodes were collected by a single operator using sterile tools within 10 minutes of termination procedure and placed into sterile containers with pre-aliquoted complete RPMI (cRPMI) media composed of: RPMI media (GIBCO) without antibiotics, 10% fetal bovine serum (GIBCO), 1 mM sodium pyruvate (Life Technologies), 2 mM L-glutamine (Life Technologies), 1× non-essential amino acids (Life Technologies), and 10 mM HEPES (Life Technologies). Sterile cotton swabs were pre-moistened with sterile 1× phosphate-buffered saline (PBS) and stored in containers until used to vigorously sample the surgical tray for 30 seconds, thus sampling both the hospital environment and any contaminants arising from the procedure; swabs were immediately snapped off into sterile tubes containing 500 μL of pre-aliquoted, sterile RNAlater. Blank swabs were prepared as described above, but immediately snapped off into RNAlater, without sampling the surgical tray. Air swabs were prepared as described above, but held in surgical room air for 30 seconds, before immediately being snapped off into RNAlater. All specimens were immediately placed on ice and transported to the laboratory. Intestinal sections were dissected to remove the mesentery and the muscularis in a sterile petri dish in a biosafety laminar flow cabinet. Separate sterile tools were used to divide the small intestine into three equal sections and new sterile tools were used to scrape internal contents, termed fetal meconium, of each section into sterile 1×PBS (FIG. 20). Fetal meconium was homogenized by vigorous pipetting in sterile 1×PBS, pelleted by centrifugation at 3000×g for 10 minutes, and re-suspended in 1 mL of sterile 1×PBS. Half of fetal meconium suspension (by volume) was added to RNAlater (Ambion), while the remainder was re-suspended in sterile 50% (v/v) glycerol. Sterile tools were used to remove kidney capsule of the fetal kidney in a sterile petri and separate sterile tools were used to biopsy the internal kidney tissue, which was immediately placed in RNAlater. Fetal meconium samples, kidney specimens, procedural swabs, and blank swabs were cryopreserved at −80° C., within 2 hours of the termination procedure. Additional splenic and intestinal samples were collected in the manner described above for ex vitro APC and T cell experiments. In total 77 fetal specimens were used in this study.

16S rRNA Gene Burden and Sequencing

### DNA Extraction.

Genomic DNA (gDNA) from fetal meconium samples, kidney specimens, procedural swabs, and blank swabs was extracted using a modified cetyltrimethylammonium bromide (CTAB)-buffer-based protocol exactly as previously described [16] along with buffer controls. Buffers were prepared using HPLC-grade chemicals in a BSL2 biosafety cabinet and autoclaved before use.

16S rRNA Gene Burden qPCR Analysis.

16S rRNA gene copy number was assessed by quantitative PCR (Q-PCR) using the 16S rRNA universal primers and TaqMan probes, as previously described [49]. Briefly, total 16S rRNA gene copy number was calculated against a standard curve of known 16S rRNA copy numbers (1×10²-1×10⁹). Q-PCR was performed in triplicate 20 μl reactions containing final concentrations of 1×TaqMan Universal Master Mix (Life Technologies), 100 ng of extracted genomic DNA, 900 nM of each primer, P891F (5′-seq-3′F) and P1033R (5′-seq-3′R) and 125 nM of UniProbe under the following conditions: 50° C. for 2 min, 95° C. for 10 min, followed by 40 cycles of denaturation at 95° C. for 15 s, and annealing and extension at 60° C. for 1 min, along with no-template control and 8 standards. Copy number was normalized either by 100ng of input DNA, when possible. When too little DNA was obtained, such as in the case of the buffers, 104 of DNA extract was added to the PCR reaction and copy number was normalized by weight of frozen sample.

### Depletion of Abundant Sequences by Hybridization (DASH).

Depletion of human 16S mitochondrial DNA (mtDNA) using single guide RNA (sgRNA) targeting of Cas9 was performed as previously described [17]. Briefly, 54 sgRNAs targeting the human mtDNA were transcribed from pooled sgRNA templates using custom T7 RNA polymerase generously provided by the DeRisi laboratory at UCSF. sgRNAs were purified and concentrated using a column-based RNA purification kit with DNAse treatment (Zymo) and incubated with purified Cas9 (Berkeley Macrolab) for 10 minutes at 37° C. sgRNA-loaded Cas9 was incubated with either meconium genomic DNA (gDNA) or pooled library of 16S rRNA V4 amplicon (see below) for 2 hours at 37° C. Cas9 was deactivated by boiling the in vitro reaction at 98° C. for 10 minutes and Ampure XP beads (Agencourt) were used to purify the amplicon DNA. To test the effects of DASH on bacterial community composition, a subset of meconium samples from our bank (n=10) was depleted of mtDNA either from individual meconium gDNA (individual DASH) prior to 30-cycle amplification or from the pooled library of 30-cycle amplicons (pooled DASH). DASH bacterial profiles were compared to 30-cycle or 35-cycle amplicons that were depleted of mtDNA by gel extraction, using a gel extraction kit (Quiagen). For sequencing of the entire bank of fetal meconium gDNA, individual DASH was implemented on all samples including buffer blanks and contamination swabs.

### Sequencing Preparation.

The V4 region of the depleted genomic DNA was amplified using primers designed by Caporaso et al [50] using PCR conditions and protocol as described in Fujimura et al [16]. Briefly, samples were amplified in heptuplicate from a single mastermix per template, aliquoted into 384-well plates, and included a negative control reaction for each template mastermix and each reverse barcoded primer. PCR reactions were performed in 254 volumes using 0.025 U Takara Hot Start ExTaq (Takara Mirus Bio Inc.), 1× Takara buffer with MgCl₂, 0.4 pmol μl−¹ of F515 and barcoded R806 primers, 0.56 mg/ml of bovine serum albumin (BSA; Roche Applied Science), 200 μM of dNTPs and 10 ng of DASH gDNA. PCR conditions were: initial denaturation (98° C., 2 min), 30 cycles of 98° C. (20 s), annealing at 50° C. (30 s), extension at 72° C. (45 s) and final extension at 72° C. (10 min), except in validation of DASH protocol (see above), where 35 cycles of amplification were also used. Amplicons were pooled and verified using a 2% TBE agarose e-gel (Life Technologies), purified using AMPure SPRI beads (Beckman Coulter), quality checked using Bioanalyzer DNA 1000 Kit (Agilent) and quantified using the Qubit 2.0 Fluorometer and the dsDNA HS Assay Kit (Life Technologies). Amplicons were pooled at equimolar amounts to create the sequencing library, with the exception of buffer controls, which did not yield enough amplicon and were pooled at the average volume. A mock community (BEI Resources HM-277D) composed of equal genomic concentration of bacterial genomic DNA was sequenced for each amplification plate to monitor and standardize data between amplification plates. Denatured libraries were diluted to 2 nM and were loaded onto the Illumina MiSeq cartridge at 5 pM with 15% (v/v) denatured 12.5 pM PhiX spike-in for sequencing. Complete fetal meconium bank of samples was sequenced on one 250×250 base pair Illumina MiSeq run.

### Sequence Data Processing and Quality Control.

Paired-end reads were assembled using FLASH v1.2.11 [51] requiring a minimum base pair overlap of 200 and de-multiplexed by barcode using QIIME (Quantitative Insights Into Microbial Ecology, v1.9.1) [52]. Quality filtering was accomplished using USEARCH v8.0.1623 to remove reads with >2 expected errors [53]. Quality reads were de-replicated at 100% sequence identity, clustered at 97% sequence identity into operational taxonomic units (OTUs), filtered of chimeric sequences, and mapped back to resulting OTUs using USEARCH. Taxonomy was assigned to the OTUs using SILVA database.

### Fetal Meconium Data Analysis.

OTUs detected in greater than 50% of extraction buffer, blank swab, and air swab controls were removed from all samples prior to further filtering. OTUs comprising fewer than 5 reads and fewer than 0.0001% of the total read counts across all samples were removed. Additional buffer contaminants were identified using decontam package [40] in R. Resulting sequence reads were normalized by multiply rarefying to 1,000 reads per sample as previously described, to assure reduced data were representative of the fuller data for each sample [16]. Dominant taxa were identified for each rarefied sample by determining the OTU with the greatest number of reads per sample.

### Post-Natal Meconium Data Analysis.

16S rRNA gene V4 amplicon sequencing profiles of meconium collected at birth was obtained from the European Nucleotide Archive (ENA) under accession number PRJEB20766 and post-processed as described above for fetal meconium. OTUs were re-picked with combined fetal and post-natal meconium datasets combined. Infant stool samples with high identity to fetal isolates were identified by first trimming the appropriate variable region (depending on study) from full-length 16S rRNA gene Lacto166, Lacto167, or Micro36 sequences. These sequences were then aligned using BLASTn to publicly available infant stool cohorts [15,16,23] with accession numbers PRJEB13896, PRJEB20766, PRJEB8463; sequences with >97% identity and >99% coverage were identified.

### Immune Cell Isolation

Uninterrupted stomach to caecum sections of the fetal small intestine were dissected in cold 1×PBS (see above). The intestine was cut into 1 cm sections and washed three times with 1 mM DTT in 1×PBS for 10 minutes at 37° C. to remove mucus. The epithelial layer was dissociated with three washes of 1 mM EDTA in 1×PBS for 20 minutes at 37° C. and the latter wash was preserved in RNAlater (Ambion) at −80° C. for RNAseq. The remaining lamina propria cells were dissociated with freshly prepared 1 mg/mL Collagenase IV (Gibco) and 10 mg mL⁻¹ DNAse (Roche) in cRPMI for 30 minutes at 37° C., in a shaking water bath at 200 rpm. Mesenteric lymph node and spleen cells were isolated by a 30-minute digestion in Collagenase IV media as described above and then gently pressed through a 70 μm strainer. Cells were separated in a 20%-40%-80% Percoll density gradient at 400× g for 40 minutes: T cells were recovered at the 40-80% interface, while antigen presenting cells were recovered at the 20-40% interface. All cells were washed twice with cRPMI media. Viability was measured with propidium iodide (Sigma Aldrich) and AQUA dye (Invitrogen) using flow cytometry.

### Epithelial Cell RNA Sequencing

Cryopreserved epithelial cell layers (in RNAlater, Ambion) were lysed using QIAshredder (QIAGEN) columns and RNA was extracted using RNAqueous kit (ThermoFisher). RNA was quantified using Qubit RNA HS Assay (ThermoFisher), normalized, and converted to cDNA using SMARTer cDNA Synthesis Kit (Takara Bio) using 7 cycles of amplification. RNA and cDNA quality was determined by Bioanalyzer (Agilent). cDNA was fragmented, ligated with Illumina adapters using Nextera XT kit (Illumina), following manufacturer's instructions, and sequenced on NovaSeq6000 sequencer using two lanes. Paired-end 100 by 100 bp reads were obtained, demultiplexed, quality filtered, removed of Illumina adapters using TrimGalore (github.com/FelixKrueger/TrimGalore), and aligned to the human genome (Hg38 release) using STAR [54] with ENCODE recommended parameters. Features were assigned to transcripts using featureCounts [55], normalized using DESEQ2 [56]. Differential expression was evaluated using DESEQ2 genes with at least 20 reads per gene in respective sample grouping. Log-normalized read counts were obtained from DESEQ2 package, genes were filtered for presence in 75% of samples per comparison group, top variable genes were identified by the coefficient of variance and used to calculate principal components of Euclidean distances.

### Fluorescence In Situ Hybridization

Murine and human fetal terminal ileum was fixed in Carnoy fixative to preserve the mucous layer [57], embedded in Tissue-Tek OCT (VWR) medium, and cryosectioned to 5 μm sections using a cryostat. Sections were thawed, were post-fixed with acetone for 15 minutes, and rinsed with 1×PBS. Slides were incubated with sterile-filtered 1004 of probe solution containing 35% formamide, as previously described [57]. Hybridizations were performed for 10 hours at 48° C., followed by a washing step for one hour at the same temperature, as previously described [57]. Hybridization probes were utilized at 0.5 μM final concentration and included fluorescently-labeled oligos eubacterial (EUB)/5Cy3/GC TGC CTC CCG TAG GAG T/3Cy3Sp/(SEQ ID NO: 8) [58] or non-targeting (NEUB)/5Cy3/AC TCC TAC GGG AGG CAG C/3Cy3Sp/(SEQ ID NO: 9) [58]. Slides were mounted in Vectashield with DAPI (Vector Laboratories) and imaged at 400× and 1000× magnification using epifluorescence Keyence Microscope BZ-X700. Quantification of images was performed in ImageJ software using the set scale function to calibrate pixels to μm units, freehand selection tool was used to trace the perimeter of each villi, and tracing lengths were measured and summed for each section. The point tool was used to manually count EUB or NEUB signal.

### Electron Microscopy

Terminal ileum of fetal intestines was dissected and ligated with sterile suture to prevent contamination of the internal lumen. Ligated samples were immediately immersed in 2.5% (v/v) electron microscopy (EM) grade glutaraldehyde fixative (Sigma Aldrich) in 1×PBS solution and incubated overnight at room temperature with agitation. Samples were washed twice with 1×PBS for 15 minutes and dehydrated with a series of ethanol baths. Samples were then critical point dried (Tousimisautosamdri-815), sliced open with a clean razorblade, mounted in conductive silver epoxy (Ted Pella, Inc.), and coated with 15-30 nm of iridium (Cressington 208-HR sputter coater). Electron micrographs were recorded using a Carl Zeiss ULTRA55 FE-SEM at accelerating voltages in the range 1.24-3.9 keV, working distances of 4.8-9.2 mm, and 20-60 μm diameter apertures with high-current mode. Post-processing of images was not performed. Specimens were stored in a vacuum chamber to avoid contamination between imaging sessions.

### Bacterial Isolation

Punch biopsies were taken from three samples of cryopreserved meconium with highest read counts each for Lactobacillus or Micrococcus using a sterile surgical punch biopsy tool (Integra Miltex, Plainsboro, N.J.) in clean biosafety cabinet. Three independent fetal meconium samples were used for isolation. Punch biopsies of Micrococcus enriched meconium were incubated in cRPMI with or without 2×10⁶ THP1 human monocyte feeder cells for 48 hours at 37° C. in ambient atmospheric stationary conditions. Single colonies were isolated after transfer to brain heart infusion (BHI; TekNova) agar plates and single colonies were picked. Punch biopsies of Lactobacillus enriched meconium were incubated anaerobically in tryptic soy broth (BD) supplemented with 5% defibrinated horse blood (TSBB; Fisher Scientific) for 48 hours at 37° C. 5% CO₂ prior to single colony isolation from tryptic soy agar (BD) supplemented with 5% defibrinated horse blood (TSBA). Colonies sequencing (Quintara Biosciences) was performed using the full length 16S rRNA gene using primer pairs 27F (5′-seq-3′) and 1492R (5′-seq-3′) [59]. Full-length gene was assembled using Clustal Omega and taxonomy was determined by SINA [60] against the curated SILVA database. Reference strains were obtained from American Type Culture Collection for Micrococcus luteus (MicroRef1, ATCC 4698; MicroRef2 ATCC 12698) and Lactobacillus iners (LactoRef, ATCC 55195) and grown by ATCC's protocol.

### Bacterial Whole Genome Sequencing and Comparative Genomics

### Whole Genome Sequencing and Assembly

Twenty-four-hour cultures of Micro36, Lacto166, and Lacto167 were obtained in media and culture conditions as described above, and DNA was extracted using CTAB-based protocol as described above. Genomic DNA (gDNA) was fragmented and Illumina adapters were ligated using Nextera XT (Illumina) kit following manufacturer's instructions. gDNA library quality was verified by gel-electrophoresis Bioanalyzer (Agilent) and was sequenced on Illumina MiSeq using a MiSeq Reagent Kit v3 (Illumina) with 300×300 bp paired-end reads. Reads were removed of adapters and quality filtered using TrimGalore. When possible, paired-end reads were assembled using FLASh [51] for use as a single-ended library for assembly using SPAdes [61] genome assembler. Genome assembly quality was determined by QUAST [62] and genomes were submitted NCBI Prokaryotic Genome Annotation Pipeline (PGAP). Annotation was performed locally using NCBI COG database in Anvi'o package [63].

### Comparative Genomics

Lactobacillus and Micrococcus genomes were downloaded from NCBI using NCBI genome download tool (github.com/kblin/ncbi-genome-download) and imported into Anvi'o pangenome analysis environment [63]. Average nucleotide identity and coverage was calculated using ANIb within pyani package (widdowquinn.githubio/pyani/) [64]. Single copy genes [65] were identified for all relevant genomes within Anvi'o environment, aligned using MUSCLE [66], phylogenetic trees were constructed using FastTree2 [67], and visualized in iTOL [68].

### Post-Natal Data Analysis

A custom kraken2 [69] database was created by adding Micro36, Lacto166, and Lacto167 genome contigs to the standard database. Maternal and infant stool and various body site bacterial metagenomic reads [24,25] and public metadata were obtained from NCBI SRA in FASTQ format using accession numbers PRJNA475246 and PRJNA352475. Percent relative abundance of M. luteus and L. jensenii per sample was obtained using kraken2 software was used to classify metagenomic reads against the custom database using a minimum base quality threshold of 20 and a confidence threshold of 95%.

### Bacterial Growth Curves

Liquid cultures of Lactobacillus and Micrococcus strains were grown for 24-48 hours at 37° C. in chopped meat carbohydrate broth (CMC, Anaerobe Systems) or BHI, respectively. Cultures were normalized to 0.05 optical density at A₆₀₀ₙₘ (OD₆₀₀) and incubated with indicated molar concentrations of progesterone (Tocris Bioscience) and 17β-estradiol (Tocris Bioscience) or equal volume of absolute ethanol vehicle (Sigma Aldrich), in respective culture media (see above). To test whether bacterial isolates were capable of growth with progesterone and 170-estradiol as the sole carbon source, bacterial growth curves were performed in freshly prepared mineral salt media [70] supplemented with 1×10⁻⁵M progesterone and 1×10⁻⁶M 17β-estradiol or equal volume of absolute ethanol vehicle at a normalized starting OD₆₀₀ of 0.1. Bacterial cultures were then incubated in a Cytation3 spectrophometer (BioTek) at 37° C. for 35 hours, and OD600 was recorded every 15 minutes.

### Gentamycin Protection Assay

Intracellular lifestyle of bacterial isolates was determined by gentamycin protection assays as described previously [71]. Primary human antigen presenting cells from fetal spleen were enriched by negative selection using Easy Step Human Biotin Isolation kit (STEMCELL Technologies) and biotin-conjugated mouse anti-human mAbs for CD3, CD56, CD19, and CD20. Isolated cells were incubated for 24h in cRPMI with penicillin and streptomycin at 4° C. Fetal antigen presenting cells or RAW 264.7 macrophage cells (ATCC) were seeded in each well of a 96-well plate and incubated for two hours at 37° C. 5% CO₂ with bacterial isolate overnight cultures at a multiplicity of infection (MOI) of 10. Non-adherent bacteria were removed by washing three times with 1×PBS and incubating for 30 minutes with cRPMI supplemented with 50 μg mL⁻¹ gentamycin. Cells were then incubated with 10 μg mL⁻¹ gentamycin supplemented cRPMI for 3, 24, 40, 48 or 50 hours at 37° C. 5% CO₂. Intracellular bacteria were recovered by lysing eukaryotic cells with sterile 1% (v/v) Triton X (Sigma Aldrich) solution for 10 minutes, with lysis was visually confirmed by light microscope. CFUs were counted from serial dilutions of lysate, grown on either BHI or TSBB (see above) agar plates for Micrococcus and Lactobacillus exposed cells, respectively. Escherichia coli strain DH10B was used as a negative control. Lysate was plated on respective media agar plates with 10 μg mL⁻¹ gentamycin to determine acquisition of antibiotic resistance.

### Antibodies and Flow Cytometry

Isolated cells were incubated in 2% FBS in PBS with 1 mM EDTA (staining buffer) with human Fc blocking antibody (STEMCELL Technologies) and stained with fluorochrome-conjugated antibodies against surface markers. Intracellular protein detection was performed on fixed, permeabilized cells using the Foxp3/Transcription Factor Staining Buffer set (Tonbo Biosciences). Mouse anti-human monoclonal antibodies used in this study include: TCRβ PerCP Cy5.5 (Clone IP26, eBioscience Cat. No. 46-9986-42), Vα7.2 BV605 (Clone 3C10, BioLegend Cat. No. 351720), CD4 APC H7 (Clone L200, BD Pharmingen Cat. No. 560837), CD8a FITC and PE Cy7 (Clone B7-1, BD Pharmingen Cat. No. 557226), CD45RA PE Cy7 (Clone HI100, BD Pharmingen Cat. No. 555489), CCR7 PE (Clone G043H7, BioLegend Cat. No. 353208), CD103 BV421 (Clone Ber-ACT8, BD Pharmingen Cat. No. 550259), PLZF-APC (Clone 6318100, R&D Cat. No. IC2944A), CD161-BV711 (Clone DX12, BD Biosciences Cat. No. 563865), CD25 FITC (Clone 2A3, BD Biosciences Cat. No. 347643), FoxP3 PE (Clone PCH101, eBioscience Cat. No. 12-4776-42), IFNγ-FITC (Clone 25723.11, BD Biosciences Cat. No. 340449) TNFα-PE Cy7 (Clone MAB11, BD Pharmingen Cat. No. 557647), CD45 APC (Clone HI30, Tonbo Cat. No. 20-0459), CD14 BV605 (Clone M5E2, BD Pharmingen Cat. No. 564054), CD11c BB515 (Clone B-ly6, BD Pharmingen Cat. No. 564491), HLA-DR APC-R700 (Clone G46-6, BD Cat. No. 565128), CD3 biotin (Clone HIT3a, BD Cat. No. 564713), CD19 biotin (Clone SJ25C1, BD Cat. No. 562947), CD20 biotin (Clone 2H7, eBioscience Cat. No. 13-0209-82), CD56 biotin (Clone NCAM16.2, BD Cat. No. 563041), LLT1 PE (Clone 402659 R&D Cat. No. FAB3480P). Streptavidin conjugated to BV711 (BD Biosciences Cat. No. 563262) was used to detect biotin antibodies. All cells were stained with Aqua LIVE/DEAD Fixable Dead Cell Stain Kit (Invitrogen) to exclude dead cells from analysis. All data were acquired with BD LSR/Fortessa Dual SORP using FACS Diva software (BD Biosciences) and analyzed with FlowJo (TreeStar) software.

Ex Vivo Intestinal Epithelial Cell Transcriptomics after Bacterial Isolates Exposure

EDTA washes containing fetal intestinal epithelial cells (see above) were washed with 1×PBS, passed through 40 μm strainer, and plated on Collagen I coated 96-well plates (Corning) in cRPMI containing 5 ng/mL epidermal growth factor (Gibco). Cells were incubated overnight at 37° C. 5% CO₂ 4% O₂, to mimic hypoxic conditions in the fetal intestine [72] and non-adherent cells were removed. Cells were allowed to differentiate for five days or until 80% confluence, with media replacement every two days. Cells were incubated with a multiplicity of infection of 10 of bacterial isolates in cRPMI for 4 at 37° C. 5% CO₂ 4% O₂. After 4h, cells were preserved in RNAlater and RNA was prepared for sequencing as described above.

Ex Vivo Antigen Presenting Cell Activation with Bacterial Isolates

Antigen presenting cells from fetal spleen were enriched by negative selection using Easy Step Human Biotin Isolation kit (STEMCELL Technologies) as described above. Cells were seeded into 96-well plates and incubated with multiplicity of infection of 10 of bacterial isolates in cRPMI for 4 hours at 37° C. 5% CO₂ 4% O₂, to mimic hypoxic conditions in the fetal intestine [72] and normalize for bacterial growth.

### Ex Vivo Autologous Mixed Lymphocyte Reactions

Lamina propria T cells were enriched using Easy Sep Human T cell isolation kit (STEMCELL Technologies), effector memory cells were sorted to >99% purity (FIG. 13I) using BD Aria Fusion SORP, and cells were labeled with cell trace violet (Invitrogen). Splenic antigen presenting cells autologous to isolated T cells were enriched as described above, sorted to >96% purity (FIG. 13J), and exposed to bacterial isolates as described above. Bacteria were removed with three washes of cRPMI supplemented with penicillin and streptomycin. Sorted, labeled effector memory T cells were incubated with pre-exposed antigen presenting cells in a 2:1 ratio in cRPMI with supplemented with 10 ng/mL IL-2 (PeproTech), 10 ng/mL IL-7 (PeproTech), 2 μg/mL purified anti-CD28 (Clone CD28.2, BD Pharmigen Cat. No. 555725), 2 μg/mL purified anti-CD49d (Clone, BD Pharmingen Cat No. 555501), and 10 μg/mL gentamycin for three days at 37° C. 5% CO₂ 4% O₂. Cells were incubated with 10 μg/mL Brefeldin A (Sigma Aldrich) in the same media for 4 hours at 37° C. 5% CO₂ 4% O₂ and were subsequently stained for intracellular cytokine production as described above. Mixed lymphocyte reactions as described above were extended to 5 days with enriched T cells and antigen presenting cells, and T cell proportions were measured using flow cytometry as described above.

### Statistical Analysis

Shannon's diversity index was calculated in Qiime and student's, Welch's, or Wicoxon t-tests were calculated in R, depending on the distribution. Bray Curtis distance matrices were calculated in QIIME to assess compositional dissimilarity between samples and visualized using principal coordinates analysis (PCoA) plots in R. Permutational multivariate analysis of variance (PERMANOVA) was performed using Adonis function of vegan package [73] in R to determine factors that significantly (p<0.05) explained variation in microbiota β-diversity. In cases where replicates were included, linear mixed effects modeling was used to determine significance using the R package lmerTest [74]. Ranked abundance curve fit to geometric or log-series functions was determined by Bayesian Information Criterion (BIC) to evaluate models generated from fitsad function in vegan R package. To determine which OTUs differed in relative abundance between contamination swab and meconium, unnormalized read counts were transformed using DESEQ2 in QIIME to identify log-fold change enrichment and corrected for multiple hypothesis testing using the false-discovery rate (q<0.05). Growth curves were modeled using a logistic regression in R package growthcurver [75], integral of the best fit regression was used to calculate the area under the curve (auc), and auc of vehicle was subtracted from hormone treatment controls according to the following formula:

$${\sum }_{{t}_{i} = 1}^{n} Hormone treatment ( {t}_{i} ) - Vehicle ( {t}_{i} ) Δ i$$

Significance in gentamycin protection assays was evaluated by transforming colony forming unit (CFU) counts using log₁₀(CFU+1) and applying a generalized linear model to transformed data. Significance in ex vivo immune cell assays was evaluated using linear mixed effect modeling to account for cell donor correlations and where indicated, residuals are plotted. Except where indicated, all analyses were performed using R statistical programming language in the Jupyter Notebook environment.

### Data Availability

16S rRNA bacterial profiling data generated in this study will be available in the EMBLI-EBI ENA repository accession #PRJEB25779 (www.ebi.ac.uk/ena). De novo assembled genomes were deposited at DDBJ/ENA/GenBank under the accession numbers VFQG00000000, VFQH00000000, and VFQL00000000 for Lacto166, Lacto167, and Micro36, respectively. The genome version described in this example is version VFQG01000000, VFQH01000000, and VFQL01000000 for Lacto166, Lacto167, and Micro36, respectively. Raw sequence reads used for genome assembly were deposited in NCBI SRA under BioProject accession #PRJNA498338, PRJNA498340, and PRJNA498337 for Lacto166, Lacto167, and Micro36, respectively. RNA sequencing dataset will be available in NCBI under PRJNA506292 accession. This data is incorporated herein, by reference.

### REFERENCES

1. Spencer, J., Dillon, S. B., Isaacson, P. G. & Macdonald, T. T. T cell subclasses in fetal ileum. Clin. Exp. Immunol. 65, 553-558 (1986).
2. Howie, D. et al. Extrathymic T Cell Differentiation in the Human Intestine Early in Life. J. Immunol. 161, 5862-5872 (1998).
3. McGovern, N. et al. Human fetal dendritic cells promote prenatal T-cell immune suppression through arginase-2. Nature (2017). doi:10.1038/nature22795
4. Spencer, J., MacDonald, T. T., Finn, T. & Isaacson, P. G. The development of gut associated lymphoid tissue in the terminal ileum of fetal human intestine. Clin. Exp. Immunol. 64, 536-43 (1986).
5. Podd, B. S. et al. T Cells in Cryptopatch Aggregates Share TCR Variable Region Junctional Sequences with T Cells in the Small Intestinal Epithelium of Mice. J. Immunol. 176, 6532-6542 (2006).
6. Halkias, J. et al. CD161 contributes to prenatal immune suppression of IFNγ-producing PLZF+ T cells. J. Clin. Invest. (2019). doi:10.1172/JCI125957
7. Schreurs, R. R. C. E. et al. Human Fetal TNF-α-Cytokine-Producing CD4+ Effector Memory T Cells Promote Intestinal Development and Mediate Inflammation Early in Life. Immunity 0, 1-15 (2019).
8. Li, N. et al. Memory CD4+ T cells are generated in the human fetal intestine. Nat. Immunol. (2019). doi:10.1038/s41590-018-0294-9
9. Aagaard, K. et al. The placenta harbors a unique microbiome. Sci. Transl. Med. 6, 237ra65 (2014).
10. Collado, M. C., Rautava, S., Aakko, J., Isolauri, E. & Salminen, S. Human gut colonisation may be initiated in utero by distinct microbial communities in the placenta and amniotic fluid. Sci. Rep. 6, 23129 (2016).
11. Steel, J. H. et al. Bacteria and Inflammatory Cells in Fetal Membranes Do Not

Always Cause Preterm Labor. Pediatr. Res. 57, 404-411 (2005).
12. Lim, E. S., Rodriguez, C. & Holtz, L. R. Amniotic fluid from healthy term pregnancies does not harbor a detectable microbial community. 4-11 (2018).
13. Lauder, A. P. et al. Comparison of placenta samples with contamination controls does not provide evidence for a distinct placenta microbiota. Microbiome 1-11 (2016). doi:10.1186/s40168-016-0172-3
14. Chu, D. M. et al. Maturation of the infant microbiome community structure and function across multiple body sites and in relation to mode of delivery. Nat. Med. 23, 314-326 13 (2017).
15. Durack, J. et al. Delayed gut microbiota development in high-risk for asthma infants is temporarily modifiable by Lactobacillus supplementation. Nat. Commun. 9, (2018).
16. Fujimura, K. E. et al. Neonatal gut microbiota associates with childhood multisensitized atopy and T cell differentiation. Nat. Med. 22, 1187-1191 (2016).
17. Gu W, Crawford ED, O'Donovan B D, Wilson M R, Chow E D, Retallack H, D. J. Depletion of Abundant Sequences by Hybridization (DASH): Using Cas9 to remove unwanted high-abundance species in sequencing libraries and molecular counting applications. Genome Biol. 17, (2016).
18. MacArthur, R. H. On the relative abundance of bird species. Proc. Natl. Acad. Sci. U.S.A. 43, 293-295 (1957).
19. Lagerholm, C. et al. Colonic epithelial cell diversity in health and inflammatory bowel disease. Nature 567, 49-55 (2019).
20. Trotter, A., Maier, L., Grill, H.-J., Wudy, S. A. & Pohlandt, F. 17β-Estradiol and Progesterone Supplementation in Extremely Low-Birth-Weight Infants. Pediatr. Res. 45, 489-493 (1999).
21. Yotis, W. & Stanke, R. Bacteriostatic action of progesterone on staphylococci and other microorganisms. J. Bacteriol. 92, 1285-1289 (1966).
22. Varghese, N. J. et al. Microbial species delineation using whole genome sequences. Nucleic Acids Res. 43, 6761-6771 (2015).
23. Arrieta, M.-C. et al. Early infancy microbial and metabolic alterations affect risk of childhood asthma. Sci. Transl. Med. 7, 307ra152-307ra152 (2015).
24. Ferretti, P. et al. Mother-to-Infant Microbial Transmission from Different Body Sites Shapes the Developing Infant Gut Microbiome. Cell Host Microbe 24, 133-145.e5 (2018).
25. Yassour, M. et al. Strain-Level Analysis of Mother-to-Child Bacterial Transmission during the First Few Months of Life. Cell Host Microbe 24, 146-154.e4 (2018).
26. Oliveira, M. J. et al. Helicobacter pylori induces gastric epithelial cell invasion in a c-Met and type IV secretion system-dependent manner. J. Biol. Chem. 281, 34888-34896 (2006).
27. Benach, J. et al. Structure of bacterial 30/170-hydroxysteroid dehydrogenase at 1.2 Å resolution: A model for multiple steroid recognition. Biochemistry 41, 14659-14668 (2002).
28. Hillas, P. J., Soto Del Alba, F., Oyarzabal, J., Wilks, A. & Ortiz De Montellano, P. R. The AhpC and AhpD antioxidant defense system of Mycobacterium tuberculosis. J. Biol. Chem. 275, 18801-18809 (2000).
29. Dons, L. E. et al. Role of the Listeria monocytogenes 2-Cys peroxiredoxin homologue in protection against oxidative and nitrosative stress and in virulence. Pathog. Dis. 70, 70-74 (2014).
30. Davis, E. A., Zhou, W. & Dailey, M. J. Evidence for a direct effect of the autonomic nervous system on intestinal epithelial stem cell proliferation. Physiol. Rep. 6, 1-8 (2018).
31. McGovern, N. et al. Human fetal dendritic cells promote prenatal T-cell immune suppression through arginase-2. Nature 546, 662-666 (2017).
32. Coombes, J. L. et al. A functionally specialized population of mucosal CD103 DCs induces Foxp3⁺ regulatory T cells via a TGF-β- and retinoic acid-dependent mechanism. J. Exp. Med. 204, 1757-1764 (2007).
33. Egea, L., Hirata, Y. & Kagnoff, M. F. GM-CSF: A role in immune and inflammatory reactions in the intestine. Expert Rev. Gastroenterol. Hepatol. 4, 723-731 (2010).
34. Krause, P. et al. IL-10-producing intestinal macrophages prevent excessive antibacterial innate immunity by limiting IL-23 synthesis. Nat. Commun. 6, (2015).
35. Denning, T. L., Wang, Y. C., Patel, S. R., Williams, I. R. & Pulendran, B.

Lamina propria macrophages and dendritic cells differentially induce regulatory and interleukin 17-producing T cell responses. Nat. Immunol. 8, 1086-1094 (2007).
36. Schreurs, R. Human Fetal TNF-α-Cytokine-Producing CD4+ Effector Memory T Cells Promote Intestinal Development and Mediate Inflammation Early in Life. Immunity 50, 1-15 (2019).
37. Lee, J. S. et al. Interleukin-23-Independent IL-17 Production Regulates Intestinal Epithelial Permeability. Immunity 43, 727-738 (2015).
38. Rosen, D. B. et al. Functional Consequences of Interactions between Human NKR-PIA and Its Ligand LLT1 Expressed on Activated Dendritic Cells and B Cells. J. Immunol. 180, 6508-6517 (2008).
39. Jervis-Bardy, J. et al. Deriving accurate microbiota profiles from human samples with low bacterial content through post-sequencing processing of Illumina MiSeq data. Microbiome 3, 1-11 (2015).
40. Davis, N. M., Proctor, D., Holmes, S. P., Reiman, D. A. & Callahan, B. J. Simple statistical identification and removal of contaminant sequences in marker-gene and metagenomics data. bioRxiv 221499 (2018). doi:10.1101/221499
41. Martin, R. et al. Characterization of indigenous vaginal lactobacilli from healthy women as probiotic candidates. Int. Microbiol. 11, 261-266 (2008).
42. Chen, C. et al. The microbiota continuum along the female reproductive tract and its relation to uterine-related diseases. Nat. Commun. 8, 875 (2017).
43. Tulchinsky, D. & Hobel, C. J. Plasma human chorionic gonadotropin, estrone, estradiol, estriol, progesterone, and 17α-hydroxyprogesterone in human pregnancy: III. Early normal pregnancy. Am. J. Obstet. Gynecol. 117, 884-893 (1973).
44. Duerkop, B. A., Vaishnava, S. & Hooper, L. V. Immune Responses to the Microbiota at the Intestinal Mucosal Surface. Immunity 31, 368-376 (2009).
45. Mold, J. E. et al. Fetal and Adult Hematopoietic Stem Cells Give Rise to Distinct T Cell Lineages in Humans. Science (80-.). 330, 1695-1699 (2010).
46. Mold, J. E. et al. Maternal Alloantigens Promote the Development of Tolerogenic Fetal Regulatory T Cells in Utero. Science (80-.). 322, 1562-1565 (2008).
47. Pastor-vargas, C. et al. Detection of major food allergens in amniotic fluid: initial allergenic encounter during pregnancy. 27, 716-720 (2016).
48. Frascoli, M. et al. Alloreactive fetal T cells promote uterine contractility in preterm labor via IFN-γ and TNF-α. Sci. Transl. Med. 10, eaan2263 (2018).
49. Iwai, S. et al. The lung microbiome of Ugandan HIV-infected pneumonia patients is compositionally and functionally distinct from that of San Franciscan patients. PLoS One 9, (2014).
50. Caporaso, J. G. et al. Global patterns of 16S rRNA diversity at a depth of millions of sequences per sample. Proc. Natl. Acad. Sci. 108, 4516-4522 (2011).
51. Magoč, T. & Salzberg, S. L. FLASH: fast length adjustment of short reads to improve genome assemblies. Bioinformatics 27, 2957-63 (2011).
52. Caporaso, J. G. et al. correspondence QIIME allows analysis of high-throughput community sequencing data Intensity normalization improves color calling in SOLiD sequencing. Nat. Publ. Gr. 7, 335-336 (2010).
53. Edgar, R. C. & Flyvbjerg, H. Error filtering, pair assembly and error correction for next-generation sequencing reads. Bioinformatics 31, 3476-3482 (2015).
54. Dobin, A. et al. STAR: Ultrafast universal RNA-seq aligner. Bioinformatics 29, 15-21 (2013).
55. Liao, Y., Smyth, G. K. & Shi, W. FeatureCounts: An efficient general purpose program for assigning sequence reads to genomic features. Bioinformatics 30, 923-930 (2014).
56. Love, M. I., Huber, W. & Anders, S. Moderated estimation of fold change and dispersion for RNA-seq data with DESeq2. Genome Biol. 15, 1-21 (2014).
57. Johansson, M. E. V & Hansson, G. C. Preservation of Mucus in Histological Sections, Immunostaining of Mucins in Fixed Tissue, and Localization of Bacteria with FISH. in Mucins: Methods and Protocols (eds. McGuckin, M. A. & Thornton, D. J.) 229-235 (Humana Press, 2012). doi:10.1007/978-1-61779-513-8_13
58. Vaishnava, S. et al. The Antibacterial Lectin RegIII. Science (80-.). 334, 255-258 (2011).
59. Weisburg, W. G., Barns, S. M., Pelletier, D. A. & Lane, D. J. 16S ribosomal DNA amplification for phylogenetic study. Weisburg, WG 173, 697-703 (1991).
60. Pruesse, E., Peplies, J. & Glockner, F. O. SINA: Accurate high-throughput multiple sequence alignment of ribosomal RNA genes. Bioinformatics 28, 1823-1829 (2012).
61. Bankevich, A. et al. SPAdes: A New Genome Assembly Algorithm and Its Applications to Single-Cell Sequencing. J. Comput. Biol. 19, 455-477 (2012).
62. Gurevich, A., Saveliev, V., Vyahhi, N. & Tesler, G. QUAST: Quality assessment tool for genome assemblies. Bioinformatics 29, 1072-1075 (2013).
63. Eren, A. M. et al. Anvi'o: an advanced analysis and visualization platform for 'omics data. PeerJ 3, e1319 (2015).
64. Pritchard, L., Glover, R. H., Humphris, S., Elphinstone, J. G. & Toth, I. K. Genomics and taxonomy in diagnostics for food security: Soft-rotting enterobacterial plant pathogens. Anal. Methods 8, 12-24 (2016).
65. Rinke, C. et al. Insights into the phylogeny and coding potential of microbial dark matter. Nature 499, 431-437 (2013).
66. Edgar, R. C. MUSCLE: Multiple sequence alignment with high accuracy and high throughput. Nucleic Acids Res. 32, 1792-1797 (2004).
67. Price, M. N., Dehal, P. S. & Arkin, A. P. FastTree 2—Approximately maximum-likelihood trees for large alignments. PLoS One 5, (2010).
68. Letunic, I. & Bork, P. Interactive tree of life (iTOL) v3: an online tool for the display and annotation of phylogenetic and other trees. Nucleic Acids Res. 44, W242-W245 (2016).
69. Wood, D. E. & Salzberg, S. L. Kraken: ultrafast metagenomic sequence classification using exact alignments. Genome Biol. 15, R46 (2014).
70. Sekar, S., Mahadevan, S., Kumar, S. S. D. & Mandal, A. B. Thermokinetic responses of the metabolic activity of Staphylococcus lentus cultivated in a glucose limited mineral salt medium. J. Therm. Anal. Calorim. 104, 149-155 (2011).
71. Subashchandrabose, S., Smith, S. N., Spurbeck, R. R., Kole, M. M. & Mobley, H. L. T. Genome-Wide Detection of Fitness Genes in Uropathogenic Escherichia coli during Systemic Infection. PLoS Pathog. 9, 1-15 (2013).
72. Ducsay, C. A. et al. Gestational Hypoxia and Developmental Plasticity. Physiol. Rev. 98, 1241-1334 (2018).
73. Oksanen, J. et al. Vegan: Community Ecology Package. R Package Version. 2.0-10. CRAN (2013).
74. Kuznetsova, A., Brockhoff, P. B. & Christensen, R. H. B. ImerTest Package:

Tests in Linear Mixed Effects Models. J. Stat. Softw. 82, (2017).
75. Sprouffske, K. & Wagner, A. Growthcurver: An R package for obtaining interpretable metrics from microbial growth curves. BMC Bioinformatics 17, 17-20 (2016).

VI. Informal sequence listing
(V4 region of the 16S rRNA gene of
the Lacto166 and Lacto 167 fetal
Lactobacillus sp. bacteria strains
identified in Example 1)
SEQ ID NO: 1
TACGTAGGTGGCAAGCGTTGTCCGGATTTATTGGG
CGTAAAGCGAGCGCAGGCGGATTGATAAGTCTGAT
GTGAAAGCCTTCGGCTCAACCGAAGAACTGCATCA
GAAACTGTCAATCTTGAGTGCAGAAGAGGAGAGTG
GAACTCCATGTGTAGCGGTGGAATGCGTAGATATA
TGGAAGAACACCAGTGGCGAAGGCGGCTCTCTGGT
CTGTAACTGACGCTGAGGCTCGAAAGCATGGGTAG
CGAACAGGATTAGATACCCTGGTAGTCCA
(V4 region of the 16S rRNA gene of
the Micro36 fetal Micrococcus sp.
bacterium identified in Example 1)
SEQ ID NO: 2
TACGTAGGGTGCGAGCGTTATCCGGAATTATTGGG
CGTAAAGAGCTCGTAGGCGGTTTGTCGCGTCTGTC
GTGAAAGTCCGGGGCTTAACCCCGGATCTGCGGTG
GGTACGGGCAGACTAGAGTGCAGTAGGGGAGACTG
GAATTCCTGGTGTAGCGGTGGAATGCGCAGATATC
AGGAGGAACACCGATGGCGAAGGCAGGTCTCTGGG
CTGTAACTGACGCTGAGGAGCGAAAGCATGGGGAG
CGAACAGGA
(16S rRNA gene of the of the Lacto166
fetal Lactobacillus sp. bacterium
identified in Example 1)
SEQ ID NO: 3
GCCTAATACATGCAAGTCGAGCGAGCTTGCCTATA
GAAATTCTTCGGAATGGACATAGATACAAGCTAGC
GGCGGATGGGTGAGTAACGCGTGGGTAACCTGCCC
TTAAGTCTGGGATACCATTTGGAAACAGATGCTAA
TACCGGATAAAAGCTACTTTCGCATGAAAGAAGTT
TAAAAGGCGGCGTAAGCTGTCGCTAAAGGATGGAC
CTGCGATGCATTAGCTAGTTGGTAAGGTAACGGCT
TACCAAGGCGATGATGCATAGCCGAGTTGAGAGAC
TGATCGGCCACATTGGGACTGAGACACGGCCCAAA
CTCCTACGGGAGGCAGCAGTAGGGAATCTTCCACA
ATGGACGAAAGTCTGATGGAGCAACGCCGCGTGAG
TGAAGAAGGTTTTCGGATCGTAAAGCTCTGTTGTT
GGTGAAGAAGGATAGAGGTAGTAACTGGCCTTTAT
TTGACGGTAATCAACCAGAAAGTCACGGCTAACTA
CGTGCCAGCAGCCGCGGTAATACGTAGGTGGCAAG
CGTTGTCCGGATTTATTGGGCGTAAAGCGAGCGCA
GGCGGATTGATAAGTCTGATGTGAAAGCCTTCGGC
TCAACCGAAGAACTGCATCAGAAACTGTCAATCTT
GAGTGCAGAAGAGGAGAGTGGAACTCCATGTGTAG
CGGTGGAATGCGTAGATATATGGAAGAACACCAGT
GGCGAAGGCGGCTCTCTGGTCTGTAACTGACGCTG
AGGCTCGAAAGCATGGGTAGCGAACAGGATTAGAT
ACCCTGGTAGTCCATGCCGTAAACGATGAGTGCTA
AGTGTTGGGAGGTTTCCGCCTCTCAGTGCTGCAGC
TAACGCATTAAGCACTCCGCCTGGGGAGTACGACC
GCAAGGTTGAAACTCAAAGGAATTGACGGGGGCCC
GCACAAGCGGTGGAGCATGTGGTTTAATTCGAAGC
AACGCGAAGAACCTTACCAGGTCTTGACATCCTTT
GACCACCTAAGAGATTAGGTTTTCCCTTCGGGGAC
AAAGAGACAGGTGGTGCATGGCTGTCGTCAGCTCG
TGTCGTGAGATGTTGGGTTAAGTCCCGCAACGAGC
GCAACCCTTGTTAATAGTTGCCAGCATTAAGTTGG
GCACTCTATTGAGACTGCCGGTGACAAACCGGAGG
AAGGTGGGGATGACGTCAAGTCATCATGCCCCTTA
TGACCTGGGCTACACACGTGCTACAATGGGCAGTA
CAACGAGAAGCGAACCTGTGAAGGCAAGCGAATCT
CTTAAAGCTGTTCTCAGTTCGGACTGTAGGCTGCA
ACTCGCCTACACGAAGCTGGAATCGCTAGTAATCG
CGGATCAGCACGCCGCGGTGAATACGTTCCCGGGC
CTTGTACACACCGCCCGTCACACCATGAGAGTTTG
TAACACCCAAAGTCGGTGAGGTAACTTTGAGCCAG
CCGCCAA
(16S rRNA gene of the Micro36 fetal
Micrococcus sp. bacterium
identified in Example 1)
SEQ ID NO: 4
AGTCGAACGATGAAGCCCAGCTTGCTGGGTGGATT
AGTGGCGAACGGGTGAGTAACACGTGAGTAACCTG
CCCTTAACTCTGGGATAAGCCTGGGAAACTGGGTC
TAATACCGGATAGGAGCGTCCACCGCATGGTGGGT
GTTGGAAAGATTTATCGGTTTTGGATGGACTCGCG
GCCTATCAGCTTGTTGGTGAGGTAATGGCTCACCA
AGGCGACGACGGGTAGCCGGCCTGAGAGGGTGACC
GGCCACACTGGGACTGAGACACGGCCCAGACTCCT
ACGGGAGGCAGCAGTGGGGAATATTGCACAATGGG
CGCAAGCCTGATGCAGCGACGCCGCGTGAGGGATG
ACGGCCTTCGGGTTGTAAACCTCTTTCAGTAGGGA
AGAAGCGAAAGTGACGGTACCTGCAGAAGAAGCAC
CGGCTAACTACGTGCCAGCAGCCGCGGTAATACGT
AGGGTGCGAGCGTTATCCGGAATTATTGGGCGTAA
AGAGCTCGTAGGCGGTTTGTCGCGTCTGTCGTGAA
AGTCCGGGGCTTAACCCCGGATCTGCGGTGGGTAC
GGGCAGACTAGAGTGCAGTAGGGGAGACTGGAATT
CCTGGTGTAGCGGTGGAATGCGCAGATATCAGGAG
GAACACCGATGGCGAAGGCAGGTCTCTGGGCTGTA
ACTGACGCTGAGGAGCGAAAGCATGGGGAGCGAAC
AGGATTAGATACCCTGGTAGTCCATGCCGTAAACG
TTGGGCACTAGGTGTGGGGACCATTCCACGGTTTC
CGCGCCGCAGCTAACGCATTAAGTGCCCCGCCTGG
GGAGTACGGCCGCAAGGCTAAAACTCAAAGGAATT
GACGGGGGCCCGCACAAGCGGCGGAGCATGCGGAT
TAATTCGATGCAACGCGAAGAACCTTACCAAGGCT
TGACATGTTCTCGATCGCCGTAGAGATACGGTTTC
CCCTTTGGGGCGGGTTCACAGGTGGTGCATGGTTG
TCGTCAGCTCGTGTCGTGAGATGTTGGGTTAAGTC
CCGCAACGAGCGCAACCCTCGTTCCATGTTGCCAG
CACGTAGTGGTGGGGACTCATGGGAGACTGCCGGG
GTCAACTCGGAGGAAGGTGAGGACGACGTCAAATC
ATCATGCCCCTTATGTCTTGGGCTTCACGCATGCT
ACAATGGCCGGTACAATGGGTTGCGATACTGTGAG
GTGGAGCTAATCCCAAAAAGCCGGTCTCAGTTCGG
ATTGGGGTCTGCAACTCGACCCCATGAAGTCGGAG
TCGCTAGTAATCGCAGATCAGCAACGCTGCGGTGA
ATACGTTCCCGGGCCTTGTACACACCGCCCGTCAA
GTCACGAAAGTCGGTAACACCCGAAGCCGGGGCCT
AACCCTTGTGG
(16S rRNA gene of the of the Lacto167
fetal Lactobacillus sp. bacterium
identified in Example 1)
SEQ ID NO: 5
GCCTAATACATGCAAGTCGAGCGAGCTTGCCTATA
GAAATTCTTCGGAATGGACATAGATACAAGCTAGC
GGCGGATGGGTGAGTAACGCGTGGGTAACCTGCCC
TTAAGTCTGGGATACCATTTGGAAACAGATGCTAA
TACCGGATAAAAGCTACTTTCGCATGAAAGAAGTT
TAAAAGGCGGCGTAAGCTGTCGCTAAAGGATGGAC
CTGCGATGCATTAGCTAGTTGGTAAGGTAACGGCT
TACCAAGGCGATGATGCATAGCCGAGTTGAGAGAC
TGATCGGCCACATTGGGACTGAGACACGGCCCAAA
CTCCTACGGGAGGCAGCAGTAGGGAATCTTCCACA
ATGGACGAAAGTCTGATGGAGCAACGCCGCGTGAG
TGAAGAAGGTTTTCGGATCGTAAAGCTCTGTTGTT
GGTGAAGAAGGATAGAGGTAGTAACTGGCCTTTAT
TTGACGGTAATCAACCAGAAAGTCACGGCTAACTA
CGTGCCAGCAGCCGCGGTAATACGTAGGTGGCAAG
CGTTGTCCGGATTTATTGGGCGTAAAGCGAGCGCA
GGCGGATTGATAAGTCTGATGTGAAAGCCTTCGGC
TCAACCGAAGAACTGCATCAGAAACTGTCAATCTT
GAGTGCAGAAGAGGAGAGTGGAACTCCATGTGTAG
CGGTGGAATGCGTAGATATATGGAAGAACACCAGT
GGCGAAGGCGGCTCTCTGGTCTGTAACTGACGCTG
AGGCTCGAAAGCATGGGTAGCGAACAGGATTAGAT
ACCCTGGTAGTCCATGCCGTAAACGATGAGTGCTA
AGTGTTGGGAGGTTTCCGCCTCTCAGTGCTGCAGC
TAACGCATTAAGCACTCCGCCTGGGGAGTACGACC
GCAAGGTTGAAACTCAAAGGAATTGACGGGGGCCC
GCACAAGCGGTGGAGCATGTGGTTTAATTCGAAGC
AACGCGAAGAACCTTACCAGGTCTTGACATCCTTT
GACCACCTAAGAGATTAGGTTTTCCCTTCGGGGAC
AAAGAGACAGGTGGTGCATGGCTGTCGTCAGCTCG
TGTCGTGAGATGTTGGGTTAAGTCCCGCAACGAGC
GCAACCCTTGTTAATAGTTGCCAGCATTAAGTTGG
GCACTCTATTGAGACTGCCGGTGACAAACCGGAGG
AAGGTGGGGATGACGTCAAGTCATCATGCCCCTTA
TGACCTGGGCTACACACGTGCTACAATGGGCAGTA
CAACGAGAAGCGAACCTGTGAAGGCAAGCGAATCT
CTTAAAGCTGTTCTCAGTTCGGACTGTAGGCTGCA
ACTCGCCTACACGAAGCTGGAATCGCTAGTAATCG
CGGATCAGCACGCCGCGGTGAATACGTTCCCGGGC
CTTGTACACACCGCCCGTCACACCATGAGAGTTTG
TAACACCCAAAGTCGGTGAGGTAACTTTGGAGCCA
GCCGCCTAAG
(V4 region of the 16S rRNA gene of OTU12)
SEQ ID NO: 6
TACGTAGGTGGCAAGCGTTGTCCGGATTTATTGGG
CGTAAAGCGAGTGCAGGCGGCTCGATAAGTCTGAT
GTGAAAGCCTTCGGCTCAACCGGAGAATTGCATCA
GAAACTGTCGAGCTTGAGTACAGAAGAGGAGAGTG
GAACTCCATGTGTAGCGGTGAAATGCGTAGATata
tGGAAGAACACCGGTGGCGAAGGCGGCTactGGTC
TGTTACTGACGCTGAGGCTCGAAAGCATGGGTAGC
GAACAGG
(V4 region of the 16S rRNA gene of OTU10)
SEQ ID NO: 7
TACGTAGGGTGCAAGCGTTATCCGGAATTATTGGG
CGTAAAGAGCTCGTAGGCGGTTTGTCGCGTCTGTC
GTGAAAGTCCGGGGCTCAACTCCGGATCTGCGGTG
GGTACGGGCAGACTAGAGTGATGTAGGGGAGACTG
GAATTCCTGGTGTAGCGGTGGAATGCGCAGATATC
AGGAGGAACACCGATGGCGAAGGCAGGTCTCTGGG
CATTAACTGACGCTGAGGAGCGAAAGCATGGGGAG
CGAACAGG

### VII. EMBODIMENTS

The present description provides the following embodiments:

1. A method of treating, preventing, or reducing the risk of an inflammatory disease in a subject in need thereof, comprising administering to the subject an effective amount of a fetal Micrococcus sp. bacterium and/or a fetal Lactobacillus sp. bacterium.

2. The method of embodiment 1, wherein the fetal Micrococcus sp. bacterium and/or the fetal Lactobacillus sp. bacterium is administered orally.

3. The method of embodiment 2, wherein the subject is a female and the fetal Micrococcus sp. bacterium and/or the fetal Lactobacillus sp. bacterium is administered vaginally.

4. The method of embodiment 3, wherein the subject is pregnant.

5. The method of any one of embodiments 1-4, wherein the subject has an increased risk for developing the inflammatory disease compared to a general population of healthy subjects.

6. The method of any one of embodiments 1-4, wherein the subject has an inflammatory disease.

7. The method of any one of embodiments 1-6, wherein the inflammatory disease is an allergy.

8. The method of embodiment 7, wherein the allergy is an allergy to milk, eggs, fish, shellfish, a tree nut, peanuts, wheat, dander from a cat, dog, or rodent, an insect sting, pollen, latex, dust mites, or soybeans.

9. The method of embodiment 7, wherein the allergy is pediatric allergic asthma, hay fever, or allergic airway sensitization.

10. The method of any one of embodiments 1-6, wherein the inflammatory disease is a chronic inflammatory disease

11. The method of embodiment 10, wherein the chronic inflammatory disease is asthma.

12. The method of any one of embodiments 1-6, wherein the inflammatory disease is an allergy, atopy, asthma, an autoimmune disease, an autoinflammatory disease, a hypersensitivity, pediatric allergic asthma, allergic asthma, inflammatory bowel disease, Celiac disease, Crohn's disease, colitis, ulcerative colitis, collagenous colitis, lymphocytic colitis, diverticulitis, irritable bowel syndrome, short bowel syndrome, stagnant loop syndrome, chronic persistent diarrhea, intractable diarrhea of infancy, Traveler's diarrhea, immunoproliferative small intestinal disease, chronic prostatitis, postenteritis syndrome, tropical sprue, Whipple's disease, Wolman disease, arthritis, rheumatoid arthritis, Behçet's disease, uveitis, pyoderma gangrenosum, erythema nodosum, traumatic brain injury, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, bullous pemphigoid, sarcoidosis, ichthyosis, Graves ophthalmopathy, Addison's disease, Vitiligo, acne vulgaris, pelvic inflammatory disease, reperfusion injury, sarcoidosis, transplant rejection, interstitial cystitis, atherosclerosis, and atopic dermatitis.

13. The method of embodiment 5, wherein the subject has at least 1, 2, 3, or 4 cousins, grandparents, parents, aunts, uncles, and/or siblings who have been diagnosed with the inflammatory disease.

14. The method of embodiment 5 or 13, wherein the mother of the subject has or has had asthma.

15. The method of embodiment 5, 13, or 14, wherein the subject has been in a room with a cat or a dog 0 times during the first month after the subject was born.

16. The method of any one of embodiments 5 or 13-15, wherein the subject has not lived in a residence with a cat or a dog for at least 7, 14, or 21 days of the first month after the subject was born.

17. The method of any one of embodiments 5 or 13-16, wherein the subject's mother has not lived in a residence with a cat or a dog for at least 30, 60, 90, 120, 150, 180, 210, 240, or 270 days between when the subject was conceived and when the subject was born.

18. The method of any one of embodiments 5 or 13-17, wherein the subject's mother has smoked at least once on a total of at least about 30, 60, 90, 120, 150, 180, 210, 240, or 270 days between when the subject was conceived and when the subject was born.

19. The method of any one of embodiments 16-18, wherein the days are consecutive days.

20. The method of any one of embodiments 5 or 13-19, wherein the subject has been fed formula in the first month of life.

21. The method of any one of embodiments 5 or 13-20, wherein the subject has not been fed breast milk in the first month of life.

22. The method of any one of embodiments 5 or 13-21, wherein the subject has a fecal level of 12,13 DiHOME of least about >398 ng/g.

23. The method of embodiment 5, wherein the subject has a fecal level of 9,10 DiHOME of at least about >425 ng/g.

24. The method of any one of embodiments 1-23, wherein the subject is a neonate.

25. The method of any one of embodiments 1-23, wherein the subject is less than about 1, 2, 3, 4, 5, 6, 7, 8, 9, 12, 18, or 24 months old.

26. The method of any one of embodiments 1-23, wherein the subject is between about 2 and about 18 years old, or is at least about 18 years old.

27. The method of any one of embodiments 1-23, wherein the subject is less than 1, 2, 3, 4, or 5 years old.

28. The method of any one of embodiments 1-23, wherein the subject is from 0 to 1 month old, from 0.5 to 2 months old, from 0 to 3 months old, 0.5 to 3 months old, from 3 to 6 months old, or from 0 to 6 months old.

29. The method of any one of embodiments 1-28, wherein less than about 10, 9, 8, 7, 6, 5, 4, 3, or 2 different species of bacteria are administered to the subject.

30. The method of any one of embodiments 1-29, wherein
(a) the nucleotide sequence of the 16S rRNA gene of the fetal Lactobacillus sp. bacterium is at least 95, 96, 97, 98, 99, or 99.5% identical to SEQ ID NO: 3;
(b) the nucleotide sequence of the 16S rRNA gene of the fetal Lactobacillus sp. bacterium is identical to SEQ ID NO: 3;
(c) the nucleotide sequence of the 16S rRNA gene of the fetal Lactobacillus sp. bacterium is at least 95, 96, 97, 98, 99, or 99.5% identical to SEQ ID NO: 5;
(d) the nucleotide sequence of the 16S rRNA gene of the fetal Lactobacillus sp. bacterium is identical to SEQ ID NO: 5;
(e) the nucleotide sequence of the V4 region of the 16S rRNA gene of the fetal Lactobacillus sp. bacterium is at least 95, 96, 97, 98, 99, or 99.5% identical to SEQ ID NO: 1;
(f) the nucleotide sequence of the V4 region of the 16S rRNA gene of the fetal Lactobacillus sp. bacterium is identical to SEQ ID NO: 1;
(g) the nucleotide sequence of the V4 region of the 16S rRNA gene of the fetal Lactobacillus sp. bacterium is at least 95, 96, 97, 98, 99, or 99.5% identical to SEQ ID NO: 6; and/or
(h) the nucleotide sequence of the V4 region of the 16S rRNA gene of the fetal Lactobacillus sp. bacterium is identical to SEQ ID NO: 6.

31. The method of any one of embodiments 1-30, wherein
(a) the nucleotide sequence of the 16S rRNA gene of the fetal Micrococcus sp. bacterium is at least 95, 96, 97, 98, 99, or 99.5% identical to SEQ ID NO: 4;
(b) the nucleotide sequence of the 16S rRNA gene of the fetal Micrococcus sp. bacterium is identical to SEQ ID NO: 4;
(c) the nucleotide sequence of the V4 region of the 16S rRNA gene of the fetal Micrococcus sp. bacterium is at least 95, 96, 97, 98, 99, or 99.5% identical to SEQ ID NO: 2;
(d) the nucleotide sequence of the V4 region of the 16S rRNA gene of the fetal Micrococcus sp. bacterium is identical to SEQ ID NO: 2;
(e) the nucleotide sequence of the V4 region of the 16S rRNA gene of the fetal Micrococcus sp. bacterium is at least 95, 96, 97, 98, 99, or 99.5% identical to SEQ ID NO: 7; and/or
(f) the nucleotide sequence of the V4 region of the 16S rRNA gene of the fetal Micrococcus sp. bacterium is identical to SEQ ID NO: 7.

32. The method of any one of embodiments 1-31, wherein the Lactobacillus sp.
(a) reduces activation of antigen presenting cells;
(b) reduces the expression of CD86 and/or CD83 on antigen presenting cells;
(c) induces expression of the tolerogenic integrin CD103 on dendritic cells;
(d) induces expression of the tolerogenic integrin CD103 on CD11c+ dendritic cells; and/or promotes regulatory T cell accumulation.

33. The method of any one of embodiments 1-32, wherein the Micrococcus sp. reduces IFNγ production by memory promyelocytic leukemia zinc finger protein (PLZF)+ T cells.

34. The method of any one of embodiments 1-33, wherein the level of PLZF+CD161+ T cells increases in the subject.

35. A method of treating, preventing, or reducing the risk of dysbiosis in a subject in need thereof, comprising administering to the subject an effective amount of a fetal Micrococcus sp. bacterium and/or a fetal Lactobacillus sp. bacterium.

36. A method of treating, preventing, or reducing the risk of inflammation in an unborn subject, comprising administering to the pregnant mother of the subject an effective amount of a fetal Micrococcus sp. bacterium and/or a fetal Lactobacillus sp. bacterium.

37. A method of promoting or increasing immune system maturation or Treg function in an unborn subject, comprising administering to the pregnant mother of the subject an effective amount of a fetal Micrococcus sp. bacterium and/or a fetal Lactobacillus sp. bacterium.

38. A method of treating, preventing, or reducing the risk of dysbiosis in an unborn subject, comprising administering to the pregnant mother of the subject an effective amount of a fetal Micrococcus sp. bacterium and/or a fetal Lactobacillus sp. bacterium.

39. A method of reducing the risk that an unborn subject will develop an inflammatory disease after birth, comprising administering to the pregnant mother of the subject an effective amount of a fetal Micrococcus sp. bacterium and/or a fetal Lactobacillus sp. bacterium.

40. A method of treating, preventing, or reducing the risk of childhood obesity in an unborn subject, comprising administering to the pregnant mother of the subject an effective amount of a fetal Micrococcus sp. bacterium and/or a fetal Lactobacillus sp. bacterium.

41. A method of treating, preventing, or reducing the risk of dysbiosis in a neonatal subject, comprising administering to the subject subject an effective amount of a fetal Micrococcus sp. bacterium and/or a fetal Lactobacillus sp. bacterium.

42. A method of reducing the risk that a neonatal subject will develop an inflammatory disease after birth, comprising administering to the subject an effective amount of a fetal Micrococcus sp. bacterium and/or a fetal Lactobacillus sp. bacterium.

43. A method of treating, preventing, or reducing the risk of childhood obesity in a neonatal subject, comprising administering to the subject an effective amount of a fetal Micrococcus sp. bacterium and/or a fetal Lactobacillus sp. bacterium.

44. The method of any one of embodiments 41-43, wherein the neonatal subject was born by caesarean section.

45. The method of any one of embodiments 41-44, wherein the neonatal subject was born after less than 40, 39, 38, 37, 36, 35, 34, 33, 32, 31, or 30 weeks of gestation.

46. The method of any one of embodiments 41-45, wherein the neonatal subject is less than 1 month old.

47. A method of reducing the risk that a pregnant subject will give birth less than 37 completed weeks of gestation, comprising administering to the subject an effective amount of a fetal Micrococcus sp. bacterium and/or a fetal Lactobacillus sp. bacterium.

48. The method of embodiment 47, wherein the subject has an increased risk of pre-term labor compared to a healthy population of pregnant subjects.

49. The method of embodiment 48, wherein the subject has given birth less than 37 completed weeks of gestation during a previous pregnancy.

50. The method of embodiment 48 or 49, wherein the subject is pregnant with multiple gestations.

51. The method of any one of embodiments 48-50, wherein the subject is less than 18 years old or more than 35 years old.

52. The method of any one of embodiments 48-51, wherein the subject has a urinary tract infection, has a sexually transmitted infection, has bacterial vaginosis, has trichomoniasis, has high blood pressure, has bleeding from the vagina, has a pregnancy resulting from in vitro fertilization, gave birth less than 6 months before the current pregnancy, has placenta previa, has diabetes, or has abnormal blood clotting.

53. A method of detecting a polynucleotide in a fetal intestine, comprising detecting whether a polynucleotide having a sequence that is at least 95, 96, 97, 98, 99, or 100% identical to SEQ ID NO: 1, SEQ ID NO: 2, SEQ ID NO: 3, SEQ ID NO: 4, SEQ ID NO: 5, SEQ ID NO: 6, or SEQ ID NO: 7 is present in a bacterium or biological sample obtained from the fetal intestine.

54. A method of detecting a polynucleotide in meconium, amniotic fluid, or a placenta, comprising detecting whether a polynucleotide having a sequence that is at least 95%, 96%, 97%, 98%, 99%, or 100% identical to SEQ ID NO: 1, SEQ ID NO: 2, SEQ ID NO: 3, SEQ ID NO: 4, SEQ ID NO: 5, SEQ ID NO: 6, or SEQ ID NO: 7 is present in a bacterium or biological sample obtained from the meconium, amniotic fluid, or placenta.

55. A method of detecting a polynucleotide in a bacterium, comprising detecting whether a polynucleotide having a sequence that is at least 95%, 96%, 97%, 98%, 99%, or 100% identical to SEQ ID NO: 1, SEQ ID NO: 2, SEQ ID NO: 3, SEQ ID NO: 4, SEQ ID NO: 5, SEQ ID NO: 6, or SEQ ID NO: 7 is present in a bacterium obtained from a fetal intestine, amniotic fluid, meconium, or a placenta.

56. A method of culturing an isolated bacterium, the method comprising obtaining a bacterium comprising a 16S rRNA gene V4 region that is at least about identical to SEQ ID NO: 1 or SEQ ID NO: 2, wherein the bacterium has been isolated from amniotic fluid or meconium, and culturing the bacterium.

57. An isolated fetal Micrococcus sp. bacterium and/or an isolated fetal Lactobacillus sp. bacterium.

58. The bacterium of embodiment 57, which is lyophilized.

59. The bacterium of embodiment 57 or 58, wherein
(a) the nucleotide sequence of the 16S rRNA gene of the fetal Lactobacillus sp. bacterium is at least 95, 96, 97, 98, 99, or 99.5% identical to SEQ ID NO: 3;
(b) the nucleotide sequence of the 16S rRNA gene of the fetal Lactobacillus sp. bacterium is identical to SEQ ID NO: 3;
(c) the nucleotide sequence of the 16S rRNA gene of the fetal Lactobacillus sp. bacterium is at least 95, 96, 97, 98, 99, or 99.5% identical to SEQ ID NO: 5;
(d) the nucleotide sequence of the 16S rRNA gene of the fetal Lactobacillus sp. bacterium is identical to SEQ ID NO: 5;
(e) the nucleotide sequence of the V4 region of the 16S rRNA gene of the fetal Lactobacillus sp. bacterium is at least 95, 96, 97, 98, 99, or 99.5% identical to SEQ ID NO: 1;
(f) the nucleotide sequence of the V4 region of the 16S rRNA gene of the fetal Lactobacillus sp. bacterium is identical to SEQ ID NO: 1;
(g) the nucleotide sequence of the V4 region of the 16S rRNA gene of the fetal Lactobacillus sp. bacterium is at least 95, 96, 97, 98, 99, or 99.5% identical to SEQ ID NO: 6;
(h) the nucleotide sequence of the V4 region of the 16S rRNA gene of the fetal Lactobacillus sp. bacterium is identical to SEQ ID NO: 6;
(i) the Lactobacillus sp. reduces activation of antigen presenting cells; the Lactobacillus sp. reduces the expression of CD86 and/or CD83 on antigen presenting cells;
(k) the Lactobacillus sp. induces expression of the tolerogenic integrin CD103 on dendritic cells; and/or
(l) induces expression of the tolerogenic integrin CD103 on CD11c+ dendritic cells; and/or promotes regulatory T cell accumulation.

60. The bacterium of embodiment 47 or 58, wherein
(a) the nucleotide sequence of the 16S rRNA gene of the fetal Micrococcus sp. bacterium is at least 95, 96, 97, 98, 99, or 99.5% identical to SEQ ID NO: 4;
(b) the nucleotide sequence of the 16S rRNA gene of the fetal Micrococcus sp. bacterium is identical to SEQ ID NO: 4;
(c) the nucleotide sequence of the V4 region of the 16S rRNA gene of the fetal Micrococcus sp. bacterium is at least 95, 96, 97, 98, 99, or 99.5% identical to SEQ ID NO: 2;
(d) the nucleotide sequence of the V4 region of the 16S rRNA gene of the fetal Micrococcus sp. bacterium is identical to SEQ ID NO: 2;
(e) the nucleotide sequence of the V4 region of the 16S rRNA gene of the fetal Micrococcus sp. bacterium is at least 95, 96, 97, 98, 99, or 99.5% identical to SEQ ID NO: 7;
(f) the nucleotide sequence of the V4 region of the 16S rRNA gene of the fetal Micrococcus sp. bacterium is identical to SEQ ID NO: 7; and/or
(g) the Micrococcus sp. reduces IFNγ production by memory promyelocytic leukemia zinc finger protein (PLZF)+ T cells.

61. A composition comprising the isolated fetal Micrococcus sp. bacterium and/or the isolated fetal Lactobacillus sp. bacterium of any one of embodiments 57-60 and a carrier that is suitable for oral or vaginal administration.

62. The composition of embodiment 61, wherein the composition comprises less than about 10, 9, 8, 7, 6, 5, 4, 3, or 2 different species of bacteria.

63. The composition of embodiment 61 or 62, which is a capsule, a tablet, a suspension, a suppository, a powder, a solid, a semi-solid, a liquid, a cream, an oil, an oil-in-water emulsion, a water-in-oil emulsion, or an aqueous solution.

64. The composition of any one of embodiments 61-63, which has a water activity (a_{w}) less than about 0.9, 0.8, 0.7, 0.6, 0.5, 0.4, 0.3, 0.2, or 0.1 at 20° C.

65. The composition of any one of embodiments 61-64, which is a food or a beverage.

66. The composition of any one of embodiments 61-65, which is a food or a beverage.

67. An artificial culture comprising the bacterium of any one of embodiments 57-60 and a medium.

68. The artificial culture of embodiment 67, further comprising a placental hormone.

69. The artificial culture of embodiment 68, wherein the placental hormone is the only source of carbon in the medium.

70. The artificial culture of embodiment 68 or 69, wherein the placental hormone is progesterone or estradiol.

71. The artificial culture of embodiment 70, wherein the estradiol is β-estradiol.

72. The artificial culture of embodiment 71, wherein the β-estradiol is 17β-estradiol.

73. The artificial culture of any one of embodiments 67-72, further comprising a monocyte.

74. The artificial culture of embodiments 67-72, further comprising a macrophage.

75. The artificial culture of embodiment 73 or 74, wherein the monocyte is a primary monocyte or the macrophage is a primary macrophage.

76. The artificial culture of embodiment 73 or 75, wherein the monocyte is a monocyte a cell line or the macrophage is a macrophage cell line.

77. The artificial culture of embodiment 76, wherein the cell line is a THP-1 is a human monocytic cell line.

78. The artificial culture of any one of embodiments 67-72, further comprising an epithelial cell.

79. The artificial culture of embodiment 78, which is a primary epithelial cell or an epithelial cell line.

80. The artificial culture of embodiment 79, which is a CACO2 cell.

81. The artificial culture of any one of embodiments 67-80, which is in a cell culture plate, a flask, or a biofermentor.

82. A method of culturing a fetal Micrococcus sp. bacterium and/or a fetal Lactobacillus sp. bacterium, the method comprising incubating the bacterium in or on a medium comprising a eukaryotic cell and/or a placental hormone.

83. A method of isolating a fetal Micrococcus sp. bacterium and/or a fetal Lactobacillus sp. bacterium, the method comprising:
(i) incubating a culture medium comprising (a) a biological sample suspected of containing the bacterium and (b) a eukaryotic cell and/or a placental hormone, thereby producing a pre-isolate culture;
(ii) streaking a portion of the pre-isolate culture onto a selection plate, and selecting a single colony of the fetal Micrococcus sp. bacterium and/or a fetal Lactobacillus sp. bacterium from the plate.

84. The method of embodiment 82 or 83, wherein the medium comprises a placental hormone.

85. The method of embodiment 84, wherein the placental hormone is the only source of carbon in the medium.

86. The method of embodiment 84 or 85, wherein the placental hormone is progesterone or estradiol.

87. The method of embodiment 86, wherein the estradiol is β-estradiol.

88. The method of embodiment 87, wherein the β-estradiol is 17β-estradiol.

89. The method of any one of embodiments 82-88, wherein the medium comprises a monocyte.

90. The method of any one of embodiments 82-88, wherein the medium comprises a macrophage.

91. The method of embodiment 89 or 90, wherein the monocyte is a primary monocyte or the macrophage is a primary macrophage.

92. The method of embodiment 89 or 90, wherein the monocyte or the macrophage is a cell line.

93. The method of embodiment 92, wherein the cell line is a THP-1 is a human monocytic cell line.

94. The method of any one of embodiments 82-88, further comprising an epithelial cell.

95. The method of embodiment 94, which is a primary epithelial cell or an epithelial cell line.

96. The method of embodiment 95, which is a CACO2 cell.

1. A method of treating, preventing, or reducing the risk of an inflammatory disease in a subject in need thereof, comprising administering to the subject an effective amount of a fetal Micrococcus sp. bacterium and/or a fetal Lactobacillus sp. bacterium.

2. The method of claim 1, wherein the fetal Micrococcus sp. bacterium and/or the fetal Lactobacillus sp. bacterium is administered orally or vaginally.

3. (canceled)

4. The method of claim 1, wherein the subject;
(a) is pregnant
(b) has an increased risk for developing the inflammatory disease compared to a general population of healthy subjects;
(c) has an inflammatory disease;
(d) has an increased risk of pre-term labor compared to a healthy population of pregnant subjects.

5. (canceled)

6. (canceled)

7. The method of claim 1, wherein the inflammatory disease is an allergy, a chronic inflammatory disease, or asthma.

8.-21. (canceled)

22. The method of claim 4, wherein the subject has a fecal level of 12,13 DiHOME of least about >398 ng/g, or a fecal level of 9,10 DiHOME of at least about >425 ng/g.

23. (canceled)

24. The method of claim 1, wherein the subject is (a) a neonate, or (b) less than about 1, 2, 3, 4, 5, 6, 7, 8, 9, 12, 18, or 24 months old.

25.-28. (canceled)

29. The method of claim 1, wherein less than about 10, 9, 8, 7, 6, 5, 4, 3, or 2 different species of bacteria are administered to the subject.

30. The method of claim 1, wherein
(a) the nucleotide sequence of the 16S rRNA gene of the fetal Lactobacillus sp. bacterium is at least 95, 96, 97, 98, 99, or 99.5% identical to SEQ ID NO: 3;
(b) the nucleotide sequence of the 16S rRNA gene of the fetal Lactobacillus sp. bacterium is identical to SEQ ID NO: 3;
(c) the nucleotide sequence of the 16S rRNA gene of the fetal Lactobacillus sp. bacterium is at least 95, 96, 97, 98, 99, or 99.5% identical to SEQ ID NO: 5;
(d) the nucleotide sequence of the 16S rRNA gene of the fetal Lactobacillus sp. bacterium is identical to SEQ ID NO: 5;
(e) the nucleotide sequence of the V4 region of the 16S rRNA gene of the fetal Lactobacillus sp. bacterium is at least 95, 96, 97, 98, 99, or 99.5% identical to SEQ ID NO: 1;
(f) the nucleotide sequence of the V4 region of the 16S rRNA gene of the fetal Lactobacillus sp. bacterium is identical to SEQ ID NO: 1;
(g) the nucleotide sequence of the V4 region of the 16S rRNA gene of the fetal Lactobacillus sp. bacterium is at least 95, 96, 97, 98, 99, or 99.5% identical to SEQ ID NO: 6; and/or
(h) the nucleotide sequence of the V4 region of the 16S rRNA gene of the fetal Lactobacillus sp. bacterium is identical to SEQ ID NO: 6.

31. The method of claim 1, wherein
(a) the nucleotide sequence of the 16S rRNA gene of the fetal Micrococcus sp. bacterium is at least 95, 96, 97, 98, 99, or 99.5% identical to SEQ ID NO: 4;
(b) the nucleotide sequence of the 16S rRNA gene of the fetal Micrococcus sp. bacterium is identical to SEQ ID NO: 4;
(c) the nucleotide sequence of the V4 region of the 16S rRNA gene of the fetal Micrococcus sp. bacterium is at least 95, 96, 97, 98, 99, or 99.5% identical to SEQ ID NO: 2;
(d) the nucleotide sequence of the V4 region of the 16S rRNA gene of the fetal Micrococcus sp. bacterium is identical to SEQ ID NO: 2;
(e) the nucleotide sequence of the V4 region of the 16S rRNA gene of the fetal Micrococcus sp. bacterium is at least 95, 96, 97, 98, 99, or 99.5% identical to SEQ ID NO: 7; and/or
(f) the nucleotide sequence of the V4 region of the 16S rRNA gene of the fetal Micrococcus sp. bacterium is identical to SEQ ID NO: 7.

32. The method of claim 1, wherein the Lactobacillus sp.
(a) reduces activation of antigen presenting cells;
(b) reduces the expression of CD86 and/or CD83 on antigen presenting cells;
(c) induces expression of the tolerogenic integrin CD103 on dendritic cells;
(d) induces expression of the tolerogenic integrin CD103 on CD11c+ dendritic cells.

33. The method of claim 1, wherein (a) the Micrococcus sp. reduces IFNγ production by memory promyelocytic leukemia zinc finger protein (PLZF)+ T cells, or (b) the level of PLZF+ CD161+ T cells increases in the subject.

34. (canceled)

35. (canceled)

36. The method of claim 1, wherein the subject is an unborn subject, and the administering comprises administering the fetal Micrococcus sp. bacterium and/or the fetal Lactobacillus sp. bacterium to the pregnant mother of the subject.

37.-56. (canceled)

57. An isolated fetal Micrococcus sp. bacterium and/or an isolated fetal Lactobacillus sp. bacterium.

58. (canceled)

59. The bacterium of claim 57, wherein
(a) the nucleotide sequence of the 16S rRNA gene of the fetal Lactobacillus sp. bacterium is at least 95, 96, 97, 98, 99, or 99.5% identical to SEQ ID NO: 3;
(b) the nucleotide sequence of the 16S rRNA gene of the fetal Lactobacillus sp. bacterium is identical to SEQ ID NO: 3;
(c) the nucleotide sequence of the 16S rRNA gene of the fetal Lactobacillus sp. bacterium is at least 95, 96, 97, 98, 99, or 99.5% identical to SEQ ID NO: 5;
(d) the nucleotide sequence of the 16S rRNA gene of the fetal Lactobacillus sp. bacterium is identical to SEQ ID NO: 5;
(e) the nucleotide sequence of the V4 region of the 16S rRNA gene of the fetal Lactobacillus sp. bacterium is at least 95, 96, 97, 98, 99, or 99.5% identical to SEQ ID NO: 1;
(f) the nucleotide sequence of the V4 region of the 16S rRNA gene of the fetal Lactobacillus sp. bacterium is identical to SEQ ID NO: 1;
(g) the nucleotide sequence of the V4 region of the 16S rRNA gene of the fetal Lactobacillus sp. bacterium is at least 95, 96, 97, 98, 99, or 99.5% identical to SEQ ID NO: 6;
(h) the nucleotide sequence of the V4 region of the 16S rRNA gene of the fetal Lactobacillus sp. bacterium is identical to SEQ ID NO: 6;
(i) the Lactobacillus sp. reduces activation of antigen presenting cells;
(j) the Lactobacillus sp. reduces the expression of CD86 and/or CD83 on antigen presenting cells;
(k) the Lactobacillus sp. induces expression of the tolerogenic integrin CD103 on dendritic cells; and/or
(l) induces expression of the tolerogenic integrin CD103 on CD11c+ dendritic cells.

60. The bacterium of claim 57, wherein
(a) the nucleotide sequence of the 16S rRNA gene of the fetal Micrococcus sp. bacterium is at least 95, 96, 97, 98, 99, or 99.5% identical to SEQ ID NO: 4;
(b) the nucleotide sequence of the 16S rRNA gene of the fetal Micrococcus sp. bacterium is identical to SEQ ID NO: 4;
(c) the nucleotide sequence of the V4 region of the 16S rRNA gene of the fetal Micrococcus sp. bacterium is at least 95, 96, 97, 98, 99, or 99.5% identical to SEQ ID NO: 2;
(d) the nucleotide sequence of the V4 region of the 16S rRNA gene of the fetal Micrococcus sp. bacterium is identical to SEQ ID NO: 2;
(e) the nucleotide sequence of the V4 region of the 16S rRNA gene of the fetal Micrococcus sp. bacterium is at least 95, 96, 97, 98, 99, or 99.5% identical to SEQ ID NO: 7;
(f) the nucleotide sequence of the V4 region of the 16S rRNA gene of the fetal Micrococcus sp. bacterium is identical to SEQ ID NO: 7; and/or
(g) the Micrococcus sp. reduces IFNγ production by memory promyelocytic leukemia zinc finger protein (PLZF)+ T cells.

61. A composition comprising the isolated fetal Micrococcus sp. bacterium and/or the isolated fetal Lactobacillus sp. bacterium of claim 57 and a carrier that is suitable for oral or vaginal administration.

62. The composition of claim 61, wherein the composition comprises less than about 10, 9, 8, 7, 6, 5, 4, 3, or 2 different species of bacteria.

63.-66. (canceled)

67. An artificial culture comprising the bacterium of any one of claim 57 and a medium.

68. The artificial culture of claim 67, further comprising a placental hormone, progesterone, estradiol, β-estradiol, or 17β-estradiol.

69.-72. (canceled)

73. The artificial culture of claim 67, further comprising a monocyte, a macrophage, a primary monocyte, a primary macrophage, a THP-1 human monocytic cell line, an epithelial cell, a primary epithelial cell, or a CACO2 cell.

74.-81. (canceled)

82. A method of culturing a fetal Micrococcus sp. bacterium and/or a fetal Lactobacillus sp. bacterium, the method comprising incubating the bacterium in or on a medium comprising a eukaryotic cell and/or a placental hormone.

83. (canceled)

84. The method of claim 82, wherein the medium comprises a placental hormone, progesterone, estradiol, β-estradiol, or 17β-estradiol.

85.-88. (canceled)

89. The method of claim 82, wherein the medium comprises a monocyte, a macrophage, a primary monocyte, a primary macrophage, a THP-1 human monocytic cell line, an epithelial cell, a primary epithelial cell, or a CACO2 cell.

90.-96. (canceled)
